(19) United States
(12) Patent Application Publication
(10) Pub. No.: US 2024/0309015 A1
(43) Pub. Date: Sep. 19, 2024

(54) Methods for the Treatment of Fibrotic Disease

(71) Applicant: SHY Therapeutics, LLC, Harrison, NY (US)

(72) Inventors: Yaron R. Hadari, Harrison, NY (US); Michael Schmertzler, St. Petersburg, FL (US); Theresa M. Williams, Harleysville, PA (US); Luca Carta, Scarsdale, NY (US); Rebecca Hutcheson, Stamford, CT (US)

(21) Appl. No.: 18/273,646

(22) PCT Filed: Jan. 27, 2022
(86) PCT No.: PCT/US2022/014053

§ 371 Date: Jul. 21, 2023

Related U.S. Application Data
(60) Provisional application No. 63/142,445, filed on Jan. 27, 2021.

(51) Int. Cl. C07D 495/04; A61K 31/519; A61K 31/5377; A61K 31/541; A61P 21/00; G01N 33/573

(52) U.S. Cl. CPC C07D 495/04; A61K 31/519; A61K 31/5377; A61K 31/541; A61P 21/00; G01N 33/573; G01N 2333/914; G01N 2470/10; G01N 2500/20

(57) ABSTRACT

Provided herein are methods and corresponding compounds for treating fibrotic disease by administering to a subject a compound of Formula I or pharmaceutically acceptable salt thereof, which inhibits phosphorylation of Smad2/3, according to Phospho-Smad2/3 Inhibition Assay, and is inactive according to MAPK p38 Activation Assay; or a compound of Formula II or III. Also provided are methods and corresponding compounds for treating cancers, inflammatory diseases, rasopathies and autoimmune leukoproliferative disorders.

### 1. PRIORITY

This application claims the benefit of priority to U.S. Ser. No. 63/142,445 filed Jan. 27, 2021, which is incorporated herein by reference in its entirety.

### 2. FIELD

Provided herein are methods for treating fibrotic disease by administering to a subject a compound of Formula I or pharmaceutically acceptable salt thereof, which inhibits phosphorylation of Smad2/3, according to Phospho-Smad2/3 Inhibition Assay, and is inactive according to MAPK p38 Activation Assay.

### 3. BACKGROUND

### Pathobiology of Fibrotic Disease

Fibrosis, or the accumulation of extracellular matrix molecules that constitute scar tissue, is a common result of tissue injury. Fibrosis can occur in many tissues within the body, typically as a result of inflammation or damage. Pulmonary fibrosis, renal fibrosis, and hepatic cirrhosis are among the common fibrotic diseases which altogether represent a large unmet medical need. (Friedman S L, Sheppard D, Duffield J S, Violette S. Sci Transl Med 2013 Jan. 9; 5(167): 167sr1).

Fibrosis, also known as fibrotic scarring, is a pathological wound healing process in which connective tissue replaces normal parenchymal tissue, leading to considerable tissue re-modeling and the formation of permanent scar tissue. Repeated injuries, chronic inflammation and repair are susceptible to fibrosis where excessive extracellular matrix (ECM) components, such as collagen and glycosaminoglycans, accumulate and lead to the formation of a permanent fibrotic scar which can interfere with normal organ and tissue functions. Mechanisms of fibrogenesis include inflammation as well as other pathways and generally involve reorganization of the actin cytoskeleton of affected cells, including epithelial cells, fibroblasts, endothelial cells, and macrophages.

Actin filament assembly and actomyosin contraction are directed by the Rho-associated coiled-coil forming protein kinase (ROCK) family of serine/threonine kinases (ROCK1 and ROCK2) and thus Rho is associated with fibrogenesis.

Tissue fibrosis is a leading cause of morbidity and mortality. 45% of deaths in the United States are attributable to fibrotic disorders. (Wynn T A. “Fibrotic Disease and the TH1/TH2 Paradigm.” Nat Rev Immunol 2004 August: 4(8): 583-594.) Treatments are generally palliative.

Idiopathic pulmonary fibrosis (IPF) is characterized by progressive lung scarring, short median survival, and limited therapeutic options, creating great need for new pharmacologic therapies. It is thought to result from repetitive environmental injury to the lung epithelium.

### Targeted Therapy of Fibrotic Diseases

Targeted therapies are a cornerstone of what is also referred to as precision medicine, a form of medicine that uses information about a person's genes and proteins to prevent, diagnose, and treat disease. Such therapeutics are sometimes called “molecularly targeted drugs,” “molecularly targeted therapies,” or similar names. The process of discovering them is often referred to as “rational drug design.” This concept can also be referred to as “personalized medicine.”

A series of actions among molecules in a cell that leads to a certain end point or cell function is referred to as a molecular pathway.

Molecularly targeted drugs interact with a particular target molecule, or structurally related set of target molecules, in a pathway; thus modulating the endpoint effect of that pathway, such as a disease-related process; and, thus, yielding a therapeutic benefit.

Molecularly targeted drugs may be small molecules or biologics, usually antibodies. They may be useful alone or in combinations with other therapeutic agents and methods.

Because they target a particular molecule, or related set of molecules, and are usually designed to minimize their interactions with other molecules, targeted therapeutics may have fewer adverse side effects.

Some treatments for fibrotic disorders, such as idiopathic pulmonary fibrosis, hepatic fibrosis, and systemic sclerosis, target inflammatory pathways.

### The Ras GTPase Family

The Ras superfamily of proteins are small GTPases with substantial amino acid sequence homology that act as signal transducers between cell surface receptors and several intracellular signaling cascades. These molecules are involved in the regulation of such essential cellular functions as cell survival, proliferation, motility, and cytoskeletal organization (see Karnoub et al., Nat. Rev. Mol. Cell Biol., 9: 517-531 (2008)).

Research has defined a number of subfamilies of the Ras superfamily, based largely on amino acid sequence homologies. These subfamilies are often referred to in an abbreviated manner based on the most commonly studied member of the class.

The GTP binding domains of one subfamily of the Ras superfamily having substantial sequence homology is commonly referred to as the Ras family or Ras.

There are four isoforms of Ras proteins, expressed from three different genes: H-Ras (Harvey sarcoma viral oncogene), N-Ras (neuroblastoma oncogene), and the splice variants K-Ras4A and K-Ras4B (Kirsten sarcoma viral oncogene) (see Karnoub et al., supra).

The GTP binding domains of another subfamily of the Ras superfamily having substantial sequence homology is commonly referred to as the Rho family and includes proteins and groups of proteins referred to as Rho, Rac and Cdc42.

### Ras Function and Pathways

All Ras isoforms share sequence identity in all of the regions that are responsible for GDP/GTP binding, GTPase activity, and effector interactions, suggesting a functional redundancy. However, studies clearly demonstrate that each Ras isoform can function in a unique, different way from the other Ras proteins in normal physiological processes as well as in pathogenesis (Quinlan et al., Future Oncol., 5: 105-116 (2009)).

Several cell surface receptors activate Ras, such as Receptor Tyrosine Kinases (RTKs), growth factor receptors, cytokine receptors and integrins.

Ras proteins cycle between ‘on’ and ‘off’ conformations that are conferred by the binding of GTP and GDP, respectively. Under physiological conditions, the transition between these two states is regulated by guanine nucleotide exchange factors (GEFs), such as Son of sevenless (Sos) (Bar-Sagi D, Trends Endocrin. Metab. 5, 165-169 (1994)), which promote the activation of Ras proteins by stimulating the exchange of GDP for GTP exchange, and by GTPase-activating proteins (GAPs), which accelerate Ras-mediated GTP hydrolysis to GDP.

The region of Sos functional for nucleotide exchange on Ras spans about 500 residues, and contains blocks of sequence that are conserved in Sos and other Ras-specific GEF's such as Cdc25, Sdc25 and Ras guanine-nucleotide-release factor (GRF) (Boguske et al, Nature 366, 643-654 (1993)).

Once activated, Ras initiates signaling of the “MAPK pathway” (also referred to as the Ras-RAF-MEK-MAPK/ERK1/2 pathway) that affects cell growth, differentiation, proliferation, apoptosis and migration. The MAPK pathway operates through a sequence of interactions among kinases. Activated by Ras in the “on”, GTP bound, state, a MAPK kinase kinase (MAPK3), such as Raf, MLK, or TAK, phosphorylates and activates a MAPK kinase, such as MEK, which then phosphorylates and increases the activity of one or more MAPKs, such as ERK1/2.

Ras activation also initiates signaling of the “Akt pathway” that affects cellular survival, proliferation, migration, anti-apoptotic and cell cycle regulation. Ras in the “on”, GTP bound, state, activates phosphoinositide 3-kinase (PI3K) which, in turn, induces the production of phosphatidylinositol (3,4,5) trisphosphates (PIP3). These lipids serve as plasma membrane docking sites for proteins that harbor pleckstrin-homology (PH) domains, including Akt (also known as protein kinase B or PKB) and its upstream activator PDK1. There are three highly related isoforms of Akt (Akt1, Akt2 and Akt3) that phosphorylate shared substrates, but isoform-specific Akt substrates have also been identified. At the membrane, Akt is phosphorylated and activated by PDK1, PDK2 and mTORC2. The Akt pathway can also be activated by receptor tyrosine kinases, integrins, B and T cell receptors, cytokine receptors and G-protein-coupled receptors that directly interact and activate PI3K.

Ras activation is also associated with signaling through other molecular pathways other than phosphoinositide 3-kinases (PI3Ks), such as Rac1 GEF and the Ral-guanine nucleotide dissociation stimulator (GDS). Regarding PI3K, that is part of the PI3K/AKT/mTOR pathway regulating intracellular signaling important for several cellular functions such as survival, anti-apoptotic and cell cycle regulation.

Ras Dysfunction is Causally Associated with Important Diseases and Disease Processes

Ras and its downstream pathways, including ERK1/2 and Akt, have been studied extensively. They are causally associated with a range of diseases, including certain cancers, inflammatory disorders, Ras-associated autoimmune leukoproliferative disorder, type II diabetes, and certain Rasopathies.

There is more than one distinct route to aberrant Ras activation including mutational activation of Ras itself, excessive activation of the wild-type protein through upstream signaling, and loss of a GAP function that is required to terminate activity of the protein. One million deaths per year are attributed in the literature to mutations in K-Ras alone. (Frank McCormick. “K-Ras protein as a drug target.” Journal of Molecular Medicine (Berlin) 2016: 94: 253-258)

Ras is causally associated with inflammatory diseases including the following: rheumatoid arthritis (Abreu J R, de Launay D, Sanders M E, Grabiec A M, Sande van de MG, Tak P P, Reedquist K A: The Ras guanine nucleotide exchange factor RasGRF1 promotes matrix metalloproteinase-3 production in rheumatoid arthritis synovial tissue (Arthritis Res Ther. 2009, 11: R121-10.1186/ar2785), which is the most common cause of disability (Hootman J M, Brault M W, Helmick C G, Theis K A, Armour B S. Prevalence and most common causes of disability among adults—United States 2005, MMWR, 2009, 58(16):421-6); atherosclerosis (Fonarow G (2003), Cleve. Clin. J. Med. 70: 431-434); inflammatory bowel disease (IBD), such as Crohn's disease (Ignacio C S, Sandvik A K, Bruland T, Andreu-Ballester J C, J. Crohns Colitis, 2017 Mar. 16. doi: 10); ulcerative colitis; spondyloarthropathies; idiopathic pulmonary fibrosis; juvenile arthritis; psoriasis; psoriatic arthritis; and others.

Ras has been causally associated with Ras-associated autoimmune leukoproliferative disorder, a nonmalignant clinical syndrome initially identified in a subset of putative autoimmune lymphoproliferative syndrome (ALPS) patients. (Katherin Calvo, et al. “JMML and RALD (Ras-associated autoimmune leukoproliferative disorder): common genetic etiology yet clinically distinct entities” Blood, 2015 Apr. 30; 125(18): 2753-2758)

Aberrant Ras signaling is causally implicated in the family of Rasopathies including neurofibromatosis type 1, Noonan's syndrome, and Costello syndrome.

### Ras as a Therapeutic Molecular Target

Interference with Ras superfamily member signaling in cell based and animal models of the aforementioned diseases modulates disease processes.

Ras superfamily proteins, and particularly Ras and downstream pathway elements, have thus long been discussed as theoretical molecular targets for the development of targeted therapeutics. In theory, a molecule could serve as a therapeutic agent in diseases associated with aberrant Ras signaling if it could disrupt such Ras signaling.

In theory, it was recognized that a mechanism for downregulating aberrant Ras signaling could be to interfere with one or more steps in the Ras signaling process involving GTP binding in a manner that left Ras in other than an “on” configuration. In theory, a molecule could serve as therapeutic agent in diseases associated with abberant Ras signaling if it could disrupt such Ras signaling.

However, while this was a theoretical therapeutic strategy based on two widely accepted findings, it has also long been accepted by the scientific community that it would not be possible to achieve.

GTP and GDP had been found to bind to the GTP binding domain of Ras with single to double digit picomolar affinities.

The cellular concentration of GTP had been found to be substantially in excess of this range.

The widely accepted findings concerning the single to double digit picomolar range of affinities of GTP and GDP for the Ras GTP binding domain were determined by kinetic and filter binding measurements between Ras and radiolabeled GDP and GTP (Feuerstein J, Kalbitzer H R, John J, Goody R S, Wittinghofer A. Eur. J. Biochem., 1987 Jan. 2, 162(1):49-55; and John J, Sohmen R, Feuerstein J, Linke R, Wittinghofer A, Goody R S. Biochemistry, 1990 Jun. 26, 29(25):6058-65).

Based on these findings, and often citing them, the GTP binding domain of Ras has widely been accepted and reported in preeminent journal editorials, reviews, and research papers to be “undruggable.” (Papke B, Der C J., Science, 2017 Mar. 17, 355(6330):1158-1163; Stephen A G, Esposito D, Bagni R K, McCormick F, Cancer Cell, 2014 Mar. 17, 25(3):272-81; and Ostrem J M, Shokat K M, Nat. Rev. Drug Discov., 2016 November, 15(11):771-785)

Accordingly, research concerning targeted Ras therapeutics has focused on domains of the Ras protein other than the GTP binding site. These efforts have included, for example, seeking to develop farnesyltransferase inhibitors (FTIs) that prevent Ras attachment to the inner side of the plasma membrane, and to develop molecules that compete with the interaction of Ras with the exchange factor Sos or downstream effectors.

Thus, it has been thought that a molecule could not be developed to reversibly compete with GTP binding to the GTP binding domain of Ras. Compounds that do so, however, would fill a need in the field.

### The Rho Family Function and Pathways

The Rho subfamily of the Ras superfamily currently includes approximately 22 proteins most of which scientists commonly divide into subgroups including those referred to as Cdc42, Rac, and Rho. (Boureux A, Vignal E, Faure S, Fort P (2007). “Evolution of the Rho family of ras-like GTPases in eukaryotes”. Mol Biol Evol 24 (1): 203-16).

The three most commonly studied members of the Rho subfamily have been Cdc42, Rac1, and RhoA.

The Cdc42 group includes Cdc42, TC10, TCL, Chip, and Wrch-1.

The Rac group includes Rac1, Rac2, Rac3, and RhoG.

The RhoA group includes RhoA, RhoB, and RhoC.

Other Rho subfamily GTPases not included in the Cdc42, Rac, or Rho groups include RhoE/Rnd3, RhoH/TTF, Rif, RhoBTB1, RhoBTB2, Miro-1, Miro-2, RhoD, Rnd1, and Rnd2.

Like other Ras superfamily proteins, the Rho subfamily GTPases cycle between ‘on’ and “off” conformations that are conferred by the binding of GTP and GDP, respectively. Under physiological conditions, the transition between these two states is regulated by guanine nucleotide exchange factors (GEFs), which promote the activation of Rho subfamily proteins by stimulating the release of GDP and the binding of GTP, and by GTPase-activating proteins (GAPs), which accelerate Rho subfamily member-mediated GTP hydrolysis to GDP. Guanine nucleotide dissociation inhibitors (GDIs) proteins form a large complex with the Rho protein, helping to prevent diffusion within the membrane and into the cytosol and thus acting as an anchor and allowing tight spatial control of Rho activation.

The Rho subfamily members are intracellular proteins that affect a large number of downstream pathways broadly involving cytoskeleton organization, cell polarity, migration, transcription and proliferation, and, more particularly, membrane and vesicular trafficking, cell cycling, microtubule stability, actin membrane linkages, actin polymerization, myosin phosphorylation, API dependent gene expression, cell adhesion, cell contractility, cell adhesion, and MTOC orientation. (Martin Schwartz. “Rho Signalling at a Glance.” Journal of Cell Science. 2004: (117:pp. 5457-5458).and (Bustelo X R, Sauzeau V, Berenjeno I M (2007). “GTP-binding proteins of the Rho/Rac family: regulation, effectors and functions in vivo” BioEssays. 29 (4): 356-370).

Rho subfamily associated kinases (ROCK1 and ROCK2) are implicated as mediators of multiple profibrotic processes including those associated with idiopathic pulmonary fibrosis. (Knipe R S, Tager E M, and Liao J K. “The Rho kinases: critical mediators of multiple profibrotic processes and rational targets for new therapies for pulmonary fibrosis.” Pharmacol Rev. 2015 67(1):103-17.)

### Rho Family Members as Therapeutic Molecular Targets

Given their roles in disease processes, Rho subfamily members have been identified as potential Therapeutic Molecular Targets.

Rho subfamily members have been identified as potential Therapeutic Molecular Targets in cancer.

Rho subfamily members have been identified as potential Therapeutic Molecular Targets in fibrotic disease.

### Pathobiology of Cancer

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites. Clinical data and molecular biologic studies indicate that cancer is a multistep process that begins with minor preneoplastic changes, which may under certain conditions progress to neoplasia. The neoplastic lesion may evolve clonally and develop an increasing capacity for invasion, growth, metastasis, and heterogeneity, especially under conditions in which the neoplastic cells escape the host's immune surveillance. (Roitt, I., Brostoff, J. and Kale, D., Immunology, 17.1-17.12 (3rd ed., Mosby, St. Louis, Mo., 1993))

Various stages of tumor development can be described generally as follows:
a) Tumor evolution commences when a cell within a normal population sustains a genetic mutation that expands its tendency to proliferate.
b) Such genetically altered cells and their offspring continue to appear normal, but they reproduce excessively and lead to a condition termed hyperplasia. The altered cells may also secrete signaling factors or other molecules that cause changes in their local cellular and extracellular environment, including without limitation, the response of the immune system to them. Such environmental effects may in turn affect the viability, proliferation, and further mutations of the altered cells. After some time (months or years) a very small fraction of these altered cells may sustain additional mutation with subsequent loss of control of cell growth and further potential effects on their environment.
c) The offspring of these cells not only proliferate excessively but also appear abnormal in shape and in orientation. The tissue is now said to exhibit a condition termed dysplasia. After some time, one or more additional mutations may further alter cell behavior and the effect of the cells on their environment.
d) The influenced and genetically altered cells turn still more abnormal in growth and appearance. If the tumor mass does not invade through any boundaries between tissues, it is termed an in situ tumor. This tumor may stay contained indefinitely, however, some cells may acquire still more mutations.
e) A malignant or invasive tumor results if the genetic changes allow the tumor mass to initiate invading underlying tissue and to cast off cells into the blood or lymph. The defector cells may install new tumors loci (metastases) throughout the body.

Metastases represent the end products of a multistep cell-biological process termed the invasion-metastasis cascade, which involves dissemination of cancer cells to anatomically distant organ sites and their subsequent adaptation to foreign tissue microenvironments. Each of these events is driven by the acquisition of genetic and/or epigenetic alterations within tumor cells and the co-option of non-neoplastic stromal cells, which together endow incipient metastatic cells with traits needed to generate macroscopic metastases. (Volastyan, S., et al., Cell, 2011, vol. 147, 275-292)

An enormous variety of cancers affect different tissues throughout the body, which are described in detail in the medical literature. Over 85% of human cancers are solid tumors, including carcinomas, sarcomas and lymphomas. Different types of solid tumors are named for the type of cells that form them. Examples include cancer of the lung, colon, rectum, pancreatic, prostate, breast, brain, and intestine. Other human tumors derive from cells involved in the formation of immune cells and other blood cells, including leukemias and myelomas.

The incidence of cancer continues to climb as the general population ages, as new cancers develop, and as susceptible populations grow. A tremendous demand therefore exists for new methods and compositions that can be used to treat subjects with cancer.

### Methods of Treating Cancer

Current cancer therapy may involve surgery, chemotherapy, hormonal therapy, biological therapy, targeted therapy, immunotherapy and/or radiation treatment to eradicate neoplastic cells in a patient (see, e.g., Stockdale, 1998, Medicine, vol. 3, Rubenstein and Federman, eds., Chapter 12, Section I V; and Baudino T A “Targeted Cancer Therapy: The Next Generation of Cancer Treatment”, Curr Drug Discov Technol. 2015; 12(1):3-20).

Such therapies may be used independently or in combinations. Choices of therapy will depend on the history and nature of the cancer, the condition of the patient, and, under the circumstances, the anticipated efficacy and adverse effects of the therapeutic agents and methods considered.

With respect to chemotherapy, there are a variety of chemotherapeutic agents and methods of delivery of such agents available for the treatment of different cancers. Most first generation chemotherapeutic agents were not tumor specific, have broad systemic effects, are toxic, and may cause significant and often dangerous side effects, including severe nausea, bone marrow depression, and immunosuppression.

Additionally, even with administration of combinations of chemotherapeutic agents, many tumor cells are or become resistant to chemotherapeutic agents. In fact, cells resistant to the particular chemotherapeutic agents used in a treatment protocol often prove to be resistant to other drugs, even if those agents act by different mechanism from those of the drugs used in the specific treatment. This phenomenon is referred to as multidrug resistance. Because of drug resistance, many cancers prove refractory to standard chemotherapeutic treatment protocols.

Thus, there exists a significant need for alternative compounds, compositions and methods for treating, preventing and managing cancer.

Further, whereas surgical resection and adjuvant therapy can cure well-confined primary tumors, metastatic disease is largely incurable because of its systemic nature and the resistance of disseminated tumor cells to existing therapeutic agents. This explains why greater than 90% of mortality from cancer is attributable to metastases, not the primary tumors from which these malignant lesions arise.

### Pathobiology of Inflammatory Disease

Inflammation is a complex protective biological response of body tissues to harmful stimuli, such as pathogens, damaged cells, or irritants, involving immune cells, blood vessels, and molecular mediators. The function of inflammation is to eliminate the initial cause of cell injury, clear out necrotic cells and tissues damaged from the original insult and the inflammatory process, and to initiate tissue repair. (Ferrero-Miliani L, Nielsen O H, Andersen P S, Girardin S E; Nielsen; Andersen; Girardin (February 2007) Clin. Exp. Immunol. 147)

Inflammation is classified as either acute or chronic. Acute inflammation is the initial response of the body to harmful stimuli and is achieved by the increased movement of plasma and leukocytes (especially granulocytes) from the blood into the injured tissues. A series of biochemical events propagates and matures the inflammatory response, involving the local vascular system, the immune system, and various cells within the injured tissue.

Prolonged inflammation, known as chronic inflammation, is characterized by simultaneous destruction and healing of the tissue from the inflammatory process. It leads to a progressive shift in the type of cells present at the site of inflammation, such as mononuclear cells, and increases in systemic concentrations of cytokines such as TNF-α, IL-6, and CRP. (Petersen, A. M.; Pedersen, B. K. (2005). J Appl Physiol. 98 (4): 1154-1162)

Many proteins are involved in inflammation. Any of them are susceptible to genetic mutation which may impair or otherwise dysregulate their normal function and expression.

### Methods of Treating Inflammatory Disease

Both small molecules and biologics are used to treat inflammatory diseases. Most treatments, however, are largely palliative.

A clear unmet medical need remains to find treatments that can mechanistically reduce chronic inflammatory diseases.

### 4. SUMMARY

Provided herein are methods for treating fibrosis in a subject having a fibrotic disease by administering to the subject a compound of Formula I:

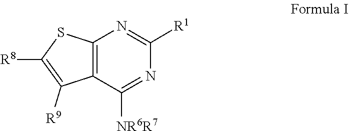

or a pharmaceutically acceptable salt thereof, wherein:
   R¹ is H, alkyl, aryl or heteroaryl;
   R⁸ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
   R⁹ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
   R³ is hydrogen, alkyl, alkenyl, alkynyl, aryl, heteroaryl, heterocyclyl, cycloalkyl, alkylcarbonyl, cycloalkylcarbonyl or arylcarbonyl;
   R⁴ is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy, aralkoxy, or NR⁶R⁷;
   each R⁵ is selected from the group consisting of hydrogen, alkyl, alkenyl, alkynyl, aryl, heteroaryl, heterocyclyl, cycloalkyl, alkylcarbonyl, cycloalkylcarbonyl, and arylcarbonyl; and
   R⁶ and R⁷ are independently selected from the group consisting of hydrogen, alkyl, alkenyl, alkynyl, aryl, heteroaryl, cycloalkyl, heterocyclyl, alkylcarbonyl, cycloalkylcarbonyl, arylcarbonyl, heteroarylcarbonyl, arylsulfonyl, heteroarylsulfonyl, cycloalkylsulfonyl and alkylsulfonyl, or R⁶ and R⁷ are combined to form a cyclic structure including the nitrogen atom to which they are both attached; and
      wherein the compound of Formula I or pharmaceutically acceptable salt thereof inhibits phosphorylation of Smad2/3, according to Phospho-Smad2/3 Inhibition Assay, and is inactive according to MAPK p38 Activation Assay. In some embodiments, the compound of Formula I or pharmaceutically acceptable salt thereof inhibits phosphorylation of Smad2/3 by 20% or more, by 30% or more, by 50% or more, by 70% or more at 10 μM, according to Phospho-Smad2/3 Inhibition Assay. In some embodiments, the compound of Formula I is a compound of Formula IA.

Provided herein are methods for treating fibrosis in a subject having a fibrotic disease by administering to the subject the compound of Formula I or pharmaceutically acceptable salt thereof, which inhibits phosphorylation of Smad2/3, for example, by 20% or more, by 30% or more, by 50% or more, by 70% or more at 10 μM, according to Phospho-Smad2/3 Inhibition Assay, and is inactive according to MAPK p38 Activation Assay, wherein the compound of Formula I also activates JNK, according to INK Activation Assay. In some embodiments, the compound of Formula I administered according to the methods disclosed herein is a modulator of Ras superfamily activity according to a Ras Superfamily Activity Assay. In some embodiments, the compound of Formula I is a compound of Formula IA.

Provided herein is a compound of Formula IA:

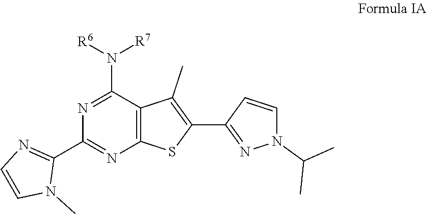

or a pharmaceutically acceptable salt thereof,
wherein:
   R⁶ and R⁷ are independently selected from the group consisting of hydrogen, alkyl, alkenyl, alkynyl, aryl, heteroaryl, cycloalkyl, heterocyclyl, alkylcarbonyl, cycloalkylcarbonyl, arylcarbonyl, heteroarylcarbonyl, arylsulfonyl, heteroarylsulfonyl, cycloalkylsulfonyl and alkylsulfonyl, or R⁶ and R⁷ are combined to form a cyclic structure including the nitrogen atom to which they are both attached.

Provided herein is a compound of Formula II:

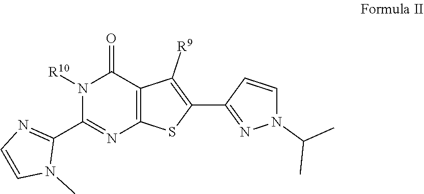

or a pharmaceutically acceptable salt thereof,
wherein:
   R⁹ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
   R¹⁰ is H, alkyl, alkenyl, alkynyl, aryl, cycloalkyl, heterocyclyl, heteroaryl, C(O)R⁴, or S(O)ₚR⁴;
   R³ is hydrogen, alkyl, alkenyl, alkynyl, aryl, heteroaryl, heterocyclyl, cycloalkyl, alkylcarbonyl, cycloalkylcarbonyl or arylcarbonyl;
   R⁴ is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy, aralkoxy, or NR⁶R⁷;
   each R⁵ is selected from the group consisting of hydrogen, alkyl, alkenyl, alkynyl, aryl, heteroaryl, heterocyclyl, cycloalkyl, alkylcarbonyl, cycloalkylcarbonyl, and arylcarbonyl; and
   R⁶ and R⁷ are independently selected from the group consisting of hydrogen, alkyl, alkenyl, alkynyl, aryl, heteroaryl, cycloalkyl, heterocyclyl, alkylcarbonyl, cycloalkylcarbonyl, arylcarbonyl, heteroarylcarbonyl, arylsulfonyl, heteroarylsulfonyl, cycloalkylsulfonyl and alkylsulfonyl, or R⁶ and R⁷ are combined to form a cyclic structure including the nitrogen atom to which they are both attached.

Provided herein is a method for treating fibrosis in a subject having a fibrotic disease, comprising administering to the subject the compound of Formula II, or pharmaceutically acceptable salt thereof, wherein the compound of Formula II or pharmaceutically acceptable salt thereof is inactive according to MAPK p38 Activation Assay.

Provided herein is a compound of Formula III:

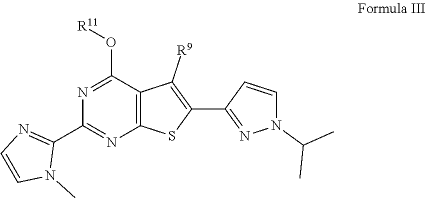

or a pharmaceutically acceptable salt thereof,
wherein:
   R⁹ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
   R¹¹ is H, alkyl, alkenyl, alkynyl, aryl, cycloalkyl, heterocyclyl, heteroaryl, C(O)R⁴, or S(O)ₚR⁴;
   R³ is hydrogen, alkyl, alkenyl, alkynyl, aryl, heteroaryl, heterocyclyl, cycloalkyl, alkylcarbonyl, cycloalkylcarbonyl or arylcarbonyl;
   R⁴ is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy, aralkoxy, or NR⁶R⁷;
   each R⁵ is selected from the group consisting of hydrogen, alkyl, alkenyl, alkynyl, aryl, heteroaryl, heterocyclyl, cycloalkyl, alkylcarbonyl, cycloalkylcarbonyl, and arylcarbonyl; and R⁶ and R⁷ are independently selected from the group consisting of hydrogen, alkyl, alkenyl, alkynyl, aryl, heteroaryl, cycloalkyl, heterocyclyl, alkylcarbonyl, cycloalkylcarbonyl, arylcarbonyl, heteroarylcarbonyl, arylsulfonyl, heteroarylsulfonyl, cycloalkylsulfonyl and alkylsulfonyl, or R⁶ and R⁷ are combined to form a cyclic structure including the nitrogen atom to which they are both attached.

Provided herein is a method for treating fibrosis in a subject having a fibrotic disease, comprising administering to the subject the compound of Formula III, or pharmaceutically acceptable salt thereof, wherein the compound of Formula III or pharmaceutically acceptable salt thereof is inactive according to MAPK p38 Activation Assay.

Provided herein are methods for treating fibrosis in a subject having a fibrotic disease by administering to the subject a compound or a pharmaceutically acceptable salt thereof identified as a modulator of Ras superfamily activity according to a Ras Superfamily Activity Assay, wherein the identified Ras superfamily modulating compound or pharmaceutically acceptable salt thereof inhibits phosphorylation of Smad2/3, according to Phospho-Smad2/3 Inhibition Assay, and is inactive according to MAPK p38 Activation Assay. In some embodiments, the identified Ras superfamily modulating compound or pharmaceutically acceptable salt thereof inhibits phosphorylation of Smad2/3, for example, by 20%, 30%, 50%, 70%, or more at 10 μM, according to Phospho-Smad2/3 Inhibition Assay. In some embodiments, the identified Ras superfamily modulating compound or pharmaceutically acceptable salt thereof activates JNK, according to JNK Activation Assay. In some embodiments, the Ras superfamily protein is a Ras protein, a Rac protein, or a Rho protein.

In some embodiments, the compound of Formula I, IA, II, or III, or pharmaceutically acceptable salt thereof, administered according to any of the methods disclosed herein treats, prevents, or inhibits fibrosis in the subject. In some embodiments, the compound of Formula I, IA, II, or III, or pharmaceutically acceptable salt thereof, administered according to any of the methods disclosed herein treats, prevents, or ameliorates one or more symptoms of a fibrotic disease in the subject. In some embodiments, the compound of Formula I, IA, II, or III, or pharmaceutically acceptable salt thereof, administered according to any of the methods disclosed herein treats, prevents, or ameliorates the fibrotic disease in the subject. In some embodiments, the fibrotic disease is selected from the group consisting of fibrosis of kidney, fibrosis of cardiovascular system, pulmonary fibrosis, cystic fibrosis, idiopathic fibrosis, fibrosis of the lung, bridging fibrosis, fibrosis of the liver, fibrosis of the intestine, fibrosis of the muscular system, fibrosis of the brain, fibrosis of the joints, fibrosis of the skin, fibrosis of the bone marrow, fibrosis of the heart, fibrosis of the soft tissue, fibrosis of the tendons, fibrosis of the lymph nodes, fibrosis of the eyes, retroperitoneum, scleroderma and surgical scarring.

### 5. BRIEF DESCRIPTION OF THE FIGURES

FIG. 1 depicts pharmacokinetic data for Compound 9 in a mouse model as described in Example 3.

FIG. 2 depicts western blot analysis of mouse heart tissue samples collected 3 days after IP injection of Compound 9 as described in Example 4.

FIG. 3 depicts western blot analysis of mouse diaphragm tissue samples collected 3 days after IP injection of Compound 9 as described in Example 4.

FIG. 4 depicts western blot analysis of mouse quadricep tissue samples collected 3 days after IP injection of Compound 9 as described in Example 4.

FIG. 5 depicts western blot analysis of mouse tibialis anterior tissue samples collected 3 days after IP injection of Compound 9 as described in Example 4.

### 6. DETAILED DESCRIPTION

### 6.1. Definitions

To facilitate understanding of the disclosure set forth herein, a number of terms are defined below.

Unless defined otherwise, all technical and scientific terms used herein have the same meaning as is commonly understood by one of ordinary skill in the art. All patents, applications, published applications and other publications are incorporated by reference in their entirety. In the event that there is a plurality of definitions for a term herein, those in this section prevail unless stated otherwise.

The singular forms “a,” “an,” and “the” include plural references, unless the context clearly dictates otherwise.

As used herein “subject” is an animal, such as a mammal, including human, such as a patient.

As used herein, biological activity refers to the in vivo activities of a compound or physiological responses that result upon in vivo administration of a compound, composition or other mixture. Biological activity, thus, encompasses therapeutic effects and pharmacokinetic behavior of such compounds, compositions and mixtures. Biological activities can be observed in in vitro systems designed to test for such activities.

As used herein, pharmaceutically acceptable derivatives of a compound include, but are not limited to, salts, esters, enol ethers, enol esters, acetals, ketals, orthoesters, hemiacetals, hemiketals, acids, bases, clathrates, solvates or hydrates thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates, and fumarates. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is alkyl, alkenyl, alkynyl, aryl, aralkyl and cycloalkyl. Pharmaceutically acceptable enol esters include, but are not limited to, derivatives of formula C═C(OC(O)R) where R is hydrogen, alkyl, alkenyl, alkynyl, aryl, aralkyl and cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating a fibrotic disease, for example DMD.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, and unless otherwise indicated, the terms “manage,” “managing” and “management” encompass preventing the recurrence of the specified disease or disorder in a subject who has already suffered from the disease or disorder, and/or lengthening the time that a subject who has suffered from the disease or disorder remains in remission. The terms encompass modulating the threshold, development and/or duration of the disease or disorder, or changing the way that a subject responds to the disease or disorder.

As used herein, the terms “fibrosis” or “fibrotic disease” may be used interchangeably and refer to any pathological wound healing process in which connective tissue replaces normal parenchymal tissue, leading to considerable tissue re-modeling and the formation of permanent scar tissue. For example, in some embodiments, the fibrotic disease may be fibrosis of the kidney, such as progressive kidney disease. In some embodiments, the fibrotic disease may be fibrosis of the cardiovascular system, such as atherosclerosis or restenosis. In some embodiments, the fibrotic disease may be pulmonary fibrosis. In some embodiments, the fibrotic disease may be cystic fibrosis. In some embodiments, the fibrotic disease may be idiopathic fibrosis, such as idiopathic pulmonary fibrosis. In some embodiments, the fibrotic disease may be fibrosis of the lung, such as progressive massive fibrosis or radiation-induced lung injury. In some embodiments, the fibrotic disease may be bridging fibrosis. In some embodiments, the fibrotic disease may be fibrosis of the liver, such as cirrhosis. In some embodiments, the fibrotic disease may be fibrosis of the intestine, such as Crohn's disease. In some embodiments, the fibrotic disease may be fibrosis of the muscular system, such as Duchenne muscular dystrophy (DMD). In some embodiments, the fibrotic disease may be fibrosis of the brain, such as glial scar. In some embodiments, the fibrotic disease may be fibrosis of the joints, such as arterial stiffness, fibrosis of the knee or fibrosis of the shoulder. In some embodiments, the fibrotic disease may be fibrosis of the skin, such as Keloid. In some embodiments, the fibrotic disease may be fibrosis of the bone marrow, such as myelofibrosis. In some embodiments, the fibrotic disease may be fibrosis of the heart, such as myocardial fibrosis. In some embodiments, the fibrotic disease may be fibrosis of the soft tissue. In some embodiments, the fibrotic disease may be fibrosis of the tendons. In some embodiments, the fibrotic disease may be fibrosis of the lymph nodes. In some embodiments, the fibrotic disease may be fibrosis of the eyes. In some embodiments, the fibrotic disease may be retroperitoneum. In some embodiments, the fibrotic disease may be scleroderma. In some embodiments, the fibrotic disease may be surgical scarring.

As used herein, “Duchenne muscular dystrophy” (“DMD”) refers to muscular dystrophy and all forms of Duchenne muscular dystrophy (DMD). For example, in some embodiments, the DMD may be Becker Muscular Dystrophy (BMD), an intermediate clinical presentation between DMD and BMD, or DMD-associated dilated cardiomyopathy (heart-disease) with little or no clinical skeletal, or voluntary, muscle disease.

As used herein, the IC₅₀ refers to an amount, concentration or dosage of a particular test compound that achieves a 50% inhibition of a maximal response in an assay that measures such response.

As used herein, the K_{d} refers to the measured equilibrium dissociation constant between a compound (or ligand) and a protein (or binding domain of a protein).

As used herein, “Smad 2/3” means the members of the receptor-regulated Smad (R-Smads) family of transcription factors, Smad2 and Smad3, collectively.

As used herein, “MAPK” means mitogen-activated protein kinase, which includes the stress-activated MAPK protein, MAPK p38, or simply p38.

As used herein, “JNK” means the stress-activated MAPK protein c-Jun NH₂-terminal kinase.

As used herein, “Ras superfamily” means the protein superfamily of small guanosine triphosphatases (GTPases) which consists of the five main families Ras, Rho, Rab, Ran and Arf, or mutants thereof. Subfamilies of the five main families are also included, e.g., the Rac subfamily of the Rho main family.

As used herein, “Ras” or “Ras family” or “Ras subfamily” or “Ras group” means DIRAS1; DIRAS2; DIRAS3; ERAS; GEM; HRAS; KRAS; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; RRAS2, or mutants thereof.

As used herein, “Rho” or “Rho family” or “Rho subfamily” or “Rho group” means RHOA; RHOB; RHOBTB1; RHOBTB2; RHOBTB3; RHOC; RHOD; RHOF; RHOG; RHOH; RHOJ; RHOQ; RHOU; RHOV; RND1; RND2; RND3; RAC1; RAC2; RAC3; CDC42, or mutants thereof.

As used herein, “Rac” or “Rac family” or “Rac subfamily” or “Rac group” means RAC1; RAC2; RAC3; RHOG, or mutants thereof.

As used herein, “GTP binding site” or “GTP binding domain” both mean the region of a protein which binds GTP, and the surrounding region of said protein in which another compound may bind, wherein such binding blocks the ability of GTP to bind to said protein.

As used herein, “GDP binding site” or “GDP binding domain” both mean the region of a protein which binds GDP, and the surrounding region of said protein in which another compound may bind, wherein such binding blocks the ability of GDP to bind to said protein.

As used herein, “guanosine binding region” means a region of a protein which is part of the GDP binding domain or GTP binding domain, that mediates interaction with the guanosine portion of GDP or GTP.

As used herein, “metal region” means a region of a protein which is part of the GDP binding domain or GTP binding domain, that is proximal to a magnesium (Mg202) binding site.

As used herein, “alternative Tyr32 conformation” means the conformation of the GTP or GDP binding domain in the region of Tyr 32 in KRas crystal structure PDB code:3gft in comparison to the KRas crystal structure PDB code:4epr.

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. As such, one of skill in the art will recognize that administration of a compound in its (R) form is equivalent, for compounds that undergo epimerization in vivo, to administration of the compound in its (S) form. Racemic compounds that contain two asymmetric centers with known relative configuration are named using the configurational descriptors R,S or R,R, preceded by the prefix rac-. When two stereocenters are present in a molecule, racemates are indicated by (R*,R*) or (R*,S*).

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter enzymatic and biological activities of the substance. Methods for purification of the compounds to produce substantially chemically pure compounds are known to those of skill in the art. A substantially chemically pure compound may, however, be a mixture of stereoisomers. In such instances, further purification might increase the specific activity of the compound. The instant disclosure is meant to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as chiral reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included. For example, Formula A includes, but is not limited to, the three tautomeric structures below.

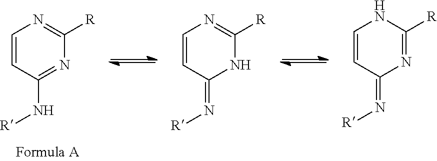

As used herein, the abbreviations for any protective groups, amino acids and other compounds, are, unless indicated otherwise, in accord with their common usage, recognized abbreviations, the IUPAC-IUB Commission on Biochemical Nomenclature (see, (1972) Biochem. 11:942-944), or the IUPAC Nomenclature of Organic Chemistry (see, Favre H A and Powell W H, Nomenclature of Organic Chemistry: IUPAC Recommendations and Preferred Names 2013, Cambridge, UK: The Royal Society of Chemistry, 2013: Print ISBN 978-0-85404-182-4, PDF eISBN 978-1-84973-306-9, DOI 10.1039/9781849733069; Nomenclature of Organic Chemistry, Sections A, B, C, D, E, F, and H, Pergamon Press, Oxford, 1979. Copyright 1979 IUPAC; and A Guide to IUPAC Nomenclature of Organic Compounds (Recommendations 1993), 1993, Blackwell Scientific publications, Copyright 1993 IUPAC).

As used herein, alkyl, alkenyl and alkynyl carbon chains, if not specified, contain from 1 to 20 carbons, or 1 to 16 carbons, and are straight or branched. Alkenyl carbon chains of from 2 to 20 carbons, in certain embodiments, contain 1 to 8 double bonds, and the alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, ethenyl, propenyl, butenyl, pentenyl, acetylenyl and hexynyl. As used herein, lower alkyl, lower alkenyl, and lower alkynyl refer to carbon chains having from about 1 or about 2 carbons up to about 6 carbons. As used herein, “alk(en)(yn)yl” refers to an alkyl group containing at least one double bond and at least one triple bond.

As used herein, “heteroalkyl” refers to a straight or branched aliphatic hydrocarbon group having, inserted in the hydrocarbon chain one or more oxygen, sulfur, including S(═O) and S(═O)₂ groups, or substituted or unsubstituted nitrogen atoms, including —NR— and —N⁺RR— groups, where the nitrogen substituent(s) is(are) alkyl, aryl, aralkyl, heteroaryl, heteroaralkyl, S(═O)₂R′ or COR′, where R′ is alkyl, aryl, aralkyl, heteroaryl, heteroaralkyl, OY or —NYY′, where Y and Y′ are each independently hydrogen, alkyl, aryl, heteroaryl, cycloalkyl or heterocyclyl, in one embodiment having from 1 to about 20 atoms, in another embodiment having from 1 to 12 atoms in the chain.

As used herein, “cycloalkyl” refers to a saturated mono- or multicyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or spiro-connected fashion. “Cycloalk(en)(yn)yl” refers to a cycloalkyl group containing at least one double bond and at least one triple bond. In some embodiments, the cycloalkyl ring is unsaturated or partially saturated.

As used herein, “carbocyclic” refers to a mono- or multicyclic ring system, in which all of the atoms composing the ring are carbon atoms, such as benzene or cyclopropane. In some embodiments, the carbocyclic ring is unsaturated or partially saturated.

As used herein, “substituted alkyl,” “substituted alkenyl,” “substituted alkynyl,” “substituted cycloalkyl,” “substituted cycloalkenyl,” and “substituted cycloalkynyl” refer to alkyl, alkenyl, alkynyl, cycloalkyl, cycloalkenyl and cycloalkynyl groups, respectively, that are substituted with one or more substituents, in certain embodiments one to three or four substituents, where the substituents are as defined herein, in one embodiment selected from Q.

As used herein, “aryl” refers to aromatic monocyclic or multicyclic groups containing from 6 to 19 carbon atoms. Aryl groups include, but are not limited to groups such as fluorenyl, substituted fluorenyl, phenyl, substituted phenyl, naphthyl and substituted naphthyl.

As used herein, “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, N-methylpyrrolyl, quinolinyl and isoquinolinyl.

As used herein, “heterocycloalkyl,” “heterocyclyl” or “heterocyclic” refers to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur. In embodiments where the heteroatom(s) is(are) nitrogen, the nitrogen is optionally substituted with hydrogen, alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, amidino, sulfonyl or the nitrogen may be quaternized to form an ammonium group where the substituents are selected as above. In some embodiments, the heterocyclyl ring is saturated. In some embodiments, the heterocyclyl ring is unsaturated or partially saturated.

As used herein, “substituted aryl,” “substituted heteroaryl” and “substituted heterocyclyl” refer to aryl, heteroaryl and heterocyclyl groups, respectively, that are substituted with one or more substituents, in certain embodiments one to three or four substituents, where the substituents are as defined herein, in one embodiment selected from Q.

As used herein, “aralkyl” or “arylalkyl” refers to an alkyl group in which one of the hydrogen atoms of the alkyl is replaced by an aryl group.

As used herein, “heteroaralkyl” refers to an alkyl group in which one of the hydrogen atoms of the alkyl is replaced by a heteroaryl group.

As used herein, “halo”, “halogen” or “halide” refers to F, Cl, Br or I.

As used herein, pseudohalides or pseudohalo groups are groups that behave substantially similar to halides. Such compounds can be used in the same manner and treated in the same manner as halides. Pseudohalides include, but are not limited to, cyano, thiocyanate, selenocyanate, trifluoromethoxy, and azide.

As used herein, “haloalkyl” refers to an alkyl group in which one or more of the hydrogen atoms are replaced by halogen. Such groups include, but are not limited to, chloromethyl, trifluoromethyl and 1-chloro-2-fluoroethyl.

As used herein, “haloalkoxy” refers to RO in which R is a haloalkyl group.

As used herein, “carboxy” refers to a divalent radical, —C(O)O—.

As used herein, “aminocarbonyl” refers to —C(O)NH₂.

As used herein, “alkylaminocarbonyl” refers to —C(O)NHR in which R is alkyl, including lower alkyl. As used herein, “dialkylaminocarbonyl” refers to —C(O)NR′R in which R′ and R are independently alkyl, including lower alkyl; “carboxamide” refers to groups of formula —NR′COR in which R′ and R are independently alkyl, including lower alkyl.

As used herein, “arylalkylaminocarbonyl” refers to —C(O)NRR′ in which one of R and R′ is aryl, including lower aryl, such as phenyl, and the other of R and R′ is alkyl, including lower alkyl.

As used herein, “arylaminocarbonyl” refers to —C(O)NHR in which R is aryl, including lower aryl, such as phenyl.

As used herein, “hydroxycarbonyl” refers to —COOH.

As used herein, “alkoxycarbonyl” refers to —C(O)OR in which R is alkyl, including lower alkyl.

As used herein, “aryloxycarbonyl” refers to —C(O)OR in which R is aryl, including lower aryl, such as phenyl.

As used herein, “alkoxy” and “alkylthio” refer to RO— and RS—, in which R is alkyl, including lower alkyl.

As used herein, “aryloxy” and “arylthio” refer to RO— and RS—, in which R is aryl, including lower aryl, such as phenyl.

Where the number of any given substituent is not specified (e.g., “haloalkyl”), there may be one or more substituents present. For example, “haloalkyl” may include one or more of the same or different halogens.

As used herein, “cyclic structure” may be a cycloalkyl, carbocyclic, heterocyclic, aryl or heteroaryl group.

Where substitution is not specified (e.g., “aryl”), there may be one or more substituents present. For example, “aryl” may include a “substituted aryl” group. In some embodiments, each alkyl, alkenyl, alkynyl, cycloalkyl, cycloalkenyl, cycloalkynyl, aryl, aralkyl, heteroaryl, and heterocyclyl is optionally substituted with one or more substituents, in one embodiment one, two, three or four substituents Q, where each Q is independently selected from (a) deuterium, cyano, halo, and nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c)—C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}—NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a}; wherein each Q^{a} is independently selected from the group consisting of (a) deuterium, cyano, halo, and nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NRC(O)OR^{f}, —NR^{e}C(O)NR^{f}R^{g}, —NR^{e}C(═NR^{h})NR^{f}R^{g}, —NRS(O)R^{h}, —NR^{e}S(O)₂R^{h}, —R^{e}S(O)NR^{f}R^{g}, —NR^{e}S(O)₂NR^{f}R^{g}, —SR, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl. In some embodiments, two Q substituents together with the atoms to which they are attached, may form a fused ring system.

As used herein, the abbreviations for any protective groups, amino acids and other compounds, are, unless indicated otherwise, in accord with their common usage, recognized abbreviations, the IUPAC-IUB Commission on Biochemical Nomenclature (see, (1972) Biochem. 11:942-944), or the IUPAC Nomenclature of Organic Chemistry (see, Favre H A and Powell W H, Nomenclature of Organic Chemistry: IUPAC Recommendations and Preferred Names 2013, Cambridge, UK: The Royal Society of Chemistry, 2013: Print ISBN 978-0-85404-182-4, PDF eISBN 978-1-84973-306-9, DOI 10.1039/9781849733069; Nomenclature of Organic Chemistry, Sections A, B, C, D, E, F, and H, Pergamon Press, Oxford, 1979. Copyright 1979 IUPAC; and A Guide to IUPAC Nomenclature of Organic Compounds (Recommendations 1993), 1993, Blackwell Scientific publications, Copyright 1993 IUPAC).

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject, in one embodiment, a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The term “therapeutically effective amount” are meant to include the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician. A therapeutically effective amount of a compound provided herein can be administered in one dose (i.e., a single dose administration) or divided and administered over time (i.e., continuous administration or multiple sub-dose administration). Single dose administration, continuous administration, or multiple sub-dose administration can be repeated, for example, to maintain the level of the compound in a biological molecule (e.g., a protein, enzyme, RNA, or DNA), cell, tissue, system, animal, or human.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 22nd ed.; Loyd et al., Eds.; The Pharmaceutical Press, 2012; Handbook of Pharmaceutical Excipients, 7th ed.; Rowe et al., Eds.; The Pharmaceutical Press, 2012; Handbook of Pharmaceutical Additives, 3rd ed.; Ash and Ash Eds.; Synapse Information Resources, Inc., 2007; Pharmaceutical Preformulation and Formulation, 2nd ed.; Gibson Ed.; CRC Press LLC, 2009.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The term “percent by weight” or “% by weight” refers to the weight of a specified component (e.g., an active compound or excipient) in a composition (e.g., a pharmaceutical composition) as a percentage of the total weight of the composition. Thus, the sum of the weight percentages of all the components in a composition is 100%.

The terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” may be an optically active isomer or an isotopic variant of a compound described herein.

The terms “drug,” “therapeutic agent,” and “chemotherapeutic agent” refer to a compound, or a pharmaceutical composition thereof, which is administered to a subject for treating, preventing, or ameliorating one or more symptoms of a condition, disorder, or disease.

In certain embodiments, “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of one enantiomer and about 5% or less of the other enantiomer based on the total weight of the racemate in question.

In describing an optically active compound, the prefixes R and S are used to denote the absolute configuration of the molecule about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the molecule, R and S.

The term “racemate” is understood to refer to an equimolar mixture of a pair of enantiomers. It does not exhibit optical activity. The chemical name or formula of a racemate is distinguished from those of the enantiomers by the prefix (±)-, or rac- (or rac. or racem-) or by the symbols RS and SR. See IUPAC Recommendations 1996, Basic Terminology of Stereochemistry, Pure & Appl. Chem., Vol. 68, No. 12, pp. 2193-2222, 1996.

Racemic compounds disclosed herein that contain two asymmetric centers with known relative configuration are named using the configurational descriptors R,S or R,R, preceded by the prefix rac-. For example, Racemic Compound A below is named rac-(1R,3S)-1-bromo-3-chlorocyclohexane and is a 1:1 mixture of enantiomers (1R,3S)-1-bromo-3-chlorocyclohexane and (1S,3R)-1-bromo-3-chlorocyclohexane.

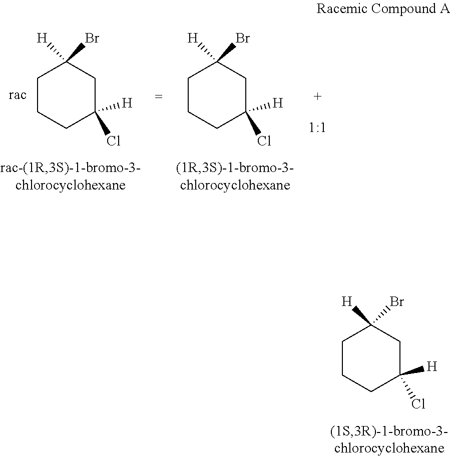 [SMILES: Cl[C@H]1CCC[C@@H](Br)C1 ; Cl[C@H]1CCC[C@@H](Br)C1 ; Cl[C@@H]1CCC[C@H](Br)C1]

Lower case r/s stereo descriptors are used to describe pseudo-asymmetric centers, according to Cahn-Ingold-Prelog Rules (see R.S. Cahn, C. K. Ingold and V. Prelog, Angew. Chem. Internat. Ed. Eng. 5, 385-415, 511 (1966); and V. Prelog and G. Helmchen, Angew. Chem. Internat. Ed. Eng. 21, 567-583 (1982)). For example, Compound B below is named (1s,4s)-1-bromo-4-chlorocyclohexane.

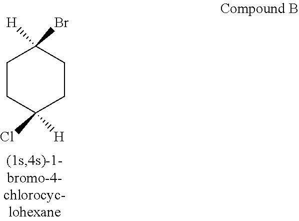

Compound names included herein were generated from the corresponding chemical structures using ChemDraw® versions 20.0.0.38 and 20.1.0,112, If there is a discrepancy between the chemical structure and the name disclosed herein, the structure shall control.

The term “isotopic variant” refers to a compound that contains an unnatural proportion of an isotope at one or more of the atoms that constitute such compounds. In certain embodiments, an “isotopic variant” of a compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (H), deuterium (H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an “isotopic variant” of a compound is in an unstable form, that is, radioactive. In certain embodiments, an “isotopic variant” of a compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I). It will be understood that, in a compound as provided herein, any hydrogen can be ²H, for example, or any carbon can be ¹³C, as example, or any nitrogen can be ¹⁵N, as example, and any oxygen can be ¹⁸O, where feasible according to the judgment of one of skill. In certain embodiments, an “isotopic variant” of a compound contains unnatural proportions of deuterium. In some embodiments, a pharmaceutically acceptable derivative of a compound is an isotopic variant.

The term “solvate” refers to a complex or aggregate formed by one or more molecules of a solute, e.g., a compound provided herein, and one or more molecules of a solvent, which present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The phrase “an isotopic variant thereof; or a pharmaceutically acceptable salt thereof; or a pharmaceutically acceptable solvate thereof” has the same meaning as the phrase “an isotopic variant of the compound referenced therein; or a pharmaceutically acceptable salt of the compound referenced therein; or a pharmaceutically acceptable salt of an isotopic variant of the compound referenced therein; or a pharmaceutically acceptable solvate of the compound referenced therein; or a pharmaceutically acceptable solvate of an isotopic variant of the compound referenced therein; or a pharmaceutically acceptable solvate of a pharmaceutically acceptable salt of the compound referenced therein; or a pharmaceutically acceptable solvate of a pharmaceutically acceptable salt of an isotopic variant of the compound referenced therein or its variant or its variant.”

### 6.2. Methods of Treating Fibrosis

Fibrosis, also known as fibrotic scarring, is a pathological wound healing process in which connective tissue replaces normal parenchymal tissue, leading to considerable tissue re-modeling and the formation of permanent scar tissue. Repeated injuries, chronic inflammation and repair are susceptible to fibrosis where excessive extracellular matrix (ECM) components, such as collagen and glycosaminoglycans., accumulate and lead to the formation of a permanent fibrotic scar which can interfere with normal organ and tissue functions.

Fibrosis can occur in many tissues within the body, typically as a result of inflammation or damage. Examples include: fibrosis of kidney, fibrosis of cardiovascular system, pulmonary fibrosis, cystic fibrosis, idiopathic fibrosis, fibrosis of the lung, bridging fibrosis, fibrosis of the liver, fibrosis of the intestine, fibrosis of the muscular system, fibrosis of the brain, fibrosis of the joints, fibrosis of the skin, fibrosis of the bone marrow, fibrosis of the heart, fibrosis of the soft tissue, fibrosis of the tendons, fibrosis of the lymph nodes, fibrosis of the eyes, retroperitoneum, scleroderma and surgical scarring.

The process of tissue repair is a complex one, with tight regulation of ECM synthesis and degradation ensuring maintenance of normal tissue architecture. However, the process can lead to a progressive irreversible fibrotic response if tissue injury is severe or repetitive, or if the wound healing response itself becomes deregulated. Fibrosis is initiated when immune cells such as macrophages and damaged tissue between surfaces called interstitium release soluble factors that stimulate fibroblasts. The best characterized pro-fibrotic mediators are the transforming growth factor-β (TGF-β ligands such as TGF-β1, -β2 and -β3, bone morphogenetic proteins (BMPs), and Activin.

The pro-fibrotic TGF-β and its related ligands bind a heteromeric complex of type I and type II trans-membrane TGF-β receptors, each equipped with an intracellular kinase domain. Upon ligand binding the type II receptor kinases phosphorylate and thereby activate the type I receptors, which are also known as activin receptor-like kinases (ALKs). Downstream of this activated complex, a canonical signaling pathway is composed of the Smad family of transcription factors, among which Smad2 and Smad3 are phosphorylated and activated by type I TGF-β receptors. Activated Smad2/3 (RSmads) form a trimeric complex with Smad4 that translocates to the nucleus to regulate target gene expression.

Other soluble mediators of fibrosis include connective tissue growth factor (CTGF), platelet-derived growth factor (PDGF), and interleukin 10 (IL-10).

Thus, inhibition of the TGF-β1 signaling pathway and Smad2 and 3 phosphorylation and activation represent potential therapeutic approaches for treating fibrosis.

Duchenne muscular dystrophy (DMD) is a genetic disorder characterized by progressive muscle degeneration and weakness due to the alterations of a protein called dystrophin that helps keep muscle cells intact. DMD is one of four conditions known as dystrophinopathies. Three diseases that belong to this group are Becker Muscular Dystrophy (BMD, a mild form of DMD); an intermediate clinical presentation between DMD and BMD; and DMD-associated dilated cardiomyopathy (heart-disease) with little or no clinical skeletal, or voluntary, muscle disease.

DMD primarily affects boys, but in rare cases it can affect girls. In Europe and North America, the prevalence of DMD is approximately 6 per 100,000 individuals.

Muscle weakness is the principal symptom of DMD. Symptom onset is in early childhood, usually between ages 2 and 3. The disease first affects the proximal muscles, and later the distal limb muscles. Usually, the lower external muscles are affected before the upper external muscles. Later on, the heart and respiratory muscles are affected. Progressive weakness and scoliosis result in impaired pulmonary function, which can eventually cause acute respiratory failure. Becker muscular dystrophy (BMD) is a similar to DMD, but with onset usually in the teens or early adulthood. The disease course for BMD is slower and less predictable compared to DMD.

DMD was first described by the French neurologist Guillaume Benjamin Amand Duchenne in the 1860s, but until the 1980s little was known about the cause of any kind of muscular dystrophy. In 1986, researchers identified a particular gene on the X chromosome that, when mutated, leads to DMD. In 1987, the protein associated with this gene was identified and named dystrophin. Lack of wild type dystrophin protein in muscle cells causes them to be fragile and easily damaged. DMD has an X-linked recessive inheritance pattern and is passed on by the mother, who is referred to as a carrier.

Until relatively recently, boys with DMD usually did not survive much beyond their teen years. Thanks to advances in cardiac and respiratory care, and targeted therapeutic products, life expectancy is increasing and survival into the early 30s is becoming more common but there is still an unmet need for therapeutics to reduce morbidity and lengthen lifespans.

When skeletal muscle is unable to regenerate due to DMD, damaged muscle is eventually replaced with fibrotic tissue. Development of this fibrosis is partially mediated by the TGF-β1 signaling pathway, which, therefore may be a therapeutic target for DMD.

A growing number of published studies also suggest that pharmacological activation of the stress-activated MAPK proteins p-38 MAPK and c-Jun NH₂-terminal kinase (JNK) may also represent a therapeutic approach for the treatment of DMD related fibrosis.

Provided herein are methods for treating fibrosis in a subject having a fibrotic disease by administering to the subject a compound of Formula I

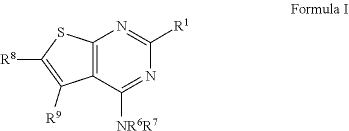

or a pharmaceutically acceptable salt thereof, wherein:
R¹ is H, alkyl, aryl or heteroaryl;
R⁸ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
R⁹ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
R³ is hydrogen, alkyl, alkenyl, alkynyl, aryl, heteroaryl, heterocyclyl, cycloalkyl, alkylcarbonyl, cycloalkylcarbonyl or arylcarbonyl;
R⁴ is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy, aralkoxy, or NR⁶R⁷;
each R⁵ is selected from the group consisting of hydrogen, alkyl, alkenyl, alkynyl, aryl, heteroaryl, heterocyclyl, cycloalkyl, alkylcarbonyl, cycloalkylcarbonyl, and arylcarbonyl; and
R⁶ and R⁷ are independently selected from the group consisting of hydrogen, alkyl, alkenyl, alkynyl, aryl, heteroaryl, cycloalkyl, heterocyclyl, alkylcarbonyl, cycloalkylcarbonyl, arylcarbonyl, heteroarylcarbonyl, arylsulfonyl, heteroarylsulfonyl, cycloalkylsulfonyl and alkylsulfonyl, or R⁶ and R⁷ are combined to form a cyclic structure including the nitrogen atom to which they are both attached; and
   wherein the compound of Formula I or pharmaceutically acceptable salt thereof inhibits phosphorylation of Smad2/3, according to Phospho-Smad2/3 Inhibition Assay, and is inactive according to MAPK p38 Activation Assay. In some embodiments, the compound of Formula I or pharmaceutically acceptable salt thereof inhibits phosphorylation of Smad2/3 by 20% or more at 10 μM, according to Phospho-Smad2/3 Inhibition Assay. In some embodiments, the compound of Formula I or pharmaceutically acceptable salt thereof inhibits phosphorylation of Smad2/3 by 30% or more at 10 μM, according to Phospho-Smad2/3 Inhibition Assay. In some embodiments, the compound of Formula I or pharmaceutically acceptable salt thereof inhibits phosphorylation of Smad2/3 by 50% or more at 10 μM, according to Phospho-Smad2/3 Inhibition Assay. In some embodiments, the compound of Formula I or pharmaceutically acceptable salt thereof inhibits phosphorylation of Smad2/3 by 70% or more at 10 μM, according to Phospho-Smad2/3 Inhibition Assay. In some embodiments, the compound of Formula I is a compound of Formula IA.

In some embodiments, the compound of Formula I administered according to the methods disclosed herein inhibits phosphorylation of Smad2/3, according to Phospho-Smad2/3 Inhibition Assay, is inactive according to MAPK p38 Activation Assay, and wherein R¹ is heteroaryl, R⁸ is aryl or heteroaryl, R⁹ is aryl or alkyl, and R⁶ and R⁷ are independently selected from the group consisting of hydrogen, alkyl, aryl, heteroaryl, cycloalkyl, and heterocyclyl, or R⁶ and R⁷ are combined to form a cyclic structure including the nitrogen atom to which they are both attached. In some embodiments, the compound of Formula I is such that R¹ is imidazolyl substituted with alkyl, R⁸ is unsubstituted phenyl or substituted pyrazolyl, and R⁹ is unsubstituted phenyl. In some embodiments, the compound of Formula I is such that R¹ is imidazolyl substituted with methyl or methoxy ethyl. In some embodiments, the compound of Formula I is such that R⁸ is an unsubstituted phenyl group. In some embodiments, the compound of Formula I is such that R⁸ is pyrazolyl substituted with alkyl. In some embodiments, the compound of Formula I is such that R⁸ is pyrazolyl substituted with methyl.

In some embodiments, the compound of Formula I is such that R⁸ is:
phenyl,

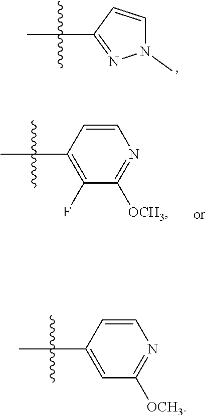

In some embodiments, the compound of Formula I is such that R⁹ is unsubstituted phenyl. In some embodiments, the compound of Formula I is such that NR⁶R⁷ is:

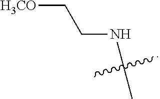 [SMILES: *NCCOC]

In some embodiments, the compound of Formula I is such that NR⁶R⁷ is:

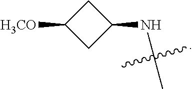 [SMILES: *N[C@H]1C[C@@H](OC)C1]

In some embodiments, the compound of Formula I is such that the —NR⁶R⁷ group depicted in Formula I is:

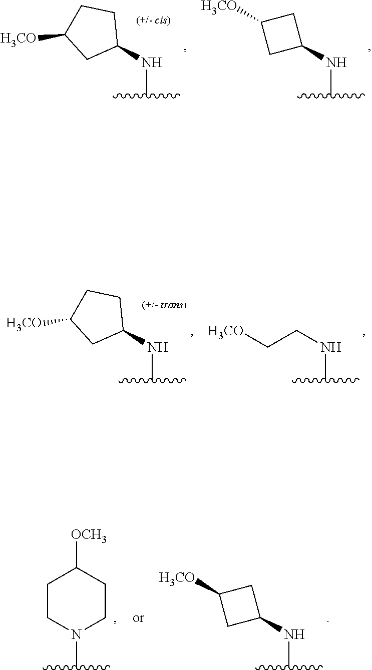 [SMILES: *N[C@@H]1CC[C@H](OC)C1 ; *N[C@H]1C[C@H](OC)C1 ; *N[C@@H]1CC[C@@H](OC)C1 ; *NCCOC ; *N1CCC(OC)CC1 ; *N[C@H]1C[C@@H](OC)C1]

In some embodiments, the compound of Formula I or pharmaceutically acceptable salt thereof inhibits phosphorylation of Smad2/3 by 20% or more at 10 μM, according to Phospho-Smad2/3 Inhibition Assay. In some embodiments, the compound of Formula I or pharmaceutically acceptable salt thereof inhibits phosphorylation of Smad2/3 by 30% or more at 10 μM, according to Phospho-Smad2/3 Inhibition Assay. In some embodiments, the compound of Formula I or pharmaceutically acceptable salt thereof inhibits phosphorylation of Smad2/3 by 50% or more at 10 μM, according to Phospho-Smad2/3 Inhibition Assay. In some embodiments, the compound of Formula I or pharmaceutically acceptable salt thereof inhibits phosphorylation of Smad2/3 by 70% or more at 10 μM, according to Phospho-Smad2/3 Inhibition Assay. In some embodiments, the compound of Formula I is a compound of Formula IA.

Provided herein are methods for treating fibrosis in a subject having a fibrotic disease by administering to the subject a compound of Formula I.

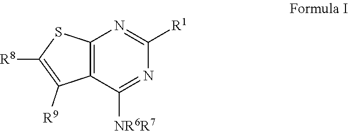

or a pharmaceutically acceptable salt thereof, which inhibits phosphorylation of Smad2/3, according to Phospho-Smad2/3 Inhibition Assay, and is inactive according to MAPK p38 Activation Assay, wherein the compound of Formula I or pharmaceutically acceptable salt thereof is selected from the group consisting of:
N-(2-Methoxyethyl)-2-(1-methyl-1H-imidazol-2-yl)-5,6-diphenylthieno[2,3-d]pyrimidin-4-amine;
N-((1s,3s)-3-ethoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine;
rac-N-((1R,3S)-3-Methoxycyclopentyl)-6-(2-methoxypyridin-4-yl)-2-(1-methyl-1H-imidazol-2-yl)-5-phenylthieno[2,3-d]pyrimidin-4-amine;
6-(3-Fluoro-2-methoxypyridin-4-yl)-N-((1r,3r)-3-methoxycyclobutyl)-2-(1-methyl-1H-imidazol-2-yl)-5-phenylthieno[2,3-d]pyrimidin-4-amine;
N-((1r,3r)-3-Methoxycyclobutyl)-6-(2-methoxypyridin-4-yl)-2-(1-methyl-1H-imidazol-2-yl)-5-phenylthieno[2,3-d]pyrimidin-4-amine; and
N-((1R,3R)-3-Methoxycyclopentyl)-6-(2-methoxypyridin-4-yl)-2-(1-methyl-1H-imidazol-2-yl)-5-phenylthieno[2,3-d]pyrimidin-4-amine.
   In some embodiments, the compound of Formula I administered according to the methods disclosed herein activates JNK, according to INK Activation Assay. In some embodiments, the compound of Formula I administered according to the methods disclosed herein is a modulator of Ras superfamily activity according to a Ras Superfamily Activity Assay. In some embodiments, the compound of Formula I is a compound of Formula IA.

Provided herein are methods for treating fibrosis in a subject having a fibrotic disease by administering to the subject a compound of Formula I or pharmaceutically acceptable salt thereof which inhibits phosphorylation of Smad2/3 by 20% or more at 10 μM, according to Phospho-Smad2/3 Inhibition Assay, and is inactive according to MAPK p38 Activation Assay.

In some embodiments, the compound of Formula I administered according to the methods disclosed herein is selected from the group consisting of:
N-(2-Methoxyethyl)-2-(1-methyl-1H-imidazol-2-yl)-5,6-diphenylthieno[2,3-d]pyrimidin-4-amine;
N-((1s,3s)-3-ethoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine;
rac-N-((1R,3S)-3-Methoxycyclopentyl)-6-(2-methoxypyridin-4-yl)-2-(1-methyl-1H-imidazol-2-yl)-5-phenylthieno[2,3-d]pyrimidin-4-amine;
6-(3-Fluoro-2-methoxypyridin-4-yl)-N-((1r,3r)-3-methoxycyclobutyl)-2-(1-methyl-1H-imidazol-2-yl)-5-phenylthieno[2,3-d]pyrimidin-4-amine;
N-((1r,3r)-3-Methoxycyclobutyl)-6-(2-methoxypyridin-4-yl)-2-(1-methyl-1H-imidazol-2-yl)-5-phenylthieno[2,3-d]pyrimidin-4-amine; and
N-((1R,3R)-3-Methoxycyclopentyl)-6-(2-methoxypyridin-4-yl)-2-(1-methyl-1H-imidazol-2-yl)-5-phenylthieno[2,3-d]pyrimidin-4-amine.
   In some embodiments, the compound of Formula I administered according to the methods disclosed herein activates INK, according to JNK Activation Assay. In some embodiments, the compound of Formula I administered according to the methods disclosed herein is a modulator of Ras superfamily activity according to a Ras Superfamily Activity Assay. In some embodiments, the compound of Formula I is a compound of Formula IA.

Provided herein are methods for treating fibrosis in a subject having a fibrotic disease by administering to the subject a compound of Formula I or pharmaceutically acceptable salt thereof which inhibits phosphorylation of Smad2/3 by 30% or more at 10 μM, according to Phospho-Smad2/3 Inhibition Assay, and is inactive according to MAPK p38 Activation Assay. In some embodiments, the compound of Formula I administered according to the methods disclosed herein is selected from the group consisting of:
N-(2-Methoxyethyl)-2-(1-methyl-1H-imidazol-2-yl)-5,6-diphenylthieno[2,3-d]pyrimidin-4-amine;
N-((1s,3s)-3-ethoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine;
rac-N-((1R,3S)-3-Methoxycyclopentyl)-6-(2-methoxypyridin-4-yl)-2-(1-methyl-1H-imidazol-2-yl)-5-phenylthieno[2,3-d]pyrimidin-4-amine;
6-(3-Fluoro-2-methoxypyridin-4-yl)-N-((1r,3r)-3-methoxycyclobutyl)-2-(1-methyl-1H-imidazol-2-yl)-5-phenylthieno[2,3-d]pyrimidin-4-amine;
N-((1r,3r)-3-Methoxycyclobutyl)-6-(2-methoxypyridin-4-yl)-2-(1-methyl-1H-imidazol-2-yl)-5-phenylthieno[2,3-d]pyrimidin-4-amine; and
N-((1R,3R)-3-Methoxycyclopentyl)-6-(2-methoxypyridin-4-yl)-2-(1-methyl-1H-imidazol-2-yl)-5-phenylthieno[2,3-d]pyrimidin-4-amine.
   In some embodiments, the compound of Formula I administered according to the methods disclosed herein activates JNK, according to INK Activation Assay. In some embodiments, the compound of Formula I administered according to the methods disclosed herein is a modulator of Ras superfamily activity according to a Ras Superfamily Activity Assay. In some embodiments, the compound of Formula I is a compound of Formula IA.

Provided herein are methods for treating fibrosis in a subject having a fibrotic disease by administering to the subject a compound of Formula I or pharmaceutically acceptable salt thereof which inhibits phosphorylation of Smad2/3 by 50% or more at 10 μM, according to Phospho-Smad2/3 Inhibition Assay, and is inactive according to MAPK p38 Activation Assay. In some embodiments, the compound of Formula I administered according to the methods disclosed herein is selected from the group consisting of:
N-(2-Methoxyethyl)-2-(1-methyl-1H-imidazol-2-yl)-5,6-diphenylthieno[2,3-d]pyrimidin-4-amine;
N-((1s,3s)-3-ethoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine;
rac-N-((1R,3S)-3-Methoxycyclopentyl)-6-(2-methoxypyridin-4-yl)-2-(1-methyl-1H-imidazol-2-yl)-5-phenylthieno[2,3-d]pyrimidin-4-amine;
6-(3-Fluoro-2-methoxypyridin-4-yl)-N-((1r,3r)-3-methoxycyclobutyl)-2-(1-methyl-1H-imidazol-2-yl)-5-phenylthieno[2,3-d]pyrimidin-4-amine;
N-((1r,3r)-3-Methoxycyclobutyl)-6-(2-methoxypyridin-4-yl)-2-(1-methyl-1H-imidazol-2-yl)-5-phenylthieno[2,3-d]pyrimidin-4-amine; and
N-((1R,3R)-3-Methoxycyclopentyl)-6-(2-methoxypyridin-4-yl)-2-(1-methyl-1H-imidazol-2-yl)-5-phenylthieno[2,3-d]pyrimidin-4-amine.
   In some embodiments, the compound of Formula I administered according to the methods disclosed herein activates JNK, according to INK Activation Assay. In some embodiments, the compound of Formula I administered according to the methods disclosed herein is a modulator of Ras superfamily activity according to a Ras Superfamily Activity Assay. In some embodiments, the compound of Formula I is a compound of Formula IA.

Provided herein are methods for treating fibrosis in a subject having a fibrotic disease by administering to the subject a compound of Formula I or pharmaceutically acceptable salt thereof which inhibits phosphorylation of Smad2/3 by 70% or more at 10 μM, according to Phospho-Smad2/3 Inhibition Assay, and is inactive according to MAPK p38 Activation Assay. In some embodiments, the compound of Formula I administered according to the methods disclosed herein is selected from the group consisting of:
N-((1s,3s)-3-ethoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine;
rac-N-((1R,3S)-3-Methoxycyclopentyl)-6-(2-methoxypyridin-4-yl)-2-(1-methyl-1H-imidazol-2-yl)-5-phenylthieno[2,3-d]pyrimidin-4-amine;
6-(3-Fluoro-2-methoxypyridin-4-yl)-N-((1r,3r)-3-methoxycyclobutyl)-2-(1-methyl-1H-imidazol-2-yl)-5-phenylthieno[2,3-d]pyrimidin-4-amine;
N-((1r,3r)-3-Methoxycyclobutyl)-6-(2-methoxypyridin-4-yl)-2-(1-methyl-1H-imidazol-2-yl)-5-phenylthieno[2,3-d]pyrimidin-4-amine; and
N-((1R,3R)-3-Methoxycyclopentyl)-6-(2-methoxypyridin-4-yl)-2-(1-methyl-1H-imidazol-2-yl)-5-phenylthieno[2,3-d]pyrimidin-4-amine.
   In some embodiments, the compound of Formula I administered according to the methods disclosed herein activates JNK, according to INK Activation Assay. In some embodiments, the compound of Formula I administered according to the methods disclosed herein is a modulator of Ras superfamily activity according to a Ras Superfamily Activity Assay. In some embodiments, the compound of Formula I is a compound of Formula IA.

Provided herein are methods for treating fibrosis in a subject having a fibrotic disease by administering to the subject a compound of Formula I or pharmaceutically acceptable salt thereof which is exclusive of compounds that do not inhibit phosphorylation of Smad2/3, according to the Phospho-Smad2/3 Inhibition Assay, and is exclusive of compounds that are active against (or activate) MAPK p38, according to the MAPK p38 Activation Assay. In some embodiments, the compound of Formula I administered according to the methods herein is exclusive of compounds selected from the group consisting of:
N-(2-Methoxyethyl)-N-methyl-2-(1-methyl-1H-imidazol-2-yl)-5,6-diphenylthieno[2,3-d]pyrimidin-4-amine;
N-(3-Methoxypropyl)-2-(1-methyl-1H-imidazol-2-yl)-5,6-diphenylthieno[2,3-d]pyrimidin-4-amine;
5-(2-Chlorophenyl)-N-(2-methoxyethyl)-2-(1-methyl-1H-imidazol-2-yl)-6-phenylthieno[2,3-d]pyrimidin-4-amine;
rac-N-((1R,3S)-3-Methoxycyclopentyl)-2-(1-methyl-1H-imidazol-2-yl)-6-(1-methyl-1H-pyrazol-3-yl)-5-phenylthieno[2,3-d]pyrimidin-4-amine;
4-(4-Methoxypiperidin-1-yl)-2-(1-methyl-1H-imidazol-2-yl)-6-(1-methyl-1H-pyrazol-3-yl)-5-phenylthieno[2,3-d]pyrimidine;
6-(2-Chloro-3-methoxyphenyl)-N-(2-methoxyethyl)-5-(4-methoxyphenyl)-2-(1-methyl-1H-imidazol-2-yl)thieno[2,3-d]pyrimidin-4-amine;
6-(2-Chloro-3-methoxyphenyl)-N-(2-methoxyethyl)-5-(4-methoxyphenyl)-2-(1-methyl-1H-imidazol-4-yl)thieno[2,3-d]pyrimidin-4-amine;
6-(2-Chloro-3-methoxyphenyl)-N-(2-methoxyethyl)-2-(1-methyl-1H-imidazol-2-yl)-5-phenylthieno[2,3-d]pyrimidin-4-amine;
rac-6-(2-Fluoro-3-methoxyphenyl)-N-((1R,3S)-3-methoxycyclopentyl)-5-methyl-2-(1-methyl-1H-imidazol-2-yl)thieno[2,3-d]pyrimidin-4-amine and rac-6-(2-Fluoro-3-methoxyphenyl)-N-((1R,3R)-3-methoxycyclopentyl)-5-methyl-2-(1-methyl-1H-imidazol-2-yl)thieno[2,3-d]pyrimidin-4-amine;
6-(1-Isopropyl-1H-pyrazol-3-yl)-N-((1S,3R)-3-methoxycyclopentyl)-2-(1-methyl-1H-imidazol-2-yl)-5-phenylthieno[2,3-d]pyrimidin-4-amine;
rac-6-(1-Isopropyl-1H-pyrazol-3-yl)-N-((1R,3S)-3-methoxycyclopentyl)-5-methyl-2-(1-methyl-1H-imidazol-2-yl)thieno[2,3-d]pyrimidin-4-amine;
rac-6-(1-Isopropyl-1H-pyrazol-3-yl)-4-(3-methoxypiperidin-1-yl)-5-methyl-2-(1-methyl-1H-imidazol-2-yl)thieno[2,3-d]pyrimidine;
4-(6-(1-Isopropyl-1H-pyrazol-3-yl)-5-methyl-2-(1-methyl-1H-imidazol-2-yl)thieno[2,3-d]pyrimidin-4-yl)-2-(methoxymethyl)morpholine;
6-(1-Isopropyl-1H-pyrazol-3-yl)-N-(3-methoxyphenyl)-5-methyl-2-(1-methyl-1H-imidazol-2-yl)thieno[2,3-d]pyrimidin-4-amine;
6-(1-Isopropyl-1H-pyrazol-3-yl)-N-(6-methoxypyridin-2-yl)-5-methyl-2-(1-methyl-1H-imidazol-2-yl)thieno[2,3-d]pyrimidin-4-amine;
6-(1-Isopropyl-1H-pyrazol-3-yl)-N-(6-methoxypyrimidin-4-yl)-5-methyl-2-(1-methyl-1H-imidazol-2-yl)thieno[2,3-d]pyrimidin-4-amine;
rac-6-(1-Isopropyl-1H-pyrazol-3-yl)-N-((1R,3S)-3-(2-methoxyethoxy)cyclopentyl)-5-methyl-2-(1-methyl-1H-imidazol-2-yl)thieno[2,3-d]pyrimidin-4-amine;
rac-6-(1-(2-Methoxyethyl)-1H-pyrazol-3-yl)-N-(((1S,2R)-2-(methoxymethyl)cyclobutyl)methyl)-5-methyl-2-(1-methyl-1H-imidazol-2-yl)thieno[2,3-d]pyrimidin-4-amine;
6-(2-Fluoro-3-methoxyphenyl)-N-((1r,3r)-3-methoxycyclobutyl)-2-(1-methyl-1H-imidazol-2-yl)-5-phenylthieno[2,3-d]pyrimidin-4-amine;
6-(2-Fluoro-3-methoxyphenyl)-N-((1R,3R)-3-methoxycyclopentyl)-2-(1-methyl-1H-imidazol-2-yl)-5-phenylthieno[2,3-d]pyrimidin-4-amine;
6-(2-Fluoro-3-methoxyphenyl)-N-((1r,3r)-3-methoxycyclobutyl)-5-methyl-2-(1-methyl-1H-imidazol-2-yl)thieno[2,3-d]pyrimidin-4-amine; and
rac-6-(2-Fluoro-3-methoxyphenyl)-N-((1R,3R)-3-methoxycyclopentyl)-5-methyl-2-(1-methyl-1H-imidazol-2-yl)thieno[2,3-d]pyrimidin-4-amine.
   In some embodiments, the compound of Formula I administered according to the methods disclosed herein activates JNK, according to INK Activation Assay. In some embodiments, the compound of Formula I administered according to the methods disclosed herein is a modulator of Ras superfamily activity according to a Ras Superfamily Activity Assay.

Provided herein are methods for treating fibrosis in a subject having a fibrotic disease by administering to the subject a compound of Formula IA:

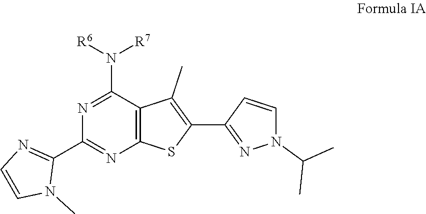

or a pharmaceutically acceptable salt thereof,
wherein:
R⁶ and R⁷ are independently selected from the group consisting of hydrogen, alkyl, alkenyl, alkynyl, aryl, heteroaryl, cycloalkyl, heterocyclyl, alkylcarbonyl, cycloalkylcarbonyl, arylcarbonyl, heteroarylcarbonyl, arylsulfonyl, heteroarylsulfonyl, cycloalkylsulfonyl and alkylsulfonyl, or R⁶ and R⁷ are combined to form a cyclic structure including the nitrogen atom to which they are both attached; and
   wherein the compound of Formula IA or pharmaceutically acceptable salt thereof inhibits phosphorylation of Smad2/3, according to Phospho-Smad2/3 Inhibition Assay, and is inactive according to MAPK p38 Activation Assay. In some embodiments, the compound of Formula IA or pharmaceutically acceptable salt thereof inhibits phosphorylation of Smad2/3 by 20% or more at 10 μM, according to Phospho-Smad2/3 Inhibition Assay. In some embodiments, the compound of Formula IA or pharmaceutically acceptable salt thereof inhibits phosphorylation of Smad2/3 by 30% or more at 10 μM, according to Phospho-Smad2/3 Inhibition Assay. In some embodiments, the compound of Formula IA or pharmaceutically acceptable salt thereof inhibits phosphorylation of Smad2/3 by 50% or more at 10 μM, according to Phospho-Smad2/3 Inhibition Assay. In some embodiments, the compound of Formula IA or pharmaceutically acceptable salt thereof inhibits phosphorylation of Smad2/3 by 70% or more at 10 μM, according to Phospho-Smad2/3 Inhibition Assay.

Provided herein are methods for treating fibrosis in a subject having a fibrotic disease, comprising administering to the subject a compound of Formula II:

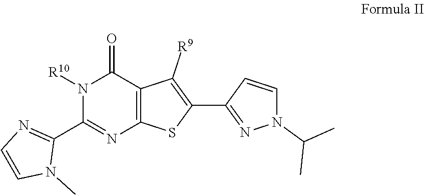

or a pharmaceutically acceptable salt thereof,
wherein:
R⁹ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
R¹⁰ is H, alkyl, alkenyl, alkynyl, aryl, cycloalkyl, heterocyclyl, heteroaryl, C(O)R⁴, or S(O)ₚR⁴;
R³ is hydrogen, alkyl, alkenyl, alkynyl, aryl, heteroaryl, heterocyclyl, cycloalkyl, alkylcarbonyl, cycloalkylcarbonyl or arylcarbonyl;
R⁴ is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy, aralkoxy, or NR⁶R⁷;
each R⁵ is selected from the group consisting of hydrogen, alkyl, alkenyl, alkynyl, aryl, heteroaryl, heterocyclyl, cycloalkyl, alkylcarbonyl, cycloalkylcarbonyl, and arylcarbonyl; and
R⁶ and R⁷ are independently selected from the group consisting of hydrogen, alkyl, alkenyl, alkynyl, aryl, heteroaryl, cycloalkyl, heterocyclyl, alkylcarbonyl, cycloalkylcarbonyl, arylcarbonyl, heteroarylcarbonyl, arylsulfonyl, heteroarylsulfonyl, cycloalkylsulfonyl and alkylsulfonyl, or R⁶ and R⁷ are combined to form a cyclic structure including the nitrogen atom to which they are both attached;
   wherein the compound of Formula II or pharmaceutically acceptable salt thereof is inactive according to MAPK p38 Activation Assay. In some embodiments, the compound of Formula II administered according to the methods disclosed herein inhibits INK, according to JNK Activation Assay. In some embodiments, the compound of Formula II administered according to the methods disclosed herein is a modulator of Ras superfamily activity according to a Ras Superfamily Activity Assay.

In some embodiments, the compound of Formula II is such that R⁹ is alkyl; and R¹⁰ is alkyl. In some embodiments, the compound of Formula II is such that R⁹ is methyl.

Provided herein are methods for treating fibrosis in a subject having a fibrotic disease by administering to the subject the compound of Formula II, or pharmaceutically acceptable salt thereof, which is inactive according to MAPK p38 Activation Assay, wherein the compound of Formula II or pharmaceutically acceptable salt thereof is selected from the group consisting of:
6-(1-Isopropyl-1H-pyrazol-3-yl)-3-(3-methoxypropyl)-5-methyl-2-(1-methyl-1H-imidazol-2-yl)thieno[2,3-d]pyrimidin-4(3H)-one;
6-(1-Isopropyl-1H-pyrazol-3-yl)-3-(4-methoxybutyl)-5-methyl-2-(1-methyl-1H-imidazol-2-yl)thieno[2,3-d]pyrimidin-4(3H)-one; and
6-(1-Isopropyl-1H-pyrazol-3-yl)-3-((6-methoxypyrimidin-4-yl)methyl)-5-methyl-2-(1-methyl-1H-imidazol-2-yl)thieno[2,3-d]pyrimidin-4(3H)-one.
   In some embodiments, the compound of Formula II administered according to the methods disclosed herein inhibits JNK, according to INK Activation Assay. In some embodiments, the compound of Formula II administered according to the methods disclosed herein is a modulator of Ras superfamily activity according to a Ras Superfamily Activity Assay.

Provided herein are methods for treating fibrosis in a subject having a fibrotic disease by administering to the subject the compound of Formula II, or pharmaceutically acceptable salt thereof, which is inactive according to MAPK p38 Activation Assay, wherein the compound of Formula II or pharmaceutically acceptable salt thereof is exclusive of compounds selected from the group consisting of:
6-(1-Isopropyl-1H-pyrazol-3-yl)-3-(2-methoxyethyl)-5-methyl-2-(1-methyl-1H-imidazol-2-yl)thieno[2,3-d]pyrimidin-4(3H)-one; and
6-(1-Isopropyl-1H-pyrazol-3-yl)-3-(3-methoxypropyl)-2-(1-methyl-1H-imidazol-2-yl)-5-phenylthieno[2,3-d]pyrimidin-4(3H)-one.

Provided herein are methods for treating fibrosis in a subject having a fibrotic disease, comprising administering to the subject a compound of Formula III:

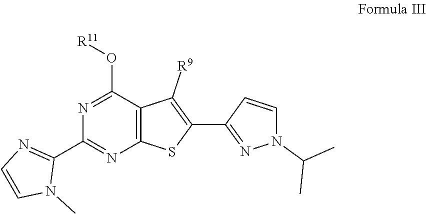

or a pharmaceutically acceptable salt thereof,
wherein:
R⁹ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
R¹¹ is H, alkyl, alkenyl, alkynyl, aryl, cycloalkyl, heterocyclyl, heteroaryl, C(O)R⁴, or S(O)ₚR⁴;
R³ is hydrogen, alkyl, alkenyl, alkynyl, aryl, heteroaryl, heterocyclyl, cycloalkyl, alkylcarbonyl, cycloalkylcarbonyl or arylcarbonyl;
R⁴ is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy, aralkoxy, or NR⁶R⁷;
each R⁵ is selected from the group consisting of hydrogen, alkyl, alkenyl, alkynyl, aryl, heteroaryl, heterocyclyl, cycloalkyl, alkylcarbonyl, cycloalkylcarbonyl, and arylcarbonyl; and
R⁶ and R⁷ are independently selected from the group consisting of hydrogen, alkyl, alkenyl, alkynyl, aryl, heteroaryl, cycloalkyl, heterocyclyl, alkylcarbonyl, cycloalkylcarbonyl, arylcarbonyl, heteroarylcarbonyl, arylsulfonyl, heteroarylsulfonyl, cycloalkylsulfonyl and alkylsulfonyl, or R⁶ and R⁷ are combined to form a cyclic structure including the nitrogen atom to which they are both attached;
   wherein the compound of Formula III or pharmaceutically acceptable salt thereof is inactive according to MAPK p38 Activation Assay. In some embodiments, the compound of Formula III administered according to the methods disclosed herein inhibits INK, according to JNK Activation Assay. In some embodiments, the compound of Formula III administered according to the methods disclosed herein is a modulator of Ras superfamily activity according to a Ras Superfamily Activity Assay.

In some embodiments, the compound of Formula III is such that R⁹ is aryl; and R¹⁰ is alkyl. In some embodiments, the compound of Formula III is such that R⁹ is methyl.

Provided herein are methods for treating fibrosis in a subject having a fibrotic disease by administering to the subject the compound of Formula III, or pharmaceutically acceptable salt thereof, which is inactive according to MAPK p38 Activation Assay, wherein the compound of Formula III or pharmaceutically acceptable salt thereof is selected from the group consisting of:
6-(1-Isopropyl-1H-pyrazol-3-yl)-4-(2-methoxyethoxy)-5-methyl-2-(1-methyl-1H-imidazol-2-yl)thieno[2,3-d]pyrimidine;
6-(1-Isopropyl-1H-pyrazol-3-yl)-4-(3-methoxypropoxy)-5-methyl-2-(1-methyl-1H-imidazol-2-yl)thieno[2,3-d]pyrimidine; and
6-(1-Isopropyl-1H-pyrazol-3-yl)-4-(4-methoxybutoxy)-5-methyl-2-(1-methyl-1H-imidazol-2-yl)thieno[2,3-d]pyrimidine.
   In some embodiments, the compound of Formula III administered according to the methods disclosed herein inhibits JNK, according to JNK Activation Assay. In some embodiments, the compound of Formula III administered according to the methods disclosed herein is a modulator of Ras superfamily activity according to a Ras Superfamily Activity Assay.

Provided herein are methods for treating fibrosis in a subject having a fibrotic disease by administering to the subject the compound of Formula III, or pharmaceutically acceptable salt thereof, which is inactive according to MAPK p38 Activation Assay, wherein the compound of Formula III or pharmaceutically acceptable salt thereof is exclusive of 6-(1-Isopropyl-1H-pyrazol-3-yl)-4-(3-methoxypropoxy)-2-(1-methyl-1H-imidazol-2-yl)-5-phenylthieno[2,3-d]pyrimidine.

Provided herein are methods for treating fibrosis in a subject having a fibrotic disease by administering to the subject a compound or a pharmaceutically acceptable salt thereof identified as a modulator of Ras superfamily activity according to a Ras Superfamily Activity Assay, wherein the identified Ras superfamily modulating compound or pharmaceutically acceptable salt thereof inhibits phosphorylation of Smad2/3, according to Phospho-Smad2/3 Inhibition Assay, and is inactive according to MAPK p38 Activation Assay. In some embodiments, the identified Ras superfamily modulating compound or pharmaceutically acceptable salt thereof inhibits phosphorylation of Smad2/3 by 20%, 30%, 50%, 70%, or more at 10 μM, according to Phospho-Smad2/3 Inhibition Assay. In some embodiments, the identified Ras superfamily modulating compound or pharmaceutically acceptable salt thereof activates JNK, according to JNK Activation Assay. In some embodiments, the Ras Superfamily Activity Assay comprises:
a) contacting the compound or pharmaceutically acceptable salt thereof with a Ras superfamily protein;
b) incubating a cyanine-labeled GTP with said compound or pharmaceutically acceptable salt thereof and the Ras superfamily protein; and
c) measuring the amount of the cyanine-labeled GTP bound to the Ras superfamily protein.
   In some embodiments, the Ras Superfamily Activity Assay is a cell-free assay. In some embodiments, the Ras Superfamily Activity Assay is a GTP-binding competition assay. In some embodiments, the Ras superfamily protein is a Ras protein, a Rac protein, or a Rho protein.

Provided herein are methods for treating fibrosis in a subject having a fibrotic disease by administering to the subject a compound or a pharmaceutically acceptable salt thereof identified as a modulator of a Ras protein activity according to a Ras Activity Assay, wherein the identified Ras modulating compound or pharmaceutically acceptable salt thereof inhibits phosphorylation of Smad2/3, according to Phospho-Smad2/3 Inhibition Assay, and is inactive according to MAPK p38 Activation Assay. In some embodiments, the identified Ras modulating compound or pharmaceutically acceptable salt thereof competitively inhibits GTP binding to the Ras GTP binding domain. In some embodiments, the identified Ras modulating compound or pharmaceutically acceptable salt thereof binds to the Ras protein GTP binding domain with greater than 25% inhibition at 20 uM. In some embodiments, the identified Ras modulating compound or pharmaceutically acceptable salt thereof has a binding affinity (K_{d}) to the Ras protein GTP binding domain of less than 10 μM. In some embodiments, the identified Ras modulating compound or pharmaceutically acceptable salt thereof inhibits the Ras activity and has an IC₅₀ value of less than 10 μM. In some embodiments, the identified Ras modulating compound or pharmaceutically acceptable salt thereof inhibits binding of the cyanine labeled GTP with an IC₅₀ value of less than 10 μM. In some embodiments, the cyanine-labeled GTP is a Cy3- or a Cy5-labeled GTP. In some embodiments, the Ras Activity Assay is a GTP-binding competition assay. In some embodiments, the Ras protein is immobilized. In some embodiments, the Ras protein is DIRAS I; DIRAS2; DIRAS3; ERAS; GEM; HRAS; KRAS; MRAS; NKIRASI; NKIRAS2; NRAS; RALA; RALB; RAPIA; RAPIB; RAP2A; RAP2B; RAP2C; RASDI; RASD2; RASLIOA; RASLIOB; RASLIIA; RASLIIB; RASL12; REMI; REM2; RERG; RERGL; RRAD; RRAS; or RRAS2. In some embodiments, the Ras protein is HRAS; KRAS; or NRAS, or a mutant thereof. In some embodiments, the Ras protein is a KRAS mutant. In some embodiments, the KRAS mutant is a KRas G12D mutant, KRas G12C mutant, or KRas Q61H mutant. In some embodiments, the Ras protein is wild-type KRas. In some embodiments, the Ras protein is HRAS or mutant thereof. In some embodiments, the Ras protein is NRAS or mutant thereof.

Provided herein are methods for treating fibrosis in a subject having a fibrotic disease by administering to the subject a compound or a pharmaceutically acceptable salt thereof identified as a modulator of a Rac protein activity according to a Rac Activity Assay, wherein the identified Rac modulating compound or pharmaceutically acceptable salt thereof inhibits phosphorylation of Smad2/3, according to Phospho-Smad2/3 Inhibition Assay, and is inactive according to MAPK p38 Activation Assay. In some embodiments, the identified Rac modulating compound or pharmaceutically acceptable salt thereof competitively inhibits GTP binding to the Rac GTP binding domain. In some embodiments, the identified Rac modulating compound or pharmaceutically acceptable salt thereof binds to the Rac protein GTP binding domain with greater than 25% inhibition at 20 μM. In some embodiments, the identified Rac modulating compound or pharmaceutically acceptable salt thereof has a binding affinity (K_{d}) to the Rac protein GTP binding domain of less than 10 μM. In some embodiments, the identified Rac modulating compound or pharmaceutically acceptable salt thereof inhibits the Rac activity and has an IC₅₀ value of less than 10 μM. In some embodiments, the identified Rac modulating compound or pharmaceutically acceptable salt thereof inhibits binding of the cyanine labeled GTP with an IC₅₀ value of less than 10 μM. In some embodiments, the cyanine-labeled GTP is a Cy3- or a Cy5-labeled GTP. In some embodiments, the Rac Activity Assay is a GTP-binding competition assay. In some embodiments, the Rac protein is immobilized. In some embodiments, the Rae protein is RAC1; RAC2; RAC3; RHOG, or a mutant thereof. In some embodiments, the Rac protein is wild-type RAC1

Provided herein are methods for treating fibrosis in a subject having a fibrotic disease by administering to the subject a compound or a pharmaceutically acceptable salt thereof identified as a modulator of a Rho protein activity according to a Rho Activity Assay, wherein the identified Rho modulating compound or pharmaceutically acceptable salt thereof inhibits phosphorylation of Smad2/3, according to Phospho-Smad2/3 Inhibition Assay, and is inactive according to MAPK p38 Activation Assay. In some embodiments, the identified Rho modulating compound or pharmaceutically acceptable salt thereof competitively inhibits GTP binding to the Rho GTP binding domain. In some embodiments, the identified Rho modulating compound or pharmaceutically acceptable salt thereof binds to the Rho protein GTP binding domain with greater than 25% inhibition at 20 μM. In some embodiments, the identified Rho modulating compound or pharmaceutically acceptable salt thereof has a binding affinity (K_{d}) to the Rho protein GTP binding domain of less than 10 μM. In some embodiments, the identified Rho modulating compound or pharmaceutically acceptable salt thereof inhibits the Rho activity and has an IC₅₀ value of less than 10 μM. In some embodiments, the identified Rho modulating compound or pharmaceutically acceptable salt thereof inhibits binding of the cyanine labeled GTP with an IC₅₀ value of less than 10 μM. In some embodiments, the cyanine-labeled GTP is a Cy3- or a Cy5-labeled GTP. In some embodiments, the Rho Activity Assay is a GTP-binding competition assay. In some embodiments, the Rho protein is immobilized. In some embodiments, the Rho protein is RHOA; RHOB; RHOBTB1; RHOBTB2; RHOBTB3; RHOC; RHOD; RHOF; RHOH; RHOJ; RHOQ; RHOU; RHOV; RND1; RND2; RND3; CDC42, or a mutant thereof. In some embodiments, the Rho protein is wild-type RHOA.

In some embodiments, the compound of Formula I, IA, II, or III, administered according to any of the methods disclosed herein treats, prevents, or inhibits fibrosis in the subject. In some embodiments, the compound of Formula I, IA, II, or III, administered according to any of the methods disclosed herein inhibits fibrosis in the liver, lung, skin, soft tissue, tendons, lymph nodes, lung, kidney, heart, eye, or retroperitoneum of said subject. In some embodiments, the compound of Formula I, IA, II, or III, administered according to any of the methods disclosed herein treats, prevents, or ameliorates one or more symptoms of a fibrotic disease in the subject. In some embodiments, the compound of Formula I, IA, II, or III, administered according to any of the methods disclosed herein treats, prevents, or ameliorates the fibrotic disease in the subject. In some embodiments, the fibrotic disease is selected from the group consisting of fibrosis of kidney, fibrosis of cardiovascular system, pulmonary fibrosis, cystic fibrosis, idiopathic fibrosis, fibrosis of the lung, bridging fibrosis, fibrosis of the liver, fibrosis of the intestine, fibrosis of the muscular system, fibrosis of the brain, fibrosis of the joints, fibrosis of the skin, fibrosis of the bone marrow, fibrosis of the heart, fibrosis of the soft tissue, fibrosis of the tendons, fibrosis of the lymph nodes, fibrosis of the eyes, retroperitoneum, scleroderma and surgical scarring. In some embodiments, the fibrotic disease is fibrosis of the kidney. In some embodiments, the fibrosis of the kidney is progressive kidney disease. In some embodiments, the fibrotic disease is fibrosis of the cardiovascular system. In some embodiments, the fibrosis of the cardiovascular system is atherosclerosis or restenosis. In some embodiments, the fibrotic disease is pulmonary fibrosis. In some embodiments, the fibrotic disease is cystic fibrosis. In some embodiments, the fibrotic disease is idiopathic fibrosis. In some embodiments, the idiopathic fibrosis is idiopathic pulmonary fibrosis. In some embodiments, the fibrotic disease is fibrosis of the lung. In some embodiments, the fibrosis of the lung is progressive massive fibrosis and radiation-induced lung injury. In some embodiments, the fibrotic disease is bridging fibrosis. In some embodiments, the fibrotic disease is fibrosis of the liver. In some embodiments, the fibrosis of the liver is cirrhosis. In some embodiments, the fibrotic disease is fibrosis of the intestine. In some embodiments, the fibrosis of the intestine is Crohn's disease. In some embodiments, the fibrotic disease is fibrosis of the muscular system. In some embodiments, the fibrosis of the muscular system is Duchenne muscular dystrophy (DMD). In some embodiments, the Duchenne muscular dystrophy is Becker Muscular Dystrophy (BMD), an intermediate clinical presentation between DMD and BMD, or DMD-associated dilated cardiomyopathy. In some embodiments, the fibrotic disease is fibrosis of the brain. In some embodiments, the fibrosis of the brain is glial scar. In some embodiments, the fibrotic disease is fibrosis of the joints. In some embodiments, the fibrosis of the joints is arterial stiffness. In some embodiments, the fibrosis of the joints is fibrosis of the knee. In some embodiments, the fibrosis of the joints is fibrosis of the shoulder. In some embodiments, the fibrotic disease is fibrosis of the skin. In some embodiments, the fibrosis of the skin is Keloid. In some embodiments, the fibrotic disease is fibrosis of the bone marrow. In some embodiments, the fibrosis of the bone marrow is Myelofibrosis. In some embodiments, the fibrotic disease is fibrosis of the heart. In some embodiments, the fibrosis of the heart is Myocardial fibrosis. In some embodiments, the fibrotic disease is fibrosis of the soft tissue. In some embodiments, the fibrotic disease is fibrosis of the tendons. In some embodiments, the fibrotic disease is fibrosis of the lymph nodes. In some embodiments, the fibrotic disease is fibrosis of the eyes. In some embodiments, the fibrotic disease is retroperitoneum. In some embodiments, the fibrotic disease is scleroderma. In some embodiments, the fibrotic disease is surgical scarring.

### 6.3. Methods of Treating Cancer

In one embodiment, provided herein is a method of treating or preventing cancer, which comprises administering to a subject a compound that inhibits the function of one or more members of the Ras superfamily. In one embodiment, provided herein is a method of treating or preventing cancer, which comprises administering to a subject a compound that inhibits the function of one or more members of the Ras superfamily by binding to the GTP binding domain of one or more members of the Ras superfamily. In one embodiment, provided herein is a method of treating or preventing cancer, which comprises administering to a subject a compound that inhibits the function of Ras by binding to a Ras GTP binding domain. In another embodiment, provided herein is a method of treating or preventing cancer, which comprises administering to a subject a compound that binds to a Ras GTP binding domain with an IC50 of less than 10 μM and a Kd of less than 10 μM. In another embodiment, provided herein is a method of treating or preventing cancer, which comprises administering to a subject a compound that binds to a Ras GTP binding domain with an IC50 of less than 1 μM and a Kd of less than 1 μM. In another embodiment, provided herein is a method of treating or preventing cancer, which comprises administering to a subject a compound that binds to a Ras GTP binding domain with an IC50 of less than 500 nM and a Kd of less than 500 nM. In another embodiment, provided herein is a method of treating or preventing cancer, which comprises administering to a subject a compound that binds to a Ras GTP binding domain with an IC50 of less than 470 nM and a Kd of less than 470 nM. In another embodiment, provided herein is a method of treating or preventing cancer, which comprises administering to a subject a compound that binds to a Ras GTP binding domain with an IC50 of less than 270 nM and a Kd of less than 270 nM. In another embodiment, provided herein is a method of treating or preventing cancer, which comprises administering to a subject a compound that binds to a Ras GTP binding domain with an IC50 of less than 200 nM and a Kd of less than 200 nM. In another embodiment, provided herein is a method of treating or preventing cancer, which comprises administering to a subject a compound that binds to a Ras GTP binding domain with an IC50 of less than 150 nM and a Kd of less than 150 nM. In another embodiment, provided herein is a method of treating or preventing cancer, which comprises administering to a subject a compound that binds to a Ras GTP binding domain with an IC50 of less than 100 nM and a Kd of less than 100 nM. In another embodiment, provided herein is a method of treating or preventing cancer, which comprises administering to a subject a compound that binds to a Ras GTP binding domain with greater than 15% inhibition at 20 μM. In another embodiment, provided herein is a method of treating or preventing cancer, which comprises administering to a subject a compound that binds to a Ras GTP binding domain with greater than 25% inhibition at 20 μM. In another embodiment, provided herein is a method of treating or preventing cancer, which comprises administering to a subject a compound that binds to a Ras GTP binding domain with greater than 50% inhibition at 20 μM. In another embodiment, provided herein is a method of treating or preventing cancer, which comprises administering to a subject a compound that binds to a Ras GTP binding domain with greater than 75% inhibition at 20 μM. In another embodiment, provided herein is a method of treating or preventing cancer, which comprises administering to a subject a compound that binds to a Ras GTP binding domain with greater than 80% inhibition at 20 μM. In another embodiment, provided herein is a method of treating or preventing cancer, which comprises administering to a subject a compound that binds to a Ras GTP binding domain with greater than 85% inhibition at 20 μM. In another embodiment, provided herein is a method of treating or preventing cancer, which comprises administering to a subject a compound that binds to a Ras GTP binding domain with greater than 90% inhibition at 20 μM. In another embodiment, provided herein is a method of treating or preventing cancer, which comprises administering to a subject a compound that binds to a Ras GTP binding domain with greater than 95% inhibition at 20 μM. In another embodiment, provided herein is a method of treating or preventing cancer, which comprises administering to a subject a compound that binds to a Ras GTP binding domain with greater than 99% inhibition at 20 μM. In one embodiment, provided herein is a method of treating or preventing cancer, which comprises administering to a subject a compound that inhibits the function of Rho. In one embodiment, provided herein is a method of treating or preventing cancer, which comprises administering to a subject a compound that inhibits the function of Rho by binding to a Rho GTP binding domain. In another embodiment, provided herein is a method of treating or preventing cancer, which comprises administering to a subject a compound that binds to a Rho GTP binding domain with an IC50 of less than 10 μM and a Kd of less than 10 μM. In another embodiment, provided herein is a method of treating or preventing cancer, which comprises administering to a subject a compound that binds to a Rho GTP binding domain with an IC50 of less than 1 μM and a Kd of less than 1 μM. In another embodiment, provided herein is a method of treating or preventing cancer, which comprises administering to a subject a compound that binds to a Rho GTP binding domain with an IC50 of less than 500 nM and a Kd of less than 500 nM. In another embodiment, provided herein is a method of treating or preventing cancer, which comprises administering to a subject a compound that binds to a Rho GTP binding domain with an IC50 of less than 270 nM and a Kd of less than 270 nM. In another embodiment, provided herein is a method of treating or preventing cancer, which comprises administering to a subject a compound that binds to a Rho GTP binding domain with an IC50 of less than 200 nM and a Kd of less than 200 nM. In another embodiment, provided herein is a method of treating or preventing cancer, which comprises administering to a subject a compound that binds to a Rho GTP binding domain with an IC50 of less than 150 nM and a Kd of less than 150 nM. In another embodiment, provided herein is a method of treating or preventing cancer, which comprises administering to a subject a compound that binds to a Rho GTP binding domain with an IC50 of less than 130 nM and a Kd of less than 130 nM. In another embodiment, provided herein is a method of treating or preventing cancer, which comprises administering to a subject a compound that binds to a Rho GTP binding domain with an IC50 of less than 100 nM and a Kd of less than 100 nM. In another embodiment, provided herein is a method of treating or preventing cancer, which comprises administering to a subject a compound that binds to a Rho GTP binding domain with greater than 15% inhibition at 20 μM. In another embodiment, provided herein is a method of treating or preventing cancer, which comprises administering to a subject a compound that binds to a Rho GTP binding domain with greater than 25% inhibition at 20 μM. In another embodiment, provided herein is a method of treating or preventing cancer, which comprises administering to a subject a compound that binds to a Rho GTP binding domain with greater than 50% inhibition at 20 μM. In another embodiment, provided herein is a method of treating or preventing cancer, which comprises administering to a subject a compound that binds to a Rho GTP binding domain with greater than 75% inhibition at 20 μM. In another embodiment, provided herein is a method of treating or preventing cancer, which comprises administering to a subject a compound that binds to a Rho GTP binding domain with greater than 80% inhibition at 20 μM. In another embodiment, provided herein is a method of treating or preventing cancer, which comprises administering to a subject a compound that binds to a Rho GTP binding domain with greater than 85% inhibition at 20 μM. In another embodiment, provided herein is a method of treating or preventing cancer, which comprises administering to a subject a compound that binds to a Rho GTP binding domain with greater than 90% inhibition at 20 DM. In another embodiment, provided herein is a method of treating or preventing cancer, which comprises administering to a subject a compound that binds to a Rho GTP binding domain with greater than 95% inhibition at 20 μM. In another embodiment, provided herein is a method of treating or preventing cancer, which comprises administering to a subject a compound that binds to a Rho GTP binding domain with greater than 99% inhibition at 20 μM. In one embodiment, provided herein is a method of treating or preventing cancer, which comprises administering to a subject a compound that inhibits the function of Rac. In one embodiment, provided herein is a method of treating or preventing cancer, which comprises administering to a subject a compound that inhibits the function of Rac by binding to a Rac GTP binding domain. In another embodiment, provided herein is a method of treating or preventing cancer, which comprises administering to a subject a compound that binds to a Rac GTP binding domain with an IC50 of less than 10 μM and a Kd of less than 10 μM. In another embodiment, provided herein is a method of treating or preventing cancer, which comprises administering to a subject a compound that binds to a Rac GTP binding domain with an IC50 of less than 1 μM and a Kd of less than 1 μM. In another embodiment, provided herein is a method of treating or preventing cancer, which comprises administering to a subject a compound that binds to a Rac GTP binding domain with an IC50 of less than 500 nM and a Kd of less than 500 nM. In another embodiment, provided herein is a method of treating or preventing cancer, which comprises administering to a subject a compound that binds to a Rac GTP binding domain with an IC50 of less than 270 nM and a Kd of less than 270 nM. In another embodiment, provided herein is a method of treating or preventing cancer, which comprises administering to a subject a compound that binds to a Rac GTP binding domain with an IC50 of less than 200 nM and a Kd of less than 200 nM. In another embodiment, provided herein is a method of treating or preventing cancer, which comprises administering to a subject a compound that binds to a Rac GTP binding domain with an IC50 of less than 170 nM and a Kd of less than 170 nM. In another embodiment, provided herein is a method of treating or preventing cancer, which comprises administering to a subject a compound that binds to a Rac GTP binding domain with an IC50 of less than 150 nM and a Kd of less than 150 nM. In another embodiment, provided herein is a method of treating or preventing cancer, which comprises administering to a subject a compound that binds to a Rac GTP binding domain with an IC50 of less than 100 nM and a Kd of less than 100 nM. In another embodiment, provided herein is a method of treating or preventing cancer, which comprises administering to a subject a compound that binds to a Rac GTP binding domain with greater than 15% inhibition at 20 QM. In another embodiment, provided herein is a method of treating or preventing cancer, which comprises administering to a subject a compound that binds to a Rac GTP binding domain with greater than 25% inhibition at 20 μM. In another embodiment, provided herein is a method of treating or preventing cancer, which comprises administering to a subject a compound that binds to a Rac GTP binding domain with greater than 50% inhibition at 20 μM. In another embodiment, provided herein is a method of treating or preventing cancer, which comprises administering to a subject a compound that binds to a Rac GTP binding domain with greater than 75% inhibition at 20 μM. In another embodiment, provided herein is a method of treating or preventing cancer, which comprises administering to a subject a compound that binds to a Rac GTP binding domain with greater than 80% inhibition at 20 μM. In another embodiment, provided herein is a method of treating or preventing cancer, which comprises administering to a subject a compound that binds to a Rac GTP binding domain with greater than 85% inhibition at 20 μM. In another embodiment, provided herein is a method of treating or preventing cancer, which comprises administering to a subject a compound that binds to a Rac GTP binding domain with greater than 90% inhibition at 20 μM. In another embodiment, provided herein is a method of treating or preventing cancer, which comprises administering to a subject a compound that binds to a Rac GTP binding domain with greater than 95% inhibition at 20 μM. In another embodiment, provided herein is a method of treating or preventing cancer, which comprises administering to a subject a compound that binds to a Rac GTP binding domain with greater than 99% inhibition at 20 μM. In some embodiments, the compound for use in the method is a compound as disclosed herein of Formula I, IA, II, or III, or a pharmaceutically acceptable salt thereof. In some embodiments, the compound as disclosed herein for use in the method is a compound selected from Compounds 1-47, such as a compound selected from compounds 1-47 of Example 2, or a pharmaceutically acceptable salt thereof.

In another embodiment, provided herein is a method of treating or preventing cancer, which comprises administering to a subject a compound that binds to one or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to two or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to three or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to four or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to five or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to six or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to seven or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to eight or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to nine or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to ten or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to eleven or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to twelve or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to thirteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to fourteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to fifteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to sixteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to seventeen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to eighteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys 17, Asp119, Leu120, Ser145, Ala146, Lys147 or Mg202 in a Ras GTP binding domain. In another embodiment, provided herein is a method of treating or preventing cancer, which comprises administering to a subject a compound that binds to nineteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to twenty or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to twenty-one or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to twenty-two or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to twenty-three or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to all of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In some embodiments, the compound for use in the method is a compound as disclosed herein of Formula I, IA, II, or III, or a pharmaceutically acceptable salt thereof. In some embodiments, the compound as disclosed herein for use in the method is a compound selected from Compounds 1-47, such as a compound selected from compounds 1-47 of Example 2, or a pharmaceutically acceptable salt thereof.

In one embodiment, the Ras is DIRAS1; DIRAS2; DIRAS3; ERAS; GEM; HRAS; KRAS; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REMI; REM2; RERG; RERGL; RRAD; RRAS; or RRAS2. In another embodiment, the Ras is HRAS, KRAS or NRAS. In one embodiment, the Ras is HRAS. In one embodiment, the Ras is KRAS. In one embodiment, the Ras is NRAS. In another embodiment, the Ras is a mutant form of a Ras described herein.

In another embodiment, provided herein is a method of treating or preventing cancer, which comprises administering to a subject a compound that binds to one or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to two or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to three or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to four or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to five or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to six or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to seven or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to eight or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to nine or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to ten or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to eleven or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to twelve or more of Gly14, Ala15, Cys16, Gly17, Lys18, Thr19, Cys20, Phe30, Pro3l, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to thirteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to fourteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to fifteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to sixteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to seventeen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds all of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In some embodiments, the compound for use in the method is a compound as disclosed herein of Formula I, IA, II, or III, or a pharmaceutically acceptable salt thereof. In some embodiments, the compound as disclosed herein for use in the method is a compound selected from Compounds 1-47, such as a compound selected from compounds 1-47 of Example 2, or a pharmaceutically acceptable salt thereof.

In one embodiment, the Rho is RHOA; RHOB; RHOBTB1; RHOBTB2; RHOBTB3; RHOC; RHOD; RHOF; RHOG; RHOH; RHOJ; RHOQ; RHOU; RHOV; RND1; RND2; RND3; RAC1; RAC2; RAC3 or CDC42. In one embodiment, the Rho is RHOA. In another embodiment, the Rho is a mutant form of a Rho described herein.

In one embodiment, provided herein is a method of treating or preventing cancer, which comprises administering to a subject a compound that binds to one or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to two or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a highly conserved Rho GTP binding domain. In one embodiment, provided herein is a method of treating or preventing cancer, which comprises administering to a subject a compound that binds to three or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to four or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to five or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to six or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to seven or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to eight or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to nine or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to ten or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to eleven or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to twelve or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to thirteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to fourteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to fifteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rae GTP binding domain. In one embodiment, provided herein is a method of treating or preventing cancer, which comprises administering to a subject a compound that binds to sixteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to seventeen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to eighteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to all of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In some embodiments, the compound for use in the method is a compound as disclosed herein of Formula I, IA, II, or III, or a pharmaceutically acceptable salt thereof. In some embodiments, the compound as disclosed herein for use in the method is a compound selected from Compounds 1-47, such as a compound selected from compounds 1-47 of Example 2, or a pharmaceutically acceptable salt thereof.

In one embodiment, the Rho is Rac. In one embodiment the Rae is RAC1; RAC2; RAC3 or RHOG. In one embodiment, the Rac is RAC1. In another embodiment, the Rac is a mutant form of a Rac described herein.

In one embodiment, the compound for use in the methods and compositions provided herein inhibit GTP binding to one or more members of the Ras superfamily. In one embodiment, the compound for use in the methods and compositions provided herein inhibit GTP binding to Ras. In one embodiment, the compounds provided herein inhibit GTP binding to Rho. In one embodiment, the compound for use in the methods and compositions provided herein inhibit GTP binding to Rac. In one embodiment, the compound for use in the methods and compositions provided herein inhibit GTP binding to Ras and Rho. In one embodiment, the compound for use in the methods and compositions provided herein inhibit GTP binding to Ras and Rac. In one embodiment, the compound for use in the methods and compositions provided herein inhibit GTP binding to Rho and Rac. In one embodiment, the compound for use in the methods and compositions provided herein inhibit GTP binding to Ras, Rho and Rac. In one embodiment, the compound for use in the methods and compositions provided herein has a molecular weight less than 2000 daltons. In one embodiment, the compound for use in the methods and compositions provided herein has a molecular weight less than 1750 daltons. In one embodiment, the compound for use in the methods and compositions provided herein has a molecular weight less than 1500 daltons. In one embodiment, the compound for use in the methods and compositions provided herein has a molecular weight less than 1250 daltons. In one embodiment, the compound for use in the methods and compositions provided herein has a molecular weight less than 1000 daltons. In one embodiment, the compound for use in the methods and compositions provided herein has a molecular weight less than 750 daltons. In one embodiment, the compound for use in the methods and compositions provided herein has a molecular weight less than 665 daltons. In one embodiment, the compound for use in the methods and compositions provided herein has a molecular weight less than 500 daltons. In some embodiments, the compound for use in the method is a compound as disclosed herein of Formula I, IA, II, or III, or a pharmaceutically acceptable salt thereof. In some embodiments, the compound as disclosed herein for use in the method is a compound selected from Compounds 1-47, such as a compound selected from compounds 1-47 of Example 2, or a pharmaceutically acceptable salt thereof.

In one embodiment, provided herein is a method of treating or preventing cancer, which comprises administering to a subject a compound provided herein, or a derivative thereof. In some embodiments, the compound for use in the method is a compound as disclosed herein of Formula I, IA, II, or III, or a pharmaceutically acceptable salt thereof. In some embodiments, the compound as disclosed herein for use in the method is a compound selected from Compounds 1-47, such as a compound selected from compounds 1-47 of Examples 1, or a pharmaceutically acceptable salt thereof.

In another embodiment, provided herein is method of managing cancer, which comprises administering to a subject a compound provided herein, or a derivative thereof. In some embodiments, the compound for use in the method is a compound as disclosed herein of Formula I, IA, II, or III, or a pharmaceutically acceptable salt thereof. In some embodiments, the compound as disclosed herein for use in the method is a compound selected from Compounds 1-47, such as a compound selected from compounds 1-47 of Example 2, or a pharmaceutically acceptable salt thereof.

Also provided herein are methods of treating subjects who have been previously treated for cancer but are non-responsive to standard therapies, as well as those who have not previously been treated. Also provided are methods of treating subjects regardless of subject's age, although some diseases or disorders are more common in certain age groups. Also provided are methods of treating subjects who have undergone surgery in an attempt to treat the disease or condition at issue, as well as those who have not. Because subjects with cancer have heterogeneous clinical manifestations and varying clinical outcomes, the treatment given to a subject may vary, depending on his/her prognosis. The skilled clinician will be able to readily determine without undue experimentation specific secondary agents, types of surgery, and types of non-drug based standard therapy that can be effectively used to treat an individual subject with cancer.

As used herein, the term “cancer” includes, but is not limited to, solid tumors and blood borne tumors. The term “cancer” refers to disease of skin tissues, organs, blood, and vessels, including, but not limited to, cancers of the bladder, bone, blood, brain, breast, cervix, chest, colon, endrometrium, esophagus, eye, head, kidney, liver, lymph nodes, lung, mouth, neck, ovaries, pancreas, prostate, rectum, stomach, testis, throat, and uterus. Specific cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant giolma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma.

In certain embodiments, the cancer is a solid tumor. In certain embodiments, the solid tumor is metastatic. In certain embodiments, the solid tumor is drug-resistant. In certain embodiments, the solid tumor is hepatocellular carcinoma, prostate cancer, pancreatic cancer, lung cancer, breast cancer, ovarian cancer, colon cancer, small intestine cancer, biliary tract cancer, endometrium cancer, skin cancer (melanoma), cervix cancer, urinary tract cancer, or glioblastoma.

In certain embodiments, the cancer is a blood borne tumor. In certain embodiments, the blood borne tumor is metastatic. In certain embodiments, the blood borne tumor is drug resistant. In certain embodiments, the cancer is leukemia.

In one embodiment, methods provided herein encompass treating, preventing or managing various types of leukemias such as chronic lymphocytic leukemia (CLL), chronic myelocytic leukemia (CML), acute lymphoblastic leukemia (ALL), acute myeloid leukemia (AML), and acute myeloblastic leukemia (AML) by administering a therapeutically effective amount of a compound provided herein or a derivative thereof.

In some embodiments, the methods provided herein encompass treating, preventing or managing acute leukemia in a subject. In some embodiments, the acute leukemia is acute myeloid leukemia (AML), which includes, but is not limited to, undifferentiated AML (M0), myeloblastic leukemia (M1), myeloblastic leukemia (M2), promyelocytic leukemia (M3 or M3 variant (M3V)), myelomonocytic leukemia (M4 or M4 variant with eosinophilia (M4E)), monocytic leukemia (M5), erythroleukemia (M6), and megakaryoblastic leukemia (M7). In one embodiment, the acute myeloid leukemia is undifferentiated AML (M0). In one embodiment, the acute myeloid leukemia is myeloblastic leukemia (M1). In one embodiment, the acute myeloid leukemia is myeloblastic leukemia (M2). In one embodiment, the acute myeloid leukemia is promyelocytic leukemia (M3 or M3 variant (M3V)). In one embodiment, the acute myeloid leukemia is myelomonocytic leukemia (M4 or M4 variant with eosinophilia (M4E)). In one embodiment, the acute myeloid leukemia is monocytic leukemia (M5). In one embodiment, the acute myeloid leukemia is erythroleukemia (M6). In one embodiment, the acute myeloid leukemia is megakaryoblastic leukemia (M7). Thus, the methods of treating, preventing or managing acute myeloid leukemia in a subject comprise the step of administering to the subject an amount of a compound provided herein or a derivative thereof effective to treat, prevent or manage acute myeloid leukemia alone or in combination. In some embodiments, the methods comprise the step of administering to the subject a compound provided herein or a derivative thereof in combination with a second active agent in amounts effective to treat, prevent or manage acute myeloid leukemia.

In some embodiments, the methods provided herein encompass treating, preventing or managing acute lymphocytic leukemia (ALL) in a subject. In some embodiments, acute lymphocytic leukemia includes leukemia that originates in the blast cells of the bone marrow (B-cells), thymus (T-cells), and lymph nodes. The acute lymphocytic leukemia can be categorized according to the French-American-British (FAB) Morphological Classification Scheme as L1-Mature-appearing lymphoblasts (T cells or pre-B-cells), L2—Immature and pleomorphic (variously shaped) lymphoblasts (T-cells or pre-B-cells), and L3—Lymphoblasts (B-cells; Burkitt's cells). In one embodiment, the acute lymphocytic leukemia originates in the blast cells of the bone marrow (B-cells). In one embodiment, the acute lymphocytic leukemia originates in the thymus (T-cells). In one embodiment, the acute lymphocytic leukemia originates in the lymph nodes. In one embodiment, the acute lymphocytic leukemia is L1 type characterized by mature-appearing lymphoblasts (T-cells or pre-B-cells). In one embodiment, the acute lymphocytic leukemia is L2 type characterized by immature and pleomorphic (variously shaped) lymphoblasts (T-cells or pre-B-cells). In one embodiment, the acute lymphocytic leukemia is L3 type characterized by lymphoblasts (B-cells; Burkitt's cells). In certain embodiments, the acute lymphocytic leukemia is T cell leukemia. In one embodiment, the T-cell leukemia is peripheral T-cell leukemia. In another embodiment, the T-cell leukemia is T-cell lymphoblastic leukemia. In another embodiment, the T-cell leukemia is cutaneous T-cell leukemia. In another embodiment, the T-cell leukemia is adult T-cell leukemia. Thus, the methods of treating, preventing or managing acute lymphocytic leukemia in a subject comprise the step of administering to the subject an amount of a compound provided herein or a derivative thereof effective to treat, prevent or manage acute lymphocytic leukemia alone or in combination with a second active agent. In some embodiments, the methods comprise the step of administering to the subject a compound provided herein or a derivative thereof in combination with a second active agent in amounts effective to treat, prevent or manage acute lymphocytic leukemia.

In some embodiments, the methods provided herein encompass treating, preventing or managing chronic myelogenous leukemia (CML) in a subject. The methods comprise the step of administering to the subject an amount of a compound provided herein or a derivative thereof effective to treat, prevent or manage chronic myelogenous leukemia. In some embodiments, the methods comprise the step of administering to the subject a compound provided herein or a derivative thereof in combination with a second active agent in amounts effective to treat, prevent or manage chronic myelogenous leukemia.

In some embodiments, the methods provided herein encompass treating, preventing or managing chronic lymphocytic leukemia (CLL) in a subject. The methods comprise the step of administering to the subject an amount of a compound provided herein or a derivative thereof effective to treat, prevent or manage chronic lymphocytic leukemia. In some embodiments, the methods comprise the step of administering to the subject a compound provided herein or a derivative thereof in combination with a second active agent in amounts effective to treat, prevent or manage chronic lymphocytic leukemia.

In certain embodiments, provided herein are methods of treating, preventing, and/or managing disease in subjects with impaired renal function. In certain embodiments, provided herein are method of treating, preventing, and/or managing cancer in subjects with impaired renal function. In certain embodiments, provided herein are methods of providing appropriate dose adjustments for subjects with impaired renal function due to, but not limited to, disease, aging, or other subject factors.

In certain embodiments, provided herein are methods of treating, preventing, and/or managing lymphoma, including non-Hodgkin's lymphoma. In some embodiments, provided herein are methods for the treatment or management of non-Hodgkin's lymphoma (NHL), including but not limited to, diffuse large B-cell lymphoma (DLBCL), using prognostic factors.

In certain embodiments, provided herein are methods of treating, preventing, and/or managing multiple myeloma, including relapsed/refractory multiple myeloma in subjects with impaired renal function or a symptom thereof, comprising administering a therapeutically effective amount of a compound provided herein, or a derivative thereof to a subject having relapsed/refractory multiple myeloma with impaired renal function.

In some embodiments, the compound administered is a compound as disclosed herein of Formula I, IA, II, or III, or a pharmaceutically acceptable salt thereof. In some embodiments, the administered compound is a compound selected from Compounds 1-47, such as a compound selected from compounds 1-47 of Example 2, or a pharmaceutically acceptable salt thereof.

In certain embodiments, the subject to be treated with one of the methods provided herein has not been treated with anticancer therapy prior to the administration of the compound provided herein, or a derivative thereof. In certain embodiments, the subject to be treated with one of the methods provided herein has been treated with anticancer therapy prior to the administration of the compound provided herein, or a derivative thereof. In certain embodiments, the subject to be treated with one of the methods provided herein has developed drug resistance to the anticancer therapy.

The methods provided herein encompass treating a patient regardless of subject's age, although some diseases or disorders are more common in certain age groups.

### 6.4. Methods of Treating Inflammation

As discussed herein, activation of MAPKs, in particular ERK1/2, is a component of the inflammatory response. Thus, the compounds provided herein, which are ERK1/2 inhibitors via inhibition of Ras and/or a Ras superfamily member, are useful in the treatment of inflammatory diseases.

As discussed herein, activation of Akt is a component of the inflammatory response. Thus, the compounds provided herein, which are Akt inhibitors via inhibition of Ras and/or a Ras superfamily member, are useful in the treatment of inflammatory diseases.

In one embodiment, provided herein is a method of treating or preventing inflammatory disease, which comprises administering to a subject a compound that inhibits the function of one or more members of the Ras superfamily. In one embodiment, provided herein is a method of treating or preventing inflammatory disease, which comprises administering to a subject a compound that inhibits the function of one or more members of the Ras superfamily by binding to the GTP binding domain or one or more members of the Ras superfamily. In one embodiment, provided herein is a method of treating or preventing inflammatory disease, which comprises administering to a subject a compound that inhibits the function of Ras by binding to a Ras GTP binding domain. In another embodiment, provided herein is a method of treating or preventing inflammatory disease, which comprises administering to a subject a compound that binds to a Ras GTP binding domain with an IC50 of less than 10 μM and a Kd of less than 10 μM. In another embodiment, provided herein is a method of treating or preventing inflammatory disease, which comprises administering to a subject a compound that binds to a Ras GTP binding domain with an IC50 of less than 1 μM and a Kd of less than 1 μM. In another embodiment, provided herein is a method of treating or preventing inflammatory disease, which comprises administering to a subject a compound that binds to a Ras GTP binding domain with an IC50 of less than 500 nM and a Kd of less than 500 nM. In another embodiment, provided herein is a method of treating or preventing inflammatory disease, which comprises administering to a subject a compound that binds to a Ras GTP binding domain with an IC50 of less than 470 nM and a Kd of less than 470 nM. In another embodiment, provided herein is a method of treating or preventing inflammatory disease, which comprises administering to a subject a compound that binds to a Ras GTP binding domain with an IC50 of less than 270 nM and a Kd of less than 270 nM. In another embodiment, provided herein is a method of treating or preventing inflammatory disease, which comprises administering to a subject a compound that binds to a Ras GTP binding domain with an IC50 of less than 200 nM and a Kd of less than 200 nM. In another embodiment, provided herein is a method of treating or preventing inflammatory disease, which comprises administering to a subject a compound that binds to a Ras GTP binding domain with an IC50 of less than 150 nM and a Kd of less than 150 nM. In another embodiment, provided herein is a method of treating or preventing inflammatory disease, which comprises administering to a subject a compound that binds to a Ras GTP binding domain with an IC50 of less than 100 nM and a Kd of less than 100 nM. In another embodiment, provided herein is a method of treating or preventing inflammatory disease, which comprises administering to a subject a compound that binds to a Ras GTP binding domain with greater than 15% inhibition at 20 μM. In another embodiment, provided herein is a method of treating or preventing inflammatory disease, which comprises administering to a subject a compound that binds to a Ras GTP binding domain with greater than 25% inhibition at 20 μM. In another embodiment, provided herein is a method of treating or preventing inflammatory disease, which comprises administering to a subject a compound that binds to a Ras GTP binding domain with greater than 50% inhibition at 20 μM. In another embodiment, provided herein is a method of treating or preventing inflammatory disease, which comprises administering to a subject a compound that binds to a Ras GTP binding domain with greater than 75% inhibition at 20 μM. In another embodiment, provided herein is a method of treating or preventing inflammatory disease, which comprises administering to a subject a compound that binds to a Ras GTP binding domain with greater than 80% inhibition at 20 μM. In another embodiment, provided herein is a method of treating or preventing inflammatory disease, which comprises administering to a subject a compound that binds to a Ras GTP binding domain with greater than 85% inhibition at 20 μM. In another embodiment, provided herein is a method of treating or preventing inflammatory disease, which comprises administering to a subject a compound that binds to a Ras GTP binding domain with greater than 90% inhibition at 20 μM. In another embodiment, provided herein is a method of treating or preventing inflammatory disease, which comprises administering to a subject a compound that binds to a Ras GTP binding domain with greater than 95% inhibition at 20 μM. In another embodiment, provided herein is a method of treating or preventing inflammatory disease, which comprises administering to a subject a compound that binds to a Ras GTP binding domain with greater than 99% inhibition at 20 μM. In one embodiment, provided herein is a method of treating or preventing inflammatory disease, which comprises administering to a subject a compound that inhibits the function of Rho. In one embodiment, provided herein is a method of treating or preventing inflammatory disease, which comprises administering to a subject a compound that inhibits the function of Rho by binding to a Rho GTP binding domain. In another embodiment, provided herein is a method of treating or preventing inflammatory disease, which comprises administering to a subject a compound that binds to a Rho GTP binding domain with an IC50 of less than 10 μM and a Kd of less than 10 μM. In another embodiment, provided herein is a method of treating or preventing inflammatory disease, which comprises administering to a subject a compound that binds to a Rho GTP binding domain with an IC50 of less than 1 μM and a Kd of less than 1 μM. In another embodiment, provided herein is a method of treating or preventing inflammatory disease, which comprises administering to a subject a compound that binds to a Rho GTP binding domain with an IC50 of less than 500 nM and a Kd of less than 500 nM. In another embodiment, provided herein is a method of treating or preventing inflammatory disease, which comprises administering to a subject a compound that binds to a Rho GTP binding domain with an IC50 of less than 270 nM and a Kd of less than 270 nM. In another embodiment, provided herein is a method of treating or preventing inflammatory disease, which comprises administering to a subject a compound that binds to a Rho GTP binding domain with an IC50 of less than 200 nM and a Kd of less than 200 nM. In another embodiment, provided herein is a method of treating or preventing inflammatory disease, which comprises administering to a subject a compound that binds to a Rho GTP binding domain with an IC50 of less than 150 nM and a Kd of less than 150 nM. In another embodiment, provided herein is a method of treating or preventing inflammatory disease, which comprises administering to a subject a compound that binds to a Rho GTP binding domain with an IC50 of less than 130 nM and a Kd of less than 130 nM. In another embodiment, provided herein is a method of treating or preventing inflammatory disease, which comprises administering to a subject a compound that binds to a Rho GTP binding domain with an IC50 of less than 100 nM and a Kd of less than 100 nM. In another embodiment, provided herein is a method of treating or preventing inflammatory disease, which comprises administering to a subject a compound that binds to a Rho GTP binding domain with greater than 15% inhibition at 20 QM. In another embodiment, provided herein is a method of treating or preventing inflammatory disease, which comprises administering to a subject a compound that binds to a Rho GTP binding domain with greater than 25% inhibition at 20 μM. In another embodiment, provided herein is a method of treating or preventing inflammatory disease, which comprises administering to a subject a compound that binds to a Rho GTP binding domain with greater than 50% inhibition at 20 μM. In another embodiment, provided herein is a method of treating or preventing inflammatory disease, which comprises administering to a subject a compound that binds to a Rho GTP binding domain with greater than 75% inhibition at 20 μM. In another embodiment, provided herein is a method of treating or preventing inflammatory disease, which comprises administering to a subject a compound that binds to a Rho GTP binding domain with greater than 80% inhibition at 20 μM. In another embodiment, provided herein is a method of treating or preventing inflammatory disease, which comprises administering to a subject a compound that binds to a Rho GTP binding domain with greater than 85% inhibition at 20 μM. In another embodiment, provided herein is a method of treating or preventing inflammatory disease, which comprises administering to a subject a compound that binds to a Rho GTP binding domain with greater than 90% inhibition at 20 μM. In another embodiment, provided herein is a method of treating or preventing inflammatory disease, which comprises administering to a subject a compound that binds to a Rho GTP binding domain with greater than 95% inhibition at 20 μM. In another embodiment, provided herein is a method of treating or preventing inflammatory disease, which comprises administering to a subject a compound that binds to a Rho GTP binding domain with greater than 99% inhibition at 20 μM. In one embodiment, provided herein is a method of treating or preventing inflammatory disease, which comprises administering to a subject a compound that inhibits the function of Rac. In one embodiment, provided herein is a method of treating or preventing inflammatory disease, which comprises administering to a subject a compound that inhibits the function of Rae by binding to a Rac GTP binding domain. In another embodiment, provided herein is a method of treating or preventing inflammatory disease, which comprises administering to a subject a compound that binds to a Rac GTP binding domain with an IC50 of less than 10 μM and a Kd of less than 10 μM. In another embodiment, provided herein is a method of treating or preventing inflammatory disease, which comprises administering to a subject a compound that binds to a Rac GTP binding domain with an IC50 of less than 1 μM and a Kd of less than 1 μM. In another embodiment, provided herein is a method of treating or preventing inflammatory disease, which comprises administering to a subject a compound that binds to a Rac GTP binding domain with an IC50 of less than 500 nM and a Kd of less than 500 nM. In another embodiment, provided herein is a method of treating or preventing inflammatory disease, which comprises administering to a subject a compound that binds to a Rac GTP binding domain with an IC50 of less than 270 nM and a Kd of less than 270 nM. In another embodiment, provided herein is a method of treating or preventing inflammatory disease, which comprises administering to a subject a compound that binds to a Rae GTP binding domain with an IC50 of less than 200 nM and a Kd of less than 200 nM. In another embodiment, provided herein is a method of treating or preventing inflammatory disease, which comprises administering to a subject a compound that binds to a Rac GTP binding domain with an IC50 of less than 170 nM and a Kd of less than 170 nM. In another embodiment, provided herein is a method of treating or preventing inflammatory disease, which comprises administering to a subject a compound that binds to a Rac GTP binding domain with an IC50 of less than 150 nM and a Kd of less than 150 nM. In another embodiment, provided herein is a method of treating or preventing inflammatory disease, which comprises administering to a subject a compound that binds to a Rac GTP binding domain with an IC50 of less than 100 nM and a Kd of less than 100 nM. In one embodiment, provided herein is a method of treating or preventing inflammatory disease, which comprises administering to a subject a compound that binds to a Rac GTP binding domain with greater than 15% inhibition at 20 μM. In another embodiment, provided herein is a method of treating or preventing inflammatory disease, which comprises administering to a subject a compound that binds to a Rac GTP binding domain with greater than 25% inhibition at 20 μM. In another embodiment, provided herein is a method of treating or preventing inflammatory disease, which comprises administering to a subject a compound that binds to a Rac GTP binding domain with greater than 50% inhibition at 20 μM. In another embodiment, provided herein is a method of treating or preventing inflammatory disease, which comprises administering to a subject a compound that binds to a Rac GTP binding domain with greater than 75% inhibition at 20 μM. In another embodiment, provided herein is a method of treating or preventing inflammatory disease, which comprises administering to a subject a compound that binds to a Rac GTP binding domain with greater than 80% inhibition at 20 μM. In another embodiment, provided herein is a method of treating or preventing inflammatory disease, which comprises administering to a subject a compound that binds to a Rae GTP binding domain with greater than 85% inhibition at 20 μM. In another embodiment, provided herein is a method of treating or preventing inflammatory disease, which comprises administering to a subject a compound that binds to a Rac GTP binding domain with greater than 90% inhibition at 20 μM. In another embodiment, provided herein is a method of treating or preventing inflammatory disease, which comprises administering to a subject a compound that binds to a Rac GTP binding domain with greater than 95% inhibition at 20 μM. In another embodiment, provided herein is a method of treating or preventing inflammatory disease, which comprises administering to a subject a compound that binds to a Rac GTP binding domain with greater than 99% inhibition at 20 μM. In some embodiments, the compound for use in the method is a compound as disclosed herein of Formula I, IA, II, or III, or a pharmaceutically acceptable salt thereof. In some embodiments, the compound as disclosed herein for use in the method is a compound selected from Compounds 1-47, such as a compound selected from compounds 1-47 of Example 2, or a pharmaceutically acceptable salt thereof.

In another embodiment, provided herein is a method of treating or preventing inflammatory disease, which comprises administering to a subject a compound that binds to one or more of Ala11, Gly12, Val14, Gly15, Lys16, Ser17, Ala18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to two or more of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to three or more of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to four or more of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to five or more of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to six or more of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to seven or more of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to eight or more of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to nine or more of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to ten or more of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to eleven or more of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to twelve or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to thirteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to fourteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to fifteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to sixteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to seventeen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to eighteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to nineteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to twenty or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to twenty-one or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to twenty-two or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys 17, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to twenty-three or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to all of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In some embodiments, the compound for use in the method is a compound as disclosed herein of Formula I, IA, II, or III, or a pharmaceutically acceptable salt thereof. In some embodiments, the compound as disclosed herein for use in the method is a compound selected from Compounds 1-47, such as a compound selected from compounds 1-47 of Example 2, or a pharmaceutically acceptable salt thereof.

In one embodiment, the Ras is DIRAS1; DIRAS2; DIRAS3; ERAS; GEM; HRAS; KRAS; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; or RRAS2. In another embodiment, the Ras is HRAS, KRAS or NRAS. In one embodiment, the Ras is HRAS. In one embodiment, the Ras is KRAS. In one embodiment, the Ras is NRAS. In another embodiment, the Ras is a mutant form of a Ras described herein.

In another embodiment, provided herein is a method of treating or preventing inflammatory disease, which comprises administering to a subject a compound that binds to one or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to two or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to three or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to four or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to five or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to six or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to seven or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to eight or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain, In another embodiment, provided herein is a method of treating or preventing inflammatory disease, which comprises administering to a subject a compound that binds to nine or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to ten or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to eleven or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to twelve or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to thirteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to fourteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to fifteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to sixteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to seventeen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds all of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In some embodiments, the compound for use in the method is a compound as disclosed herein of Formula I, IA, II, or III, or a pharmaceutically acceptable salt thereof. In some embodiments, the compound as disclosed herein for use in the method is a compound selected from Compounds 1-47, such as a compound selected from compounds 1-47 of Example 2, or a pharmaceutically acceptable salt thereof.

In one embodiment, the Rho is RHOA; RHOB; RHOBTB1; RHOBTB2; RHOBTB3; RHOC; RHOD; RHOF; RHOG; RHOH; RHOJ; RHOQ; RHOU; RHOV; RND1; RND2; RND3; RAC1; RAC2; RAC3 or CDC42. In one embodiment, the Rho is RHOA. In another embodiment, the Rho is a mutant form of a Rho described herein.

In one embodiment, provided herein is a method of treating or preventing inflammatory disease, which comprises administering to a subject a compound that binds to one or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to two or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to three or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to four or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to five or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to six or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rae GTP binding domain. In one embodiment, provided herein is a method of treating or preventing inflammatory disease, which comprises administering to a subject a compound that binds to seven or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to eight or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to nine or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rae GTP binding domain. In one embodiment, provided herein is a method of treating or preventing inflammatory disease, which comprises administering to a subject a compound that binds to ten or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to eleven or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to twelve or more of Gly12, Ala 13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to thirteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to fourteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to fifteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rae GTP binding domain. In one embodiment, provided herein is a method of treating or preventing inflammatory disease, which comprises administering to a subject a compound that binds to sixteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to seventeen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to eighteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rae GTP binding domain. In one embodiment, provided herein is a method of treating or preventing inflammatory disease, which comprises administering to a subject a compound that binds to all of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In some embodiments, the compound for use in the method is a compound as disclosed herein of Formula I, IA, II, or III, or a pharmaceutically acceptable salt thereof. In some embodiments, the compound as disclosed herein for use in the method is a compound selected from Compounds 1-47, such as a compound selected from compounds 1-47 of Example 2, or a pharmaceutically acceptable salt thereof.

In one embodiment, the Rho is Rac. In one embodiment the Rac is RAC1; RAC2; RAC3 or RHOG. In one embodiment, the Rac is RAC1. In another embodiment, the Rac is a mutant form of a Rac described herein.

In one embodiment, the compound for use in the methods and compositions provided herein inhibit GTP binding to one or more members of the Ras superfamily. In one embodiment, the compound for use in the methods and compositions provided herein inhibit GTP binding to Ras. In one embodiment, the compounds provided herein inhibit GTP binding to Rho. In one embodiment, the compound for use in the methods and compositions provided herein inhibit GTP binding to Rac. In one embodiment, the compound for use in the methods and compositions provided herein inhibit GTP binding to Ras and Rho. In one embodiment, the compound for use in the methods and compositions provided herein inhibit GTP binding to Ras and Rac. In one embodiment, the compound for use in the methods and compositions provided herein inhibit GTP binding to Rho and Rac. In one embodiment, the compound for use in the methods and compositions provided herein inhibit GTP binding to Ras, Rho and Rac.

In one embodiment, the compound for use in the methods and compositions provided herein has a molecular weight less than 2000 daltons. In one embodiment, the compound for use in the methods and compositions provided herein has a molecular weight less than 1750 daltons. In one embodiment, the compound for use in the methods and compositions provided herein has a molecular weight less than 1500 daltons. In one embodiment, the compound for use in the methods and compositions provided herein has a molecular weight less than 1250 daltons. In one embodiment, the compound for use in the methods and compositions provided herein has a molecular weight less than 1000 daltons. In one embodiment, the compound for use in the methods and compositions provided herein has a molecular weight less than 750 daltons. In one embodiment, the compound for use in the methods and compositions provided herein has a molecular weight less than 665 daltons. In one embodiment, the compound for use in the methods and compositions provided herein has a molecular weight less than 500 daltons. In some embodiments, the compound for use in the method is a compound as disclosed herein of Formula I, IA, II, or III, or a pharmaceutically acceptable salt thereof. In some embodiments, the compound as disclosed herein for use in the method is a compound selected from Compounds 1-47, such as a compound selected from compounds 1-47 of Example 2, or a pharmaceutically acceptable salt thereof.

In one embodiment, the inflammatory disease is inflammation-associated cancer development. As disclosed here, the compounds provided herein are useful in treatment of cancer. It is well recognized that the immune inflammatory state serves as a key mediator of the middle stages of tumor development. It is also well known that chronic inflammation can predispose an individual to cancer. Chronic inflammation is caused by a variety of factors, including bacterial, viral, and parasitic infections. The longer the inflammation persists, the higher the risk of associated carcinogenesis. Anti-inflammatory cancer therapy prevents premalignant cells from turning fully cancerous or impedes existing tumors from spreading to distant sites in the body. Thus, in one embodiment, the compounds provided herein are useful in treating inflammatory cancers. Such cancers, and the chronic inflammatory conditions that predispose susceptible cells to neoplastic transformation, include gastric adenocarcinoma (gastritis), mucosa-associated lymphoid tissue (MALT) lymphoma (gastritis), bladder, liver and rectal carcinomas (schistosomiasis), cholangiocarcinoma and colon carcinoma (cholangitis), gall bladder cancer (chronic cholecystitis), ovarian and cervical carcinoma (pelvic inflammatory disease, chronic cervicitis), skin carcinoma (osteomyelitis), colorectal carcinoma (inflammatory bowel disease), esophageal carcinoma (reflux esophagitis, Barrett's esophagus), bladder cancer (bladder inflammation (cystitis)), mesothelioma and lung carcinoma (asbestosis, silicosis), oral squamous cell carcinoma (gingivitis, lichen planus), pancreatic carcinoma (pancreatitis, protease mutation), vulvar squamous cell carcinoma (lichen sclerosis), salivary gland carcinoma (slaladenitis), lung carcinoma (bronchitis) and MALT lymphoma (Sjogren syndrome, Hashimoto's thyroiditis). Shacter, et al., 2002, Oncology, 16(2), 217-26.

In certain embodiments, the compounds provided herein are useful in treating inflammatory diseases in the airways, such as nonspecific bronchial hyper-reactivity, chronic bronchitis, cystic fibrosis, and acute respiratory distress syndrome (ARDS).

In certain embodiments, the compounds provided herein are useful in treating asthma and idiopathic lung fibrosis or idiopathic pulmonary fibrosis (IPF), pulmonary fibrosis, and interstitial lung disease. As known to one of skill in the art, the differentiation of fibroblasts into cell types called myofibroblasts occurs during wound healing, when the cells contribute to the deposition of extracellular matrix (ECM) in the transient process of wound repair. In chronic inflammatory diseases such as asthma, pathological tissue remodeling often occurs, and is mediated by the functions of increased numbers of myofibroblasts in the diseased tissue, see Hinz, B. et al. Am J Pathol. 2007; 170: 1807-1816. In certain embodiments, the compounds provided herein prevent or reduce TGF-β-induced myofibroblast differentiation, as measured by the expression of alpha smooth muscle actin (α-SMA), a hallmark of myofibroblast differentiation (Serini, G. and Gabbiani, G. 1999; Exp. Cell Res. 250: 273-283).

In certain embodiments, the compounds provided herein are useful in treating psoriasis, chronic plaque psoriasis, psoriatic arthritis, acanthosis, atopic dermatitis, various forms of eczema, contact dermatitis (includes allergic dermatitis), systemic sclerosis (scleroderma), wound healing, and drug eruption.

In one embodiment, the disease is inflammation, arthritis, rheumatoid arthritis, spondylarthropathies, gouty arthritis, osteoarthritis, juvenile arthritis, and other arthritic conditions, systemic lupus erthematosus (SLE), skin-related conditions, eczema, Sjogren's syndrome, burns, dermatitis, neuroinflammation, allergy pain, autoimmune myositis, neuropathic pain, fever, pulmonary disorders, lung inflammation, adult respiratory distress syndrome, pulmonary sarcoisosis, asthma, silicosis, chronic pulmonary inflammatory disease, and chronic obstructive pulmonary disease (COPD), cardiovascular disease, arteriosclerosis, myocardial infarction (including post-myocardial infarction indications), thrombosis, congestive heart failure, cardiac reperfusion injury, as well as complications associated with hypertension and/or heart failure such as vascular organ damage, restenosis, cardiomyopathy, stroke including ischemic and hemorrhagic stroke, reperfusion injury, renal reperfusion injury, ischemia including stroke and brain ischemia, and ischemia resulting from cardiac/coronary bypass, neurodegenerative disorders, liver disease and nephritis, gastrointestinal conditions, inflammatory bowel disease, Crohn's disease, gastritis, irritable bowel syndrome, ulcerative colitis, ulcerative diseases, gastric ulcers, viral and bacterial infections, sepsis, septic shock, gram negative sepsis, malaria, meningitis, HIV infection, opportunistic infections, cachexia secondary to infection or malignancy, cachexia secondary to acquired immune deficiency syndrome (AIDS), AIDS, ARC (AIDS related complex), pneumonia, herpes virus, myalgias due to infection, influenza, autoimmune disease, graft vs. host reaction and allograft rejections, treatment of bone resorption diseases, osteoporosis, multiple sclerosis, acute gout, pneumonitis, myocarditis, pericarditis, myositis, eczema, alopecia, vitiligo, bullous skin diseases, atherosclerosis, depression, retinitis, uveitis, scleritis, hepatitis, pancreatitis, primary biliary cirrhosis, sclerosing cholangitis, Addison's disease, hypophysitis, thyroiditis, type I diabetes, giant cell arteritis, nephritis including lupus nephritis, vasculitis with organ involvement such as glomerulonephritis, vasculitis including giant cell arteritis, Wegener's granulomatosis, Polyarteritis nodosa, Behcet's disease, Kawasaki disease, Takayasu's Arteritis, vasculitis with organ involvement, acute rejection of transplanted organs. endotoxaemia, systemic inflammatory response syndrome (SIRS), multi-organ dysfunction syndrome, toxic shock syndrome, acute lung injury, ARDS (adult respiratory distress syndrome), acute renal failure, fulminant hepatitis, burns, acute pancreatitis, postsurgical syndromes, sarcoidosis, Herxheimer reactions, encephalitis, myelitis, SIRS associated with viral infections such as influenza, herpes zoster, herpes simplex, coronavirus or dry eye syndrome (or keratoconjunctivitis sicca (KCS)).

In certain embodiments, the compounds provided herein are useful in treating neuropathic and nociceptive pain, chronic or acute, such as, without limitation, allodynia, inflammatory pain, inflammatory hyperalgesia, post herpetic neuralgia, neuropathies, neuralgia, diabetic neuropathy, HIV-related neuropathy, nerve injury, rheumatoid arthritic pain, osteoarthritic pain, burns, back pain, ocular pain, visceral pain, cancer pain, dental pain, headache, migraine, carpal tunnel syndrome, fibromyalgia, neuritis, sciatica, pelvic hypersensitivity, pelvic pain, post operative pain, post stroke pain, and menstrual pain.

In certain embodiments, the compounds provided herein are useful in treating Alzheimer's disease (AD), mild cognitive impairment (MCI), age-associated memory impairment (AAMI), multiple sclerosis, Parkinson's disease, vascular dementia, senile dementia, AIDS dementia, Pick's disease, dementia caused by cerebrovascular disorders, corticobasal degeneration, amyotrophic lateral sclerosis (ALS), Huntington's disease, diminished CNS function associated with traumatic brain injury.

In one embodiment, the compounds provided herein are useful in treating Alzheimer's disease (AD), ankylosing spondylitis, arthritis (osteoarthritis, rheumatoid arthritis (RA), psoriatic arthritis), asthma, atherosclerosis, Crohn's disease, colitis, dermatitis, diverticulitis, fibromyalgia, hepatitis, irritable bowel syndrome (IBS), systemic lupus, erythematous (SLE), nephritis, Parkinson's disease, ulcerative colitis.

### 6.5. Pharmaceutical Compositions

The pharmaceutical compositions provided herein contain therapeutically effective amounts of one or more of compounds provided herein and a pharmaceutically acceptable carrier, diluent or excipient.

The compounds can be formulated into suitable pharmaceutical preparations such as solutions, suspensions, tablets, dispersible tablets, pills, capsules, powders, sustained release formulations or elixirs, for oral administration or in sterile solutions or suspensions for ophthalmic or parenteral administration, as well as transdermal patch preparation and dry powder inhalers. Typically the compounds described above are formulated into pharmaceutical compositions using techniques and procedures well known in the art (see, e.g., Ansel Introduction to Pharmaceutical Dosage Forms, Seventh Edition 1999).

In the compositions, effective concentrations of one or more compounds or pharmaceutically acceptable salts is (are) mixed with a suitable pharmaceutical carrier or vehicle. In certain embodiments, the concentrations of the compounds in the compositions are effective for delivery of an amount, upon administration, that treats, prevents, or ameliorates one or more of the symptoms and/or progression of a disease or disorder disclosed herein.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the subject treated. The therapeutically effective concentration may be determined empirically by testing the compounds in in vitro and in vivo systems described herein and then extrapolated therefrom for dosages for humans. In some embodiments, the active compound is administered in a method to achieve a therapeutically effective concentration of the drug. In some embodiments, a companion diagnostic (see, e.g., Olsen D and Jorgensen J T, Front. Oncol., 2014 May 16, 4:105, doi: 10.3389/fonc.2014.00105) is used to determine the therapeutic concentration and safety profile of the active compound in specific subjects or subject populations.

The concentration of active compound in the pharmaceutical composition will depend on absorption, tissue distribution, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of a disease or disorder disclosed herein.

In certain embodiments, a therapeutically effective dosage should produce a serum concentration of active ingredient of from about 0.1 ng/mL to about 50-100 μg/mL. In one embodiment, the pharmaceutical compositions provide a dosage of from about 0.001 mg to about 2000 mg of compound per kilogram of body weight per day. Pharmaceutical dosage unit forms are prepared to provide from about 1 mg to about 1000 mg and in certain embodiments, from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of one or more of the compounds described herein or pharmaceutically acceptable salts thereof are mixed with a suitable pharmaceutical carrier or vehicle for systemic, topical or local administration to form pharmaceutical compositions. Compounds are included in an amount effective for ameliorating one or more symptoms of, or for treating, retarding progression, or preventing. The concentration of active compound in the composition will depend on absorption, tissue distribution, inactivation, excretion rates of the active compound, the dosage schedule, amount administered, particular formulation as well as other factors known to those of skill in the art.

The compositions are intended to be administered by a suitable route, including but not limited to oral, parenteral, subcutaneous, intravenous, intramuscular, intraperitoneal, intrathecal, mucosal, dermal, transdermal, buccal, rectal, topical, local, nasal or inhalation. For oral administration, capsules and tablets can be formulated. The compositions are in liquid, semi-liquid or solid form and are formulated in a manner suitable for each route of administration.

Solutions or suspensions used for parenteral, intradermal, subcutaneous, or topical application can include any of the following components. a sterile diluent, such as water for injection, saline solution, fixed oil, polyethylene glycol, glycerine, propylene glycol, dimethyl acetamide or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, pens, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable salts thereof. The pharmaceutically therapeutically active compounds and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include iontophoresis patches, polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Dosage forms or compositions containing active ingredient in the range of 0.005% to 100% with the balance made up from non toxic carrier may be prepared. For oral administration, a pharmaceutically acceptable non toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001% to 100% active ingredient, in certain embodiments, about 0.1 85% or about 75-95%.

The active compounds or pharmaceutically acceptable salts may be prepared with carriers that protect the compound against rapid elimination from the body, such as time release formulations or coatings.

The compositions may include other active compounds to obtain desired combinations of properties. The compounds provided herein, or pharmaceutically acceptable salts thereof as described herein, may also be advantageously administered for therapeutic or prophylactic purposes together with another pharmacological agent known in the general art to be of value in treating one or more of the diseases or medical conditions referred to hereinabove, such as diseases related to oxidative stress. It is to be understood that such combination therapy constitutes a further aspect of the compositions and methods of treatment provided herein.

Lactose-free compositions provided herein can contain excipients that are well known in the art and are listed, for example, in the U.S. Pharmocopia (USP) SP (XXI)/NF (XVI). In general, lactose-free compositions contain an active ingredient, a binder/filler, and a lubricant in pharmaceutically compatible and pharmaceutically acceptable amounts. Exemplary lactose-free dosage forms contain an active ingredient, microcrystalline cellulose, pre-gelatinized starch and magnesium stearate.

Further encompassed are anhydrous pharmaceutical compositions and dosage forms containing a compound provided herein. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, NY, 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs and strip packs.

### Oral Dosage Forms

Oral pharmaceutical dosage forms are either solid, gel or liquid. The solid dosage forms are tablets, capsules, granules, and bulk powders. Types of oral tablets include compressed, chewable lozenges and tablets which may be enteric coated, sugar coated or film coated. Capsules may be hard or soft gelatin capsules, while granules and powders may be provided in non-effervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable salt thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric coated tablets, because of the enteric coating, resist the action of stomach acid and dissolve or disintegrate in the neutral or alkaline intestines. Sugar coated tablets are compressed tablets to which different layers of pharmaceutically acceptable substances are applied. Film coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil in-water or water in oil. In some embodiments, the suspension is a suspension of microparticles or nanoparticles. In some embodiments, the emulsion is an emulsion of microparticles or nanoparticles.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

### Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. In some embodiments, the suspension is a suspension of microparticles or nanoparticles. In some embodiments, the emulsion is an emulsion of microparticles or nanoparticles. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow release or sustained release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the subject or animal as is known in the art.

The unit dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

### Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (including but not limited to 10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected compound. Such amount can be empirically determined.

### Topical Administration

Topical mixtures are prepared as described for the local and systemic administration. The resulting mixture may be a solution, suspension, emulsion or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable salts thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of less than 50 microns or less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

### Compositions for Other Routes of Administration

Other routes of administration, such as topical application, transdermal patches, and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin gelatin, carbowax (polyoxyethylene glycol) and appropriate mixtures of mono, di and triglycerides of fatty acids. Combinations of the various bases may be used. Agents to raise the melting point of suppositories include spermaceti and wax. Rectal suppositories may be prepared either by the compressed method or by molding. An exemplary weight of a rectal suppository is about 2 to 3 grams.

Tablets and capsules for rectal administration are manufactured using the same pharmaceutically acceptable substance and by the same methods as for formulations for oral administration.

### Sustained Release Compositions

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461,6,419,961, 6,589,548, 6,613,358, 6,699,500 and 6,740,634, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. In one embodiment, the use of an optimally designed controlled-release preparation in medical treatment is characterized by a minimum of drug substance being employed to cure or control the condition in a minimum amount of time. In certain embodiments, advantages of controlled-release formulations include extended activity of the drug, reduced dosage frequency, and increased subject compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

### Targeted Formulations

The compounds provided herein, or pharmaceutically acceptable salts thereof, may also be formulated to be targeted to a particular tissue, receptor, or other area of the body of the subject to be treated, including liposome-, resealed erythrocyte-, and antibody-based delivery systems. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, the antibody-based delivery system is an antibody-drug conjugate (“ADC”), e.g., as described in Hamilton G S, Biologicals, 2015 September, 43(5):318-32; Kim E G and Kim K M, Biomol. Ther. (Seoul), 2015 November, 23(6):493-509; and Peters C and Brown S, Biosci. Rep., 2015 Jun. 12, 35(4) pii: e00225, each of which is incorporated herein by reference.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

### Articles of Manufacture

The compounds or pharmaceutically acceptable salts can be packaged as articles of manufacture containing packaging material, a compound or pharmaceutically acceptable salt thereof provided herein, which is used for treatment, prevention or amelioration of one or more symptoms or progression of a disease or disorder disclosed herein, and a label that indicates that the compound or pharmaceutically acceptable salt thereof is used for treatment, prevention or amelioration of one or more symptoms or progression of a disease or disorder disclosed herein.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, pens, bottles, and any packaging material suitable for a selected formulation and intended mode of administration and treatment. A wide array of formulations of the compounds and compositions provided herein are contemplated.

In certain embodiments, provided herein also are kits which, when used by the medical practitioner, can simplify the administration of appropriate amounts of active ingredients to a subject. In certain embodiments, the kit provided herein includes a container and a dosage form of a compound provided herein, including a single enantiomer or a mixture of diastereomers thereof; or a pharmaceutically acceptable salt, solvate, or prodrug thereof.

In certain embodiments, the kit includes a container comprising a dosage form of the compound provided herein, including a single enantiomer or a mixture of diastereomers thereof; or a pharmaceutically acceptable salt, solvate, or prodrug thereof, in a container comprising one or more other therapeutic agent(s) described herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

### 6.6. Dosing

The compounds and pharmaceutical compositions provided herein may be dosed in certain therapeutically or prohylactically effective amounts, certain time intervals, certain dosage forms, and certain dosage administration methods as described below.

In certain embodiments, a therapeutically or prophylactically effective amount of the compound is from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 0.02 to about 25 mg per day, from about 0.05 to about 10 mg per day, from about 0.05 to about 5 mg per day, from about 0.1 to about 5 mg per day, or from about 0.5 to about 5 mg per day.

In certain embodiments, the therapeutically or prophylactically effective amount is about 0.1, about 0.2, about 0.5, about 1, about 2, about 3, about 4, about 5, about 6, about 7, about 8, about 9, about 10, about 15, about 20, about 25, about 30, about 40, about 45, about 50, about 60, about 70, about 80, about 90, about 100, or about 150 mg per day.

In one embodiment, the recommended daily dose range of the compound provided herein, or a derivative thereof, for the conditions described herein lie within the range of from about 0.5 mg to about 50 mg per day, in one embodiment given as a single once-a-day dose, or in divided doses throughout a day. In some embodiments, the dosage ranges from about 1 mg to about 50 mg per day. In other embodiments, the dosage ranges from about 0.5 to about 5 mg per day. Specific doses per day include 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg per day.

In a specific embodiment, the recommended starting dosage may be 0.5, 1, 2, 3, 4, 5, 10, 15, 20, 25 or 50 mg per day. In another embodiment, the recommended starting dosage may be 0.5, 1, 2, 3, 4, or 5 mg per day. The dose may be escalated to 15, 20, 25, 30, 35, 40, 45 and 50 mg/day. In a specific embodiment, the compound can be administered in an amount of about 25 mg/day. In a particular embodiment, the compound can be administered in an amount of about 10 mg/day. In a particular embodiment, the compound can be administered in an amount of about 5 mg/day. In a particular embodiment, the compound can be administered in an amount of about 4 mg/day. In a particular embodiment, the compound can be administered in an amount of about 3 mg/day.

In certain embodiments, the therapeutically or prophylactically effective amount is from about 0.001 to about 100 mg/kg/day, from about 0.01 to about 50 mg/kg/day, from about 0.01 to about 25 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, from about 0.01 to about 1 mg/kg/day, or from about 0.01 to about 0.05 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, e.g., Nair A B, Jacob S. A simple practice guide for dose conversion between animals and human. J Basic Clin Pharma 2016; 7.27-31). For example, a dose of 1 mg/kg/day for a 60 kg human is approximately equal to 37 mg/m²/day.

In certain embodiments, the amount of the compound administered is sufficient to provide a plasma concentration of the compound at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 M.

In other embodiments, the amount of the compound administered is sufficient to provide a plasma concentration of the compound at steady state, ranging from about 5 to about 100 nM, about 5 to about 50 nM, about 10 to about 100 nM, about 10 to about 50 nM or from about 50 to about 100 nM.

As used herein, the term “plasma concentration at steady state” is the concentration reached after a period of administration of a compound provided herein, or a derivative thereof. Once steady state is reached, there are minor peaks and troughs on the time dependent curve of the plasma concentration of the compound.

In certain embodiments, the amount of the compound administered is sufficient to provide a maximum plasma concentration (peak concentration) of the compound, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 M.

In certain embodiments, the amount of the compound administered is sufficient to provide a minimum plasma concentration (trough concentration) of the compound, ranging from about 0.001 to about 500 M, about 0.002 to about 200 μM, about 0.005 to about 100 μM, about 0.01 to about 50 M, from about 1 to about 50 μM, about 0.01 to about 25 μM, from about 0.01 to about 20 μM, from about 0.02 to about 20 μM, from about 0.02 to about 20 μM, or from about 0.01 to about 20 μM.

In certain embodiments, the amount of the compound administered is sufficient to provide an area under the curve (AUC) of the compound, ranging from about 100 to about 100,000 ng*hr/mL, from about 1,000 to about 50,000 ng*hr/mL, from about 5,000 to about 25,000 ng*hr/mL, or from about 5,000 to about 10,000 ng*hr/mL.

The methods provided herein encompass treating a patient regardless of subject's age, although some diseases or disorders are more common in certain age groups.

Depending on the disease to be treated and the subject's condition, the compound provided herein, or a derivative thereof, may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. The compound provided herein, or a derivative thereof, may be formulated, alone or together, in suitable dosage unit with pharmaceutically acceptable excipients, carriers, adjuvants and vehicles, appropriate for each route of administration.

In one embodiment, the compound provided herein, or a derivative thereof, is administered orally. In another embodiment, the compound provided herein, or a derivative thereof, is administered parenterally. In yet another embodiment, the compound provided herein, or a derivative thereof, is administered intravenously.

The compound provided herein, or a derivative thereof, can be delivered as a single dose such as, e.g., a single bolus injection, or oral tablets or pills; or over time, such as, e.g., continuous infusion over time or divided bolus doses over time. The compound can be administered repeatedly if necessary, for example, until the subject experiences stable disease or regression, or until the subject experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 92(3): 205 216 (2000). Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

The compound provided herein, or a derivative thereof, can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as the compound provided herein, or a derivative thereof, is administered once or more than once each day, for example, for a period of time. The term “continuous” is intended to mean that a therapeutic compound, such as the compound provided herein or a derivative thereof, is administered daily for an uninterrupted period of at least 10 days to 52 weeks. The term “intermittent” or “intermittently” as used herein is intended to mean stopping and starting at either regular or irregular intervals. For example, intermittent administration of the compound provided herein or a derivative thereof is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as the compound provided herein or a derivative thereof, is administered daily or continuously but with a rest period. In some such embodiments, administration is once a day for two to six days, then a rest period with no administration for five to seven days.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, the compound provided herein, or a derivative thereof, is administered once a day. In another embodiment, the compound provided herein, or a derivative thereof, is administered twice a day. In yet another embodiment, the compound provided herein, or a derivative thereof, is administered three times a day. In still another embodiment, the compound provided herein, or a derivative thereof, is administered four times a day.

In certain embodiments, the compound provided herein, or a derivative thereof, is administered once per day from one day to six months, from one week to three months, from one week to four weeks, from one week to three weeks, or from one week to two weeks. In certain embodiments, the compound provided herein, or a derivative thereof, is administered once per day for one week, two weeks, three weeks, or four weeks. In one embodiment, the compound provided herein, or a derivative thereof, is administered once per day for 4 days. In one embodiment, the compound provided herein, or a derivative thereof, is administered once per day for 5 days. In one embodiment, the compound provided herein, or a derivative thereof, is administered once per day for 6 days. In one embodiment, the compound provided herein, or a derivative thereof, is administered once per day for one week. In another embodiment, the compound provided herein, or a derivative thereof, is administered once per day for two weeks. In yet another embodiment, the compound provided herein, or a derivative thereof, is administered once per day for three weeks. In still another embodiment, the compound provided herein, or a derivative thereof, is administered once per day for four weeks.

Combination Therapy with a Second Active Agent

The compound provided herein, or a derivative thereof, can also be combined or used in combination with other therapeutic agents useful in the treatment and/or prevention of cancers, inflammatory diseases, rasopathies, or fibrotic disease.

In one embodiment, provided herein is a method of treating, preventing, or managing cancers, inflammatory diseases, rasopathies, and fibrotic disease, comprising administering to a subject a compound provided herein, or a derivative thereof, in combination with one or more second active agents.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein, a compound provided herein, e.g., the compound provided herein, or a derivative thereof) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

Administration of the compound provided herein, or a derivative thereof and one or more second active agents to a subject can occur simultaneously or sequentially by the same or different routes of administration. The suitability of a particular route of administration employed for a particular active agent will depend on the active agent itself (e.g., whether it can be administered orally without decomposing prior to entering the blood stream) and the disease or disorder being treated.

The route of administration of the compound provided herein, or a derivative thereof, is independent of the route of administration of a second therapy. In one embodiment, the compound provided herein, or a derivative thereof, is administered orally. In another embodiment, the compound provided herein, or a derivative thereof, is administered intravenously. Thus, in accordance with these embodiments, the compound provided herein, or a derivative thereof, is administered orally or intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In one embodiment, the compound provided herein, or a derivative thereof, and a second therapy are administered by the same mode of administration, orally or by IV. In another embodiment, the compound provided herein, or a derivative thereof, is administered by one mode of administration, e.g., by IV, whereas the second agent is administered by another mode of administration, e.g., orally.

In one embodiment, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated or managed, the severity and stage of disease, and the amount of the compound provided herein, or a derivative thereof, and any optional additional active agents concurrently administered to the subject.

One or more second active ingredients or agents can be used together with the compound provided herein, or a derivative thereof, in the methods and compositions provided herein. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies, particularly, therapeutic antibodies to cancer antigens. Typical large molecule active agents are biological molecules, such as naturally occurring or synthetic or recombinant proteins.

In one embodiment, the compound provided herein, or a derivative thereof, can be administered in an amount ranging from about 0.1 to about 150 mg, from about 1 to about 25 mg, or from about 2 to about 10 mg orally and daily alone, or in combination with a second active agent, prior to, during, or after the use of conventional therapy.

### 7. EXAMPLES

The present disclosure will now be described with reference to specific example(s) which should not be construed as in any way limiting.

### Example 1

### Synthesis of Compounds 20-47

### Compound 20

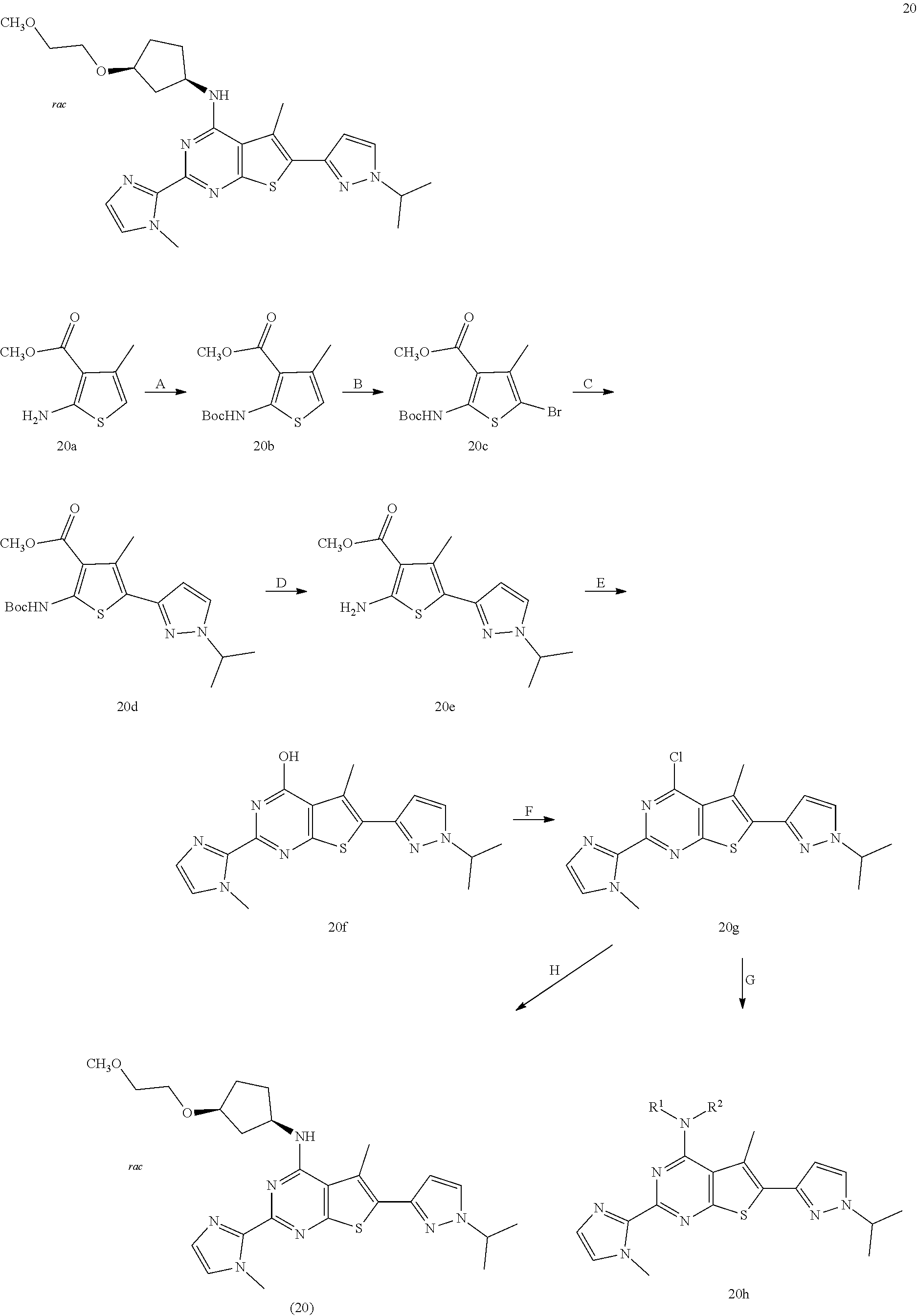

### Step A: Methyl 2-[(tert-butoxy)carbonyl]amino-4-methylthiophene-3-carboxylate 20b

DMAP (642.19 mg, 5.26 mmol) was added to a stirred solution of methyl 2-amino-4-methylthiophene-3-carboxylate 20a (9.0 g, 52.56 mmol) and Boc₂O (13.77 g, 63.08 mmol) in 108 mL of dioxane. The mixture was refluxed overnight. After 16 h reaction was complete (monitored by NMR). The mixture was concentrated under reduced pressure and purified by column chromatography (Eluted by Hex:EtOAc 10:1). Methyl 2-[(tert-butoxy)carbonyl]amino-4-methylthiophene-3-carboxylate 20b (8.1 g, 29.85 mmol, 56.8% yield) was obtained as white solid.

### Step B: Methyl 5-bromo-2-[(tert-butoxy)carbonyl]amino-4-methylthiophene-3-carboxylate 20c

To a solution of methyl 2-[(tert-butoxy)carbonyl]amino-4-methylthiophene-3-carboxylate 20b (8.1 g, 29.85 mmol) in 160 mL of DMF, N-bromosuccinimide (6.38 g, 35.82 mmol) was added in one portion. The resulting mixture was stirred at room temperature overnight. Then, the mixture was allowed to cool to room temperature and poured in ice water (350 mL). The obtained precipitate was filtered, washed 3 times with water and dried on air. Methyl 5-bromo-2-[(tert-butoxy)carbonyl]amino-4-methylthiophene-3-carboxylate 20c (9.2 g, 26.27 mmol, 88% yield) was obtained as light-yellow powder.

### Step C: Methyl 2-((tert-butoxycarbonyl)amino)-5-(1-isopropyl-1H-pyrazol-3-yl)-4-methylthiophene-3-carboxylate 20d

Methyl 5-bromo-2-[(tert-butoxy)carbonyl]amino-4-methylthiophene-3-carboxylate 20c (2.0 g, 5.71 mmol), 1-(propan-2-yl)-3-(tetramethyl-1,3,2-dioxaborolan-2-yl)-1H-pyrazole (1.62 g, 6.85 mmol), cesium carbonate (3.72 g, 11.42 mmol) and (2-dicyclohexylphosphino-2′,4′,6′-triisopropyl-1,1′-biphenyl)[2-(2′-amino-1,1′-biphenyl)]palladium(II) methanesulfonate (966.74 mg, 1.14 mmol) were mixed together in degassed dioxane:H₂O (40 mL:2 mL) and refluxed overnight. Then, the mixture was allowed to cool to room temperature, filtered and concentrated under reduced pressure. The residue was purified by column chromatography (SiO₂; Elute by Hexane:EtOAc 10:1 to 1:10). Methyl 2-((tert-butoxycarbonyl)amino)-5-(1-isopropyl-1H-pyrazol-3-yl)-4-methylthiophene-3-carboxylate 20d (1.2 g, 90.0% purity, 2.85 mmol, 49.8% yield) was obtained as orange powder.

### Step D: Methyl 2-amino-5-(1-isopropyl-1H-pyrazol-3-yl)-4-methylthiophene-3-carboxylate 20e

Methyl 2-((tert-butoxycarbonyl)amino)-5-(1-isopropyl-1H-pyrazol-3-yl)-4-methylthiophene-3-carboxylate 20d (1.2 g, 3.16 mmol) was dissolved in 5 mL of dioxane and 6M dioxane*HCl was added dropwise at 0° C. The reaction mixture was stirred overnight at room temperature. Then, the volatiles were removed in vacuo and the residue was purified by flash chromatography (SiO2; Eluted by EtOAc). Methyl 2-amino-5-(1-isopropyl-1H-pyrazol-3-yl)-4-methylthiophene-3-carboxylate 20e (530.0 mg, 90.0% purity, 1.71 mmol, 57.3% yield) was obtained as dark-grey gum.

### Step E: 6-(1-Isopropyl-1H-pyrazol-3-yl)-5-methyl-2-(1-methyl-1H-imidazol-2-yl)thieno[2,3-d]pyrimidin-4-ol 20f

Methyl 2-amino-5-(1-isopropyl-1H-pyrazol-3-yl)-4-methylthiophene-3-carboxylate 20e (430.35 mg, 1.54 mmol), 1-methyl-1H-imidazole-2-carbonitrile (247.51 mg, 2.31 mmol) and potassium 2-methylpropan-2-olate (691.45 mg, 6.16 mmol) were dissolved in dry MeOH (5 mL). The reaction mixture was stirred overnight at room temperature. The residue was evaporated under reduced pressure and HOAc (conc) was added to pH 5. Then, the solution was mixed with EtOAc (15 mL) and extracted. The organic phase was dried over Na₂SO₄ and concentrated under reduced pressure to afford 6-(1-isopropyl-1H-pyrazol-3-yl)-5-methyl-2-(1-methyl-1H-imidazol-2-yl)thieno[2,3-d]pyrimidin-4-ol 20f (400.0 mg, 80.0% purity, 902.86 μmol, 58.6% yield).

### Step F: 4-Chloro-6-(1-isopropyl-1H-pyrazol-3-yl)-5-methyl-2-(1-methyl-1H-imidazol-2-yl)thieno[2,3-d]pyrimidine 20g

The crude material from the previous step (6-(1-isopropyl-1H-pyrazol-3-yl)-5-methyl-2-(1-methyl-1H-imidazol-2-yl)thieno[2,3-d]pyrimidin-4-ol 20f) (100.0 mg, 282.14 μmol)) was suspended in POCl₃ (2 mL) and diisopropylethylamine (0.6 mL) was added at room temperature. The reaction mixture was stirred under reflux for 16 h. The mixture was allowed to cool to the room temperature and evaporated under reduced pressure. The residue was poured into ice and basified with liquid ammonia (20 mL, 20-25% of ammonia) and filtered on to afford 4-chloro-6-(1-isopropyl-1H-pyrazol-3-yl)-5-methyl-2-(1-methyl-1H-imidazol-2-yl)thieno[2,3-d]pyrimidine 20g (100.0 mg, 55.0% purity, 147.5 μmol, 52.4% yield) as brown liquid.

### Step G: General Procedure: To the solution of 3-[4-chloro-5-methyl-2-(1-methyl-1H-imidazol-2-yl)thieno[2,3-d]pyrimidin-6-yl]-1-(propan-2-yl)-1H-pyrazole (20g) (1.07 mmol) in DMSO (7 mL), the appropriate corresponding amine (1.61 mmol) and ethylbis(propan-2-yl)amine (3.22 mmol) were added at room temperature. The reaction mixture was stirred at 100° C. overnight. Then, the resulting mixture was cooled and purified by HPLC

### Step H: rac 6-(1-Isopropyl-1H-pyrazol-3-yl)-N-((1R,3S)-3-(2-methoxyethoxy)cyclopentyl)-5-methyl-2-(1-methyl-1H-imidazol-2-yl)thieno[2,3-d]pyrimidin-4-amine 20

Following the general procedure in Step G above, a solution of 3-[4-chloro-5-methyl-2-(1-methyl-1H-imidazol-2-yl)thieno[2,3-d]pyrimidin-6-yl]-1-(propan-2-yl)-1H-pyrazole (20g) (1.07 mmol) in DMSO (7 mL), was reacted with the amine rac (1R,3S′)-3-(2-methoxyethoxy)cyclopentan-1-amine (1.61 mmol) to give rac 6-(1-isopropyl-1H-pyrazol-3-yl)-N-((1R,3S)-3-(2-methoxyethoxy)cyclopentyl)-5-methyl-2-(1-methyl-1H-imidazol-2-yl)thieno[2,3-d]pyrimidin-4-amine 20 as a yellow gum (30.0 mg, 5.4% yield). ¹H NMR (DMSO-d6, 400 MHz): δ (ppm) 1.46 (m, 6H), 1.88 (m, 5H), 2.07 (m, 3H), 2.78 (s, 3H), 3.21 (s, 3H), 3.53 (m, 2H), 4.29 (s, 3H), 6.64 (m, 1H), 6.95 (d, 1H), 7.77 (m, 1H), 7.89 (d, 1H), 7.97 (d, 1H).

### Compound 21-Compound 27

Following the general procedure described for Compound 20, Step G, 4-chloro-6-(1-isopropyl-1H-pyrazol-3-yl)-5-methyl-2-(1-methyl-1H-imidazol-2-yl)thieno[2,3-d]pyrimidine 20g (1.07 mmol) was treated with the appropriate corresponding amine (1.61 mmol) to produce Compounds 21-27 shown in Table 1.

TABLE 1

| | | | Step G | |
|---|---|---|---|---|
| | | | Amount | |
| Compound | Structure | Name | (Yield) | ¹H NMR |
| 21 | 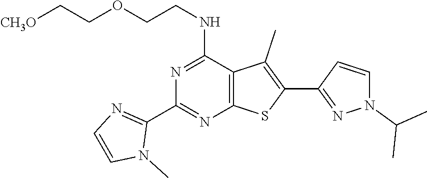 | 6-(1-Isopropyl-1H- pyrazol-3-yl)-N- (2-(2- methoxyethoxy) ethyl)-5-methyl-2- (1-methyl-1H- imidazol-2-yl) thieno[2,3-d pyrimidin-4- amine | 34.0 mg (13.2%) | (DMSO-d6, 400 MHz): δ (ppm) 1.47 (d, 6H), 2.75 (s, 3H), 3.23 (s, 3H), 3.41 (m, 1H), 3.55 (m, 2H), 3.69 (m, 2H), 3.75 (m, 2H), 4.02 (s, 3H), 4.57 (m, 1H), 6.62 (d, 1H), 6.87 (m, 1H), 7.02 (d, 1H), 7.31 (d, 1H), 7.93 (d, 1H), 8.15 (m, 1H). |
| 22 | 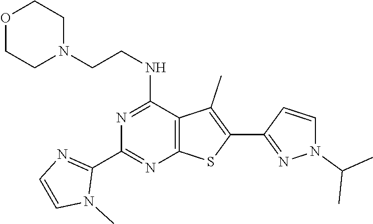 [SMILES: Cc1c(-c2ccn(C(C)C)n2)sc2nc(-c3nccn3C)nc(NCCN3CCOCC3)c12] | 6-(1-Isopropyl-1H- pyrazol-3-yl)-5- methyl-2-(1- methyl-1H- imidazol-2-yl)-N- (2-morpholino- ethyl)thieno[2,3-d] pyrimidin-4-amine | 32.0 mg (12.2%) | (CD3OD, 400 MHz): δ (ppm) 1.56 (d, 6H), 2.80 (m, 7H), 2.95 (m, 2H), 3.80 (m, 4H), 3.87 (m, 2H), 4.21 (s, 3H), 4.60 (m, 1H), 6.56 (m, 1H), 7.22 (d, 1H), 7.32 (d, 1H), 7.78 (m, 1H), 8.41 (d, 1H). |
| 23 | 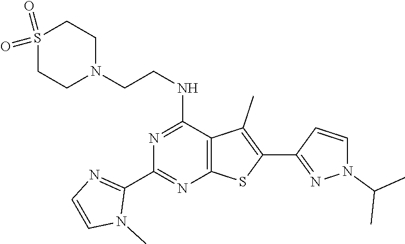 [SMILES: Cc1c(-c2ccn(C(C)C)n2)sc2nc(-c3nccn3C)nc(NCCN3CCS(=O)(=O)CC3)c12] | 4-(2-((6-(1- Isopropyl-1H- pyrazol-3-yl)-5- methyl-2-(1- methyl-1H- imidazol-2-yl) thieno[2,3-d] pyrimidin-4-yl) amino)ethyl) thiomorpholine 1,1-dioxide | 17.0 mg (5.9%) | (CD3OD, 400 MHz): δ (ppm) 1.56 (d, 6H), 2.84-2.93 (m, 5H), 3.13 (m, 8H), 3.85 (t, 2H), 4.27 (s, 3H), 4.60 (m, 1H) 6.58 (d, 1H), 7.31 (d, 1H), 7.41 (d, 1H), 7.79 (d, 1H), 8.28 (d, 1H). |
| 24 | 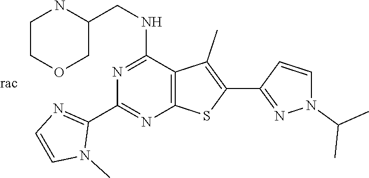 [SMILES: Cc1c(-c2ccn(C(C)C)n2)sc2nc(-c3nccn3C)nc(NCC3COCCN3)c12] | rac 6-(1- Isopropyl-1H- pyrazol-3-yl)-5- methyl-2-(1- methyl-1H- imidazol-2-yl)-N- ((4-methyl- morpholin-3- yl)methyl)thieno [2,3-d]pyrimidin- 4-amine | 28.0 mg (11.2%) | (DMSO-d6, 400 MHz): δ (ppm) 1.46 (d, 6H), 2.34 (s, 3H), 2.78 (m, 5H), 3.39-3.58 (m, 7H), 4.05 (s, 3H), 4.57 (m, 1H), 6.62 (d, 1H), 6.81 (m, 1H), 7.02 (m, 1H), 7.31 (m, 1H), 7.93 (d, 1H) |
| 25 | 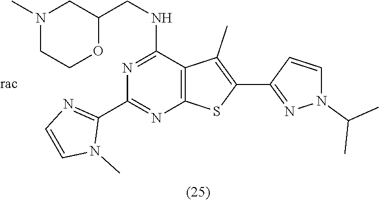 [SMILES: Cc1c(-c2ccn(C(C)C)n2)sc2nc(-c3nccn3C)nc(NCC3CN(C)CCO3)c12] | rac 6-(1- Isopropyl-1H- pyrazol-3-yl)-5- methyl-2-(1- methyl-1H- imidazol-2-yl)-N- ((4-methyl- morpholin-2- yl)methyl)thieno [2,3-d]pyrimidin- 4-amine | 25.0 mg (9.5%) | (CD3OD, 400 MHz): δ (ppm) 1.57 (d, 6H), 2.55 (m, 3H), 2.78 (m, 6H), 3.50 (m, 2H), 3.77 (m, 2H), 4.08 (m, 3H), 4.20 (s, 3H), 6.54 (m, 1H), 7.24 (m, 1H), 7.78 (d, 1H), 8.34 (s, 1H). |
| 26 | 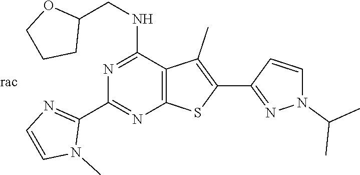 [SMILES: Cc1c(-c2ccn(C(C)C)n2)sc2nc(-c3nccn3C)nc(NCC3CCCO3)c12] | rac 6-(1- Isopropyl-1H- pyrazol-3-yl)-5- methyl-2-(1- methyl-1H- imidazol-2-yl)-N- ((tetrahydrofuran- 2-yl)methyl) thieno[2,3-d] pyrimidin-4- amine | 35.0 mg (18.9%) | (DMSO-d6, 400 MHz): δ (ppm) 1.47 (d, 6H), 1.69-1.95 (m, 4H), 2.75 (s, 3H), 3.65-3.67 (m, 3H), 3.82-3.83 (m, 1H), 4.0 (s, 3H), 4.16-4.19 (m, 1H), 4.56-4.59 (sept., 1H), 6.61 (d, 1H), 6.85 (m, 1H), 7.02 (m, 1H), 7.30 (m, 1H), 7.93 (d, 1H). |
| 27 | 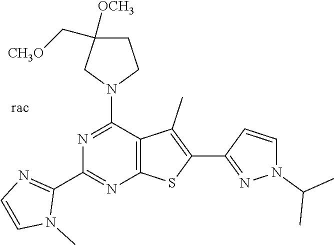 | rac 6-(1- Isopropyl-1H- pyrazol-3-yl)-4- (3-methoxy-3- (methoxymethyl) pyrrolidin-1-yl)- 5-methyl-2-(1- methyl-1H- imidazol-2- yl)thieno[2,3-d] pyrimidine | 38.0 mg (14.0%) | (DMSO-d6, 400 MHz): δ (ppm) 1.46 (d, 6H), 1.85-2.80 (m, 2H), 2.60 (s, 3H), 3.14 (s, 3H), 3.47 (m, 1H), 3.56-3.67 (m, 2H), 3.85-3.96 (m, 2H), 4.03 (s, 3H), 4.54-4.59 (m, 1H), 6.64 (d, 1H), 7.01 (m, 1H), 7.30 (m, 1H), 7.92 (d, 1H). |

### Compound 28

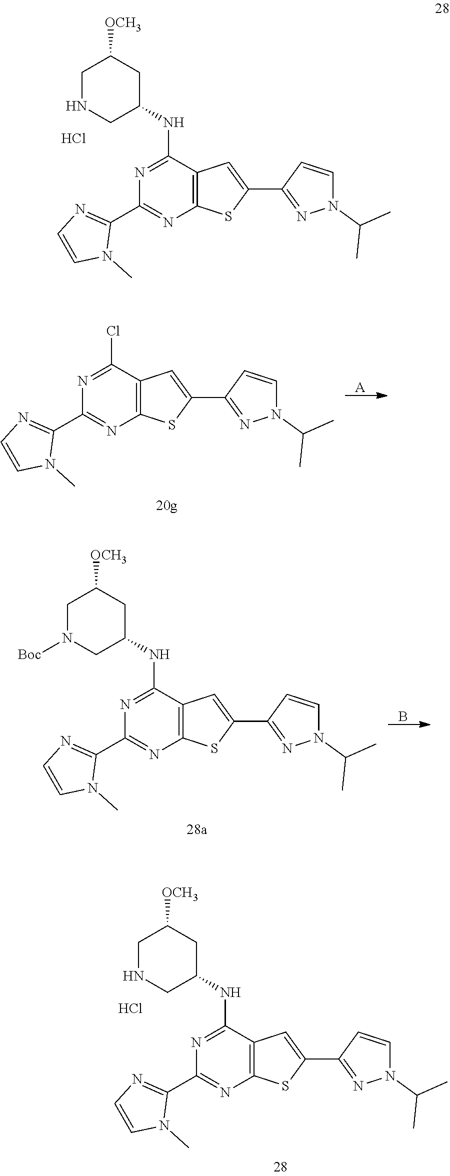

### Step A: tert-Butyl (3S,5R)-3-((6-(1-isopropyl-1H-pyrazol-3-yl)-2-(1-methyl-1H-imidazol-2-yl)thieno[2,3-d]pyrimidin-4-yl)amino)-5-methoxypiperidine-1-carboxylate 28a

Following the general procedure described for Compound 20, Step G, 4-chloro-6-(1-isopropyl-1H-pyrazol-3-yl)-5-methyl-2-(1-methyl-1H-imidazol-2-yl)thieno[2,3-d]pyrimidine 20g (1.07 mmol) was treated with the appropriate corresponding amine tert-butyl (3S,5R)-3-amino-5-methoxypiperidine-1-carboxylate (1.61 mmol) to produce tert-butyl (3S,5R)-3-((6-(1-isopropyl-1H-pyrazol-3-yl)-2-(1-methyl-1H-imidazol-2-yl)thieno[2,3-d]pyrimidin-4-yl)amino)-5-methoxypiperidine-1-carboxylate 28a as a brown gum (170.0 mg, 55.9% yield).

### Step B: 6-(1-Isopropyl-1H-pyrazol-3-yl)-N-((3S,5R)-5-methoxypiperidin-3-yl)-2-(1-methyl-1H-imidazol-2-yl)thieno[2,3-d]pyrimidin-4-amine hydrochloride salt 28

To an ice-cold solution of tert-butyl (3S,5R)-3-((6-(1-isopropyl-1H-pyrazol-3-yl)-2-(1-methyl-1H-imidazol-2-yl)thieno[2,3-d]pyrimidin-4-yl)amino)-5-methoxypiperidine-1-carboxylate 28a (149.39 mg, 263.61 μmol) in methanol (424.0 mg, 13.23 mmol, 530.0 μL, 50.0 equiv) acetyl chloride (103.46 mg, 1.32 mmol, 90.0 μl, 5.0 equiv) was added dropwise and allowed to stir at RT overnight. After evaporation of the solvent the residue was triturated with diethyl ether (15 mL), filtered and dried in vacuo to yield 6-(1-isopropyl-1H-pyrazol-3-yl)-N-((3S,5R)-5-methoxypiperidin-3-yl)-2-(1-methyl-1H-imidazol-2-yl)thieno[2,3-d]pyrimidin-4-amine hydrochloride salt 28 (37.0 mg, 28.6% yield) after HPLC as yellow gum. Formula weight: 503. Mass Found: [M+2]/2=234.0.

### Compound 29 and Compound 30

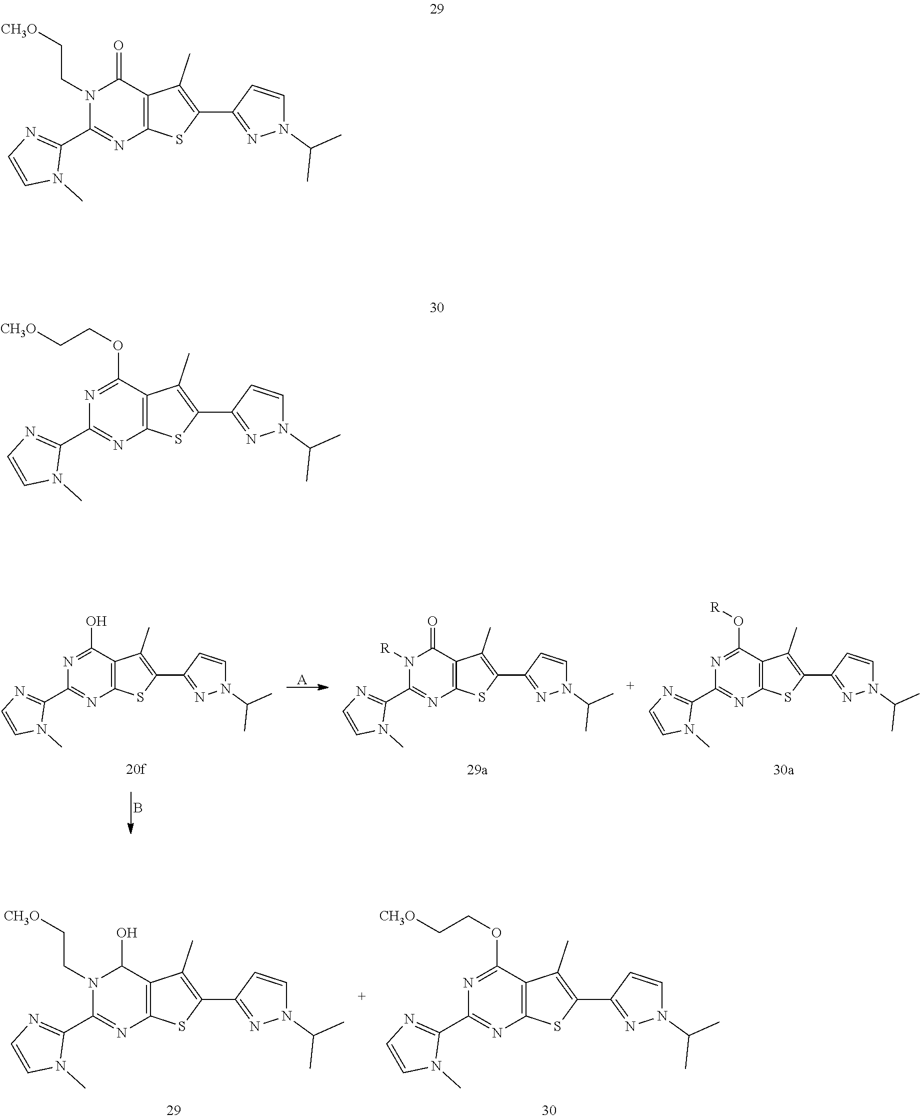

Step A: General Procedure: The appropriate corresponding halogenide R—X (281 μmol, 1.0 equiv) was added in one portion to the solution of 6-(1-isopropyl-1H-pyrazol-3-yl)-5-methyl-2-(1-methyl-1H-imidazol-2-yl)thieno[2,3-d]pyrimidin-4-ol 20f (prepared as described for Compound 20, Step E) (100.0 mg, 281 μmol) and potassium carbonate (77.8 mg, 562.92 μmol) in DMF (2.0 mL). The mixture was heated to 100° C. and stirred at this temperature for 16 h. The mixture was filtered off and purified by HPLC. Two isomers corresponding to structures 29a and 29b were separated and purified with good yields.

Step B: 6-(1-Isopropyl-1H-pyrazol-3-yl)-3-(2-methoxyethyl)-5-methyl-2-(1-methyl-1H-imidazol-2-yl)thieno[2,3-d]pyrimidin-4(3H)-one 29 and 6-(1-isopropyl-1H-pyrazol-3-yl)-4-(2-methoxyethoxy)-5-methyl-2-(1-methyl-1H-imidazol-2-yl)thieno[2,3-d]pyrimidine 30.

Following the general procedure described in Step A, 6-(1-isopropyl-1H-pyrazol-3-yl)-5-methyl-2-(1-methyl-1H-imidazol-2-yl)thieno[2,3-d]pyrimidin-4-ol 20f was treated with the appropriate corresponding R—X to produce a mixture of two compounds which were separated by HPLC to give 6-(1-isopropyl-1H-pyrazol-3-yl)-3-(2-methoxyethyl)-5-methyl-2-(1-methyl-1H-imidazol-2-yl)thieno[2,3-d]pyrimidin-4(3H)-one 29 as a beige solid (35.1 mg, 30.0% yield) (retention time 1.183 min), and 6-(1-isopropyl-1H-pyrazol-3-yl)-4-(2-methoxyethoxy)-5-methyl-2-(1-methyl-1H-imidazol-2-yl)thieno[2,3-d]pyrimidine 30 as a light brown gum (51.0 mg, 44% yield) (retention time 1.211 min).

¹H NMR of 29: (DMSO-d6, 400 MHz): δ (ppm) 1.48 (d, 6H), 2.73 (s, 3H), 3.36 (s, 3H), 3.80-3.82 (m, 2H), 4.08 (s, 3H), 4.57-4.61 (sept., 111), 4.69-4.71 (m, 2H), 6.69 (d, 111), 7.08 (m, 1H), 7.38 (m, 1H), 7.96 (d, 1H).

¹H NMR of 30: (DMSO-d6, 400 MHz): δ (ppm) 1.46 (d, 6H), 2.74 (s, 3H), 3.02 (s, 3H), 3.46-3.49 (t, 2H), 3.77 (s, 3H), 4.53-4.60 (m, 3H), 6.63 (d, 111), 7.11 (m, 111), 7.42 (m, 1H), 7.93 (d, 111).

### Compound 31-Compound 35

Following the general procedure described for Compounds 29 and 30, Step B, 6-(1-isopropyl-1H-pyrazol-3-yl)-5-methyl-2-(1-methyl-1H-imidazol-2-yl)thieno[2,3-d]pyrimidin-4-ol 20f (prepared as described for Compound 20, Step E) (100.0 mg, 281 μmol) was treated with the appropriate corresponding R—X (281 μmol, 1.0 equiv) to produce an isomeric mixture of 31 and 32, which was separated by HPLC to give Compounds 31 and 32. In the same way, a mixture of 33 and 34 was separated by HPLC to give Compounds 33 and 34. Compound 35 was the only isomer isolated. Compounds 31-35 are shown in Table 2.

TABLE 2

| | | | Step G | |
|---|---|---|---|---|
| | | | Amount | |
| | | | (Yield) | |
| | | | (HPLC | |
| Compound | Structure | Name | retention time) | ¹H NMR |
| 31 | 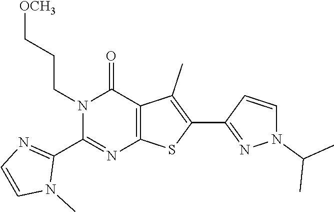 [SMILES: COCCCn1c(-c2nccn2C)nc2sc(-c3ccn(C(C)C)n3)c(C)c2c1=O] | 6-(1-Isopropyl-1H- pyrazol-3-yl)-3-(3- methoxypropyl)-5- methyl-2-(1- methyl-1H- imidazol-2-yl) thieno[2,3-d] pyrimidin-4(3H)- one | 25.3 mg (20.7%) (1.392 min) | (DMSO-d6, 400 MHz): δ (ppm) 1.46 (d, 6H), 1.86-1.93 (m, 2H), 2.74 (s, 3H), 3.13 (s, 3H), 3.30 (m, 2H), 3.80 (s, 3H), 4.28-4.31 (t, 2H), 4.53-4.58 (sept., 1H), 6.63 (d, 1H), 7.13 (m, 1H), 7.44 (m, 1H), 7.93 (m, 1H). |
| 32 | 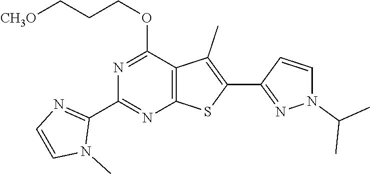 | 6-(1-Isopropyl-1H- pyrazol-3-yl)-4-(3- methoxypropoxy)- 5-methyl-2-(1- methyl-1H- imidazol-2-yl) thieno[2,3-d] pyrimidine | 27.7 mg (22.7%) (1.231 min) | (DMSO-d6, 400 MHz): δ (ppm) 1.47 (d, 6H), 2.08- 2.11 (t, 2H), 2.73 (s, 3H), 3.27 (s, 3H), 3.55-3.58 (t, 2H), 4.08 (s, 3H), 4.56- 4.64 (m, 3H), 6.69 (d, 1H), 7.08 (d, 1H), 7.38 (d, 1H), 7.96 (d, 1H). |
| 33 | 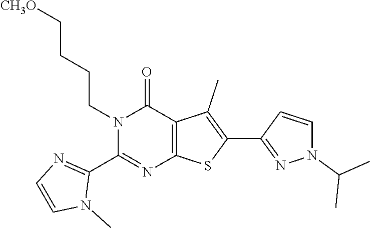 | 6-(1-Isopropyl-1H- pyrazol-3-yl)-3-(4- methoxybutyl)-5- methyl-2-(1- methyl-1H- imidazol-2-yl) thieno[2,3-d] pyrimidin-4(3H)- one | 25.8 mg (20.8%) (1.432 min) | (DMSO-d6, 400 MHz): δ (ppm) 1.38 (t, 2H), 1.46 (d, 6H), 1.66 (m, 2H), 2.74 (s, 3H), 3.17 (s, 3H), 3.22 (t, 2H), 3.80 (s, 3H), 4.26 (t, 2H), 4.51-4.59 (sept., 1H), 6.62 (d, 1H), 7.14 (d, 1H), 7.45 (d, 1H), 7.93 (d, 1H). |
| 34 | 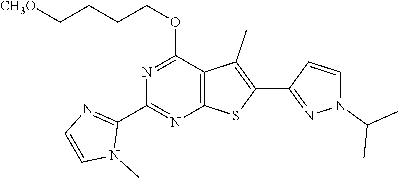 | 6-(1-Isopropyl-1H- pyrazol-3-yl)-4-(4- methoxybutoxy)- 5-methyl-2-(1- methyl-1H- imidazol-2-yl) thieno[2,3-d] pyrimidine | 54.7 mg (44.0%) (1.308 min) | (DMSO-d6, 400 MHz): δ (ppm) 1.47 (d, 6H), 1.76 (t, 2H), 1.90 (t, 2H), 2.73 (s, 3H), 3.25 (s, 3H), 3.42 (t, 2H), 4.09 (s, 3H), 4.59 (m, 3H), 6.69 (d, 1H), 7.08 (d, 1H), 7.36 (d, 1H), 7.96 (d, 1H). |
| 35 | 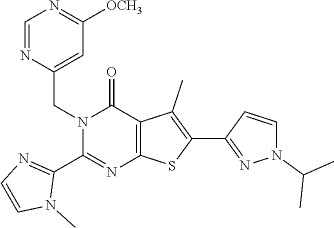 [SMILES: COc1cc(Cn2c(-c3nccn3C)nc3sc(-c4ccn(C(C)C)n4)c(C)c3c2=O)ncn1] | 6-(1-Isopropyl-1H- pyrazol-3-yl)-3-((6- methoxypyrimidin- 4-yl)methyl)-5- methyl-2-(1- methyl-1H- imidazol-2- yl)thieno[2,3- d]pyrimidin- 4(3H)-one | 31.0 mg (27.0%) (1.416 min) | (DMSO-d6, 400 MHz): δ (ppm) 1.47 (d, 6H), 2.70 (s, 3H), 3.78 (s, 3H), 3.88 (s, 3H), 4.53-4.60 (m, 1H), 5.71 (s, 2H), 6.63 (m, 1H), 6.78 (m, 1H), 7.01 (m, 1H), 7.34 (m, 1H), 7.93 (d, 1H), 8.62 (s, 1H). |

### Compound 36 and Compound 37

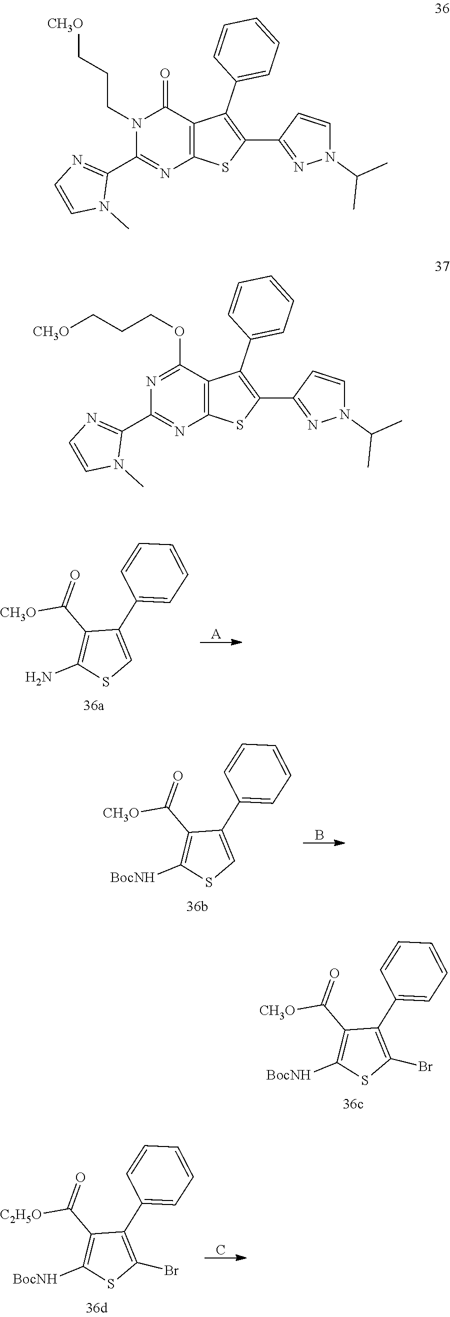

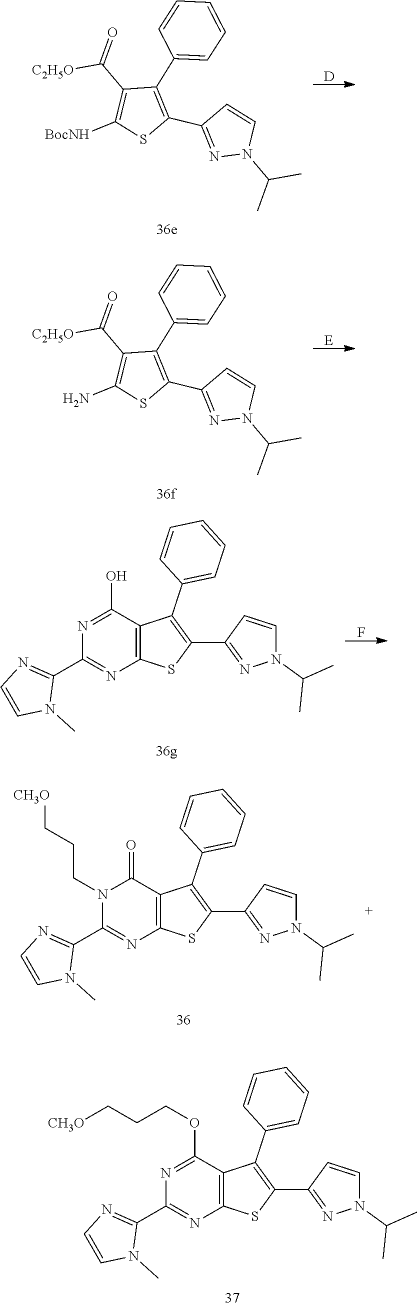

### Step A: Methyl 2-((tert-butoxycarbonyl)amino)-4-phenylthiophene-3-carboxylate 36b

To solution of methyl 2-amino-4-phenylthiophene-3-carboxylate 36a (35.0 g, 150.02 mmol) and N,N-dimethylpyridin-4-amine (1.83 g, 15.0 mmol) in dioxane (540 mL) di-tert-butyl dicarbonate (39.29 g, 180.02 mmol, 41.4 mL, 1.2 equiv) was added dropwise at 0° C. Reaction mixture was stirred at RT overnight then evaporated and purified by column chromatography to give methyl 2-((tert-butoxycarbonyl)amino)-4-phenylthiophene-3-carboxylate 36b (18.2 g, 95.0% purity, 51.86 mmol, 34.6% yield).

### Step B: Methyl 5-bromo-2-((tert-butoxycarbonyl)amino)-4-phenylthiophene-3-carboxylate 36c

To solution of methyl 2-((tert-butoxycarbonyl)amino)-4-phenylthiophene-3-carboxylate 36b (18.2 g, 54.59 mmol) in DMF (280 mL) N-bromosuccinimide (11.66 g, 65.51 mmol) was added under cooling with ice. Reaction mixture was stirred at RT overnight then poured into water. Precipitate was filtered, washed with water two times and dried on air to give methyl 5-bromo-2-((tert-butoxycarbonyl)amino)-4-phenylthiophene-3-carboxylate 36c (20.6 g, 90.0% purity, 44.97 mmol, 82.4% yield).

### Step C: Ethyl 2-((tert-butoxycarbonyl)amino)-5-(1-isopropyl-1H-pyrazol-3-yl)-4-phenylthiophene-3-carboxylate 36e

Ethyl 5-bromo-2-[(tert-butoxy)carbonyl]amino-4-phenylthiophene-3-carboxylate 36d (prepared according to the procedures described for Steps A and B above, except starting with ethyl 2-amino-4-phenylthiophene-3-carboxylate in place of methyl 2-amino-4-phenylthiophene-3-carboxylate 36a) (27.0 g, 63.33 mmol), 1-(propan-2-yl)-3-(tetramethyl-1,3,2-dioxaborolan-2-yl)-1H-pyrazole (16.45 g, 69.66 mmol), cesium carbonate (41.27 g, 126.66 mmol) and Pddppf (5.17 g, 6.33 mmol) was dissolved in degassed dioxane (500 ml) under Ar. 25 mL of water was added via syringe. The reaction mixture was heated to 80 C and stirred at this temperature for 16 h. The mixture was cooled to room temperature, filtered through celite and concentrated. The residue was purified by flash (Eluted by Hex:EtOAc 20:1 to 1:1). Crude ethyl 2-((tert-butoxycarbonyl)amino)-5-(1-isopropyl-1H-pyrazol-3-yl)-4-phenylthiophene-3-carboxylate 36e (15.0 g, 80.0% purity, 26.34 mmol, 41.6% yield) was obtained as light-yellow powder and used in next step without further purification.

### Step D: Ethyl 2-amino-5-(1-isopropyl-1H-pyrazol-3-yl)-4-phenylthiophene-3-carboxylate 36f

Dioxane*HCl (100 mL, 6M) was added dropwise to the solution of ethyl 2-((tert-butoxycarbonyl)amino)-5-(1-isopropyl-1H-pyrazol-3-yl)-4-phenylthiophene-3-carboxylate 36e (15.0 g, 32.93 mmol) in dichloromethane (150 mL) at 0° C. The mixture was stirred for 16 h at room temperature. The solution was concentrated in vacuo, triturated in MTBE (100 mL) and collected by filtration and dried on air. Ethyl 2-amino-5-(1-isopropyl-1H-pyrazol-3-yl)-4-phenylthiophene-3-carboxylate 36f (8.1 g, 22.79 mmol, 86.5% yield) was obtained.

### Step E: 6-(1-Isopropyl-1H-pyrazol-3-yl)-2-(1-methyl-1H-imidazol-2-yl)-5-phenylthieno[2,3-d]pyrimidin-4-ol 36g

Sodium hydride (146.45 mg, 6.1 mmol) (60% in mineral oil) was added portionwise (during 1 min) to the solution of ethyl 2-amino-5-(1-isopropyl-1H-pyrazol-3-yl)-4-phenylthiophene-3-carboxylate 36f (500.0 mg, 1.46 mmol) and 1-methyl-1H-imidazole-2-carbonitrile (235.32 mg, 2.2 mmol) in dioxane (10 mL). The reaction mixture was heated to reflux and stirred at this temperature for 16 h. Then the mixture was cooled to the room temperature, poured in ice water and neutralized by acetic acid. The formed participate was collected by filtration, washed by water (2*10 mL) and dried in vacuo. 6-(1-Isopropyl-1H-pyrazol-3-yl)-2-(1-methyl-1H-imidazol-2-yl)-5-phenylthieno[2,3-d]pyrimidin-4-ol 36g (200.0 mg, 480.19 μmol, 32.8% yield) was obtained and used in next step without further purification.

### Step F: 6-(1-Isopropyl-1H-pyrazol-3-yl)-3-(3-methoxypropyl)-2-(1-methyl-1H-imidazol-2-yl)-5-phenylthieno[2,3-d]pyrimidin-4(3H)-one 36 and 6-(1-isopropyl-1H-pyrazol-3-yl)-4-(3-methoxypropoxy)-2-(1-methyl-1H-imidazol-2-yl)-5-phenylthieno[2,3-d]pyrimidine 37

1-Iodo-3-methoxypropane (57.09 mg, 285.4 μmol, 30.0 μl, 1.0 equiv) was added in one portion to the solution of 6-(1-isopropyl-1H-pyrazol-3-yl)-2-(1-methyl-1H-imidazol-2-yl)-5-phenylthieno[2,3-d]pyrimidin-4-ol 36g (101.38 mg, 286.03 μmol) and potassium carbonate (79.06 mg, 572.05 μmol) in DMF (2 mL). The mixture was heated to 100° C. and stirred at this temperature for 16 h. The mixture was filtered off and purified by HPLC (2-10 min 40-60% methanol+HCl flow 30 mL/min (loading pump 4 mL methanol), column: Sun Fire C18). The two isomers were separated to give 6-(1-isopropyl-1H-pyrazol-3-yl)-3-(3-methoxypropyl)-2-(1-methyl-1H-imidazol-2-yl)-5-phenylthieno[2,3-d]pyrimidin-4(3H)-one 36 as a yellow solid (23.0 mg, 19.7%) (retention time 1.361 min) and 6-(1-isopropyl-1H-pyrazol-3-yl)-4-(3-methoxypropoxy)-2-(1-methyl-1H-imidazol-2-yl)-5-phenylthieno[2,3-d]pyrimidine 37 as a yellow solid (31.0 mg, 27.0%) (retention time 1.505 min).

¹H NMR of 36: (DMSO-d6, 400 MHz): δ (ppm) 1.35 (d, 6H), 1.55 (m, 2H), 2.84 (t, 2H), 3.07 (s, 3H), 4.24 (s, 3H), 4.35 (t, 2H), 4.50 (m, 1H), 5.26 (m, 1H), 7.36 (m, 2H), 7.50 (m, 3H), 7.69 (m, 1H), 7.85 (m, 1H), 7.97 (m, 1H).

¹H NMR of 37: (DMSO-d6, 400 MHz): δ (ppm) 1.45 (d, 6H), 1.77 (m, 2H), 3.06 (s, 3H), 3.19 (t, 2H), 3.81 (s, 3H), 4.15 (t, 2H), 4.45 (m, 1H), 5.16 (m, 1H), 7.09 (m, 1H), 7.29 (m, 2H), 7.40 (m, 4H), 7.52 (m, 1H).

### Compound 38

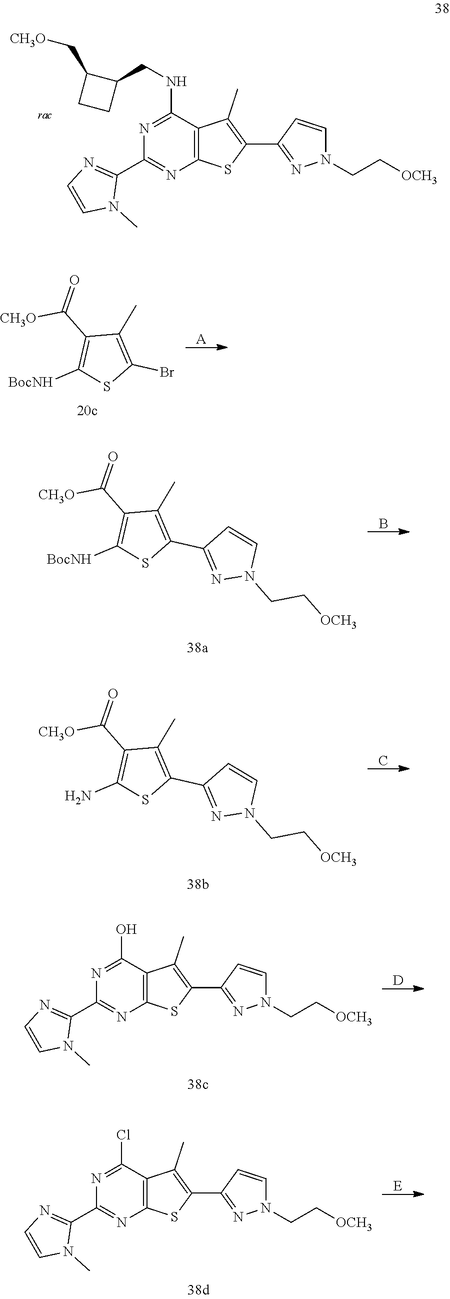

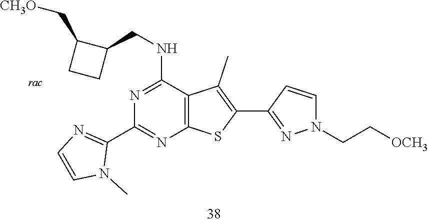

### Step A: Methyl 2-((tert-butoxycarbonyl)amino)-5-(1-(2-methoxyethyl)-1H-pyrazol-3-yl)-4-methylthiophene-3-carboxylate 38a

A mixture of methyl 5-bromo-2-((tert-butoxycarbonyl)amino)-4-methylthiophene-3-carboxylate 20 (prepared as described for Compound 20, Step B) (5.0 g, 12.13 mmol), 2-fluoro-3-methoxyphenylboronic acid (2.06 g, 12.13 mmol), cesium carbonate (7.9 g, 24.25 mmol) and [1,1′-bis(diphenylphosphino)ferrocene]dichloropalladium(II) dichloromethane adduct (990.29 mg, 1.21 mmol) was heated at 100° C. under Argon atmosphere overnight. The reaction mixture was evaporated and crude compound was purified by flash chromatography to give methyl 2-((tert-butoxycarbonyl)amino)-5-(1-(2-methoxyethyl)-1H-pyrazol-3-yl)-4-methylthiophene-3-carboxylate 38a (3.1 g, 72.0% purity, 4.88 mmol, 40.2% yield).

### Step B: Methyl 2-amino-5-(1-(2-methoxyethyl)-1H-pyrazol-3-yl)-4-methylthiophene-3-carboxylate 38b

To solution of methyl 2-((tert-butoxycarbonyl)amino)-5-(1-(2-methoxyethyl)-1H-pyrazol-3-yl)-4-methylthiophene-3-carboxylate 38a (3.1 g, 6.78 mmol) in chloroform (15 mL), HCl in dioxane (4.0M solution, 15 mL) was added dropwise at 0° C. The reaction mixture was stirred at RT overnight, then concentrated under reduce pressure. Product was crystalized from MTBE, filtered and dried on air to give methyl 2-amino-5-(1-(2-methoxyethyl)-1H-pyrazol-3-yl)-4-methylthiophene-3-carboxylate 38b (1.9 g, 80.5% purity, 4.28 mmol, 63.2% yield),

### Step C: 6-(1-(2-Methoxyethyl)-1H-pyrazol-3-yl)-5-methyl-2-(1-methyl-1H-imidazol-2-yl)thieno[2,3-d]pyrimidin-4-ol 38c

2-Amino-5-(1-(2-methoxyethyl)-1H-pyrazol-3-yl)-4-methylthiophene-3-carboxylate 38b (100.0 mg, 279.79 μmol) and 1-methyl-1H-imidazole-2-carbonitrile (29.97 mg, 279.79 μmol) was dissolved in DMF (25 mL) and sodium hydride (33.57 mg, 1.4 mmol) was added and mixture was heated at 80° C. 16 h. Then, the mixture was evaporated, diluted with water (20 mL) and extracted with EtOAc (3*30 mL). Organic layer dried and evaporated under reduce pressure to give 6-(1-(2-methoxyethyl)-1H-pyrazol-3-yl)-5-methyl-2-(1-methyl-1H-imidazol-2-yl)thieno[2,3-d]pyrimidin-4-ol 38c (200.0 mg, 14.4% purity, 66.59 μmol, 23.8% yield).

### Step D: 4-Chloro-6-(1-(2-methoxyethyl)-1H-pyrazol-3-yl)-5-methyl-2-(1-methyl-1H-imidazol-2-yl)thieno[2,3-d]pyrimidine 38d

6-(1-(2-Methoxyethyl)-1H-pyrazol-3-yl)-5-methyl-2-(1-methyl-1H-imidazol-2-yl)thieno[2,3-d]pyrimidin-4-ol 38c (198.53 mg, 459.07 μmol) was suspended in phosphoryl chloride (703.9 mg, 4.59 mmol, 430.0 μl, 10.0 equiv) and ethylbis(propan-2-yl)amine (178.08 mg, 1.38 mmol, 240.0 μL, 3.0 equiv) was added at RT. The reaction mixture was heated at 100° C. for 16 h, the solution was cooled to RT, evaporated under reduced pressure, poured into ice and diluted with ice-cold ammonia (20 mL, 20-25% of ammonia), the product was extracted with chloroform (3.20 mL) and evaporated. Crude compound was purified by HPLC (SunFire C18 Column; 2-10 min 45-60% acetonitrile+NH₃) to give 4-chloro-6-(1-(2-methoxyethyl)-1H-pyrazol-3-yl)-5-methyl-2-(1-methyl-1H-imidazol-2-yl)thieno[2,3-d]pyrimidine 38d (17.0 mg, 98.3% purity, 37.06 μmol, 8.1% yield).

### Step E: rac 6-(1-(2-Methoxyethyl)-1H-pyrazol-3-yl)-N-(((1S,2R)-2-(methoxymethyl)cyclobutyl)methyl)-5-methyl-2-(1-methyl-1H-imidazol-2-yl)thieno[2,3-d]pyrimidin-4-amine 38

4-Chloro-6-(1-(2-methoxyethyl)-1H-pyrazol-3-yl)-5-methyl-2-(1-methyl-1H-imidazol-2-yl)thieno[2,3-d]pyrimidine 38d (17.23 mg, 38.21 μmol) was dissolved in DMSO (5 mL) and rac ((1S,2R)-2-(methoxymethyl)cyclobutyl)methanamine hydrochloride salt (5.26 mg, 38.21 μmol) with ethylbis(propan-2-yl)amine (14.84 mg, 114.82 μmol, 20.0 μl, 3.0 equiv) were added. The mixture was heated at 100° C. overnight, cooled and purified by HPLC (SunFire C18 Column; 2-10 min 50-100% acetonitrile+NH₃) to give rac 6-(1-(2-Methoxyethyl)-1H-pyrazol-3-yl)-N-(((1S,2R)-2-(methoxymethyl)cyclobutyl)methyl)-5-methyl-2-(1-methyl-1H-imidazol-2-yl)thieno[2,3-d]pyrimidin-4-amine 38 (6.8 mg, 32.8% yield). ¹H-NMR (CD₃OD, 400 MHz): δ (ppm) 1.88-2.12 (m, 7H), 2.72 (s, 3H), 3.36-3.38 (m, 6H), 3.80 (t, 2H), 4.05 (m, 1H), 4.17 (s, 3H), 4.37 (t, 2H), 4.95 (m, 1H), 6.55 (d, 1H), 7.10 (m, 1H), 7.22 (m, 1H), 7.74 (d, 1H).

### Compound 39

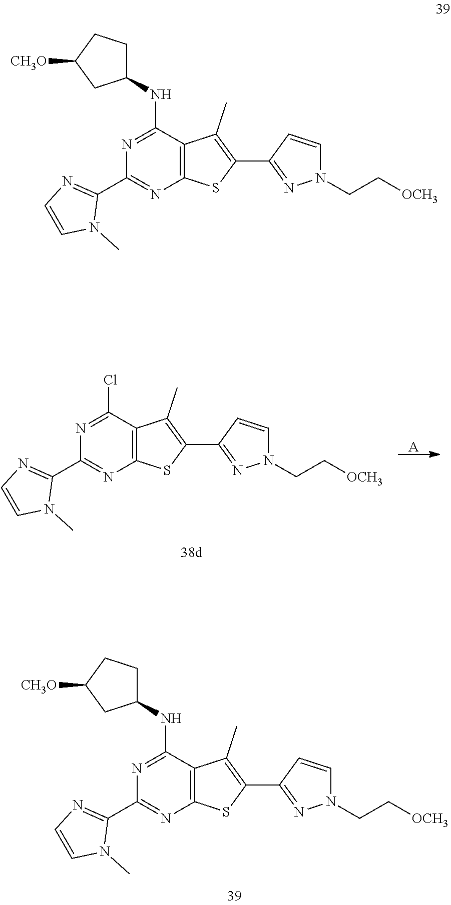

### Step A: N-((1R,3S)-3-Methoxycyclopentyl)-6-(1-(2-methoxyethyl)-1H-pyrazol-3-yl)-5-methyl-2-(1-methyl-1H-imidazol-2-yl)thieno[2,3-d]pyrimidin-4-amine 39

4-Chloro-6-(1-(2-methoxyethyl)-1H-pyrazol-3-yl)-5-methyl-2-(1-methyl-1H-imidazol-2-yl)thieno[2,3-d]pyrimidine 38d (prepared as described for Compound 38, Step D) (396.74 mg, 879.86 μmol) was dissolved in DMSO (5 mL) and rac (1R,3S)-3-methoxycyclopentan-1-amine hydrochloride (133.42 mg, 879.86 μmol) with ethylbis(propan-2-yl)amine (341.32 mg, 2.64 mmol, 460.0 μL, 3.0 equiv) were added. The mixture was heated at 100° C. overnight, cooled and purified by HPLC to give N-((1R,3S)-3-methoxycyclopentyl)-6-(1-(2-methoxyethyl)-1H-pyrazol-3-yl)-5-methyl-2-(1-methyl-1H-imidazol-2-yl)thieno[2,3-d]pyrimidin-4-amine 39 (4.8 mg, 1% yield). ¹H NMR (CD₃OD, 400 MHz): δ (ppm) 1.54-1.59 (m, 1H), 1.81-1.88 (m, 3H), 2.73 (s, 3H), 2.82-2.89 (m, 2H), 3.32-3.49 (m, 9H), 3.80 (m, 3H), 4.10 (m, 1H), 4.17 (s, 3H), 4.37 (t, 2H), 6.54 (d, 1H), 7.13 (m, 1H), 7.25 (m, 1H), 7.74 (d, 1H).

### Compound 40

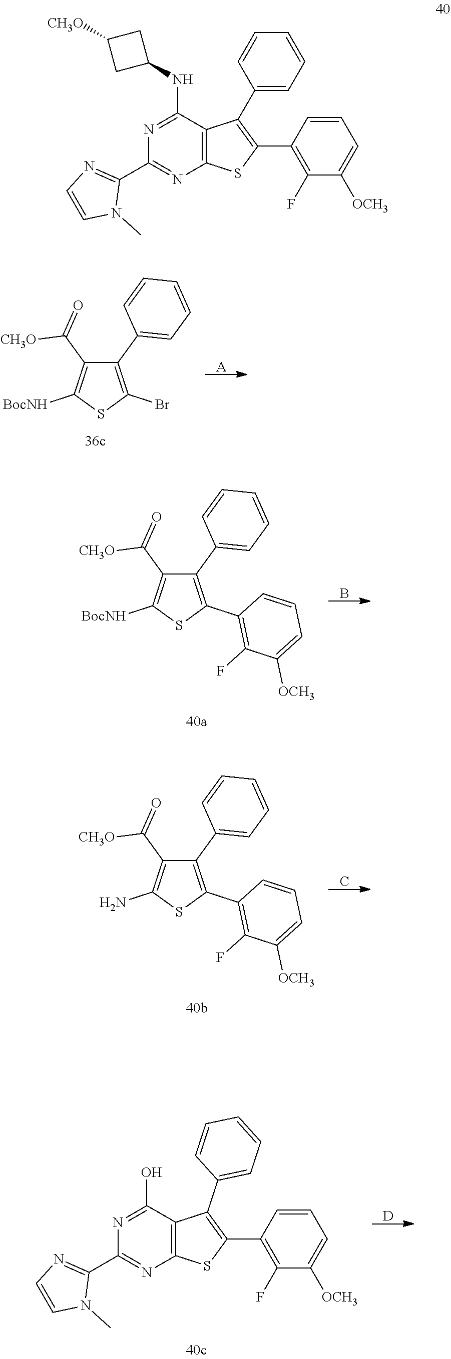

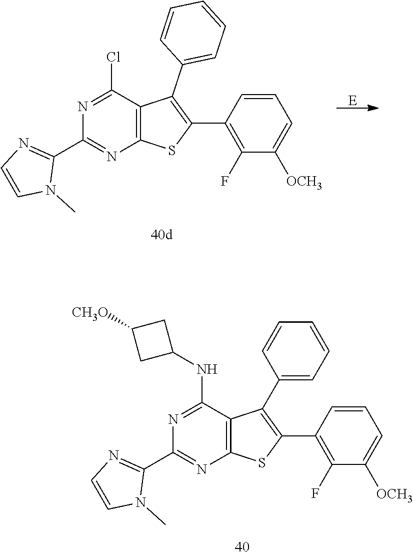

### Step A: Methyl 2-((tert-butoxycarbonyl)amino)-5-(2-fluoro-3-methoxyphenyl)-4-phenylthiophene-3-carboxylate 40a

A mixture of methyl 5-bromo-2-((tert-butoxycarbonyl)amino)-4-phenylthiophene-3-carboxylate 36c (prepared as described for Compound 36, Step B) (5.0 g, 12.13 mmol), 2-fluoro-3-methoxyphenylboronic acid (2.06 g, 12.13 mmol), cesium carbonate (7.9 g, 24.25 mmol) and [1,1′-bis(diphenylphosphino)ferrocene]dichloropalladium(II) dichloromethane adduct (990.29 mg, 1.21 mmol) was heated at 100° C. under Argon atmosphere overnight. Reaction mixture was evaporated and crude compound was purified by flash chromatography to give methyl 2-((tert-butoxycarbonyl)amino)-5-(2-fluoro-3-methoxyphenyl)-4-phenylthiophene-3-carboxylate 40a (3.1 g, 72.0% purity, 4.88 mmol, 40.2% yield).

### Step B: Methyl 2-amino-5-(2-fluoro-3-methoxyphenyl)-4-phenylthiophene-3-carboxylate 40b

To solution of methyl 2-((tert-butoxycarbonyl)amino)-5-(2-fluoro-3-methoxyphenyl)-4-phenylthiophene-3-carboxylate 40a (3.1 g, 6.78 mmol) in chloroform (15 mL) HCl in dioxane (15 mL) was added dropwise at 0° C. Reaction mixture was stirred at RT overnight then concentrated under reduce pressure. Product was crystalized from MTBE, filtered and dried on air to give methyl 2-amino-5-(2-fluoro-3-methoxyphenyl)-4-phenylthiophene-3-carboxylate 40b (1.9 g, 80.5% purity, 4.28 mmol, 63.2% yield).

### Step C: 6-(2-Fluoro-3-methoxyphenyl)-2-(1-methyl-1H-imidazol-2-yl)-5-phenylthieno[2,3-d]pyrimidin-4-ol 40c

Methyl 2-amino-5-(2-fluoro-3-methoxyphenyl)-4-phenylthiophene-3-carboxylate 40b (100.0 mg, 279.79 μmol) and 1-methyl-11-imidazole-2-carbonitrile (29.97 mg, 279.79 μmol) was dissolved in DMF (25 mL) and sodium hydride (33.57 mg, 1.4 mmol) was added and mixture was heated at 80° C. 16 h. Then mixture was evaporated, diluted with water (20 mL) and extracted with EtOAc (30 mL*3). Organic layer dried and evaporated under reduce pressure to give 6-(2-fluoro-3-methoxyphenyl)-2-(1-methyl-1H-imidazol-2-yl)-5-phenylthieno[2,3-d]pyrimidin-4-ol 40c (200.0 mg, 14.4% purity, 66.59 μmol, 23.8% yield).

### Step D: 4-Chloro-6-(2-fluoro-3-methoxyphenyl)-2-(1-methyl-1H-imidazol-2-yl)-5-phenylthieno[2,3-d]pyrimidine 40d

The starting material 6-(2-fluoro-3-methoxyphenyl)-2-(1-methyl-1H-imidazol-2-yl)-5-phenylthieno[2,3-d]pyrimidin-4-ol 40c (198.53 mg, 459.07 μmol) was suspended in phosphoroyl trichloride (703.9 mg, 4.59 mmol, 430.0 μL, 10.0 equiv) and ethylbis(propan-2-yl)amine (178.08 mg, 1.38 mmol, 240.0 μl, 3.0 equiv) was added at RT. The reaction mixture was heated at 100° C. for 16 h, the solution was cooled to RT, evaporated under reduced pressure, poured into ice and diluted with ice-cold ammonia (20 mL, 20-25% of ammonia), the product was extracted with chloroform (3*20 mL) and evaporated. Crude compound was purified by HPLC to give 4-chloro-6-(2-fluoro-3-methoxyphenyl)-2-(1-methyl-1H-imidazol-2-yl)-5-phenylthieno[2,3-d]pyrimidine 40d (17.0 mg, 98.3% purity, 37.06 μmol, 8.1% yield),

### Step E: 6-(2-Fluoro-3-methoxyphenyl)-N-((1r,3r)-3-methoxycyclobutyl)-2-(1-methyl-1H-imidazol-2-yl)-5-phenylthieno[2,3-d]pyrimidin-4-amine 40

4-Chloro-6-(2-fluoro-3-methoxyphenyl)-2-(1-methyl-1H-imidazol-2-yl)-5-phenylthieno[2,3-d]pyrimidine 40d (17.23 mg, 38.21 μmol) was dissolved in DMSO (5 mL) and (1r,3r)-3-methoxycyclobutan-1-amine hydrochloride (5.26 mg, 38.21 μmol) with ethylbis(propan-2-yl)amine (14.84 mg, 114.82 μmol, 20.0 μl, 3.0 equiv) were added. The mixture was heated at 100° C. overnight, cooled and purified by HPLC to give 6-(2-fluoro-3-methoxyphenyl)-N-((1r,3r)-3-methoxycyclobutyl)-2-(1-methyl-1H-imidazol-2-yl)-5-phenylthieno[2,3-d]pyrimidin-4-amine 40 (6.8 mg, 32.8% yield). ¹H NMR (CDCl3, 400 MHz): δ (ppm) 1.26 (m, 2H), 2.79 (m, 2H), 3.16 (s, 3H), 3.70 (t, 1H), 3.82 (s, 3H), 4.30 (s, 3H), 4.75 (m, 1H), 5.04 (m, 1H), 6.73 (t, 1H), 6.86-6.91 (m, 2H), 7.10 (m, 1H), 7.31 (m, 2H), 7.41 (m, 3H), 7.49 (m, 1H).

### Compound 41

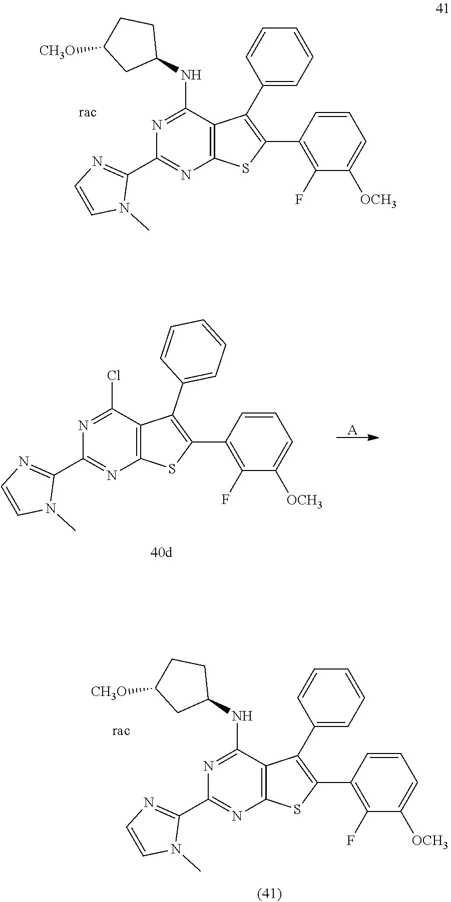

### Step A: 6-(2-Fluoro-3-methoxyphenyl)-N-((1R,3R)-3-methoxycyclopentyl)-2-(1-methyl-1H-imidazol-2-yl)-5-phenylthieno[2,3-d]pyrimidin-4-amine 41

4-Chloro-6-(2-fluoro-3-methoxyphenyl)-2-(1-methyl-1H-imidazol-2-yl)-5-phenylthieno[2,3-d]pyrimidine 40d (prepared as described for Compound 40, Step D) (396.74 mg, 879.86 μmol) was dissolved in DMSO (5 mL) and rac (1R,3R)-3-methoxycyclopentan-1-amine hydrochloride (133.42 mg, 879.86 μmol) with ethylbis(propan-2-yl)amine (341.32 mg, 2.64 mmol, 460.0 μL, 3.0 equiv) were added. The mixture was heated at 100° C. overnight, cooled and purified by HPLC to give 6-(2-fluoro-3-methoxyphenyl)-N-((1R,3R)-3-methoxycyclopentyl)-2-(1-methyl-1H-imidazol-2-yl)-5-phenylthieno[2,3-d]pyrimidin-4-amine 41 (4.8 mg, 1% yield). ¹H NMR (CD₃OD, 400 MHz): δ (ppm) 1.10-1.17 (m, 1H), 1.30-1.37 (m, 1H), 1.66-1.71 (m, 2H), 2.04-2.11 (m, 2H), 3.25 (s, 3H), 3.73 (m, 1H), 3.84 (s, 3H), 4.87 (s, 3H), 4.60-4.63 (m, 1H), 6.84 (t, 1H), 7.01-7.11 (m, 2H), 7.39-7.41 (m, 2H), 7.51-7.53 (m, 3H), 7.68 (d, 1H), 7.75 (d, 1H).

### Compound 42

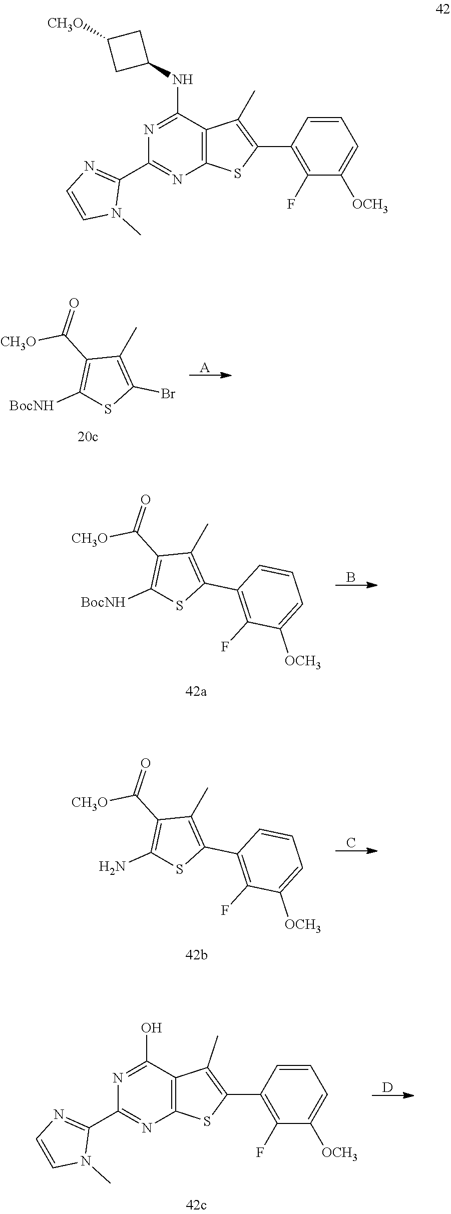

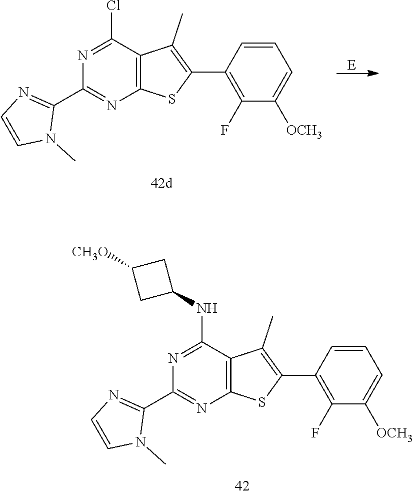

### Step A: Methyl 2-((tert-butoxycarbonyl)amino)-5-(2-fluoro-3-methoxyphenyl)-4-methylthiophene-3-carboxylate 42a

A mixture of methyl 5-bromo-2-((tert-butoxycarbonyl)amino)-4-methylthiophene-3-carboxylate 20c (prepared as described for Compound 20, Step B) (6.0 g, 17.13 mmol) (3), 2-fluoro-3-methoxyphenylboronic acid (2.91 g, 17.13 mmol), cesium carbonate (11.16 g, 34.27 mmol) and [1,1′-bis(diphenylphosphino)ferrocene]dichloropalladium(II) dichloromethane adduct (1.4 g, 1.71 mmol) in dioxane/H₂O (120 mL/6 mL) was heated at 100° C. under Argon atmosphere overnight. The reaction mixture was evaporated and crude compound was purified by flash chromatography to give methyl 2-((tert-butoxycarbonyl)amino)-5-(2-fluoro-3-methoxyphenyl)-4-methylthiophene-3-carboxylate 42a (4.44 g, 85.8% purity, 9.63 mmol, 56.2% yield).

### Step B: Methyl 2-amino-5-(2-fluoro-3-methoxyphenyl)-4-methylthiophene-3-carboxylate 42b

To the solution of methyl 2-((tert-butoxycarbonyl)amino)-5-(2-fluoro-3-methoxyphenyl)-4-methylthiophene-3-carboxylate 42a (4.44 g, 11.23 mmol) in chloroform (20 mL) HCl in dioxane (20 mL) was added dropwise at 0° C. The reaction mixture was stirred at RT overnight then concentrated under reduce pressure. Product was crystalized from MTBE, filtered and dried on air to give methyl 2-amino-5-(2-fluoro-3-methoxyphenyl)-4-methylthiophene-3-carboxylate 42b (2.2 g, 95.0% purity, 7.08 mmol, 66.6% yield).

### Step C: 6-(2-Fluoro-3-methoxyphenyl)-5-methyl-2-(1-methyl-1H-imidazol-2-yl)thieno[2,3-d]pyrimidin-4-ol 42c

1-Methyl-1H-imidazole-2-carbonitrile (239.44 mg, 2.24 mmol) and methyl 2-amino-5-(2-fluoro-3-methoxyphenyl)-4-methylthiophene-3-carboxylate 42b (440.0 mg, 1.49 mmol) was dissolved in DMF (10 mL) and sodium hydride (119.21 mg, 4.97 mmol) was added. The reaction mixture was stirred at 80° C. 16 h. Then, the mixture was evaporated, diluted with water (10 mL) and extracted with EtOAc (3*15 mL). Organic layer was dried over Na₂SO₄ and evaporated under reduced pressure to give 6-(2-fluoro-3-methoxyphenyl)-5-methyl-2-(1-methyl-1H-imidazol-2-yl)thieno[2,3-d]pyrimidin-4-ol 42c (570.0 mg, 67.0% purity, 1.03 mmol, 69.2% yield).

### Step D: 4-Chloro-6-(2-fluoro-3-methoxyphenyl)-5-methyl-2-(1-methyl-1H-imidazol-2-yl)thieno[2,3-d]pyrimidine 42d

6-(2-Fluoro-3-methoxyphenyl)-5-methyl-2-(1-methyl-1H-imidazol-2-yl)thieno[2,3-d]pyrimidin-4-ol 42c (673.46 mg, 1.82 mmol) was suspended in phosphoroyl trichloride (2.79 g, 18.18 mmol, 1.69 mL, 10.0 equiv) and ethylbis(propan-2-yl)amine (704.9 mg, 5.45 mmol, 950.0 μL, 3.0 equiv) was added at RT The reaction mixture was heated at 100 C for 16 h. Then, the solution was cooled to RT, evaporated under reduced pressure, poured in ice (200 mL) and diluted with ice-cold ammonia (200 mL, 20-25% of ammonia), the product was extracted with chloroform (2-200 mL) and evaporated. 4-Chloro-6-(2-fluoro-3-methoxyphenyl)-5-methyl-2-(1-methyl-1H-imidazol-2-yl)thieno[2,3-d]pyrimidine 42d (720.0 mg, 63.0% purity, 1.17 mmol, 64.2% yield) was obtained as brown slurry.

### Step E: 6-(2-Fluoro-3-methoxyphenyl)-N-((1r,3r)-3-methoxycyclobutyl)-5-methyl-2-(1-methyl-1H-imidazol-2-yl)thieno[2,3-d]pyrimidin-4-amine 42

4-Chloro-6-(2-fluoro-3-methoxyphenyl)-5-methyl-2-(1-methyl-1H-imidazol-2-yl)thieno[2,3-d]pyrimidine 42d (200.63 mg, 515.95 μmol) was dissolved in DMSO (7 mL) and (1r,3r)-3-methoxycyclobutan-1-amine hydrochloride (106.5 mg, 773.93 μmol) with ethylbis(propan-2-yl)amine (200.34 mg, 1.55 mmol, 270.0 μL, 3.0 equiv) were added at room temperature. The mixture was heated at 100° C. overnight, cooled and purified by HPLC (SunFire C18 Column; 2-10 min 60-75% methanol+NH₃; 30 mL/min). 6-(2-Fluoro-3-methoxyphenyl)-N-((1r,3r)-3-methoxycyclobutyl)-5-methyl-2-(1-methyl-1H-imidazol-2-yl)thieno[2,3-d]pyrimidin-4-amine 42 was obtained as yellow gum (18.0 mg, 7.3% yield). ¹H NMR (DMSO-d6, 400 MHz): δ (ppm) 2.30 (m, 4H), 2.50 (s, 3H), 3.18 (s, 3H), 3.93 (s, 3H), 4.05 (m, 4H), 4.71-4.77 (m, 1H), 6.85 (d, 1H), 7.03 (m, 2H), 7.28-7.32 (m, 3H).

### Compound 43

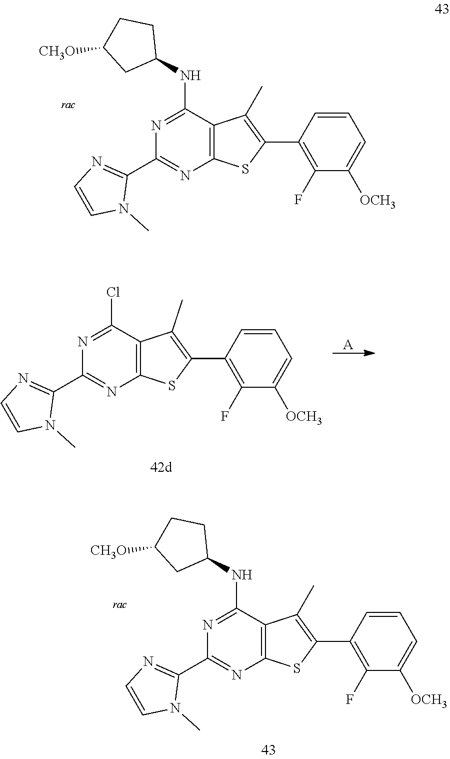

### Step A: 6-(2-Fluoro-3-methoxyphenyl)-N-((1R,3R)-3-methoxycyclopentyl)-5-methyl-2-(1-methyl-1H-imidazol-2-yl)thieno[2,3-d]pyrimidin-4-amine 43

4-Chloro-6-(2-fluoro-3-methoxyphenyl)-5-methyl-2-(1-methyl-1H-imidazol-2-yl)thieno[2,3-d]pyrimidine 42d (prepared as described for Compound 42, Step D) (201.26 mg, 517.58 μmol) was dissolved in DMSO (7 mL) and rac (1R,3R)-3-methoxycyclopentan-1-amine (89.42 mg, 776.37 μmol) with ethylbis(propan-2-yl)amine (267.12 mg, 2.07 mmol, 360.0 μl, 4.0 equiv) were added at room temperature. The mixture was heated at 100° C. overnight, cooled and purified by HPLC (SunFire C18 Column; 2-10 min 10-50% MeCN+NH₃; 30 mL/min). 6-(2-Fluoro-3-methoxyphenyl)-N-((1R,3R)-3-methoxycyclopentyl)-5-methyl-2-(1-methyl-1H-imidazol-2-yl)thieno[2,3-d]pyrimidin-4-amine 43 (31.0 mg, 12.2% yield) was obtained as yellow gum. ¹H NMR (DMSO-d6, 400 MHz): δ (ppm) 1.63-1.70 (m, 2H), 1.80-1.87 (m, 1H), 1.99-2.18 (m, 3H), 2.48 (m, 3H), 3.20 (m, 4H), 3.91 (s, 3H), 4.06 (s, 3H), 4.71-4.76 (m, 1H), 6.51 (d, 1H), 7.03 (m, 2H), 7.32 (m, 3H).

### Compound 44

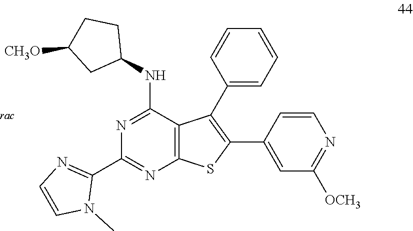

rac N-((1R,3S)-3-Methoxycyclopentyl)-6-(2-methoxypyridin-4-yl)-2-(1-methyl-1H-imidazol-2-yl)-5-phenylthieno[2,3-d]pyrimidin-4-amine 44 was prepared using the method described for Compound 40, except using the appropriate reagents. Formula weight: 513. Mass observed: 513.2.

### Compound 45

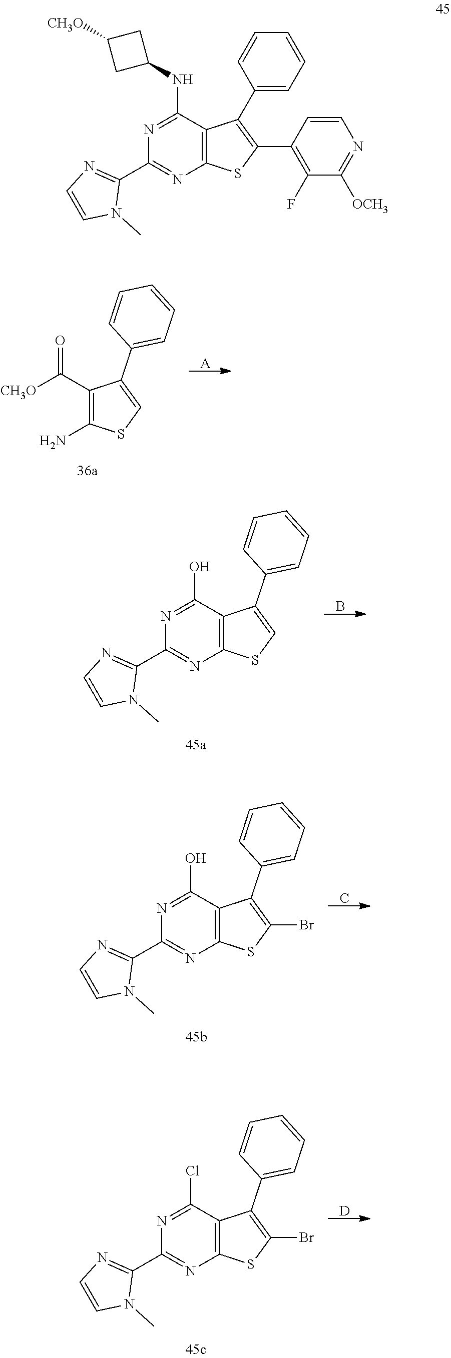

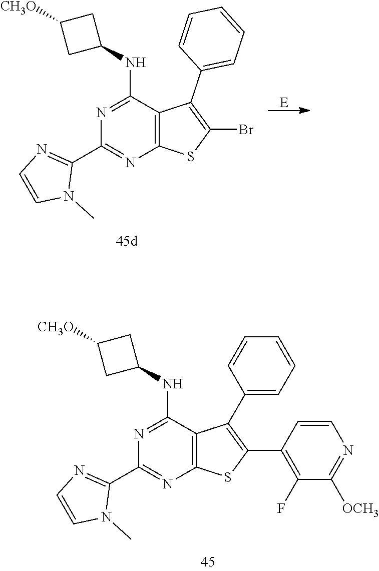

### Step A: 2-(1-Methyl-1H-imidazol-2-yl)-5-phenylthieno[2,3-d]pyrimidin-4-ol 45a

Methyl 2-amino-4-phenylthiophene-3-carboxylate 36a (22.5 g, 96.45 mmol), 1-methyl-1H-imidazole-2-carbonitrile (15.5 g, 144.67 mmol, 15.5 ml, 1.5 equiv), and potassium t-butoxide (86.58 g, 771.57 mmol) was dissolved in dry methanol (220 mL) and stirred overnight at room temperature. The resulting mixture was evaporated and acetic acid was added to pH 5. Then it was extracted with EtOAc (250 mL), the organic layer was separated, dried over Na₂SO₄ and evaporated to give compound 45a, 2-(1-methyl-1H-imidazol-2-yl)-5-phenylthieno[2,3-d]pyrimidin-4-ol, (17.0 g, 55.13 mmol, 57.2% yield), which was used without additional purification in a further step.

### Step B: 6-Bromo-2-(1-methyl-1H-imidazol-2-yl)-5-phenylthieno[2,3-d]pyrimidin-4-ol 45b

Compound 45a, 2-(1-methyl-1H-imidazol-2-yl)-5-phenylthieno[2,3-d]pyrimidin-4-ol, (25.0 g, 81.07 mmol) was dissolved in DMF (250 mL), then N-bromosuccinimide (21.64 g, 121.61 mmol) was added at room temperature. Reaction mixture was heated at 65° C. overnight, cooled to room temperature and poured into ice (500 mL). The resulting precipitate was filtered and dried in air at 60° C. to give compound 45b, 6-bromo-2-(1-methyl-1H-imidazol-2-yl)-5-phenylthieno[2,3-d]pyrimidin-4-ol, (20.0 g, 80.0% purity, 41.32 mmol, 51% yield) as yellow solid.

### Step C: 6-Bromo-4-chloro-2-(1-methyl-1H-imidazol-2-yl)-5-phenylthieno[2,3-d]pyrimidine 45c

Compound 45b 6-bromo-2-(1-methyl-1H-imidazol-2-yl)-5-phenylthieno[2,3-d]pyrimidin-4-ol, (23.0 g, 59.4 mmol) was suspended in P(O)Cl₃ (91.08 g, 594.03 mmol, 55.37 mL, 10.0 eq) and N,N-diisopropylethylamine (23.03 g, 178.21 mmol, 31.04 mL, 3.0 equiv) was added at room temperature. The reaction mixture was refluxed for 16 h, the solution was cooled to room temperature, evaporated under reduced pressure, poured in ice, diluted with ice-cold ammonia (200 mL, 20-25% of ammonia). The product was extracted with chloroform (2×500 mL) and evaporated. 6-Bromo-4-chloro-2-(1-methyl-1H-imidazol-2-yl)-5-phenylthieno[2,3-d]pyrimidine 45c (22.0 g, 43.0% purity, 23.32 mmol, 39.3% yield) was obtained as yellow solid.

### Step D: 6-Bromo-N-((1r,3r)-3-methoxycyclobutyl)-2-(1-methyl-1H-imidazol-2-yl)-5-phenylthieno[2,3-d]pyrimidin-4-amine 45d

Diisopropylethylamine (717.12 mg, 5.55 mmol) was added in one portion to the solution of 6-bromo-4-chloro-2-(1-methyl-1H-imidazol-2-yl)-5-phenylthieno[2,3-d]pyrimidine 45c (750.0 mg, 1.85 mmol) and (1r,3r)-3-methoxycyclobutan-1-amine hydrochloride (305.41 mg, 2.22 mmol) in DMF (10 mL). The mixture was heated to 100° C. and stirred at this temperature for 16 h. The mixture was purified by HPLC (2-10 min; 40-70% H₂O-MeOH+NH₃, flow 30 mL/min ((loading pump 4 mL MeOH); column: YMC-ACTUS TRIART C18 100*20 5 microM). 6-Bromo-N-((1r,3r)-3-methoxycyclobutyl)-2-(1-methyl-1H-imidazol-2-yl)-5-phenylthieno[2,3-d]pyrimidin-4-amine 45d (200.0 mg, 425.18 μmol, 23% yield) was obtained.

### Step E: 6-(3-Fluoro-2-methoxypyridin-4-yl)-N-((1r,3r)-3-methoxycyclobutyl)-2-(1-methyl-1H-imidazol-2-yl)-5-phenylthieno[2,3-d]pyrimidin-4-amine 45

6-Bromo-N-((1r,3r)-3-methoxycyclobutyl)-2-(1-methyl-1H-imidazol-2-yl)-5-phenylthieno[2,3-d]pyrimidin-4-amine 45d (155.8 mg, 331.22 μmol), (3-fluoro-2-methoxypyridin-4-yl)boronic acid (107.87 mg, 631.06 μmol), Pd(dppf)Cl₂ (25.77 mg, 31.55 μmol) and cesium carbonate (308.42 mg, 946.59 μmol) was dissolved in degassed dioxane (3.1 ml) under Ar. Then 0.16 mL of water was added via syringe. The reaction mixture was heated to 100° C. and stirred at this temperature for 48 h. The mixture was cooled to room temperature, filtered through celite and concentrated. The residue was purified by HPLC (2-10 min; 10-50% H₂O-MeOH+FA, flow 30 mL/min (loading pump 4 mL MeOH); column: Chromatorex 100*19 mm, 5 microM). 6-(3-Fluoro-2-methoxypyridin-4-yl)-N-((1r,3r)-3-methoxycyclobutyl)-2-(1-methyl-1H-imidazol-2-yl)-5-phenylthieno[2,3-d]pyrimidin-4-amine 45 (4.9 mg, 9.49 μmol, 3% yield) was obtained. ¹H NMR (CD₃OD, 400 MHz) δ 1.80 (m, 2H), 2.30 (m, 2H), 3.19 (s, 3H), 3.80 (quin, 1H), 3.96 (s, 3H), 4.19 (s, 3H), 4.52 (quin, 1H), 6.79 (dd, 1H), 7.28 (m, 2H), 7.57 (m, 6H), 7.78 (d, 1H). ¹H NMR (CD₃OD, 400 MHz) δ 1.80 (m, 2H), 2.30 (m, 2H), 3.19 (s, 3H), 3.80 (quin, 1H), 3.96 (s, 3H), 4.19 (s, 3H), 4.52 (quin, 1H), 6.79 (dd, 1H), 7.28 (m, 2H), 7.57 (m, 6H), 7.78 (d, 1H).

### Compound 46

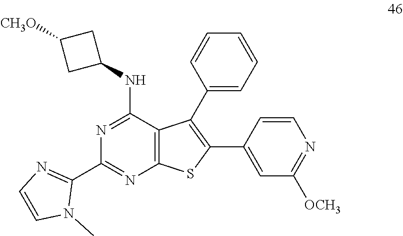

N-((1r,3r)-3-Methoxycyclobutyl)-6-(2-methoxypyridin-4-yl)-2-(1-methyl-1H-imidazol-2-yl)-5-phenylthieno[2,3-d]pyrimidin-4-amine 46 was prepared using the method described for Compound 45, except using the appropriate reagents. Formula weight: 499. Mass observed: 499.2.

### Compound 47

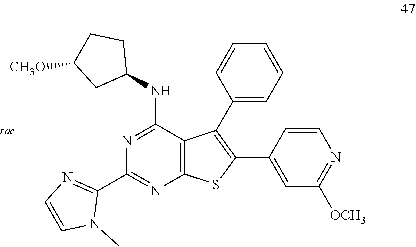

rac N-((1R,3R)-3-Methoxycyclopentyl)-6-(2-methoxypyridin-4-yl)-2-(1-methyl-1H-imidazol-2-yl)-5-phenylthieno[2,3-d]pyrimidin-4-amine 47 was prepared using the method described for Compound 45, except using the appropriate reagents. ¹H NMR (CD₃OD, 400 MHz,) δ 1.05 (quin., 1H), 1.24 (quin, 1H), 1.58 (m, 2H), 2.00 (m, 2H), 3.20 (s, 3H), 3.63 (m, 1H), 3.79 (s, 3H), 4.12 (s, 3H), 4.51 (m, 1H), 6.52 (s, 1H), 6.73 (d, 1H), 7.20 (m, 2H), 7.42 (m, 2H), 7.61 (m, 3H), 7.92 (d, 1H).

### Example 2

### Protocols for Cell-Based Phosphorylation Assays

### Protocol for Phospho-Smad2/3 Inhibition Assay

Cell lines: Human tumor-derived pancreatic cancer cell line Panc-1 and mouse muscle myoblast C2C12 were purchased from American Type Culture Collection and grown in complete DMEM-High Glucose, supplemented with penicillin (100 U/mL), streptomycin (100 μg/mL), and 10% heat-inactivated FBS at 37° C. in a humidified incubator with 5% CO₂.

Method: Cells were plated at 350000 cells/well density in a 12-well plate, allowed 3 hours to adhere to the plate, then starved in the appropriate medium in the presence of 0.5% FBS overnight. The small molecules to be tested were added to the cells in the final concentration of 10 μM in the presence of 0.3% DMSO for 3 hours incubation at 37° C. For IC₅₀ value determination, serial dilutions of compounds were added to cells under the same conditions. Next, Panc-1 cells were stimulated with 10 ng/ml TGF-b1 for 1 hour, and C2C12 cells were stimulated with 10 ng/ml TGF-b1 for 20 minutes (recombinant human TGF-b1, R&D Systems). After stimulation cells were lysed with lysis buffer containing 1% Triton X-100, EDTA, and Halt™ Protease & Phosphatase Inhibitor Cocktail (Thermo Scientific). Protein concentration was assessed by BCA protein assay (Thermo Scientific). Phosphorylation level of Smad2/Smad3 was determined by western blot.

Western blot protocol: Equal amounts of protein (15-50 μg) were separated by SDS-PAGE and transferred to nitrocellulose membranes (Invitrogen by Thermo Fisher Scientific). The membrane was stained with Ponceau S Stain (Boston BioProducts) to verify uniform protein loading. Membranes were blocked with 10% milk and phosphorylation levels of Smad2/Smad3 were assessed by incubating overnight at 4° C. with the following antibodies: Phospho-Smad2 (Ser465/467) and Phosph-Smad3 (Ser423/425), both from Cell Signaling Technology. Then the membranes were incubated with HRP-conjugated secondary antibody (Jackson Immunoresearch, West Grove, PA). Bands were incubated in Amersham ECL Prime Western Blotting Detection Reagent (GE Healthcare) and visualized using the ChemiDoc MP imaging system (Bio-Rad).

### Protocol for JNK Activation Assay

Cell lines: Human tumor-derived pancreatic cancer cell line Panc-1 and mouse muscle myoblast C2C12 were purchased from American Type Culture Collection and grown in complete DMEM-High Glucose, supplemented with penicillin (100 U/mL), streptomycin (100 μg/mL), and 10% heat-inactivated FBS at 37° C. in a humidified incubator with 5% CO₂.

Method: Cells were plated at 350000 cells/well density in a 12-well plate, allowed 3 hours to adhere to the plate, then starved in the appropriate medium in the presence of 0.5% FBS overnight. The small molecules to be tested were added to the cells in the final concentration of 10 μM in the presence of 0.3% DMSO for 3 hours incubation at 37° C. For IC50 value determination, serial dilutions of compounds were added to cells under the same conditions. Next, Panc-1 cells were stimulated with 10 ng/ml TGF-b1 for 1 hour, and C2C12 cells were stimulated with 10 ng/ml TGF-b1 for 20 minutes (recombinant human TGF-b1, R&D Systems). After stimulation cells were lysed with lysis buffer containing 1% Triton X-100, EDTA, and Halt™ Protease & Phosphatase Inhibitor Cocktail (Thermo Scientific). Protein concentration was assessed by BCA protein assay (Thermo Scientific). Phosphorylation level of JNK was determined by western blot.

Western blot protocol: Equal amounts of protein (15-50 μg) were separated by SDS-PAGE and transferred to nitrocellulose membranes (Invitrogen by Thermo Fisher Scientific). The membrane was stained with Ponceau S Stain (Boston BioProducts) to verify uniform protein loading. Membranes were blocked with 10% milk and phosphorylation levels of Smad2, Smad3, JNK and p38 were assessed by incubating overnight at 4° C. with the following antibody: Phospho-SAPK/JNK (Thr183/Tyr185), from Cell Signaling Technology. Then the membranes were incubated with HRP-conjugated secondary antibody (Jackson Immunoresearch, West Grove, PA). Bands were incubated in Amersham ECL Prime Western Blotting Detection Reagent (GE Healthcare) and visualized using the ChemiDoc MP imaging system (Bio-Rad).

### Protocol for MAPK p38 Activation Assay

Cell lines: Human tumor-derived pancreatic cancer cell line Panc-1 and mouse muscle myoblast C2C12 were purchased from American Type Culture Collection and grown in complete DMEM-High Glucose, supplemented with penicillin (100 U/mL), streptomycin (100 μg/mL), and 10% heat-inactivated FBS at 37° C. in a humidified incubator with 5% C02.

Method: Cells were plated at 350000 cells/well density in a 12-well plate, allowed 3 hours to adhere to the plate, then starved in the appropriate medium in the presence of 0.5% FBS overnight. The small molecules to be tested were added to the cells in the final concentration of 10 μM in the presence of 0.3% DMSO for 3 hours incubation at 37° C. For IC50 value determination, serial dilutions of compounds were added to cells under the same conditions. Next, Panc-1 cells were stimulated with 10 ng/ml TGF-b1 for 1 hour, and C2C12 cells were stimulated with 10 ng/ml TGF-b1 for 20 minutes (recombinant human TGF-b1, R&D Systems). After stimulation cells were lysed with lysis buffer containing 1% Triton X-100, EDTA, and Halt™ Protease & Phosphatase Inhibitor Cocktail (Thermo Scientific). Protein concentration was assessed by BCA protein assay (Thermo Scientific). Phosphorylation level of MAPK p38 was determined by western blot.

Western blot protocol: Equal amounts of protein (15-50 μg) were separated by SDS-PAGE and transferred to nitrocellulose membranes (Invitrogen by Thermo Fisher Scientific). The membrane was stained with Ponceau S Stain (Boston BioProducts) to verify uniform protein loading. Membranes were blocked with 10% milk and phosphorylation levels of Smad2/Smad3 were assessed by incubating overnight at 4° C. with the following antibody: Phospho-p38 MAPK (Thr180/Tyr182), from Cell Signaling Technology. Then the membranes were incubated with HRP-conjugated secondary antibody (Jackson Immunoresearch, West Grove, PA). Bands were incubated in Amersham ECL Prime Western Blotting Detection Reagent (GE Healthcare) and visualized using the ChemiDoc MP imaging system (Bio-Rad).

### Protocol for Western ERK1/2 Cell-Based Phosphorylation Assay

Cell lines: Human tumor-derived pancreatic cancer cell line Panc-1 was purchased from American Type Culture Collection and grown in complete DMEM-High Glucose, supplemented with penicillin (100 U/mL), streptomycin (100 μg/mL), and 10% heat-inactivated FBS at 37° C. in a humidified incubator with 5% CO2.

Method: Cells were plated at 350000 cells/well density in a 12-well plate, allowed 3 hours to adhere to the plate, then starved in the appropriate medium in the presence of 0.5% FBS overnight. The small molecules to be tested were added to the cells in the final concentration of 10 μM in the presence of 0.3% DMSO for 6 hours incubation at 37° C. For IC50 value determination, serial dilutions of compounds were added to cells under the same conditions. Next, cells were stimulated with 1.5 ng/ml EGF for 15 minutes then cells were lysed with lysis buffer containing 1% Triton X-100, EDTA, and Halt™ Protease & Phosphatase Inhibitor Cocktail (Thermo Scientific). Protein concentration was assessed by BCA protein assay (Thermo Scientific). Phosphorylation level of ERK1/2 was determined by western blot.

Western blot protocol: Equal amounts of protein (15-50 μg) were separated by SDS-PAGE and transferred to nitrocellulose membranes (Invitrogen by Thermo Fisher Scientific). The membrane was stained with Ponceau S Stain (Boston BioProducts) to verify uniform protein loading. Membranes were blocked with 10% milk and phosphorylation levels of ERK1/2 were assessed by incubating overnight at 4° C. with anti-phospho-p44/42 (Thr202/Tyr204) antibody (Cell Signaling) followed by HRP-conjugated secondary antibody (Jackson Immunoresearch, West Grove, PA). Bands were incubated in Amersham ECL Prime Western Blotting Detection Reagent (GE Healthcare) and visualized using the ChemiDoc MP imaging system (Bio-Rad).

### Protocol for Western AKT Cell-Based Phosphorylation Assay

Human tumor-derived pancreatic cancer cell line Panc-1 was purchased from American Type Culture Collection and grown in complete DMEM-High Glucose supplemented with penicillin (100 U/mL), streptomycin (100 μg/mL), and 10% heat-inactivated FBS at 37° C. in a humidified incubator with 5% CO2.

Method: Cells were plated at 350000 cells/well density in a 12-well plate, allowed 3 hours to adhere to the plate, then starved in the appropriate medium in the presence of 0.5% FBS overnight. The small molecules to be tested were added to the cells in the final concentration of 10 μM in the presence of 0.3% DMSO for 6 hours incubation at 37° C. Next, cells were stimulated with 1.5 ng/ml EGF for 15 minutes then cells were lysed with lysis buffer containing 1% Triton X-100, EDTA, and Halt™ Protease & Phosphatase Inhibitor Cocktail (Thermo Scientific). Protein concentration was assessed by BCA protein assay (Thermo Scientific). Phosphorylation level of Akt was determined by western blot.

Western blot protocol: Equal amounts of protein (15-50 μg) were separated by SDS-PAGE and transferred to nitrocellulose membranes (Invitrogen by Thermo Fisher Scientific). The membrane was stained with Ponceau S Stain (Boston BioProducts) to verify uniform protein loading. Membranes were blocked with 10% milk and phosphorylation levels of Akt were assessed by incubating overnight at 4° C. with anti-phospho-Akt (Ser473) antibody (Cell Signaling) followed by HRP-conjugated secondary antibody (Jackson Immunoresearch, West Grove, PA). Bands were incubated in Amersham ECL Prime Western Blotting Detection Reagent (GE Healthcare) and visualized using the ChemiDoc MP imaging system (Bio-Rad).

Table 7 shows inhibition data for selected compounds tested in one or more of the cellular assays described above.

### Protocol for Mouse TNF Alpha and IL6 Quantification Assay

Cell lines: Abelson murine leukemia virus transformed macrophage cell line RAW 264.7 was purchased from ATCC and grown in complete DMEM-High Glucose medium supplemented with penicillin (100 U/mL), streptomycin (100 μg/mL), and 10% heat-inactivated FBS at 37° C. in a humidified incubator with 5% CO₂.

Method: Cells were plated at 40000 cells/well density in a 96-wells plate. After a 3-hour incubation, macrophages were starved with DMEM plus 0.5% FBS o/n. The next day the small molecules to be tested were added to the cells in the final concentration of 30 μM (with 0.3% DMSO) 3 hours prior to LPS stimulation (100 ng/ml). After LPS stimulation cells were incubated at 37° C. for 16 h. At the end of the incubation period, culture media were collected and production of LPS-induced TNFα and IL6 cytokine was measured using ELISA detection kits.

Sandwich ELISA: The ELISA Immunoassays Quantikine Mouse TNF-alpha (catalog number MTA00B) and IL6 (catalog number M60001B) were purchased from R&D Systems Inc., Minneapolis, MN, These 4.5 hours solid phase ELISAs were used to measure mouse TNF□ or IL6 levels in macrophages culture supernatants. Assays were executed according to the manufacturer specifications.

Table 3 shows the compounds evaluated in the screening assays described above, Tables 4 and 5 show the data for these selected compounds tested in one or more of the cellular assays described above, such as in Human tumor-derived pancreatic cancer cell line Panc-1, and Table 6 shows the data for these selected compounds tested in one or more of the cellular assays described above in mouse muscle myoblast C2C12.

TABLE 3
Compounds

| Compound No. | Structure | Name |
|---|---|---|
| 1 | 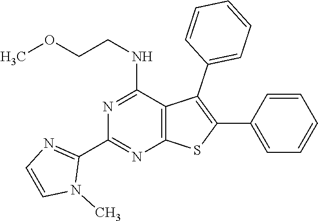 [SMILES: COCCNc1nc(-c2nccn2C)nc2sc(-c3ccccc3)c(-c3ccccc3)c12] | N-(2-Methoxyethyl)-2-(1- methyl-1H-imidazol-2-yl)-5,6- diphenylthieno[2,3-d]pyrimidin- 4-amine |
| 2 | 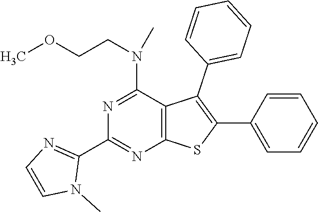 [SMILES: COCCN(C)c1nc(-c2nccn2C)nc2sc(-c3ccccc3)c(-c3ccccc3)c12] | N-(2-Methoxyethyl)-N-methyl- 2-(1-methyl-1H-imidazol-2-yl)- 5,6-diphenylthieno[2,3- d]pyrimidin-4-amine |
| 3 | 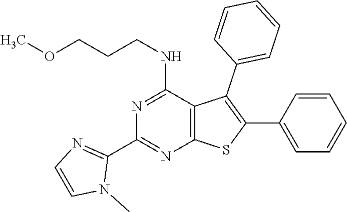 [SMILES: COCCCNc1nc(-c2nccn2C)nc2sc(-c3ccccc3)c(-c3ccccc3)c12] | N-(3-Methoxypropyl)-2-(1- methyl-1H-imidazol-2-yl)-5,6- diphenylthieno[2,3-d]pyrimidin- 4-amine |
| 4 | 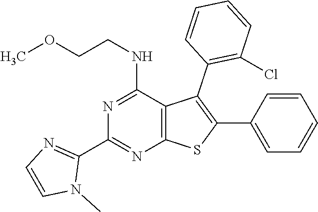 [SMILES: COCCNc1nc(-c2nccn2C)nc2sc(-c3ccccc3)c(-c3ccccc3Cl)c12] | 5-(2-Chlorophenyl)-N-(2- methoxyethyl)-2-(1-methyl-1H- imidazol-2-yl)-6- phenylthieno[2,3-d]pyrimidin-4- amine |
| 5 | 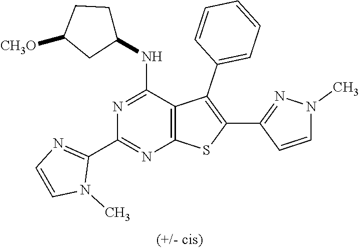 | rac-N-((1R,3S)-3- Methoxycyclopentyl)-2-(1- methyl-1H-imidazol-2-yl)-6-(1- methyl-1H-pyrazol-3-yl)-5- phenylthieno[2,3-d]pyrimidin-4- amine |
| 6 | 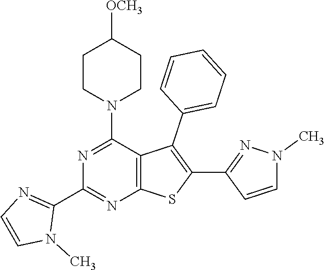 [SMILES: COC1CCN(c2nc(-c3nccn3C)nc3sc(-c4ccn(C)n4)c(-c4ccccc4)c23)CC1] | 4-(4-Methoxypiperidin-1-yl)-2- (1-methyl-1H-imidazol-2-yl)-6- (1-methyl-1H-pyrazol-3-yl)-5- phenylthieno[2,3-d]pyrimidine |
| 7 | 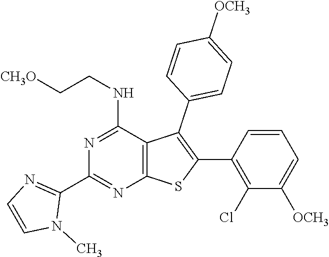 | 6-(2-Chloro-3-methoxyphenyl)- N-(2-methoxyethyl)-5-(4- methoxyphenyl)-2-(1-methyl- 1H-imidazol-4-yl)thieno[2,3- d]pyrimidin-4-amine |
| 8 | 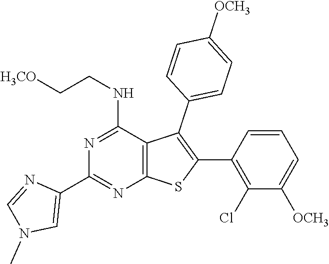 [SMILES: COCCNc1nc(-c2cn(C)cn2)nc2sc(-c3cccc(OC)c3Cl)c(-c3ccc(OC)cc3)c12] | 6-(2-Chloro-3-methoxyphenyl)- N-(2-methoxyethyl)-5-(4- methoxyphenyl)-2-(1-methyl- 1H-imidazol-4-yl)thieno[2,3- d]pyrimidin-4-amine |
| 9 | 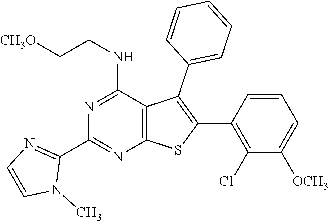 | 6-(2-Chloro-3-methoxyphenyl)- N-(2-methoxyethyl)-2-(1- methyl-1H-imidazol-2-yl)-5- phenylthieno[2,3-d]pyrimidin-4- amine |
| 10 | 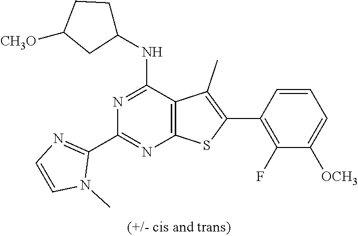 | rac-6-(2-Fluoro-3- methoxyphenyl)-N-((1R,3S)-3- methoxycyclopentyl)-5-methyl- 2-(1-methyl-1H-imidazol-2- yl)thieno[2,3-d]pyrimidin-4- amine and rac-6-(2-Fluoro-3- methoxyphenyl)-N-((1R,3R)-3- methoxycyclopentyl)-5-methyl- 2-(1-methyl-1H-imidazol-2- yl)thieno[2,3-d]pyrimidin-4- amine |
| 11 | 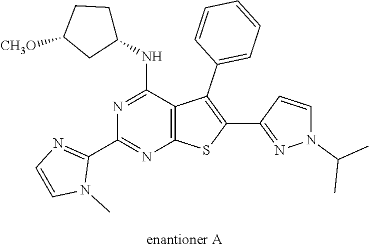 | 6-(1-Isopropyl-1H-pyrazol-3-yl)- N-((1S,3R)-3- methoxycyclopentyl)-2-(1- methyl-1H-imidazol-2-yl)-5- phenylthieno[2,3-d]pyrimidin-4- amine |
| 12 | 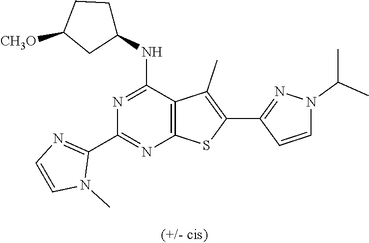 | rac-6-(1-Isopropyl-1H-pyrazol- 3-yl)-N-((1R,3S)-3- methoxycyclopentyl)-5-methyl- 2-(1-methyl-1H-imidazol-2- yl)thieno[2,3-d]pyrimidin-4- amine |
| 13 | 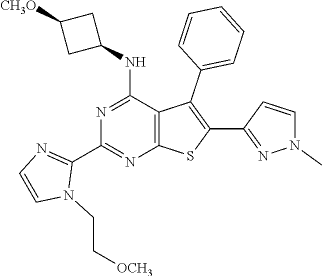 | N-((1s,3s)-3- methoxycyclobutyl)-2-(1-(2- methoxyethyl)-1H-imidazol-2- yl)-6-(1-methyl-1H-pyrazol-3- yl)-5-phenylthieno[2,3- d]pyrimidin-4-amine |
| 14 | 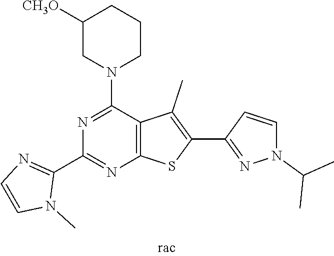 | rac-6-(1-Isopropyl-1H-pyrazol- 3-yl)-4-(3-methoxypiperidin-1- yl)-5-methyl-2-(1-methyl-1H- imidazol-2-yl)thieno[2,3- d]pyrimidine |
| 15 | 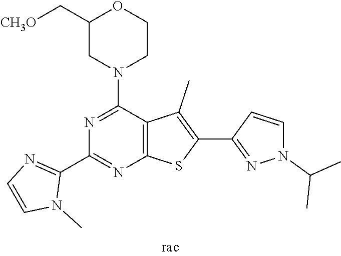 | rac-4-(6-(1-Isopropyl-1H- pyrazol-3-yl)-5-methyl-2-(1- methyl-1H-imidazol-2- yl)thieno[2,3-d]pyrimidin-4- yl)-2-(methoxymethyl) morpholine |
| 16 | 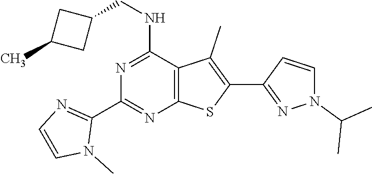 [SMILES: Cc1c(-c2ccn(C(C)C)n2)sc2nc(-c3nccn3C)nc(NC[C@H]3C[C@H](C)C3)c12] | 6-(1-Isopropyl-1H-pyrazol-3-yl)- N-(((1r,3r)-3- methoxycyclobutyl)methyl)-5- methyl-2-(1-methyl-1H- imidazol-2-yl)thieno[2,3- d]pyrimidin-4-amine |
| 17 | 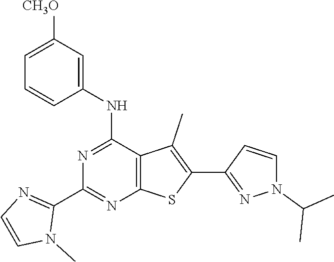 | 6-(1-Isopropyl-1H-pyrazol-3-yl)- N-(3-methoxyphenyl)-5-methyl- 2-(1-methyl-1H-imidazol-2- yl)thieno[2,3-d]pyrimidin-4- amine |
| 18 | 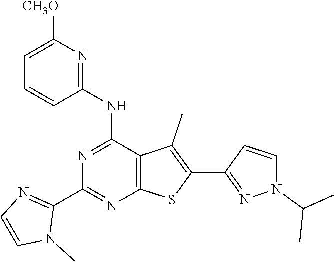 | 6-(1-Isopropyl-1H-pyrazol-3-yl)- N-(6-methoxypyridin-2-yl)-5- methyl-2-(1-methyl-1H- imidazol-2-yl)thieno[2,3- d]pyrimidin-4-amine |
| 19 | 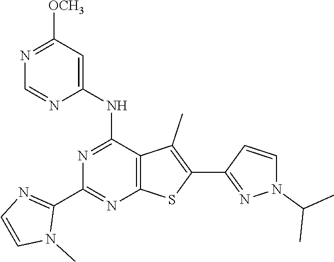 [SMILES: COc1cc(Nc2nc(-c3nccn3C)nc3sc(-c4ccn(C(C)C)n4)c(C)c23)ncn1] | 6-(1-Isopropyl-1H-pyrazol-3-yl)- N-(6-methoxypyrimidin-4-yl)-5- methyl-2-(1-methyl-1H- imidazol-2-yl)thieno[2,3- d]pyrimidin-4-amine |
| 20 | 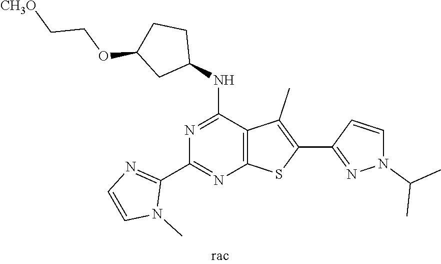 | rac-6-(1-Isopropyl-1H-pyrazol- 3-yl)-N-((1R,3S)-3-(2- methoxyethoxy)cyclopentyl)-5- methyl-2-(1-methyl-1H- imidazol-2-yl)thieno[2,3- d]pyrimidin-4-amine |
| 21 | 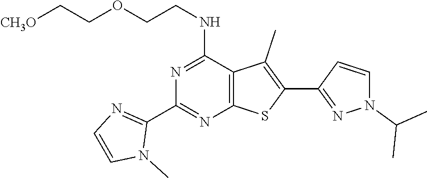 | 6-(1-Isopropyl-1H-pyrazol-3-yl)- N-(2-(2-methoxyethoxy)ethyl)- 5-methyl-2-(1-methyl-1H- imidazol-2-yl)thieno[2,3- d]pyrimidin-4-amine |
| 22 | 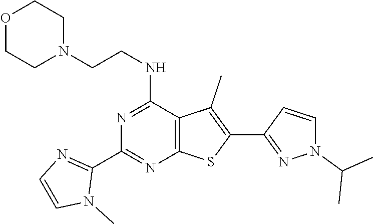 [SMILES: Cc1c(-c2ccn(C(C)C)n2)sc2nc(-c3nccn3C)nc(NCCN3CCOCC3)c12] | 6-(1-Isopropyl-1H-pyrazol-3-yl)- 5-methyl-2-(1-methyl-1H- imidazol-2-yl)-N-(2- morpholinoethyl)thieno[2,3- d]pyrimidin-4-amine |
| 23 | 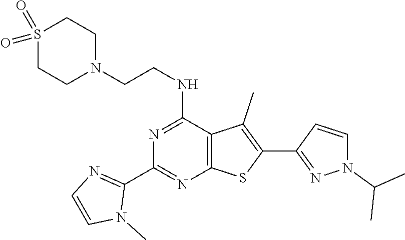 [SMILES: Cc1c(-c2ccn(C(C)C)n2)sc2nc(-c3nccn3C)nc(NCCN3CCS(=O)(=O)CC3)c12] | 4-(2-((6-(1-Isopropyl-1H- pyrazol-3-yl)-5-methyl-2-(1- methyl-1H-imidazol-2- yl)thieno[2,3-d]pyrimidin-4- yl)amino)ethyl)thiomorpholine 1,1-dioxide |
| 24 | 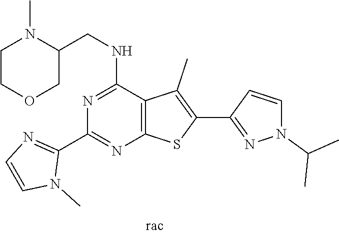 [SMILES: Cc1c(-c2ccn(C(C)C)n2)sc2nc(-c3nccn3C)nc(NCC3COCCN3C)c12] | rac-6-(1-Isopropyl-1H-pyrazol- 3-yl)-5-methyl-2-(1-methyl-1H- imidazol-2-yl)-N-((4- methylmorpholin-3- yl)methyl)thieno[2,3- d]pyrimidin-4-amine |
| 25 | 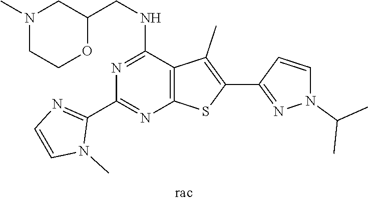 [SMILES: Cc1c(-c2ccn(C(C)C)n2)sc2nc(-c3nccn3C)nc(NCC3CN(C)CCO3)c12] | rac-6-(1-Isopropyl-1H-pyrazol- 3-yl)-5-methyl-2-(1-methyl-1H- imidazol-2-yl)-N-((4- methylmorpholin-2- yl)methyl)thieno[2,3- d]pyrimidin-4-amine |
| 26 | 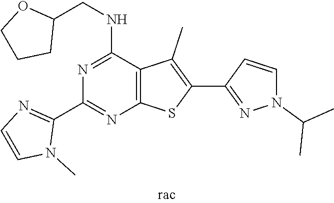 [SMILES: Cc1c(-c2ccn(C(C)C)n2)sc2nc(-c3nccn3C)nc(NCC3CCCO3)c12] | rac-6-(1-Isopropyl-1H-pyrazol- 3-yl)-5-methyl-2-(1-methyl-1H- imidazol-2-yl)-N- ((tetrahydrofuran-2- yl)methyl)thieno[2,3- d]pyrimidin-4-amine |
| 27 | 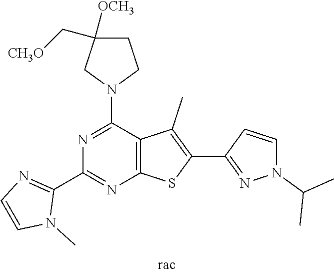 | rac-6-(1-Isopropyl-1H-pyrazol- 3-yl)-4-(3-methoxy-3- (methoxymethyl)pyrrolidin-1- yl)-5-methyl-2-(1-methyl-1H- imidazol-2-yl)thieno[2,3- d]pyrimidine |
| 28 | 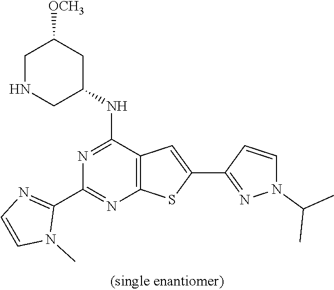 [SMILES: CO[C@H]1CNC[C@@H](Nc2nc(-c3nccn3C)nc3sc(-c4ccn(C(C)C)n4)cc23)C1] | 6-(1-Isopropyl-1H-pyrazol-3-yl)- N-((3S,5R)-5-methoxypiperidin- 3-yl)-2-(1-methyl-1H-imidazol- 2-yl)thieno[2,3-d]pyrimidin-4- amine |
| 29 | 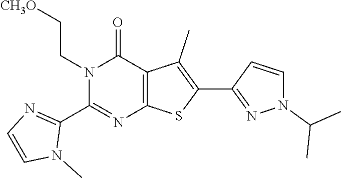 | 6-(1-Isopropyl-1H-pyrazol-3-yl)- 3-(2-methoxyethyl)-5-methyl-2- (1-methyl-1H-imidazol-2- yl)thieno[2,3-d]pyrimidin- 4(3H)-one |
| 30 | 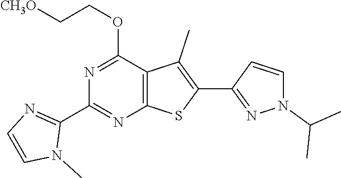 | 6-(1-Isopropyl-1H-pyrazol-3-yl)- 4-(2-methoxyethoxy)-5-methyl- 2-(1-methyl-1H-imidazol-2- yl)thieno[2,3-d]pyrimidine |
| 31 | 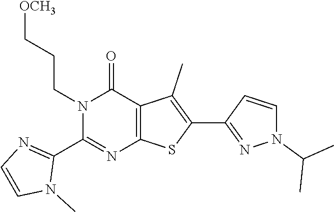 [SMILES: COCCCn1c(-c2nccn2C)nc2sc(-c3ccn(C(C)C)n3)c(C)c2c1=O] | 6-(1-Isopropyl-1H-pyrazol-3-yl)- 3-(3-methoxypropyl)-5-methyl- 2-(1-methyl-1H-imidazol-2- yl)thieno[2,3-d]pyrimidin- 4(3H)-one |
| 32 | 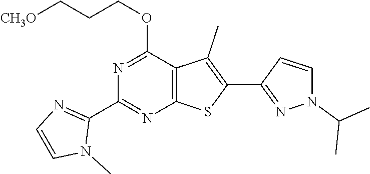 | 6-(1-Isopropyl-1H-pyrazol-3-yl)- 4-(3-methoxypropoxy)-5- methyl-2-(1-methyl-1H- imidazol-2-yl)thieno[2,3- d]pyrimidine |
| 33 | 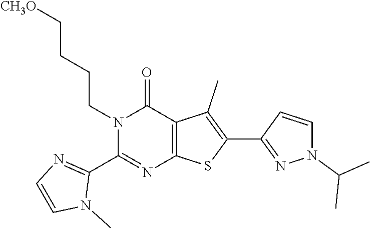 | 6-(1-Isopropyl-1H-pyrazol-3-yl)- 3-(4-methoxybutyl)-5-methyl-2- (1-methyl-1H-imidazol-2- yl)thieno[2,3-d]pyrimidin- 4(3H)-one |
| 34 | 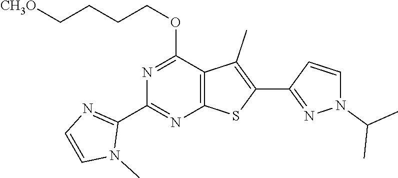 | 6-(1-Isopropyl-1H-pyrazol-3-yl)- 4-(4-methoxybutoxy)-5-methyl- 2-(1-methyl-1H-imidazol-2- yl)thieno[2,3-d]pyrimidine |
| 35 | 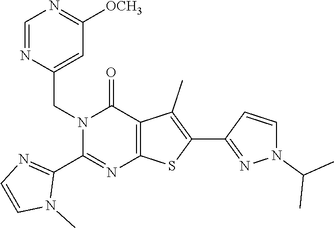 [SMILES: COc1cc(Cn2c(-c3nccn3C)nc3sc(-c4ccn(C(C)C)n4)c(C)c3c2=O)ncn1] | 6-(1-Isopropyl-1H-pyrazol-3-yl)- 3-((6-methoxypyrimidin-4- yl)methyl)-5-methyl-2-(1- methyl-1H-imidazol-2- yl)thieno[2,3-d]pyrimidin- 4(3H)-one |
| 36 | 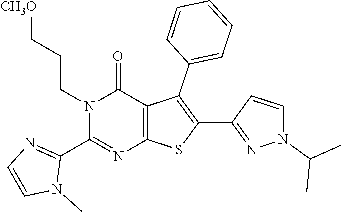 | 6-(1-Isoproyl-1H-pyrazol-3-yl)- 3-(3-methoxypropyl)-2-(1- methyl-1H-imidazol-2-yl)-5- phenylthieno[2,3-d]pyrimidin- 4(3H)-one |
| 37 | 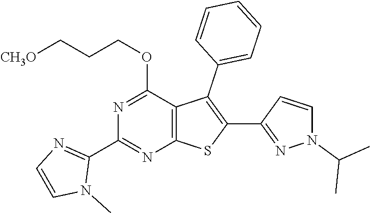 | 6-(1-Isopropyl-1H-pyrazol-3-yl)- 4-(3-methoxypropoxy)-2-(1- methyl-1H-imidazol-2-yl)-5- phenylthieno[2,3-d]pyrimidine |
| 38 | 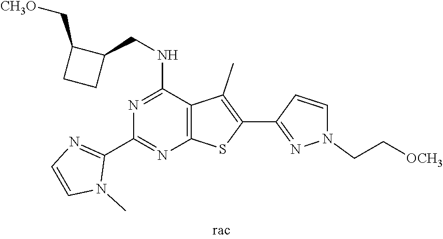 | rac-6-(1-(2-Methoxyethyl)-1H- pyrazol-3-yl)-N-(((1S,2R)-2- (methoxymethyl)cyclobutyl) methyl)-5-methyl-2-(1- methyl-1H-imidazol-2-yl) thieno[2,3-d]pyrimidin-4-amine |
| 39 | 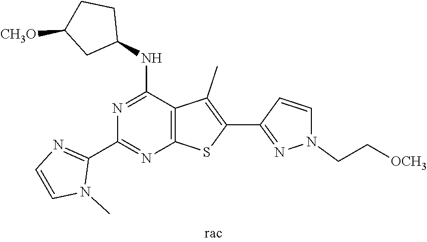 | rac-N-((1R,3S)-3- Methoxycyclopentyl)-6-(1-(2- methoxyethyl)-1H-pyrazol-3-yl)- 5-methyl-2-(1-methyl-1H- imidazol-2-yl)thieno[2,3- d]pyrimidin-4-amine |
| 40 | 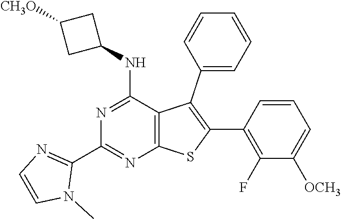 | 6-(2-Fluoro-3-methoxyphenyl)- N-((1r,3r)-3- methoxycyclobutyl)-2-(1- methyl-1H-imidazol-2-yl)-5- phenylthieno[2,3-d]pyrimidin-4- amine |
| 41 | 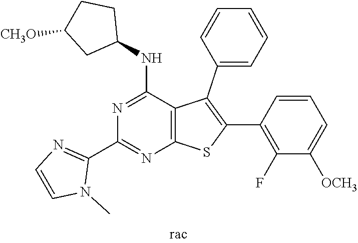 | 6-(2-Fluoro-3-methoxyphenyl)- N-((1R,3R)-3- methoxycyclopentyl)-2-(1- methyl-1H-imidazol-2-yl)-5- phenylthieno[2,3-d]pyrimidin-4- amine |
| 42 | 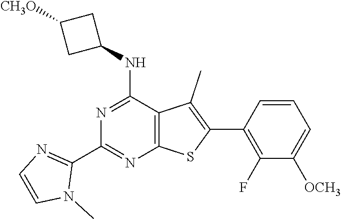 | 6-(2-Fluoro-3-methoxyphenyl)- N-((1r,3r)-3- methoxycyclobutyl)-5-methyl-2- (1-methyl-1H-imidazol-2- yl)thieno[2,3-d]pyrimidin-4- amine |
| 43 | 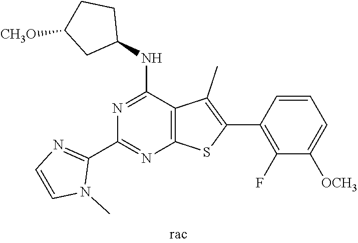 | rac-6-(2-Fluoro-3- methoxyphenyl)-N-((1R,3R)-3- methoxycyclopentyl)-5-methyl- 2-(1-methyl-1H-imidazol-2- yl)thieno[2,3-d]pyrimidin-4- amine |
| 44 | 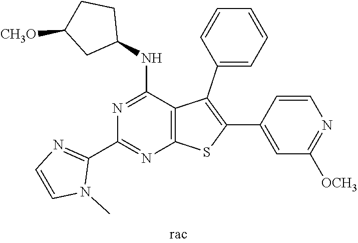 | rac-N-((1R,3S)-3- Methoxycyclopentyl)-6-(2- methoxypyridin-4-yl)-2-(1- methyl-1H-imidazol-2-yl)-5- phenylthieno[2,3-d]pyrimidin-4- amine |
| 45 | 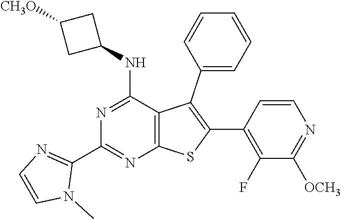 | 6-(3-Fluoro-2-methoxypyridin- 4-yl)-N-((1r,3r)-3- methoxycyclobutyl)-2-(1- methyl-1H-imidazol-2-yl)-5- phenylthieno[2,3-d]pyrimidin-4- amine |
| 46 | 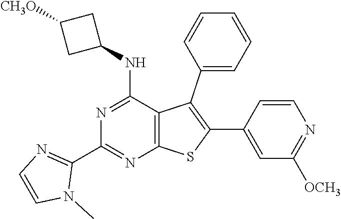 | N-((1r,3r)-3- Methoxycyclobutyl)-6-(2- methoxypyridin-4-yl)-2-(1- methyl-1H-imidazol-2-yl)-5- phenylthieno[2,3-d]pyrimidin- 4-amine |
| 47 | 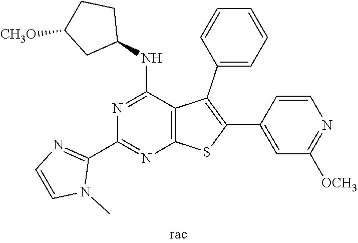 | N-((1R,3R)-3- Methoxycyclopentyl)-6-(2- methoxypyridin-4-yl)-2-(1- methyl-1H-imidazol-2-yl)-5- phenylthieno[2,3-d]pyrimidin-4- amine |

TABLE 4
Inhibition of Smad2/3, Activation of JNK (at 10 μM) and Activation of
MAPK p38 (at 10 μM), each in PANC-1 pancreatic cancer cell line

| | % inh of Smad2/3 | Phospho-Smad2/3 | | Activation of |
|---|---|---|---|---|
| | phosphorylation | Inhibition: IC50 | Activation of | MAPK p38 in |
| Compound | in PANC-1 | (uM) | JNK in PANC-1 | PANC-1 |
| 1 | C | ND | Y | N |
| 2 | 0 | ND | Y | Y |
| 3 | 0 | ND | Y | Y |
| 4 | B | ND | Y | Y |
| 5 | D | J | Y | Y |
| 6 | 0 | ND | Y | N |
| 7 | 0 | J | Y | Y |
| 8 | D | J | Y | Y |
| 9 | D | K | Y | Y |
| 10 | A | ND | Y | Y |
| 11 | C | ND | Y | Y |
| 12 | A | ND | Y | Y |
| 13 | C | ND | Y | N |
| 14 | C | ND | Y | Y |
| 15 | B | ND | Y | Y |
| 16 | A* | ND | Y | Y |
| 17 | C | ND | Y | Y |
| 18 | C | ND | Y | Y |
| 19 | D | ND | Y | Y |
| 20 | A | ND | Y | Y |
| 21 | 0 | ND | Y | Y |
| 22 | 0 | ND | Y | Y |
| 23 | 0 | ND | N | Y |
| 24 | 0 | ND | Y | Y |
| 25 | 0 | ND | Y | Y |
| 26 | 0 | ND | Y | Y |
| 27 | 0 | ND | Y | Y |
| 28 | 0 | ND | Y | Y |
| 29 | 0 | ND | Y | Y |
| 30 | 0 | ND | N | N |
| 31 | 0 | ND | N | N |
| 32 | 0 | ND | Y | N |
| 33 | 0 | ND | N | N |
| 34 | 0 | ND | Y | N |
| 35 | 0 | ND | N | N |
| 36 | 0 | ND | Y | Y |
| 37 | 0 | ND | N | Y |
| 38 | C | ND | Y | Y |
| 39 | 0 | ND | Y | Y |
| 40 | D | ND | Y | Y |
| 41 | D | ND | Y | Y |
| 42 | A | ND | Y | Y |
| 43 | D | ND | Y | Y |
| 44 | D | ND | Y | N |
| 45 | D | ND | Y | N |
| 46 | D | ND | Y | N |
| 47 | D | ND | Y | N |

0 = 0% inhibition, A = 1-25% inhibition, B = 26-45% inhibition, C = 46-75% inhibition, D = 76-100% inhibition, A* = 1-25% activation, B* = 26-45% activation, C* = 46-75% activation, D* = 76-100% activation, I = >10 uM IC₅₀, J = 5-10 uM IC₅₀, K = 1-4.99 uM IC₅₀, L = <1 uM IC₅₀, ND = not determined, Y = yes, N = No.

TABLE 5
% Inhibition at 10 μM of ERK 1/2, AKT, IL-6, TNFα and MiaPaca2 proliferation

| | % inh of ERK1/2 | % inh of AKT | % inh of | Proliferation | | |
|---|---|---|---|---|---|---|
| | phosphorylation | phosphorylation | proliferation | in MiaPaca2: | IL-6: | TNFα: |
| Compound | in PANC-1 | in PANC-1 | in MiaPaca2 | IC50 (uM) | % inh. | % inh. |
| 14 | D | C | D | J | D | B |
| 15 | C | D* | C | J | C | D |
| 16 | D | 0 | D | L | C | A |
| 17 | D | C | D | J | D | D |
| 18 | 0 | B | D | L | B | 0 |
| 19 | D | D | D | K | D | D |
| 20 | C | B* | D | K | D | C |
| 21 | 0 | A* | D | J | C | A |
| 22 | 0 | A* | D | J | D | A |
| 23 | 0 | 0 | 0 | I | C | 0 |
| 24 | 0 | 0 | D | J | D | A |
| 25 | 0 | A* | C | J | D | C |
| 26 | 0 | 0 | C | I | D | 0 |
| 27 | A | B* | D | J | D | D |
| 28 | C | B* | D | J | D | C |
| 29 | 0 | 0 | C | J | D | D |
| 30 | 0 | 0 | A | I | 0 | 0 |
| 31 | 0 | 0 | 0 | I | C | 0 |
| 32 | 0 | A* | D | J | D | D |
| 33 | 0 | 0 | 0 | I | C | 0 |
| 34 | 0 | 0 | B | I | D | 0 |
| 35 | 0 | 0 | 0 | I | A | A |
| 36 | 0 | 0 | D | J | C | 0 |
| 37 | 0 | 0 | A | I | D | 0 |
| 38 | C | 0 | D | J | D | D |
| 39 | A | D* | D | J | B | 0 |
| 40 | D | D | D | K | D | D |
| 41 | D | D | D | K | D | D |
| 42 | C | B* | D | J | C | 0 |
| 43 | C | 0 | C | J | D | B |
| 44 | C | D* | D | J | ND | ND |
| 45 | D | D | D | K | ND | ND |
| 46 | D | A | D | K | ND | ND |
| 47 | D | D | D | L | ND | ND |

0 = 0% inhibition, A = 1-25% inhibition, B = 26-45% inhibition, C = 46-75% inhibition, D = 76-100% inhibition, A* = 1-25% activation, B* = 26-45% activation, C* = 46-75% activation, D* = 76-100% activation, I = >10 uM IC₅₀, J = 5-10 uM IC₅₀, K = 1-4.99 uM IC₅₀, L = <1 uM IC₅₀, ND = not determined.

TABLE 6
Inhibition of Smad2/3, Activation of JNK (at 10 μM) and Activation of
MAPK p38 (at 10 μM), each in mouse muscle myoblast C2C12 cell line

| | % inh of Smad2/3 | Phospho-Smad2/3 | | Activation of |
|---|---|---|---|---|
| | phosphorylation | Inhibition: IC50 | Activation of JNK | MAPK p38 in |
| Compound | in C2C12 | (uM) | in C2C12 | C2C12 |
| 8 | ND | K | Y | Y |
| 9 | D | K | Y | Y |
| 17 | ND | J | ND | ND |
| 18 | ND | J | ND | ND |
| 19 | ND | I | ND | ND |

0 = 0% inhibition, A = 1-25% inhibition, B = 26-45% inhibition, C = 46-75% inhibition, D = 76-100% inhibition, I = >10 uM IC₅₀, J = 5-10 uM IC₅₀, K = 1-4.99 uM IC₅₀, L = <1 uM IC₅₀, ND = not determined, Y = yes, N = No.

### Example 3

### Ras GTP Binding Domain Inhibition Assay

A Ras Superfamily Activity Assay (as disclosed in International Application No. PCT/US2018/038613, filed Jun. 20, 2018, which is incorporated by reference in its entirety) was used as a specific assay to evaluate compounds against the following for the following Ras Superfamily proteins: KRas wild type, KRas Q61H mutant, KRas G12C mutant, KRas G12D mutant, Rac-1, and Rho-A.

Buffer-I:
25 mM Tris-HCl, pH 7.4
27 mM KCl
137 mM NaCl
1 mM MgCl₂
1 mM DTT

Buffer-IL:
50 mM Tris-HCl, pH 7.0
1 mM MgCl₂
1 mM DTT

The small GTPases proteins: KRas wild type. KRas Q61H mutant, KRas G12C mutant, KRas G12D mutants, Rac-1, and Rho-A were expressed as His-tagged proteins. In addition, the Guanosine nucleotide Exchange Factor (GEF) Sos protein (residues 556-1049) was expressed as a His-tagged protein. In cells, the guanine nucleotide exchange factor Sos protein promotes activation of Ras proteins by stimulating an exchange of GDP for GTP. The inclusion of Sos to the Ras GTP binding domain inhibition assay may be considered as an alternative representation of physiological cellular conditions for evaluating the inhibitory activity of some of the tested small molecules.

For the assay, all purified small GTPases proteins were diluted in Buffer-I or Buffer-II to a final concentration of 10-30 μg/mL. 200 μL of each diluted protein was added to a nickel-coated 96-well plate and incubated overnight at 4° C. Then the protein solution was discarded and 200 μL of Buffer-I or Buffer-II was added to each well in the presence of 1% DMSO. Compounds to be tested were added to the protein-coated wells at final concentration of 20 μM, and incubated for 3 hours at room temperature with and without 10-30 μg/mL of Sos added to the final hour of the incubation. When performing IC₅₀ measurements a serial dilution of all tested concentrations was added. Then Cy3-GTP or Cy5-GTP was added to each well to a final concentration of 100 nM. The labeled GTP was incubated for 45 minutes at room temperature. Following GTP incubation, wells were washed 3× in Buffer-I or Buffer-II and 200 μL of Buffer-I or Buffer-II was added to each well. Following washes, the amount of bound labeled-GTP was measured using a SpectraMax M3 (Molecular Devices).

### Protocol for cdc42 and Rheb GTP Binding Domain Inhibition Assay

The following method was developed as specific assay for cdc42 and Rheb proteins.

Buffer-I:
25 mM Tris-HCl, pH 7.4
27 mM KCl
137 mM NaCl
1 mM MgCl₂
1 mM DTT

Buffer-II:
50 mM Tris-HCl, pH 7.0
1 mM MgCl2
1 mM DTT

The small GTPases proteins cdc42 and Rheb were expressed as His-tagged proteins. For the assay, the cdc42 and Rheb purified small GTPases proteins were diluted in Buffer-I or Buffer-II to a final concentration of 10-30 μg/mL. 200 μL of each diluted protein was added to a nickel-coated 96-well plate and incubated overnight at 40 C. Then the protein solution was discarded and 200 μL of Buffer-I or Buffer-II was added to each well in the presence of 1% DMSO. Compounds to be tested were added to the protein-coated wells at final concentration of 20 μM, and incubated for 3 hours at room temperature with and without 10-30 μg/mL of Sos added to the final hour of the incubation. When performing IC50 measurements a serial dilution of all tested concentrations was added. Then Cy3-GTP or Cy5-GTP was added to each well to a final concentration of 100 nM. The labeled GTP was incubated for 45 minutes at room temperature. Following GTP incubation, wells were washed 3× in Buffer-I or Buffer-II and 200 μL of Buffer-I or Buffer-II was added to each well. Following washes, the amount of bound labeled-GTP was measured using a SpectraMax M3 (Molecular Devices).

Tables 7-9 show % inhibition data, and Table 10 shows IC₅₀ measurements, for selected compounds tested in the screening assays described above.

TABLE 7
% Inhibition at 20 μM of K-Ras mutant and wild-type protein

| | | KRas | | KRas | | KRas | | KRas |
|---|---|---|---|---|---|---|---|---|
| | KRas | wild | KRas | G12D | KRas | G12C | KRas | Q61H |
| | wild | type + | G12D | mutant + | G12C | mutant + | Q61H | mutant + |
| | type: | SOS: | mutant: | SOS: | mutant: | SOS: | mutant: | SOS: |
| Compound | % inh. | % inh. | % inh. | % inh. | % inh. | % inh. | % inh. | % inh. |
| 1 | A | A | D | A | C | ND | C | C |
| 2 | C | ND | C | ND | B | ND | ND | ND |
| 3 | C | A | C | A | C | ND | C | C |
| 4 | A | ND | B | ND | B | ND | ND | ND |
| 5 | B | D | A | C | B | D | B | D |
| 6 | 0 | B | B | B | B | C | C | C |
| 7 | C | D | B | B | B | D | B | D |
| 8 | C | D | B | B | B | D | C | D |
| 9 | C | D | C | C | B | D | C | D |
| 10 | C | C | D | C | D | C | C | C |
| 11 | C | D | C | C | C | D | C | D |
| 12 | D | D | C | C | C | D | D | D |
| 13 | D | D | C | D | D | D | D | D |
| 14 | 0 | D | ND | ND | B | D | B | D |
| 15 | D | D | D | D | C | D | D | D |
| 16 | D | C | B | C | C | C | C | C |
| 17 | B | C | C | C | C | D | B | C |
| 18 | C | C | D | C | C | C | C | C |
| 19 | D | D | B | C | C | D | D | D |
| 20 | D | D | D | D | D | D | C | D |
| 21 | D | D | D | D | C | D | D | D |
| 22 | D | D | D | D | D | D | D | D |
| 23 | D | D | D | D | C | D | D | D |
| 24 | B | D | C | D | C | D | C | D |
| 25 | D | D | D | D | D | D | D | D |
| 26 | D | D | D | C | D | D | D | D |
| 27 | D | D | C | D | D | D | C | D |
| 28 | D | D | C | D | D | D | C | D |
| 29 | D | D | C | C | C | D | C | D |
| 30 | A | 0 | 0 | 0 | A | 0 | 0 | A |
| 31 | A | A | 0 | 0 | 0 | 0 | 0 | 0 |
| 32 | D | D | C | C | C | C | C | C |
| 33 | 0 | A | 0 | 0 | 0 | A | 0 | A |
| 34 | A | 0 | A | 0 | 0 | A | 0 | A |
| 35 | A | A | A | 0 | 0 | A | A | A |
| 36 | A | A | 0 | 0 | 0 | A | 0 | 0 |
| 37 | 0 | A | 0 | 0 | 0 | 0 | 0 | 0 |
| 38 | C | D | B | D | C | D | C | D |
| 39 | D | D | C | D | D | D | D | D |
| 40 | C | D | C | D | C | D | D | D |
| 41 | B | D | A | C | B | D | B | D |
| 42 | D | D | B | D | C | D | C | D |
| 43 | B | D | A | D | B | D | B | D |
| 44 | C | D | D | C | B | D | B | D |
| 45 | C | D | C | C | B | D | C | D |
| 46 | C | D | C | C | B | D | D | D |
| 47 | D | D | C | D | C | D | C | D |

0 = 0% inhibition,
A = 1-25% inhibition,
B = 26-45% inhibition,
C = 46-75% inhibition,
D = 76-100% inhibition,
ND = not determined

TABLE 8
% Inhibition at 20 μM of KRas
G12D mutant, Rac-1 and Rho-A protein

| | | KRas | | | | |
|---|---|---|---|---|---|---|
| | KRas | G12D | | | | |
| | G12D | mutant + | | Rac-1 + | | Rho-A + |
| Com- | mutant: | SOS: | Rac-1: | SOS: | Rho-A: | SOS: |
| pound | % inh. | % inh. | % inh. | % inh. | % inh. | % inh. |
| 1 | D | A | D | ND | C | ND |
| 2 | C | ND | B | ND | B | ND |
| 3 | C | A | C | ND | C | ND |
| 4 | B | ND | B | ND | B | ND |
| 5 | A | C | C | C | C | D |
| 6 | B | B | B | B | 0 | 0 |
| 7 | B | B | C | C | B | C |
| 8 | B | B | C | C | C | C |
| 9 | C | C | C | C | B | B |
| 10 | D | C | C | C | D | D |
| 11 | C | C | A | B | B | B |
| 12 | C | C | C | C | D | D |
| 13 | C | D | D | D | D | D |
| 14 | ND | ND | C | ND | B | ND |
| 15 | D | D | D | ND | D | ND |
| 16 | B | C | B | ND | C | ND |
| 17 | C | C | B | ND | C | ND |
| 18 | D | C | C | ND | D | ND |
| 19 | B | C | C | ND | D | ND |
| 20 | D | D | C | ND | C | ND |
| 21 | D | D | D | ND | D | ND |
| 22 | D | D | D | ND | D | ND |
| 23 | D | D | C | ND | C | ND |
| 24 | C | D | D | ND | C | ND |
| 25 | D | D | D | ND | D | ND |
| 26 | D | C | C | ND | D | ND |
| 27 | C | D | A | ND | C | ND |
| 28 | C | D | C | ND | C | ND |
| 29 | C | C | 0 | ND | D | ND |
| 30 | 0 | 0 | 0 | ND | 0 | ND |
| 31 | 0 | 0 | 0 | ND | 0 | ND |
| 32 | C | C | 0 | ND | D | ND |
| 33 | 0 | 0 | 0 | ND | 0 | ND |
| 34 | A | 0 | 0 | ND | 0 | ND |
| 35 | A | 0 | 0 | ND | 0 | ND |
| 36 | 0 | 0 | 0 | ND | 0 | ND |
| 37 | 0 | 0 | 0 | ND | 0 | ND |
| 38 | B | D | A | ND | B | ND |
| 39 | C | D | B | ND | C | ND |
| 40 | C | D | B | ND | B | ND |
| 41 | A | C | A | ND | 0 | ND |
| 42 | B | D | 0 | ND | C | ND |
| 43 | A | D | A | ND | B | ND |
| 44 | D | C | 0 | ND | 0 | ND |
| 45 | C | C | C | ND | C | ND |
| 46 | C | C | C | ND | C | ND |
| 47 | C | D | C | ND | C | ND |

0 = 0% inhibition, A = 1-25% inhibition, B = 26-45% inhibition, C = 46-75% inhibition, D = 76-100% inhibition, ND = not determined.

TABLE 9
% Inhibition at 20 μM of Cdc42 and Rheb

| | Cdc42: | Rheb: |
|---|---|---|
| Compound | % inh. | % inh. |
| 40 | B | D |
| 44 | A | B |
| 45 | C | D |
| 46 | C | D |
| 47 | C | D |

0 = 0% inhibition, A = 1-25% inhibition, B = 26-45% inhibition, C = 46-75% inhibition, D = 76-100% inhibition.

TABLE 10
KRas Q61H mutant IC₅₀ (uM) values

| | KRas Q61H |
|---|---|
| | mutant + SOS: |
| Compound | IC50 (uM) |
| 11 | K |
| 12 | J |
| 13 | K |

I = >10 uM IC₅₀,
J = 5-10 uM IC₅₀,
K = 1-4.99 uM IC₅₀,
L = <1 uM IC₅₀

### EXAMPLE 4

### Assessment of Plasma Exposure of Compound 9 after IP Administration in DBA/2J Mice

### Protocol for Assessment of Plasma Exposure

Experimental Design: Compound 9 was suspended in 0.5% Hydroxypropyl Methylcellulose (HPMC)/0.1% Tween® 80 to a final concentration of 10 mg/mL. The dosing solutions were prepared prior to the start of the test. Male DBA/2J mice (Jackson Stock No: 000671) were housed in solid bottom cages with standard chow and water ad libitum. Dosing was initiated when animals were 7-8 weeks old. Mice were dosed at a volume of 10 mL/kg by intraperitoneal (IP) injection at 100 mg/kg or vehicle. Each animal was observed for mortality and signs of pain or distress. No findings of overt toxicity were observed. Blood was collected at 15 min, 30 min, 1 h, 2 h, 4 h, 7 h, 16 h, 24 h by cardiac puncture, mixed gently 8 times and put on ice until centrifuged within 30 minutes of collection. The plasma was transferred to a 96-well plate for analysis of test compound levels.

| Group | Test Compound | Dose (mg/kg) | Route | Number of Mice |
|---|---|---|---|---|
| 1 | VEH 1 | — | PO | 18 |
| 2 | Compound 9/VEH 1 | 25 | PO | 18 |
| 3 | Compound 9/VEH 1 | 100 | PO | 18 |
| 4 | Compound 9/VEH 2 | 100 | PO | 18 |
| 5 | Compound 9/VEH 1 | 100 | IP | 24 |

Vehicle 1: 0.5% HPMC with 0.1% Tween 80; Vehicle 2: Solubilized Lipid.

Preparation of Standard Solutions for Calibration of UPLC: Assay: 2 mg/mL in DMSO Stock solutions of reference standard of Compound 9 was freshly prepared. 15 uL of the 2 mg/mL DMSO stock solution of Compound 9 was spiked into a vial and diluted to a final volume of 1 mL (30 μg/mL). 100 uL of the 30 μg/mL solution was mixed with 900 uL methanol (final conc. 3 μg/mL). A calibration curve was obtained from solutions having concentrations of 0.001, 0.003, 0.01, 0.03, 0.1, 0.5 and 1 μg/mL, which were prepared further dilution of the 3 μg/mL solution with methanol. An internal standard of D3-desacetylDFZ (500 ng/mL in methanol) was used.

### UPLC Conditions:

Column: Waters UPLC Acquity BEH C18 Column 2.1*50, 1.7 uM

Mobile phase: A: 0.1% formic acid in water; B: 0.1% formic acid in 100% acetonitrile

### Gradient:

| | Flow rate | | | |
|---|---|---|---|---|
| Time (min) | (mL/min) | % A | % B | Curve |
| 0 | 0.45 | 80 | 20 | |
| 0.4 | 0.45 | 80 | 20 | 1 |
| 1.2 | 0.45 | 2 | 98 | 6 |
| 1.6 | 0.45 | 2 | 98 | 6 |
| 1.7 | 0.45 | 80 | 20 | 11 |

Temperature: 50° C.

Run time: 2 min

Injection volume=2 uL

Instrument: Waters TQ-s

Desolvation Temperature: 500° C.

Desolvation gas flow: 1000 L/hr

Capillary Cone voltage: 0.5

Reference batch: 21-hydroxy emflaza, Compound 9

Preparation of Samples for UPLC:: Protein precipitation method using Millipore hydrophilic plate (MSRLN04) to filter the precipitated protein
1) Plasma calibration curve for positively charged compound: all in the filter plate added
   a) IS-410000 in acetonitrile (0.1 μg/mL) 200 uL
   b) Calibration curve in methanol: 20 uL
   c) Methanol: 20 uL
   d) Control mouse plasma: 20 uL
2) Test plasma
   a) IS-410000 in acetonitrile (0.1 μg/mL) 200 uL
   b) Calibration curve in methanol: 20 uL
   c) Test plasma: 20 uL

Placed sample on automatic shaker for at least 5 min

Centrifuged at 2000 rpm (Eppendorf 5804) for 2 min. 2-2.5 uL supernantant were injected onto LCMS system

### Results for Assessment of Plasma Exposure with Compound 9

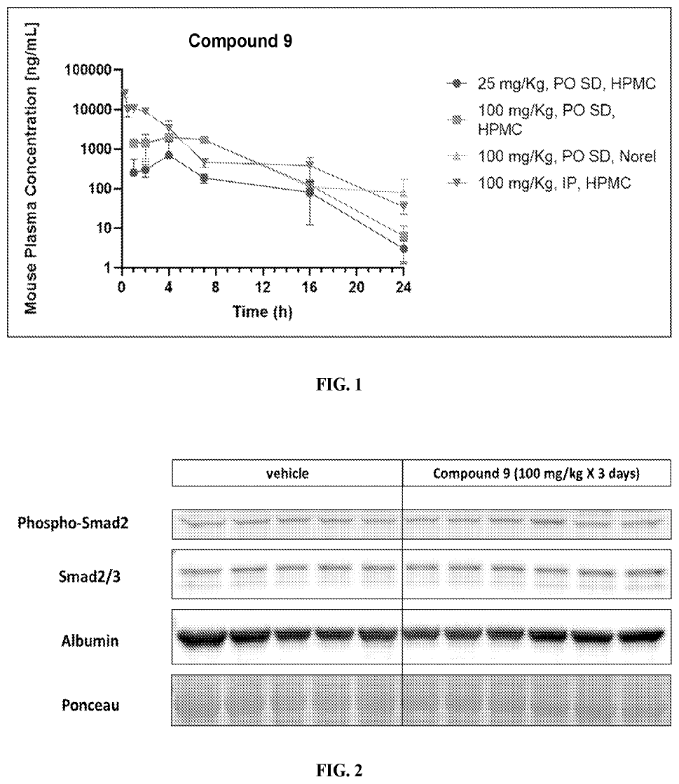

The results obtained from this study are summarized in FIG. 1 and in the table below.

| Group | t_{1/2} (h) | Tₘₐₓ (h) | Cₘₐₓ (ng/mL) | AUCₗₐₛₜ (hr*ng/mL) | AUCINF_{obs} (hr*ng/mL) |
|---|---|---|---|---|---|
| 2 | 2.90 | 4 | 703 | 3883 | 3896 |
| 3 | 2.11 | 4 | 1956 | 16677 | 16696 |
| 4 | 2.30 | 4 | 2090 | 17120 | 17155 |
| 5 | 2.96 | 0.25 | 25974 | 42991 | 43170 |

Exposures (AUC) were similar when using Vehicle 1 compared to Vehicle 2 for oral dosing (PO). Based on the results from Groups 2 and 3, exposures (AUC) were observed to be dose proportional. Exposures were higher for intraperitoneal (IP) injection (Group 5) compared to oral dosing (Group 3).

### Example 5

### Assessment of the Pharmacodynamics of Compound 9 in the D2-mdx Mouse Model of Duchenne Muscular Dystrophy

The experiment described herein was designed to monitor the effects of Compound 9 on two biomarkers related to fibrosis in D2-mdx mice. The first biomarker is Smad2 phosphorylation, and the second is the levels of serum albumin in the different muscle tissues. Fibrosis causes disruption of the fine transparent tubular sheath which envelops the fibers of skeletal muscles, this can result in various molecules from the blood and interstitial fluid leaking into myofibres. A classic example is serum albumin, which can be identified in tissue sections and considered to be hallmark of myofibre permeability.

### Protocol

Experimental Design: Compound 9 was suspended in 0.5% Hydroxypropyl Methylcellulose (HPMC)/0.1% Tween® 80 to a final concentration of 10 mg/mL. The dosing solutions were prepared prior to the start of the test and were stored in the dark and maintained in frozen aliquots. Doses were thawed prior to administration. Male D2-mdx (JAX Stock No: 013141) mice were sourced from The Jackson Laboratory and housed in solid bottom cages with standard chow and water ad libitum. Dosing was initiated when animals were −3 weeks old. Animals were dosed daily by intraperitoneal (IP) injection at 100 mg/kg or vehicle for 3 days. Each animal was observed for mortality and signs of pain or distress. No findings of overt toxicity were observed. Mice were sacrificed by CO₂ euthanasia approximately 6 hours after their day 3 dosing. Heart, diaphragm, quadriceps, tibialis anterior (TA) samples were collected at termination on day 3 and snap frozen. These samples were shipped on dry ice for analysis, and were stored at −80° C.

Tissue protein extraction: Frozen tissues were placed on ice in prechilled tubes with 30 μL/mg with of ice-cold RIPA buffer+Halt™ Protease & Phosphatase Inhibitor Cocktail. Tissue was homogenized with a manual tissue homogenizer then centrifuged at 14,000 RPM at 4° C. for 15 min. Supernatant was removed and protein concentration determined by BCA assay.

Western blot protocol: Equal amounts of protein (15-50 μg) were separated by SDS-PAGE and transferred to nitrocellulose membranes (Invitrogen by Thermo Fisher Scientific). The membrane was stained with Ponceau S Stain (Boston BioProducts) to verify uniform protein loading. Membranes were blocked with 10% milk and albumin levels were assessed by incubating overnight at 4° C. with albumin antibody (Cell Signaling Technology #4929). Then the membranes were incubated with HRP-conjugated secondary antibody (Jackson Immunoresearch, West Grove, PA). Bands were incubated in Amersham ECL Prime Western Blotting Detection Reagent (GE Healthcare) and visualized using the ChemiDoc MP imaging system (Bio-Rad).

### Results

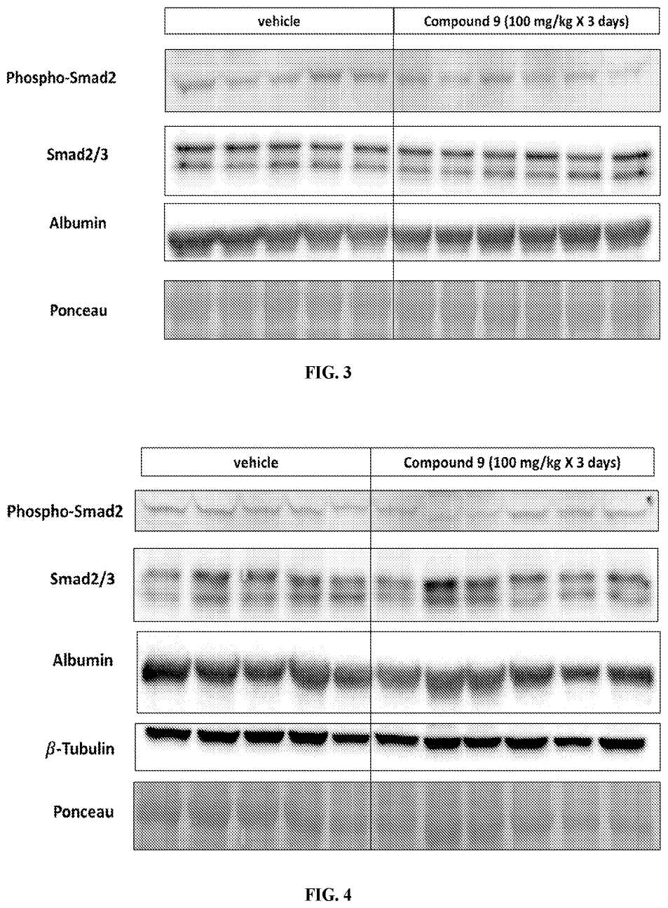

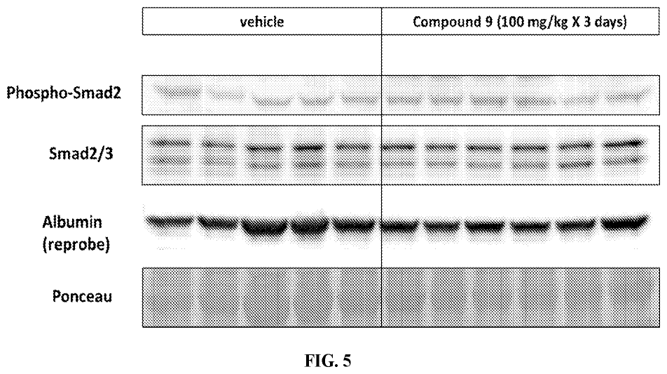

Western blot images for samples collected from heart (FIG. 2), diaphragm (FIG. 3), quadriceps (FIG. 4), and tibialis anterior (TA, FIG. 5) tissues following termination on day 3 were analyzed, providing the results summarized herein below (derived by densitometry).

Phospho-Smad2 Analysis

| | |
|---|---|
| Diaphragm | Quadriceps |

| | Average | Median | Average | Median |
|---|---|---|---|---|
| Vehicle | 0.27 ± 0.07 | 0.29 | 0.25 ± 0.14 | 0.18 |
| Treated | 0.18 ± 0.06 | 0.19 | 0.22 ± 0.11 | 0.28 |
| Ratio | 1.53 | 1.54 | 1.10 | 0.64 |

Albumin Analysis

| | Tibialis anterior | | Quadriceps | |
|---|---|---|---|---|
| | Average | Median | Average | Median |
| Vehicle | 40754 ± 12036 | 42364 | 40945 ± 7457 | 41883 |
| Treated | 34522 ± 4321 | 33494 | 32650 ± 4998 | 34329 |
| Ratio | 1.18 | 1.26 | 1.25 | 1.21 |

In the diaphragm, inhibition of Smad2 phosphorylation in mice dosed with Compound 9 was demonstrated as compared to control vehicle mice. A clear decrease in serum albumin levels was detected in the tibialis anterior and quadricep muscles. No changes were detected in the heart for both Smad2 phosphorylation and serum albumin levels. It should be noted that the study was performed on 3 weeks old mice, and thus, this observation is consistent with the normal progression of DMD where the heart is the last organ to be affected by the fibrotic process.

Overall, the data clearly indicates potential therapeutic benefits associated with Compound 9, and similar species, in the treatment of DMD in particular, and fibrosis in general, especially considering the fact that the mice were dosed for only 3 days.

### EXEMPLARY EMBODIMENTS

A1. A method of treating fibrosis in a subject having a fibrotic disease, comprising administering to the subject a compound of Formula I:

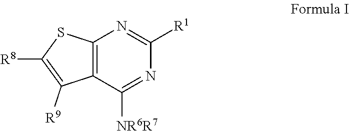

or a pharmaceutically acceptable salt thereof,
   wherein:
R¹ is H, alkyl, aryl or heteroaryl;
R⁸ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
R⁹ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
R³ is hydrogen, alkyl, alkenyl, alkynyl, aryl, heteroaryl, heterocyclyl, cycloalkyl, alkylcarbonyl, cycloalkylcarbonyl or arylcarbonyl;
R⁴ is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy, aralkoxy, or NR⁶R⁷;
each R⁵ is selected from the group consisting of hydrogen, alkyl, alkenyl, alkynyl, aryl, heteroaryl, heterocyclyl, cycloalkyl, alkylcarbonyl, cycloalkylcarbonyl, and arylcarbonyl; and
R⁶ and R⁷ are independently selected from the group consisting of hydrogen, alkyl, alkenyl, alkynyl, aryl, cycloalkyl, alkylcarbonyl, cycloalkylcarbonyl, arylcarbonyl, heteroarylcarbonyl, arylsulfonyl, heteroarylsulfonyl, cycloalkylsulfonyl and alkylsulfonyl, or
R⁶ and R⁷ are combined to form a cyclic structure including the nitrogen atom to which they are both attached;
   wherein the compound of Formula I or pharmaceutically acceptable salt thereof inhibits phosphorylation of Smad2/3, according to Phospho-Smad2/3 Inhibition Assay, and is inactive according to MAPK p38 Activation Assay.

A2. The method of embodiment A1, wherein:
R¹ is heteroaryl;
R⁸ is aryl or heteroaryl;
R⁹ is aryl; and
R⁶ and R⁷ are independently selected from the group consisting of hydrogen, alkyl and cycloalkyl.

A3. The method of embodiment A1 or embodiment A2, wherein the compound of Formula I or pharmaceutically acceptable salt thereof is selected from the group consisting of:
N-(2-Methoxyethyl)-2-(1-methyl-1H-imidazol-2-yl)-5,6-diphenylthieno[2,3-d]pyrimidin-4-amine; and
N-((1s,3s)-3-ethoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine.

A4. The method of any one of embodiments A1-A3, wherein the compound of Formula I or pharmaceutically acceptable salt thereof inhibits phosphorylation of Smad2/3 by 20% or more at 10 μM, according to Phospho-Smad2/3 Inhibition Assay.

A5. The method of embodiment A4, wherein the compound of Formula I or pharmaceutically acceptable salt thereof is selected from the group consisting of:
N-(2-Methoxyethyl)-2-(1-methyl-1H-imidazol-2-yl)-5,6-diphenylthieno[2,3-d]pyrimidin-4-amine; and
N-((1s,3s)-3-methoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine.
A6. The method of any one of embodiments A1-A3, wherein the compound of Formula I or pharmaceutically acceptable salt thereof inhibits phosphorylation of Smad2/3 by 30% or more at 10 μM, according to Phospho-Smad2/3 Inhibition Assay.
A7. The method of embodiment A6, wherein the compound of Formula I or pharmaceutically acceptable salt thereof is selected from the group consisting of:
N-(2-Methoxyethyl)-2-(1-methyl-1H-imidazol-2-yl)-5,6-diphenylthieno[2,3-d]pyrimidin-4-amine; and
N-((1s,3s)-3-methoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine.

A8. The method of any one of embodiments A1-A3, wherein the compound of Formula I or pharmaceutically acceptable salt thereof inhibits phosphorylation of Smad2/3 by 50% or more at 10 μM, according to Phospho-Smad2/3 Inhibition Assay.

A9. The method of embodiment A8, wherein the compound of Formula I or pharmaceutically acceptable salt thereof is selected from the group consisting of:
N-(2-Methoxyethyl)-2-(1-methyl-1H-imidazol-2-yl)-5,6-diphenylthieno[2,3-d]pyrimidin-4-amine; and
N-((1s,3s)-3-methoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine.

A10. The method of any one of embodiments A1-A3, wherein the compound of Formula I or pharmaceutically acceptable salt thereof inhibits phosphorylation of Smad2/3 by 70% or more at 10 μM, according to Phospho-Smad2/3 Inhibition Assay.

A11. The method of embodiment A10, wherein the compound of Formula I or pharmaceutically acceptable salt thereof is selected from the group consisting of:
N-((1s,3s)-3-methoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine.

A12. The method of any one of embodiments A1-All, wherein the compound of Formula I or pharmaceutically acceptable salt thereof is exclusive of compounds selected from the group consisting of:
N-(2-Methoxyethyl)-N-methyl-2-(1-methyl-1H-imidazol-2-yl)-5,6-diphenylthieno[2,3-d]pyrimidin-4-amine;
N-(3-Methoxypropyl)-2-(1-methyl-1H-imidazol-2-yl)-5,6-diphenylthieno[2,3-d]pyrimidin-4-amine;
5-(2-Chlorophenyl)-N-(2-methoxyethyl)-2-(1-methyl-1H-imidazol-2-yl)-6-phenylthieno[2,3-d]pyrimidin-4-amine;
rac-N-((1R,3S)-3-Methoxycyclopentyl)-2-(1-methyl-1H-imidazol-2-yl)-6-(1-methyl-1H-pyrazol-3-yl)-5-phenylthieno[2,3-d]pyrimidin-4-amine;
4-(4-Methoxypiperidin-1-yl)-2-(1-methyl-1H-imidazol-2-yl)-6-(1-methyl-1H-pyrazol-3-yl)-5-phenylthieno[2,3-d]pyrimidine;
6-(2-Chloro-3-methoxyphenyl)-N-(2-methoxyethyl)-5-(4-methoxyphenyl)-2-(1-methyl-1H-imidazol-2-yl)thieno[2,3-d]pyrimidin-4-amine;
6-(2-Chloro-3-methoxyphenyl)-N-(2-methoxyethyl)-5-(4-methoxyphenyl)-2-(1-methyl-1H-imidazol-4-yl)thieno[2,3-d]pyrimidin-4-amine;
6-(2-Chloro-3-methoxyphenyl)-N-(2-methoxyethyl)-2-(1-methyl-1H-imidazol-2-yl)-5-phenylthieno[2,3-d]pyrimidin-4-amine;
rac-6-(2-Fluoro-3-methoxyphenyl)-N-((1R,3S)-3-methoxycyclopentyl)-5-methyl-2-(1-methyl-1H-imidazol-2-yl)thieno[2,3-d]pyrimidin-4-amine and rac-6-(2-Fluoro-3-methoxyphenyl)-N-((1R,3R)-3-methoxycyclopentyl)-5-methyl-2-(1-methyl-1H-imidazol-2-yl)thieno[2,3-d]pyrimidin-4-amine;
6-(1-Isopropyl-1H-pyrazol-3-yl)-N-((1S,3R)-3-methoxycyclopentyl)-2-(1-methyl-1H-imidazol-2-yl)-5-phenylthieno[2,3-d]pyrimidin-4-amine; and
rac-6-(1-Isopropyl-1H-pyrazol-3-yl)-N-((1R,3S)-3-methoxycyclopentyl)-5-methyl-2-(1-methyl-1H-imidazol-2-yl)thieno[2,3-d]pyrimidin-4-amine.

A13. The method of any one of embodiments A1-A12, wherein the compound of Formula I or pharmaceutically acceptable salt thereof activates JNK, according to JNK Activation Assay.

A14. The method of any one of embodiments A1-A13, wherein the compound of Formula I or pharmaceutically acceptable salt thereof is a modulator of Ras superfamily activity according to a Ras Superfamily Activity Assay.

A15. A method of treating fibrosis in a subject having a fibrotic disease, comprising administering to the subject a compound or a pharmaceutically acceptable salt thereof identified as a modulator of Ras superfamily activity according to a Ras Superfamily Activity Assay; wherein the identified Ras superfamily modulating compound or pharmaceutically acceptable salt thereof inhibits phosphorylation of Smad2/3, according to Phospho-Smad2/3 Inhibition Assay, and is inactive according to MAPK p38 Activation Assay.

A16. The method of embodiment A15, wherein the identified Ras superfamily modulating compound or pharmaceutically acceptable salt thereof inhibits phosphorylation of Smad2/3 by 20%, 30%, 50%, 70%, or more at 10 μM, according to Phospho-Smad2/3 Inhibition Assay.

A17. The method of embodiment A15 or embodiment A16, wherein the identified Ras superfamily modulating compound or pharmaceutically acceptable salt thereof activates INK, according to JNK Activation Assay.

A18. The method of any one of embodiments A15-A17, wherein the Ras Superfamily Activity Assay comprises:
a) contacting the compound or pharmaceutically acceptable salt thereof with a Ras superfamily protein;
b) incubating a cyanine-labeled GTP with said compound or pharmaceutically acceptable salt thereof and the Ras superfamily protein; and
c) measuring the amount of the cyanine-labeled GTP bound to the Ras superfamily protein.

A19. The method of embodiment A18, wherein the Ras Superfamily Activity Assay is a cell-free assay.

A20. The method of embodiment A18 or embodiment A19, wherein the Ras Superfamily Activity Assay is a GTP-binding competition assay.

A21. The method of any one of embodiments A16-A20, wherein the Ras superfamily protein is a Ras protein, a Rac protein, or a Rho protein.

A22. The method of embodiment A21, wherein the Ras superfamily protein is a Ras protein.

A23. The method of embodiment A22, wherein the Ras Superfamily Activity Assay is a Ras Activity Assay.

A24. The method of embodiment A22 or embodiment A23, wherein the identified Ras superfamily modulating compound or pharmaceutically acceptable salt thereof is identified as a Ras modulating compound or a pharmaceutically acceptable salt thereof.

A25. The method of embodiment A24, wherein the identified Ras modulating compound or pharmaceutically acceptable salt thereof competitively inhibits GTP binding to the Ras GTP binding domain.

A26. The method of embodiment A24 or embodiment A25, wherein the identified Ras modulating compound or pharmaceutically acceptable salt thereof binds to the Ras protein GTP binding domain with greater than 25% inhibition at 20 uM.

A27. The method of any one of embodiments A24-A26, wherein the identified Ras modulating compound or pharmaceutically acceptable salt thereof has a binding affinity (Kd) to the Ras protein GTP binding domain of less than 10 uM.

A28. The method of any one of embodiments A24-A27, wherein the identified Ras modulating compound or pharmaceutically acceptable salt thereof inhibits the Ras activity and has an IC50 value of less than 10 uM.

A29. The method of any one of embodiments A24-A28, wherein the identified Ras modulating compound or pharmaceutically acceptable salt thereof inhibits binding of the cyanine labeled GTP with an IC50 value of less than 10 uM.

A30. The method of any one of embodiments A24-A29, wherein the cyanine-labeled GTP is a Cy3- or a Cy5-labeled GTP.

A31. The method of any one of embodiments A24-A30, wherein the Ras Activity Assay is a GTP-binding competition assay.

A32. The method of any one of embodiments A23-A31, wherein the Ras protein is immobilized.

A33. The method of any one of embodiments A23-A32, wherein the Ras protein is DIRAS I; DIRAS2; DIRAS3; ERAS; GEM; HRAS; KRAS; MRAS; NKIRASI; NKIRAS2; NRAS; RALA; RALB; RAPIA; RAPIB; RAP2A; RAP2B; RAP2C; RASDI; RASD2; RASLIOA; RASLIOB; RASLIIA; RASLIIB; RASL12; REMI; REM2; RERG; RERGL; RRAD; RRAS; or RRAS2.

A34. The method of any one of embodiments A23-A33, wherein the Ras protein is HRAS; KRAS; or NRAS, or a mutant thereof.

A35. The method of embodiment A34, wherein the Ras protein is a KRAS mutant.

A36. The method of embodiment A35, wherein the KRAS mutant is a KRas G12D mutant, KRas G12C mutant, or KRas Q61H mutant.

A37. The method of embodiment A34, wherein the Ras protein is wild-type KRas.

A38. The method of embodiment A34, wherein the Ras protein is HRAS or mutant thereof.

A39. The method of embodiment A34, wherein the Ras protein is NRAS or mutant thereof.

A40. The method of embodiment A21, wherein the Ras superfamily protein is a Rac protein.

A41. The method of embodiment A40, wherein the Ras Superfamily Activity Assay is a Rac Activity Assay.

A42. The method of embodiment A40 or embodiment A41, wherein the identified Ras superfamily modulating compound or pharmaceutically acceptable salt thereof is identified as a Rae modulating compound or a pharmaceutically acceptable salt thereof.

A43. The method of embodiment A42, wherein the identified Rac modulating compound or pharmaceutically acceptable salt thereof competitively inhibits GTP binding to the Rae GTP binding domain.

A44. The method of embodiment A42 or embodiment A43, wherein the identified Rae modulating compound or pharmaceutically acceptable salt thereof binds to the Rae protein GTP binding domain with greater than 25% inhibition at 20 uM.

A45. The method of any one of embodiments A42-A44, wherein the identified Rac modulating compound or pharmaceutically acceptable salt thereof has a binding affinity (Kd) to the Rae protein GTP binding domain of less than 10 uM.

A46. The method of any one of embodiments A42-A45, wherein the identified Rae modulating compound or pharmaceutically acceptable salt thereof inhibits the Rac activity and has an IC50 value of less than 10 uM.

A47. The method of any one of embodiments A42-A46, wherein the identified Rae modulating compound or pharmaceutically acceptable salt thereof inhibits binding of the cyanine labeled GTP with an IC50 value of less than 10 uM.

A48. The method of any one of embodiments A42-A47, wherein the cyanine-labeled GTP is a Cy3- or a Cy5-labeled GTP.

A49. The method of any one of embodiments A41-A48, wherein the Rae Activity Assay is a GTP-binding competition assay.

A50. The method of any one of embodiments A41-A49, wherein the Rae protein is immobilized.

A51. The method of any one of embodiments A41-A50, wherein the Rae protein is RAC1; RAC2; RAC3; RHOG, or a mutant thereof.

A52. The method of embodiment A51, wherein the Rae protein is wild-type RAC1.

A53. The method of embodiment A21, wherein the Ras superfamily protein is a Rho protein.

A54. The method of embodiment A53, wherein the Ras Superfamily Activity Assay is a Rho Activity Assay.

A55. The method of embodiment A53 or embodiment A54, wherein the identified Ras superfamily modulating compound or pharmaceutically acceptable salt thereof is identified as a Rho modulating compound or a pharmaceutically acceptable salt thereof.

A56. The method of embodiment A55, wherein the identified Rho modulating compound or pharmaceutically acceptable salt thereof competitively inhibits GTP binding to the Rho GTP binding domain.

A57. The method of embodiment A55 or embodiment A56, wherein the identified Rho modulating compound or pharmaceutically acceptable salt thereof binds to the Rho protein GTP binding domain with greater than 25% inhibition at 20 uM.

A58. The method of any one of embodiments A55-A57, wherein the identified Rho modulating compound or pharmaceutically acceptable salt thereof has a binding affinity (Kd) to the Rho protein GTP binding domain of less than 10 uM.

A59. The method of any one of embodiments A55-A58, wherein the identified Rho modulating compound or pharmaceutically acceptable salt thereof inhibits the Rho activity and has an IC50 value of less than 10 uM.

A60. The method of any one of embodiments A55-A59, wherein the identified Rho modulating compound or pharmaceutically acceptable salt thereof inhibits binding of the cyanine labeled GTP with an IC50 value of less than 10 uM.

A61. The method of any one of embodiments A55-A60, wherein the cyanine-labeled GTP is a Cy3- or a Cy5-labeled GTP.

A62. The method of any one of embodiments A54-A61, wherein the Rho Activity Assay is a GTP-binding competition assay.

A63. The method of any one of embodiments A54-A62, wherein the Rho protein is immobilized.

A64. The method of any one of embodiments A54-A63, wherein the Rho protein is RHOA; RHOB; RHOBTB1; RHOBTB2; RHOBTB3; RHOC; RHOD; RHOF; RHOH; RHOJ; RHOQ; RHOU; RHOV; RND1; RND2; RND3; CDC42, or a mutant thereof.

A65. The method of any one of embodiment A64, wherein the Rho protein is wild-type RHOA.

A66. The method of any one of embodiments A1-A65, wherein the method treats, prevents, or ameliorates one or more symptoms of the fibrotic disease in the subject.

A67. The method of any one of embodiments A1-A66, wherein the fibrotic disease is selected from the group consisting of fibrosis of kidney, fibrosis of cardiovascular system, pulmonary fibrosis, cystic fibrosis, idiopathic fibrosis, fibrosis of the lung, bridging fibrosis, fibrosis of the liver, fibrosis of the intestine, fibrosis of the muscular system, fibrosis of the brain, fibrosis of the joints, fibrosis of the skin, fibrosis of the bone marrow, fibrosis of the heart, fibrosis of the soft tissue, fibrosis of the tendons, fibrosis of the lymph nodes, fibrosis of the eyes, retroperitoneum, scleroderma and surgical scarring.

A68. The method of embodiment A67, wherein the fibrotic disease is fibrosis of the kidney.

A69. The method of embodiment A67, wherein the fibrotic disease is fibrosis of the cardiovascular system.

A70. The method of embodiment A67, wherein the fibrotic disease is pulmonary fibrosis.

A71. The method of embodiment A67, wherein the fibrotic disease is cystic fibrosis.

A72. The method of embodiment A67, wherein the fibrotic disease is idiopathic fibrosis.

A73. The method of embodiment A67, wherein the fibrotic disease is fibrosis of the lung.

A74. The method of embodiment A73, wherein the fibrosis of the lung is progressive massive fibrosis and radiation-induced lung injury.

A75. The method of embodiment A67, wherein the fibrotic disease is bridging fibrosis.

A76. The method of embodiment A67, wherein the fibrotic disease is fibrosis of the liver.

A77. The method of embodiment A76, wherein the fibrosis of the liver is cirrhosis.

A78. The method of embodiment A67, wherein the fibrotic disease is fibrosis of the intestine.

A79. The method of embodiment A78, wherein the fibrosis of the intestine is Crohn's disease.

A80. The method of embodiment A67, wherein the fibrotic disease is fibrosis of the muscular system.

A81. The method of embodiment A80, wherein the fibrosis of the muscular system is Duchenne muscular dystrophy (DMD).

A82. The method of embodiment A67, wherein the fibrotic disease is fibrosis of the brain.

A83. The method of embodiment A82, wherein the fibrosis of the brain is glial scar.

A84. The method of embodiment A67, wherein the fibrotic disease is fibrosis of the joints.

A85. The method of embodiment A84, wherein the fibrosis of the joints is arterial stiffness.

A86. The method of embodiment A84 or embodiment A85, wherein the fibrosis of the joints is fibrosis of the knee.

A87. The method of embodiment A84 or embodiment A85, wherein the fibrosis of the joints is fibrosis of the shoulder.

A88. The method of embodiment A67, wherein the fibrotic disease is fibrosis of the skin.

A89. The method of embodiment A88, wherein the fibrosis of the skin is Keloid.

A90. The method of embodiment A67, wherein the fibrotic disease is fibrosis of the bone marrow.

A91. The method of embodiment A90, wherein the fibrosis of the bone marrow is Myelofibrosis.

A92. The method of embodiment A67, wherein the fibrotic disease is fibrosis of the heart.

A93. The method of embodiment A92, wherein the fibrosis of the heart is Myocardial fibrosis.

A94. The method of embodiment A67, wherein the fibrotic disease is fibrosis of the soft tissue.

A95. The method of embodiment A67, wherein the fibrotic disease is fibrosis of the tendons.

A96. The method of embodiment A67, wherein the fibrotic disease is fibrosis of the lymph nodes.

A97. The method of embodiment A67, wherein the fibrotic disease is fibrosis of the eyes.

A98. The method of embodiment A67, wherein the fibrotic disease is retroperitoneum.

A99. The method of embodiment A67, wherein the fibrotic disease is scleroderma.

A100. The method of embodiment A67, wherein the fibrotic disease is surgical scarring.

A101. The method of embodiment A81, wherein the Duchenne muscular dystrophy (DMD) is Becker Muscular Dystrophy (BMD), an intermediate clinical presentation between DMD and BMD, or DMD-associated dilated cardiomyopathy.

A102. The method of any one of embodiments A66-A101, wherein the method treats, prevents, or ameliorates the fibrotic disease in the subject.

A103. The method of any one of embodiments A66-A102, wherein the method treats, prevents, or ameliorates one or more symptoms of said fibrotic disease in the subject.

A104. The method of any one of embodiments A1-A103, wherein the method treats, prevents, or inhibits fibrosis in the subject.

A105. The method of embodiment A103, wherein the method inhibits fibrosis in the liver, lung, skin, soft tissue, tendons, lymph nodes, lung, kidney, heart, eye, or retroperitoneum of said subject.

B1. A method of treating fibrosis in a subject having a fibrotic disease, comprising administering to the subject a compound of Formula I:

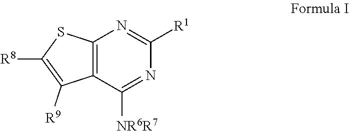

or a pharmaceutically acceptable salt thereof,
wherein:
R¹ is H, alkyl, aryl or heteroaryl;
R⁸ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
R⁹ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
R³ is hydrogen, alkyl, alkenyl, alkynyl, aryl, heteroaryl, heterocyclyl, cycloalkyl, alkylcarbonyl, cycloalkylcarbonyl or arylcarbonyl;
R⁴ is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy, aralkoxy, or NR⁶R⁷;
each R⁵ is selected from the group consisting of hydrogen, alkyl, alkenyl, alkynyl, aryl, heteroaryl, heterocyclyl, cycloalkyl, alkylcarbonyl, cycloalkylcarbonyl, and arylcarbonyl; and
R⁶ and R⁷ are independently selected from the group consisting of hydrogen, alkyl, alkenyl, alkynyl, aryl, heteroaryl, cycloalkyl, heterocyclyl, alkylcarbonyl, cycloalkylcarbonyl, arylcarbonyl, heteroarylcarbonyl, arylsulfonyl, heteroarylsulfonyl, cycloalkylsulfonyl and alkylsulfonyl, or R⁶ and R⁷ are combined to form a cyclic structure including the nitrogen atom to which they are both attached;
   wherein the compound of Formula I or pharmaceutically acceptable salt thereof inhibits phosphorylation of Smad2/3, according to Phospho-Smad2/3 Inhibition Assay, and is inactive according to MAPK p38 Activation Assay.

B2. The method of embodiment B1, wherein:
R¹ is heteroaryl;
R⁸ is aryl or heteroaryl;
R⁹ is aryl or alkyl; and
R⁶ and R⁷ are independently selected from the group consisting of hydrogen, alkyl, aryl, heteroaryl, cycloalkyl, and heterocyclyl, or R⁶ and R⁷ are combined to form a cyclic structure including the nitrogen atom to which they are both attached.

B3. The method of embodiment B1 or embodiment B2, wherein R¹ is:

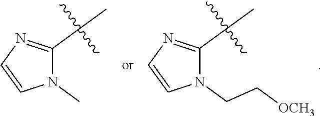

B4. The method of any one of embodiments B1-B3, wherein R⁸ is: phenyl,

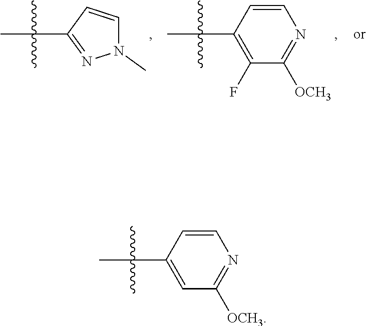

B5. The method of any one of embodiments B1-B4, wherein R⁹ is phenyl.

B6. The method of any one of embodiments B1-B5, wherein the —NR⁶R⁷ group depicted in Formula I is:

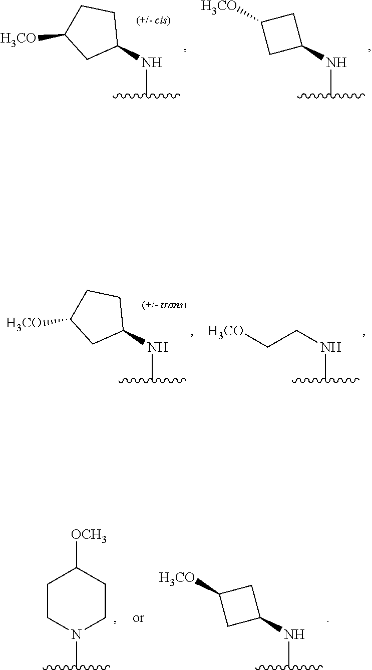 [SMILES: *N[C@@H]1CC[C@H](OC)C1 ; *N[C@H]1C[C@H](OC)C1 ; *N[C@@H]1CC[C@@H](OC)C1 ; *NCCOC ; *N1CCC(OC)CC1 ; *N[C@H]1C[C@@H](OC)C1]

B7. The method of any one of embodiments B1-B6, wherein the compound of Formula I or pharmaceutically acceptable salt thereof is selected from the group consisting of:
N-(2-Methoxyethyl)-2-(1-methyl-1H-imidazol-2-yl)-5,6-diphenylthieno[2,3-d]pyrimidin-4-amine;
N-((1s,3s)-3-methoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine;
rac-N-((1R,3S)-3-Methoxycyclopentyl)-6-(2-methoxypyridin-4-yl)-2-(1-methyl-1H-imidazol-2-yl)-5-phenylthieno[2,3-d]pyrimidin-4-amine;
6-(3-Fluoro-2-methoxypyridin-4-yl)-N-((1r,3r)-3-methoxycyclobutyl)-2-(1-methyl-1H-imidazol-2-yl)-5-phenylthieno[2,3-d]pyrimidin-4-amine;
N-((1r,3r)-3-Methoxycyclobutyl)-6-(2-methoxypyridin-4-yl)-2-(1-methyl-1H-imidazol-2-yl)-5-phenylthieno[2,3-d]pyrimidin-4-amine; and
N-((1R,3R)-3-Methoxycyclopentyl)-6-(2-methoxypyridin-4-yl)-2-(1-methyl-1H-imidazol-2-yl)-5-phenylthieno[2,3-d]pyrimidin-4-amine.

B8. The method of any one of embodiments B1-B7, wherein the compound of Formula I or pharmaceutically acceptable salt thereof inhibits phosphorylation of Smad2/3 by 20% or more at 10 μM, according to Phospho-Smad2/3 Inhibition Assay.

B9. The method of embodiment B8, wherein the compound of Formula I or pharmaceutically acceptable salt thereof is selected from the group consisting of:
N-(2-Methoxyethyl)-2-(1-methyl-1H-imidazol-2-yl)-5,6-diphenylthieno[2,3-d]pyrimidin-4-amine;
N-((1s,3s)-3-methoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine;
rac-N-((1R,3S)-3-Methoxycyclopentyl)-6-(2-methoxypyridin-4-yl)-2-(1-methyl-1H-imidazol-2-yl)-5-phenylthieno[2,3-d]pyrimidin-4-amine;
6-(3-Fluoro-2-methoxypyridin-4-yl)-N-((1r,3r)-3-methoxycyclobutyl)-2-(1-methyl-1H-imidazol-2-yl)-5-phenylthieno[2,3-d]pyrimidin-4-amine;
N-((1r,3r)-3-Methoxycyclobutyl)-6-(2-methoxypyridin-4-yl)-2-(1-methyl-1H-imidazol-2-yl)-5-phenylthieno[2,3-d]pyrimidin-4-amine; and
N-((1R,3R)-3-Methoxycyclopentyl)-6-(2-methoxypyridin-4-yl)-2-(1-methyl-1H-imidazol-2-yl)-5-phenylthieno[2,3-d]pyrimidin-4-amine.

B10. The method of any one of embodiments B1-B7, wherein the compound of Formula I or pharmaceutically acceptable salt thereof inhibits phosphorylation of Smad2/3 by 30% or more at 10 μM, according to Phospho-Smad2/3 Inhibition Assay.

B11. The method of embodiment B10, wherein the compound of Formula I or pharmaceutically acceptable salt thereof is selected from the group consisting of:
N-(2-Methoxyethyl)-2-(1-methyl-1H-imidazol-2-yl)-5,6-diphenylthieno[2,3-d]pyrimidin-4-amine;
N-((1s,3s)-3-methoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine;
rac-N-((1R,3S)-3-Methoxycyclopentyl)-6-(2-methoxypyridin-4-yl)-2-(1-methyl-1H-imidazol-2-yl)-5-phenylthieno[2,3-d]pyrimidin-4-amine;
6-(3-Fluoro-2-methoxypyridin-4-yl)-N-((1r,3r)-3-methoxycyclobutyl)-2-(1-methyl-1H-imidazol-2-yl)-5-phenylthieno[2,3-d]pyrimidin-4-amine;
N-((1r,3r)-3-Methoxycyclobutyl)-6-(2-methoxypyridin-4-yl)-2-(1-methyl-1H-imidazol-2-yl)-5-phenylthieno[2,3-d]pyrimidin-4-amine; and
N-((1R,3R)-3-Methoxycyclopentyl)-6-(2-methoxypyridin-4-yl)-2-(1-methyl-1H-imidazol-2-yl)-5-phenylthieno[2,3-d]pyrimidin-4-amine.

B12. The method of any one of embodiments B1-B7, wherein the compound of Formula I or pharmaceutically acceptable salt thereof inhibits phosphorylation of Smad2/3 by 50% or more at 10 μM, according to Phospho-Smad2/3 Inhibition Assay.

B13. The method of embodiment B12, wherein the compound of Formula I or pharmaceutically acceptable salt thereof is selected from the group consisting of:
N-(2-Methoxyethyl)-2-(1-methyl-1H-imidazol-2-yl)-5,6-diphenylthieno[2,3-d]pyrimidin-4-amine;
N-((1s,3s)-3-methoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine;
rac-N-((1R,3S)-3-Methoxycyclopentyl)-6-(2-methoxypyridin-4-yl)-2-(1-methyl-1H-imidazol-2-yl)-5-phenylthieno[2,3-d]pyrimidin-4-amine;
6-(3-Fluoro-2-methoxypyridin-4-yl)-N-((1r,3r)-3-methoxycyclobutyl)-2-(1-methyl-1H-imidazol-2-yl)-5-phenylthieno[2,3-d]pyrimidin-4-amine;
N-((1r,3r)-3-Methoxycyclobutyl)-6-(2-methoxypyridin-4-yl)-2-(1-methyl-1H-imidazol-2-yl)-5-phenylthieno[2,3-d]pyrimidin-4-amine; and
N-((1R,3R)-3-Methoxycyclopentyl)-6-(2-methoxypyridin-4-yl)-2-(1-methyl-1H-imidazol-2-yl)-5-phenylthieno[2,3-d]pyrimidin-4-amine.

B14. The method of any one of embodiments B1-B7, wherein the compound of Formula I or pharmaceutically acceptable salt thereof inhibits phosphorylation of Smad2/3 by 70% or more at 10 μM, according to Phospho-Smad2/3 Inhibition Assay.

B15. The method of embodiment B14, wherein the compound of Formula I or pharmaceutically acceptable salt thereof is selected from the group consisting of:
N-((1s,3s)-3-methoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine;
rac-N-((1R,3S)-3-Methoxycyclopentyl)-6-(2-methoxypyridin-4-yl)-2-(1-methyl-1H-imidazol-2-yl)-5-phenylthieno[2,3-d]pyrimidin-4-amine;
6-(3-Fluoro-2-methoxypyridin-4-yl)-N-((1r,3r)-3-methoxycyclobutyl)-2-(1-methyl-1H-imidazol-2-yl)-5-phenylthieno[2,3-d]pyrimidin-4-amine;
N-((1r,3r)-3-Methoxycyclobutyl)-6-(2-methoxypyridin-4-yl)-2-(1-methyl-1H-imidazol-2-yl)-5-phenylthieno[2,3-d]pyrimidin-4-amine; and
N-((1R,3R)-3-Methoxycyclopentyl)-6-(2-methoxypyridin-4-yl)-2-(1-methyl-1H-imidazol-2-yl)-5-phenylthieno[2,3-d]pyrimidin-4-amine.

B16. The method of any one of embodiments B1-B15, wherein the compound of Formula I or pharmaceutically acceptable salt thereof is exclusive of compounds selected from the group consisting of:
N-(2-Methoxyethyl)-N-methyl-2-(1-methyl-1H-imidazol-2-yl)-5,6-diphenylthieno[2,3-d]pyrimidin-4-amine;
N-(3-Methoxypropyl)-2-(1-methyl-1H-imidazol-2-yl)-5,6-diphenylthieno[2,3-d]pyrimidin-4-amine;
5-(2-Chlorophenyl)-N-(2-methoxyethyl)-2-(1-methyl-1H-imidazol-2-yl)-6-phenylthieno[2,3-d]pyrimidin-4-amine;
rac-N-((1R,3S)-3-Methoxycyclopentyl)-2-(1-methyl-1H-imidazol-2-yl)-6-(1-methyl-1H-pyrazol-3-yl)-5-phenylthieno[2,3-d]pyrimidin-4-amine;
4-(4-Methoxypiperidin-1-yl)-2-(1-methyl-1H-imidazol-2-yl)-6-(1-methyl-1H-pyrazol-3-yl)-5-phenylthieno[2,3-d]pyrimidine;
6-(2-Chloro-3-methoxyphenyl)-N-(2-methoxyethyl)-5-(4-methoxyphenyl)-2-(1-methyl-1H-imidazol-2-yl)thieno[2,3-d]pyrimidin-4-amine;
6-(2-Chloro-3-methoxyphenyl)-N-(2-methoxyethyl)-5-(4-methoxyphenyl)-2-(1-methyl-1H-imidazol-4-yl)thieno[2,3-d]pyrimidin-4-amine;
6-(2-Chloro-3-methoxyphenyl)-N-(2-methoxyethyl)-2-(1-methyl-1H-imidazol-2-yl)-5-phenylthieno[2,3-d]pyrimidin-4-amine;
rac-6-(2-Fluoro-3-methoxyphenyl)-N-((1R,3S)-3-methoxycyclopentyl)-5-methyl-2-(1-methyl-1H-imidazol-2-yl)thieno[2,3-d]pyrimidin-4-amine and rac-6-(2-Fluoro-3-methoxyphenyl)-N-((1R,3R)-3-methoxycyclopentyl)-5-methyl-2-(1-methyl-1H-imidazol-2-yl)thieno[2,3-d]pyrimidin-4-amine;
6-(1-Isopropyl-1H-pyrazol-3-yl)-N-((1S,3R)-3-methoxycyclopentyl)-2-(1-methyl-1H-imidazol-2-yl)-5-phenylthieno[2,3-d]pyrimidin-4-amine;
rac-6-(1-Isopropyl-1H-pyrazol-3-yl)-N-((1R,3S)-3-methoxycyclopentyl)-5-methyl-2-(1-methyl-1H-imidazol-2-yl)thieno[2,3-d]pyrimidin-4-amine;
rac-6-(1-Isopropyl-1H-pyrazol-3-yl)-4-(3-methoxypiperidin-1-yl)-5-methyl-2-(1-methyl-1H-imidazol-2-yl)thieno[2,3-d]pyrimidine;
4-(6-(1-Isopropyl-1H-pyrazol-3-yl)-5-methyl-2-(1-methyl-1H-imidazol-2-yl)thieno[2,3-d]pyrimidin-4-yl)-2-(methoxymethyl)morpholine;
6-(1-Isopropyl-1H-pyrazol-3-yl)-N-(3-methoxyphenyl)-5-methyl-2-(1-methyl-1H-imidazol-2-yl)thieno[2,3-d]pyrimidin-4-amine;
6-(1-Isopropyl-1H-pyrazol-3-yl)-N-(6-methoxypyridin-2-yl)-5-methyl-2-(1-methyl-1H-imidazol-2-yl)thieno[2,3-d]pyrimidin-4-amine;
6-(1-Isopropyl-1H-pyrazol-3-yl)-N-(6-methoxypyrimidin-4-yl)-5-methyl-2-(1-methyl-1H-imidazol-2-yl)thieno[2,3-d]pyrimidin-4-amine;
rac-6-(1-Isopropyl-1H-pyrazol-3-yl)-N-((1R,3S)-3-(2-methoxyethoxy)cyclopentyl)-5-methyl-2-(1-methyl-1H-imidazol-2-yl)thieno[2,3-d]pyrimidin-4-amine;
rac-6-(1-(2-Methoxyethyl)-1H-pyrazol-3-yl)-N-(((1S,2R)-2-(methoxymethyl)cyclobutyl)methyl)-5-methyl-2-(1-methyl-1H-imidazol-2-yl)thieno[2,3-d]pyrimidin-4-amine;
6-(2-Fluoro-3-methoxyphenyl)-N-((1r,3r)-3-methoxycyclobutyl)-2-(1-methyl-1H-imidazol-2-yl)-5-phenylthieno[2,3-d]pyrimidin-4-amine;
6-(2-Fluoro-3-methoxyphenyl)-N-((1R,3R)-3-methoxycyclopentyl)-2-(1-methyl-1H-imidazol-2-yl)-5-phenylthieno[2,3-d]pyrimidin-4-amine;
6-(2-Fluoro-3-methoxyphenyl)-N-((1r,3r)-3-methoxycyclobutyl)-5-methyl-2-(1-methyl-1H-imidazol-2-yl)thieno[2,3-d]pyrimidin-4-amine; and
rac-6-(2-Fluoro-3-methoxyphenyl)-N-((1R,3R)-3-methoxycyclopentyl)-5-methyl-2-(1-methyl-1H-imidazol-2-yl)thieno[2,3-d]pyrimidin-4-amine.

B17. The method of any one of embodiments B1-B16, wherein the compound of Formula I or pharmaceutically acceptable salt thereof inhibits MAPK p38, according to MAPK p38 Activation Assay.

B18. The method of any one of embodiments B1-B17, wherein the compound of Formula I or pharmaceutically acceptable salt thereof activates INK, according to JNK Activation Assay.

B19. The method of any one of embodiments B1-B18, wherein the compound of Formula I or pharmaceutically acceptable salt thereof is a modulator of Ras superfamily activity according to a Ras Superfamily Activity Assay.

B20. A method of treating fibrosis in a subject having a fibrotic disease, comprising administering to the subject a compound of Formula II:

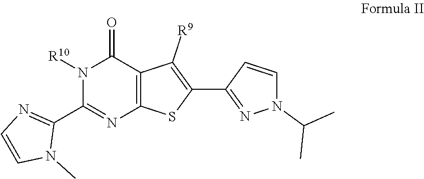

or a pharmaceutically acceptable salt thereof,
wherein:
R⁹ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
R¹⁰ is H, alkyl, alkenyl, alkynyl, aryl, cycloalkyl, heterocyclyl, heteroaryl, C(O)R⁴, or S(O)ₚR⁴;
R³ is hydrogen, alkyl, alkenyl, alkynyl, aryl, heteroaryl, heterocyclyl, cycloalkyl, alkylcarbonyl, cycloalkylcarbonyl or arylcarbonyl;
R⁴ is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy, aralkoxy, or NR⁶R⁷;
each R⁵ is selected from the group consisting of hydrogen, alkyl, alkenyl, alkynyl, aryl, heteroaryl, heterocyclyl, cycloalkyl, alkylcarbonyl, cycloalkylcarbonyl, and arylcarbonyl; and
R⁶ and R⁷ are independently selected from the group consisting of hydrogen, alkyl, alkenyl, alkynyl, aryl, heteroaryl, cycloalkyl, heterocyclyl, alkylcarbonyl, cycloalkylcarbonyl, arylcarbonyl, heteroarylcarbonyl, arylsulfonyl, heteroarylsulfonyl, cycloalkylsulfonyl and alkylsulfonyl, or R⁶ and R⁷ are combined to form a cyclic structure including the nitrogen atom to which they are both attached;
   wherein the compound of Formula II or pharmaceutically acceptable salt thereof is inactive according to MAPK p38 Activation Assay.

B21. The method of embodiment B20, wherein:
R⁹ is alkyl; and
R¹⁰ is alkyl.

B22. The method of embodiment B20 or embodiment B21, wherein R⁹ is methyl.

B23. The method of any one of embodiments B20-B22, wherein the compound of Formula II or pharmaceutically acceptable salt thereof is selected from the group consisting of:
6-(1-Isopropyl-1H-pyrazol-3-yl)-3-(3-methoxypropyl)-5-methyl-2-(1-methyl-1H-imidazol-2-yl)thieno[2,3-d]pyrimidin-4(3H)-one;
6-(1-Isopropyl-1H-pyrazol-3-yl)-3-(4-methoxybutyl)-5-methyl-2-(1-methyl-1H-imidazol-2-yl)thieno[2,3-d]pyrimidin-4(3H)-one; and
6-(1-Isopropyl-1H-pyrazol-3-yl)-3-((6-methoxypyrimidin-4-yl)methyl)-5-methyl-2-(1-methyl-1H-imidazol-2-yl)thieno[2,3-d]pyrimidin-4(3H)-one.

B24. The method of any one of embodiments B20-B23, wherein the compound of Formula II or pharmaceutically acceptable salt thereof is exclusive of compounds selected from the group consisting of:
6-(1-Isopropyl-1H-pyrazol-3-yl)-3-(2-methoxyethyl)-5-methyl-2-(1-methyl-1H-imidazol-2-yl)thieno[2,3-d]pyrimidin-4(3H)-one; and
6-(1-Isopropyl-1H-pyrazol-3-yl)-3-(3-methoxypropyl)-2-(1-methyl-1H-imidazol-2-yl)-5-phenylthieno[2,3-d]pyrimidin-4(3H)-one.

B25. The method of any one of embodiments B20-B24, wherein the compound of Formula II or pharmaceutically acceptable salt thereof inhibits MAPK p38, according to MAPK p38 Activation Assay.

B26. The method of any one of embodiments B20-B25, wherein the compound of Formula II or pharmaceutically acceptable salt thereof inhibits JNK, according to JNK Activation Assay.

B27. The method of any one of embodiments B20-B26, wherein the compound of Formula II or pharmaceutically acceptable salt thereof is a modulator of Ras superfamily activity according to a Ras Superfamily Activity Assay.

B28. A method of treating fibrosis in a subject having a fibrotic disease, comprising administering to the subject a compound of Formula III:

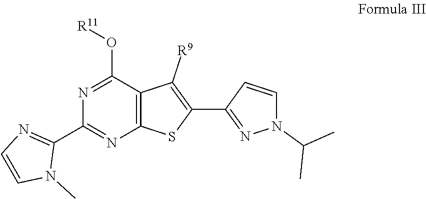

or a pharmaceutically acceptable salt thereof,
wherein:
R⁹ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
R¹¹ is H, alkyl, alkenyl, alkynyl, aryl, cycloalkyl, heterocyclyl, heteroaryl, C(O)R⁴, or S(O)ₚR⁴;
R³ is hydrogen, alkyl, alkenyl, alkynyl, aryl, heteroaryl, heterocyclyl, cycloalkyl, alkylcarbonyl, cycloalkylcarbonyl or arylcarbonyl;
R⁴ is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy, aralkoxy, or NR⁶R⁷;
each R⁵ is selected from the group consisting of hydrogen, alkyl, alkenyl, alkynyl, aryl, heteroaryl, heterocyclyl, cycloalkyl, alkylcarbonyl, cycloalkylcarbonyl, and arylcarbonyl; and
R⁶ and R⁷ are independently selected from the group consisting of hydrogen, alkyl, alkenyl, alkynyl, aryl, heteroaryl, cycloalkyl, heterocyclyl, alkylcarbonyl, cycloalkylcarbonyl, arylcarbonyl, heteroarylcarbonyl, arylsulfonyl, heteroarylsulfonyl, cycloalkylsulfonyl and alkylsulfonyl, or R⁶ and R⁷ are combined to form a cyclic structure including the nitrogen atom to which they are both attached;
   wherein the compound of Formula III or pharmaceutically acceptable salt thereof is inactive according to MAPK p38 Activation Assay.

B29. The method of embodiment B28, wherein:
R⁹ is aryl; and
R¹¹ is alkyl.

B30. The method of embodiment B28 or embodiment B29, wherein R⁹ is methyl.

B31. The method of any one of embodiments B28-B30, wherein the compound of Formula III or pharmaceutically acceptable salt thereof is selected from the group consisting of.
6-(1-Isopropyl-1H-pyrazol-3-yl)-4-(2-methoxyethoxy)-5-methyl-2-(1-methyl-1H-imidazol-2-yl)thieno[2,3-d]pyrimidine;
6-(1-Isopropyl-1H-pyrazol-3-yl)-4-(3-methoxypropoxy)-5-methyl-2-(1-methyl-1H-imidazol-2-yl)thieno[2,3-d]pyrimidine; and
6-(1-Isopropyl-1H-pyrazol-3-yl)-4-(4-methoxybutoxy)-5-methyl-2-(1-methyl-1H-imidazol-2-yl)thieno[2,3-d]pyrimidine.

B32. The method of any one of embodiments B28-B31, wherein the compound of Formula III or pharmaceutically acceptable salt thereof is exclusive of 6-(1-Isopropyl-1H-pyrazol-3-yl)-4-(3-methoxypropoxy)-2-(1-methyl-1H-imidazol-2-yl)-5-phenylthieno[2,3-d]pyrimidine.

B33. The method of any one of embodiments B28-B32, wherein the compound of Formula III or pharmaceutically acceptable salt thereof inhibits INK, according to INK Activation Assay.

B34. The method of any one of embodiments B28-B32, wherein the compound of Formula III or pharmaceutically acceptable salt thereof activates INK, according to INK Activation Assay.

B35. The method of any one of embodiments B28-B34, wherein the compound of Formula III or pharmaceutically acceptable salt thereof is a modulator of Ras superfamily activity according to a Ras Superfamily Activity Assay.

B36. A method of treating fibrosis in a subject having a fibrotic disease, comprising administering to the subject a compound or a pharmaceutically acceptable salt thereof identified as a modulator of Ras superfamily activity according to a Ras Superfamily Activity Assay; wherein the identified Ras superfamily modulating compound or pharmaceutically acceptable salt thereof inhibits phosphorylation of Smad2/3, according to Phospho-Smad2/3 Inhibition Assay, and is inactive according to MAPK p38 Activation Assay.

B37. The method of embodiment B36, wherein the identified Ras superfamily modulating compound or pharmaceutically acceptable salt thereof inhibits phosphorylation of Smad2/3 by 20%, 30%, 50%, 70%, or more at 10 μM, according to Phospho-Smad2/3 Inhibition Assay.

B38. The method of embodiment B36 or embodiment B37, wherein the identified Ras superfamily modulating compound or pharmaceutically acceptable salt thereof activates INK, according to JNK Activation Assay.

B39. The method of any one of embodiments B36-B38, wherein the Ras Superfamily Activity Assay comprises:
a) contacting the compound or pharmaceutically acceptable salt thereof with a Ras superfamily protein;
b) incubating a cyanine-labeled GTP with said compound or pharmaceutically acceptable salt thereof and the Ras superfamily protein; and
c) measuring the amount of the cyanine-labeled GTP bound to the Ras superfamily protein.

B40. The method of embodiment B39, wherein the Ras Superfamily Activity Assay is a cell-free assay.

B41. The method of embodiment B39 or embodiment B40, wherein the Ras Superfamily Activity Assay is a GTP-binding competition assay.

B42. The method of any one of embodiments B36-B41, wherein the Ras superfamily protein is a Ras protein, a Rac protein, or a Rho protein.

B43. The method of embodiment B42, wherein the Ras superfamily protein is a Ras protein.

B44. The method of embodiment B43, wherein the Ras Superfamily Activity Assay is a Ras Activity Assay.

B45. The method of embodiment B43 or embodiment B44, wherein the identified Ras superfamily modulating compound or pharmaceutically acceptable salt thereof is identified as a Ras modulating compound or a pharmaceutically acceptable salt thereof.

B46. The method of embodiment B45, wherein the identified Ras modulating compound or pharmaceutically acceptable salt thereof competitively inhibits GTP binding to the Ras GTP binding domain.

B47. The method of embodiment B45 or embodiment B46, wherein the identified Ras modulating compound or pharmaceutically acceptable salt thereof binds to the Ras protein GTP binding domain with greater than 25% inhibition at 20 uM.

B48. The method of any one of embodiments B45-B47, wherein the identified Ras modulating compound or pharmaceutically acceptable salt thereof has a binding affinity (Kd) to the Ras protein GTP binding domain of less than 10 uM.

B49. The method of any one of embodiments B45-B48, wherein the identified Ras modulating compound or pharmaceutically acceptable salt thereof inhibits the Ras activity and has an IC50 value of less than 10 uM.

B50. The method of any one of embodiments B45-B49, wherein the identified Ras modulating compound or pharmaceutically acceptable salt thereof inhibits binding of the cyanine labeled GTP with an IC50 value of less than 10 uM.

B51. The method of any one of embodiments B45-B50, wherein the cyanine-labeled GTP is a Cy3- or a Cy5-labeled GTP.

B52. The method of any one of embodiments B45-B51, wherein the Ras Activity Assay is a GTP-binding competition assay.

B53. The method of any one of embodiments B44-B52, wherein the Ras protein is immobilized.

B54. The method of any one of embodiments B44-B53, wherein the Ras protein is DIRAS I; DIRAS2; DIRAS3; ERAS; GEM; HRAS; KRAS; MRAS; NKIRASI; NKIRAS2; NRAS; RALA; RALB; RAPIA; RAPIB; RAP2A; RAP2B; RAP2C; RASDI; RASD2; RASLIOA; RASLIOB; RASLIIA; RASLIIB; RASL12; REMI; REM2; RERG; RERGL; RRAD; RRAS; or RRAS2.

B55. The method of any one of embodiments B44-B54, wherein the Ras protein is HRAS; KRAS; or NRAS, or a mutant thereof.

B56. The method of embodiment B55, wherein the Ras protein is a KRAS mutant.

B57. The method of embodiment B56, wherein the KRAS mutant is a KRas G12D mutant, KRas G12C mutant, or KRas Q61H mutant.

B58. The method of embodiment B55, wherein the Ras protein is wild-type KRas.

B59. The method of embodiment B55, wherein the Ras protein is HRAS or mutant thereof.

B60. The method of embodiment B55, wherein the Ras protein is NRAS or mutant thereof.

B61. The method of embodiment B42, wherein the Ras superfamily protein is a Rac protein.

B62. The method of embodiment B61, wherein the Ras Superfamily Activity Assay is a Rac Activity Assay.

B63. The method of embodiment B61 or embodiment B62, wherein the identified Ras superfamily modulating compound or pharmaceutically acceptable salt thereof is identified as a Rac modulating compound or a pharmaceutically acceptable salt thereof.

B64. The method of embodiment B63, wherein the identified Rac modulating compound or pharmaceutically acceptable salt thereof competitively inhibits GTP binding to the Rac GTP binding domain.

B65. The method of embodiment B63 or embodiment B64, wherein the identified Rac modulating compound or pharmaceutically acceptable salt thereof binds to the Rac protein GTP binding domain with greater than 25% inhibition at 20 uM.

B66. The method of any one of embodiments B63-B65, wherein the identified Rae modulating compound or pharmaceutically acceptable salt thereof has a binding affinity (Kd) to the Rac protein GTP binding domain of less than 10 uM.

B67. The method of any one of embodiments B63-B66, wherein the identified Rac modulating compound or pharmaceutically acceptable salt thereof inhibits the Rac activity and has an IC50 value of less than 10 uM.

B68. The method of any one of embodiments B63-B67, wherein the identified Rac modulating compound or pharmaceutically acceptable salt thereof inhibits binding of the cyanine labeled GTP with an IC50 value of less than 10 uM.

B69. The method of any one of embodiments B63-B68, wherein the cyanine-labeled GTP is a Cy3- or a Cy5-labeled GTP.

B70. The method of any one of embodiments B62-B69, wherein the Rac Activity Assay is a GTP-binding competition assay.

B71. The method of any one of embodiments B62-B70, wherein the Rac protein is immobilized.

B72. The method of any one of embodiments B62-B71, wherein the Rac protein is RAC1; RAC2; RAC3; RHOG, or a mutant thereof.

B73. The method of embodiment B72, wherein the Rac protein is wild-type RAC1.

B74. The method of embodiment B63, wherein the Ras superfamily protein is a Rho protein.

B75. The method of embodiment B74, wherein the Ras Superfamily Activity Assay is a Rho Activity Assay.

B76. The method of embodiment B74 or embodiment B75, wherein the identified Ras superfamily modulating compound or pharmaceutically acceptable salt thereof is identified as a Rho modulating compound or a pharmaceutically acceptable salt thereof.

B77. The method of embodiment B76, wherein the identified Rho modulating compound or pharmaceutically acceptable salt thereof competitively inhibits GTP binding to the Rho GTP binding domain.

B78. The method of embodiment B76 or embodiment B77, wherein the identified Rho modulating compound or pharmaceutically acceptable salt thereof binds to the Rho protein GTP binding domain with greater than 25% inhibition at 20 uM.

B79. The method of any one of embodiments B76-B78, wherein the identified Rho modulating compound or pharmaceutically acceptable salt thereof has a binding affinity (Kd) to the Rho protein GTP binding domain of less than 10 uM.

B80. The method of any one of embodiments B76-B79, wherein the identified Rho modulating compound or pharmaceutically acceptable salt thereof inhibits the Rho activity and has an IC50 value of less than 10 uM.

B81. The method of any one of embodiments B76-B80, wherein the identified Rho modulating compound or pharmaceutically acceptable salt thereof inhibits binding of the cyanine labeled GTP with an IC50 value of less than 10 uM.

B82. The method of any one of embodiments B76-B81, wherein the cyanine-labeled GTP is a Cy3- or a Cy5-labeled GTP.

B83. The method of any one of embodiments B75-B82, wherein the Rho Activity Assay is a GTP-binding competition assay.

B84. The method of any one of embodiments B75-B83, wherein the Rho protein is immobilized.

B85. The method of any one of embodiments B75-B84, wherein the Rho protein is RHOA; RHOB; RHOBTB1; RHOBTB2; RHOBTB3; RHOC; RHOD; RHOF; RHOH; RHOJ; RHOQ; RHOU; RHOV; RND1; RND2; RND3; CDC42, or a mutant thereof.

B86. The method of any one of embodiments B75-B85, wherein the Rho protein is wild-type RHOA.

B87. The method of any one of embodiments B1-B86, wherein the method treats, prevents, or ameliorates the fibrotic disease in the subject or treats, prevents, or ameliorates one or more symptoms of said fibrotic disease in the subject.

B88. The method of any one of embodiments B1-B87, wherein the fibrotic disease is selected from the group consisting of fibrosis of kidney, fibrosis of cardiovascular system, pulmonary fibrosis, cystic fibrosis, idiopathic fibrosis, fibrosis of the lung, bridging fibrosis, fibrosis of the liver, fibrosis of the intestine, fibrosis of the muscular system, fibrosis of the brain, fibrosis of the joints, fibrosis of the skin, fibrosis of the bone marrow, fibrosis of the heart, fibrosis of the soft tissue, fibrosis of the tendons, fibrosis of the lymph nodes, fibrosis of the eyes, retroperitoneum, scleroderma and surgical scarring.

B89. The method of embodiment B88, wherein the fibrotic disease is fibrosis of the kidney.

B90. The method of embodiment B88, wherein the fibrotic disease is fibrosis of the cardiovascular system.

B91. The method of embodiment B88, wherein the fibrotic disease is pulmonary fibrosis.

B92. The method of embodiment B88, wherein the fibrotic disease is cystic fibrosis.

B93. The method of embodiment B88, wherein the fibrotic disease is idiopathic fibrosis.

B94. The method of embodiment B88, wherein the fibrotic disease is fibrosis of the lung.

B95. The method of embodiment B94, wherein the fibrosis of the lung is progressive massive fibrosis and radiation-induced lung injury.

B96. The method of embodiment B88, wherein the fibrotic disease is bridging fibrosis.

B97. The method of embodiment B88, wherein the fibrotic disease is fibrosis of the liver.

B98. The method of embodiment B97, wherein the fibrosis of the liver is cirrhosis.

B99. The method of embodiment B88, wherein the fibrotic disease is fibrosis of the intestine.

B100. The method of embodiment B99, wherein the fibrosis of the intestine is Crohn's disease.

B101. The method of embodiment B88, wherein the fibrotic disease is fibrosis of the muscular system.

B102. The method of embodiment B101, wherein the fibrosis of the muscular system is Duchenne muscular dystrophy (DMD).

B103. The method of embodiment B88, wherein the fibrotic disease is fibrosis of the brain.

B104. The method of embodiment B103, wherein the fibrosis of the brain is glial scar.

B105. The method of embodiment B88, wherein the fibrotic disease is fibrosis of the joints.

B106. The method of embodiment B105, wherein the fibrosis of the joints is arterial stiffness.

B107. The method of embodiment B105 or embodiment B106, wherein the fibrosis of the joints is fibrosis of the knee.

B108. The method of embodiment B105 or embodiment B106, wherein the fibrosis of the joints is fibrosis of the shoulder.

B109. The method of embodiment B88, wherein the fibrotic disease is fibrosis of the skin.

B110. The method of embodiment B109, wherein the fibrosis of the skin is Keloid.

B111. The method of embodiment B88, wherein the fibrotic disease is fibrosis of the bone marrow.

B112. The method of embodiment B111, wherein the fibrosis of the bone marrow is Myelofibrosis.

B113. The method of embodiment B88, wherein the fibrotic disease is fibrosis of the heart.

B114. The method of embodiment B113, wherein the fibrosis of the heart is Myocardial fibrosis.

B115. The method of embodiment B88, wherein the fibrotic disease is fibrosis of the soft tissue.

B116. The method of embodiment B88, wherein the fibrotic disease is fibrosis of the tendons.

B117. The method of embodiment B88, wherein the fibrotic disease is fibrosis of the lymph nodes.

B118. The method of embodiment B88, wherein the fibrotic disease is fibrosis of the eyes.

B119. The method of embodiment B88, wherein the fibrotic disease is retroperitoneum.

B120. The method of embodiment B88, wherein the fibrotic disease is scleroderma.

B121. The method of embodiment B88, wherein the fibrotic disease is surgical scarring.

B122. The method of embodiment B102, wherein the Duchenne muscular dystrophy (DMD) is Becker Muscular Dystrophy (BMD), an intermediate clinical presentation between DMD and BMD, or DMD-associated dilated cardiomyopathy.

B123. The method of any one of embodiments B87-B122, wherein the method treats, prevents, or ameliorates the fibrotic disease in the subject.

B124. The method of any one of embodiments B87-B123, wherein the method treats, prevents, or ameliorates one or more symptoms of said fibrotic disease in the subject.

B125. The method of any one of embodiments B1-B124, wherein the method treats, prevents, or inhibits fibrosis in the subject.

B126. The method of embodiment B125, wherein the method inhibits fibrosis in the liver, lung, skin, soft tissue, tendons, lymph nodes, lung, kidney, heart, eye, or retroperitoneum of said subject.

B127. A compound of Formula IA:

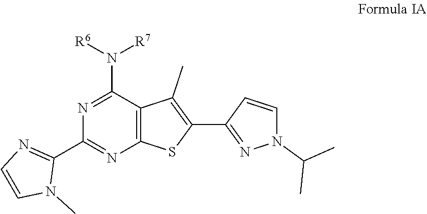

or a pharmaceutically acceptable salt thereof,
wherein:
R⁶ and R⁷ are independently selected from the group consisting of hydrogen, alkyl, alkenyl, alkynyl, aryl, heteroaryl, cycloalkyl, heterocyclyl, alkylcarbonyl, cycloalkylcarbonyl, arylcarbonyl, heteroarylcarbonyl, arylsulfonyl, heteroarylsulfonyl, cycloalkylsulfonyl and alkylsulfonyl, or R⁶ and R⁷ are combined to form a cyclic structure including the nitrogen atom to which they are both attached.

B128. The compound of embodiment B127, wherein:
R⁶ and R⁷ are independently selected from the group consisting of hydrogen, alkyl, aryl, heteroaryl, cycloalkyl, and heterocyclyl, or R⁶ and R⁷ are combined to form a cyclic structure including the nitrogen atom to which they are both attached.

B129. The compound of embodiment B127 or embodiment B128, wherein the NR⁶R⁷ group is:

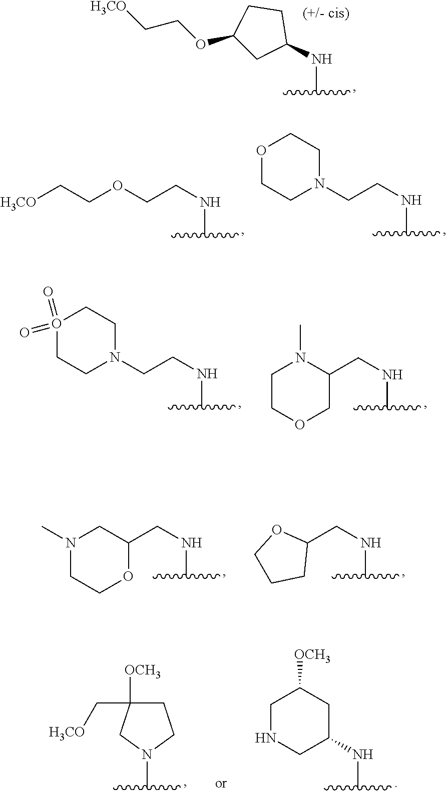

B130. A compound selected from the group consisting of:

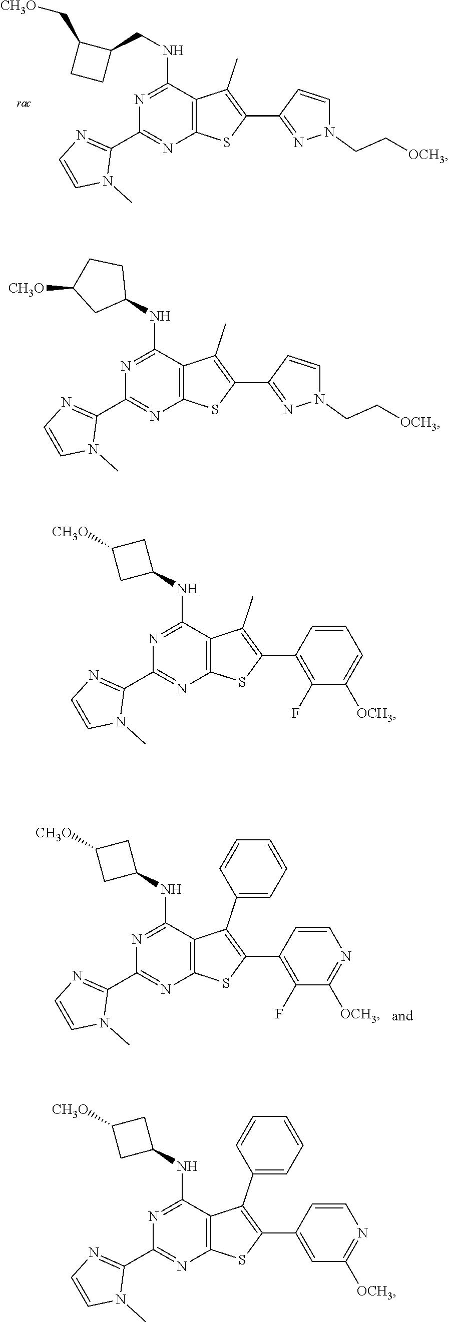

or an enantiomer or a pharmaceutically acceptable salt thereof.

B131. A compound of Formula II:

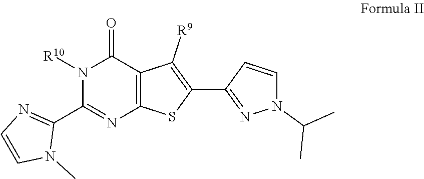

or a pharmaceutically acceptable salt thereof,
wherein:
R⁹ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
R¹⁰ is H, alkyl, alkenyl, alkynyl, aryl, cycloalkyl, heterocyclyl, heteroaryl, C(O)R⁴, or S(O)ₚR⁴;
R³ is hydrogen, alkyl, alkenyl, alkynyl, aryl, heteroaryl, heterocyclyl, cycloalkyl, alkylcarbonyl, cycloalkylcarbonyl or arylcarbonyl;
R⁴ is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy, aralkoxy, or NR⁶R⁷;
each R⁵ is selected from the group consisting of hydrogen, alkyl, alkenyl, alkynyl, aryl, heteroaryl, heterocyclyl, cycloalkyl, alkylcarbonyl, cycloalkylcarbonyl, and arylcarbonyl; and
R⁶ and R⁷ are independently selected from the group consisting of hydrogen, alkyl, alkenyl, alkynyl, aryl, heteroaryl, cycloalkyl, heterocyclyl, alkylcarbonyl, cycloalkylcarbonyl, arylcarbonyl, heteroarylcarbonyl, arylsulfonyl, heteroarylsulfonyl, cycloalkylsulfonyl and alkylsulfonyl, or R⁶ and R⁷ are combined to form a cyclic structure including the nitrogen atom to which they are both attached.

B132. The compound of embodiment B131, wherein:
R⁹ is aryl or alkyl; and
R¹⁰ is alkyl.

B133. The compound of embodiment B131 or embodiment B132, wherein R⁹ is methyl or phenyl.

B134. A compound of Formula III:

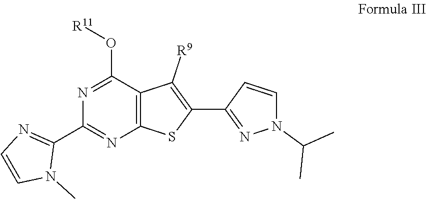

or a pharmaceutically acceptable salt thereof,
wherein:
R⁹ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
R¹¹ is H, alkyl, alkenyl, alkynyl, aryl, cycloalkyl, heterocyclyl, heteroaryl, C(O)R⁴, or S(O)ₚR⁴;
R³ is hydrogen, alkyl, alkenyl, alkynyl, aryl, heteroaryl, heterocyclyl, cycloalkyl, alkylcarbonyl, cycloalkylcarbonyl or arylcarbonyl;
R⁴ is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy, aralkoxy, or NR⁶R⁷;
each R⁵ is selected from the group consisting of hydrogen, alkyl, alkenyl, alkynyl, aryl, heteroaryl, heterocyclyl, cycloalkyl, alkylcarbonyl, cycloalkylcarbonyl, and arylcarbonyl; and
R⁶ and R⁷ are independently selected from the group consisting of hydrogen, alkyl, alkenyl, alkynyl, aryl, heteroaryl, cycloalkyl, heterocyclyl, alkylcarbonyl, cycloalkylcarbonyl, arylcarbonyl, heteroarylcarbonyl, arylsulfonyl, heteroarylsulfonyl, cycloalkylsulfonyl and alkylsulfonyl, or R⁶ and R⁷ are combined to form a cyclic structure including the nitrogen atom to which they are both attached.

B135. The compound of embodiment B134, wherein:
R⁹ is aryl or alkyl; and
R¹¹ is alkyl.

B136. The compound of embodiment B134 or embodiment B135, wherein R⁹ is methyl or phenyl.

B137. A compound which binds to the GTP binding domain of one or more members of the Ras superfamily and inhibits the one or more members of the Ras superfamily with an IC₅₀ value of less than 10 micromolar, wherein the compound is the compound or pharmaceutically acceptable derivative of any one of embodiments B127-136.

B138. The compound of embodiment B137, wherein one or more members of the Ras superfamily is Ras.

B139. The compound of embodiment B137, wherein one or more members of the Ras superfamily is Rho.

B140. The compound of embodiment B137, wherein one or more members of the Ras superfamily is Rac.

B141. The compound of embodiment B138, wherein the Ras is DIRAS1; DIRAS2; DIRAS3; ERAS; GEM; HRAS; KRAS; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; or RRAS2.

B142. The compound of embodiment B141, wherein the Ras is HRAS, KRAS, NRAS, or a mutant thereof.

B143. The compound of embodiment B142, wherein the Ras is HRAS or a mutant thereof.

B144. The compound of embodiment B142, wherein the Ras is KRAS or a mutant thereof.

B145. The compound of embodiment B142, wherein the Ras is NRAS or a mutant thereof.

B146. The compound of embodiment B139, wherein the Rho is RHOA; RHOB; RHOBTB1; RHOBTB2; RHOBTB3; RHOC; RHOD; RHOF; RHOG; RHOH; RHOJ; RHOQ; RHOU; RHOV; RND1; RND2; RND3; RAC1; RAC2; RAC3; CDC42, or a mutant thereof.

B147. The compound of embodiment B139, wherein the Rho is Rac.

B148. The compound of embodiment B140 or B147, wherein the Rac is RAC1; RAC2; RAC3; RHOG, or a mutant thereof.

B149. The compound or pharmaceutically acceptable derivative of any one of embodiments B127-B136, wherein the pharmaceutically acceptable derivative of the compound is a pharmaceutically acceptable salt of said compound.

B150. A method of inhibiting the function of one or more members of the Ras superfamily, comprising administering to a subject a compound which inhibits the one or more members of the Ras superfamily with an IC50 value of less than 10 PM, wherein the compound is the compound or pharmaceutically acceptable derivative of any one of embodiments B127-B136 or the compound is the compound or pharmaceutically acceptable salt of embodiment B149.

B151. The method of embodiment B150, wherein one or more members of the Ras superfamily is Ras.

B152. The method of embodiment B150, wherein one or more members of the Ras superfamily is Rho.

B153. The method of embodiment B150, wherein one or more members of the Ras superfamily is Rac.

B154. The method of embodiment B150, wherein the Ras is DIRAS1; DIRAS2; DIRAS3; ERAS; GEM; HRAS; KRAS; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REMI; REM2; RERG; RERGL; RRAD; RRAS; or RRAS2.

B155. The method of embodiment B154, wherein the Ras is HRAS, KRAS, NRAS or a mutant thereof.

B156. The method of embodiment B154, wherein the Ras is HRAS or a mutant thereof.

B157. The method of embodiment B154, wherein the Ras is KRAS or a mutant thereof.

B158. The method of embodiment B154, wherein the Ras is NRAS or a mutant thereof.

B159. The method of embodiment B152, wherein the Rho is RHOA; RHOB; RHOBTB1; RHOBTB2; RHOBTB3; RHOC; RHOD; RHOF; RHOG; RHOH; RHOJ; RHOQ; RHOU; RHOV; RND1; RND2; RND3; RAC1; RAC2; RAC3; CDC42, or a mutant thereof.

B160. The method of embodiment B169, wherein the Rho is Rae.

B161. The method of embodiment B153 or B160, wherein the Rac is RAC1; RAC2; RAC3; RHOG, or a mutant thereof.

B162. The method of embodiment B150, wherein the inhibiting the function of one or more members of the Ras superfamily is a treatment, prevention or amelioration of one or more symptoms of cancer.

B163. The method of any of embodiments B151 or B154-B158, wherein the inhibiting the function of Ras is a treatment, prevention or amelioration of one or more symptoms of cancer.

B164. The method of any of embodiments B152 or B159-B160, wherein the inhibiting the function of Rho is a treatment, prevention or amelioration of one or more symptoms of cancer.

B165. The method of any of embodiments B153 or B160-B161, wherein the inhibiting the function of Rac is a treatment, prevention or amelioration of one or more symptoms of cancer.

B166. The method of any of embodiments B162-B165, wherein the cancer is a solid tumor.

B167. The method of embodiment B166, wherein the solid tumor is hepatocellular carcinoma, prostate cancer, pancreatic cancer, lung cancer, breast cancer, ovarian cancer, colon cancer, small intestine cancer, biliary tract cancer, endometrium cancer, skin cancer (melanoma), cervix cancer, urinary tract cancer, or glioblastoma.

B168. The method of embodiment B167, wherein the solid tumor is pancreatic cancer.

B169. The method of embodiment B167, wherein the solid tumor is colon cancer.

B170. The method of embodiment B167, wherein the solid tumor is small intestine cancer.

B171. The method of embodiment B167, wherein the solid tumor is biliary tract cancer.

B172. The method of embodiment B167, wherein the solid tumor is endometrium cancer.

B173. The method of embodiment B167, wherein the solid tumor is lung cancer.

B174. The method of embodiment B167, wherein the solid tumor is breast cancer.

B175. The method of embodiment B167, wherein the solid tumor is skin cancer.

B176. The method of embodiment B167, wherein the solid tumor is cervix cancer.

B177. The method of embodiment B167, wherein the solid tumor is urinary tract cancer.

B178. The method of any of embodiments B162-B165, wherein the cancer is a blood borne tumor.

B179. The method of embodiment B178, wherein the blood borne tumor is a leukemia.

B180. The method of embodiment B178, wherein the blood borne tumor is chronic lymphocytic leukemia (CLL), chronic myelocytic leukemia (CML), acute lymphoblastic leukemia (ALL), acute myeloid leukemia (AML), or acute myeloblastic leukemia (AML).

B181. The method of any one of embodiments B178-B180, wherein the blood borne tumor is metastatic.

B182. The method of embodiment B150, wherein the inhibiting the function of one or more members of the Ras superfamily is a treatment, prevention or amelioration of one or more symptoms of an inflammatory disease.

B183. The method of any of embodiments B151 or B154-B158, wherein inhibiting the function of Ras is a treatment, prevention or amelioration of one or more symptoms of an inflammatory disease.

B184. The method of any of embodiments B152 or B159-B160, wherein the inhibiting the function of Rho is a treatment, prevention or amelioration of one or more symptoms of inflammatory disease.

B185. The method of any of embodiments B153 or B160-B161, wherein the inhibiting the function of Rac is a treatment, prevention or amelioration of one or more symptoms of inflammatory disease.

B186. The method of any of embodiments B182-B185, wherein the inflammatory disease is gastritis, schistosomiasis, cholangitis, chronic cholecystitis, pelvic inflammatory disease, chronic cervicitis, osteomyelitis, inflammatory bowel disease, reflux esophagitis, Barrett's esophagus, bladder inflammation (cystitis), asbestosis, silicosis, gingivitis, lichen planus, pancreatitis, protease mutation, lichen sclerosis, slaladenitis, bronchitis, Sjogren syndrome or Hashimoto's thyroiditis.

B187. The method of any of embodiments B182-B185, wherein the inflammatory disease is Alzheimer's disease (AD), ankylosing spondylitis, arthritis (osteoarthritis, rheumatoid arthritis (RA), psoriatic arthritis), asthma, atherosclerosis, Crohn's disease, colitis, dermatitis, diverticulitis, fibromyalgia, hepatitis, irritable bowel syndrome (IBS), systemic lupus, erythematous (SLE), nephritis, Parkinson's disease, ulcerative colitis.

B188. The method of embodiment B187, wherein the inflammatory disease is Alzheimer's disease (AD).

B189. The method of embodiment B187, wherein the inflammatory disease is ankylosing spondylitis.

B190. The method of embodiment B187, wherein the inflammatory disease is arthritis (osteoarthritis, rheumatoid arthritis (RA), psoriatic arthritis).

B191. The method of embodiment B187, wherein the inflammatory disease is asthma.

B192. The method of embodiment B187, wherein the inflammatory disease is atherosclerosis.

B193. The method of embodiment B187, wherein the inflammatory disease is Crohn's disease.

B194. The method of embodiment B187, wherein the inflammatory disease is colitis.

B195. The method of embodiment B187, wherein the inflammatory disease is dermatitis.

B196. The method of embodiment B187, wherein the inflammatory disease is diverticulitis.

B197. The method of embodiment B187, wherein the inflammatory disease is fibromyalgia.

B198. The method of embodiment B187, wherein the inflammatory disease is hepatitis.

B199. The method of embodiment B187, wherein the inflammatory disease is irritable bowel syndrome (IBS).

B200. The method of embodiment B187, wherein the inflammatory disease is systemic lupus.

B201. The method of embodiment B187, wherein the inflammatory disease is erythematous (SLE).

B202. The method of embodiment B187, wherein the inflammatory disease is nephritis.

B203. The method of embodiment B187, wherein the inflammatory disease is Parkinson's disease.

B204. The method of embodiment B187, wherein the inflammatory disease is ulcerative colitis.

B205. The method of embodiment B150, wherein the inhibiting the function of one or more members of the Ras superfamily is a treatment, prevention or amelioration of one or more symptoms of a rasopathy.

B206. The method of any of embodiments B151 or B154-B158, wherein the inhibiting the function of Ras is a treatment for a rasopathy.

B207. The method of any of embodiments B152 or B159-B160, wherein the inhibiting the function of Rho is a treatment for a rasopathy.

B208. The method of any of embodiments B153 or B160-B161, wherein the inhibiting the function of Rac is a treatment for a rasopathy.

B209. The method of any of embodiments B205-B208, wherein the rasopathy is neurofibromatosis type 1, Noonan's syndrome or Costello syndrome.

B210. The method of any of embodiments B151 or B154-B158, wherein the inhibiting the function of Ras is a treatment for Ras-associated autoimmune leukoproliferative disorder.

B211. The method of embodiment B150, wherein the inhibiting the function of one or more members of the Ras superfamily is a treatment, prevention or amelioration of one or more symptoms of a fibrotic disease.

B212. The method of any of embodiments B151 or B154-B158, wherein the inhibiting the function of Ras is a treatment, prevention or amelioration of one or more symptoms of a fibrotic disease.

B213. The method of any of embodiments B152 or B159-B160, wherein the inhibiting the function of Rho is a treatment, prevention or amelioration of one or more symptoms of a fibrotic disease.

B214. The method of any of embodiments B152 or B159-B160, wherein the inhibiting the function of Rac is a treatment, prevention or amelioration of one or more symptoms of a fibrotic disease.

B215. The method of any one of embodiments B162, B182, B205, or B211, wherein one or more members of the Ras superfamily is Ras.

B216. The method of any one of embodiments B162, B182, B205, or B211, wherein one or more members of the Ras superfamily is Rho.

B217. The method of any one of embodiments B162, B182, B205, or B211, wherein one or more members of the Ras superfamily is Rac.

B218. A pharmaceutical composition, comprising the compound or pharmaceutically acceptable derivative of any one of embodiments B127-B136, and a pharmaceutically acceptable carrier.

B219. The pharmaceutical composition of embodiment B218, wherein the pharmaceutical composition comprises a therapeutic amount of said compound or pharmaceutically acceptable derivative thereof.

B220. A pharmaceutical composition, comprising the compound or pharmaceutically acceptable salt of embodiment B149, and a pharmaceutically acceptable carrier.

B221. The pharmaceutical composition of embodiment B220, wherein the pharmaceutical composition comprises a therapeutic amount of said compound or pharmaceutically acceptable salt thereof.

B222. A method of inhibiting the function of one or more members of the Ras superfamily, comprising administering to a subject the pharmaceutical composition of any one of embodiments B218-B221.

B223. A method of inhibiting the function of one or more members of the Ras superfamily, comprising administering to a subject the compound or pharmaceutically acceptable derivative of any one of embodiments B127-B136.

This disclosure is not to be limited in scope by the embodiments disclosed in the examples which are intended as single illustrations of individual aspects, and any methods which are functionally equivalent are within the scope of this disclosure. Indeed, various modifications in addition to those shown and described herein will become apparent to those skilled in the art from the foregoing description. Such modifications are intended to fall within the scope of the appended claims. This disclosure is not to be limited in scope, or cabined in any way, by the use of any subheadings, which are provided only for the convenience of the reader.

All references, such as publications, patents, and patent applications, mentioned in this specification are herein incorporated by reference in their entirety to the same extend as if each individual publication, patent, and patent application, was specifically and individually indicated to incorporated by reference in its entirety.

What is claimed is:

1. A method of treating fibrosis in a subject having a fibrotic disease, comprising administering to the subject a compound of Formula I: 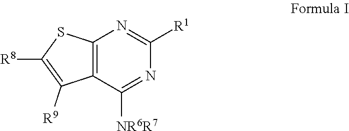
or a pharmaceutically acceptable salt thereof,
wherein:
R¹ is H, alkyl, aryl or heteroaryl;
R⁸ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
R⁹ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
R³ is hydrogen, alkyl, alkenyl, alkynyl, aryl, heteroaryl, heterocyclyl, cycloalkyl, alkylcarbonyl, cycloalkylcarbonyl or arylcarbonyl;
R⁴ is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy, aralkoxy, or NR⁶R⁷;
each R⁵ is selected from the group consisting of hydrogen, alkyl, alkenyl, alkynyl, aryl, heteroaryl, heterocyclyl, cycloalkyl, alkylcarbonyl, cycloalkylcarbonyl, and arylcarbonyl; and
R⁶ and R⁷ are independently selected from the group consisting of hydrogen, alkyl, alkenyl, alkynyl, aryl, heteroaryl, cycloalkyl, heterocyclyl, alkylcarbonyl, cycloalkylcarbonyl, arylcarbonyl, heteroarylcarbonyl, arylsulfonyl, heteroarylsulfonyl, cycloalkylsulfonyl and alkylsulfonyl, or R⁶ and R⁷ are combined to form a cyclic structure including the nitrogen atom to which they are both attached;
wherein the compound of Formula I or pharmaceutically acceptable salt thereof inhibits phosphorylation of Smad2/3, according to Phospho-Smad2/3 Inhibition Assay, and is inactive according to MAPK p38 Activation Assay.

2. The method of claim 1, wherein:
R¹ is heteroaryl;
R⁸ is aryl or heteroaryl;
R⁹ is aryl or alkyl; and
R⁶ and R⁷ are independently selected from the group consisting of hydrogen, alkyl, aryl, heteroaryl, cycloalkyl, and heterocyclyl, or R⁶ and R⁷ are combined to form a cyclic structure including the nitrogen atom to which they are both attached.

3. The method of claim 1 or claim 2, wherein R¹ is:

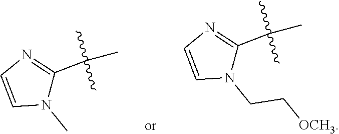

4. The method of any one of claims 1-3, wherein R⁸ is:
phenyl,

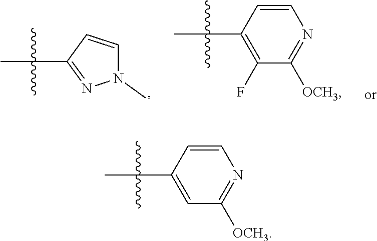

5. The method of any one of claims 1-4, wherein R⁹ is phenyl.

6. The method of any one of claims 1-5, wherein the —NR⁶R⁷ group depicted in Formula I is:

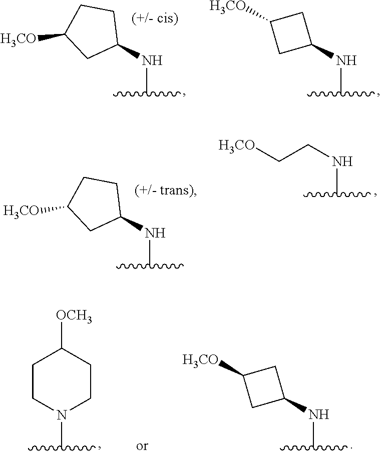 [SMILES: *N[C@@H]1CC[C@H](OC)C1 ; *N[C@H]1C[C@H](OC)C1 ; *N[C@@H]1CC[C@@H](OC)C1 ; *NCCOC ; *N1CCC(OC)CC1 ; *N[C@H]1C[C@@H](OC)C1]

7. The method of any one of claims 1-6, wherein the compound of Formula I or pharmaceutically acceptable salt thereof is selected from the group consisting of:
N-(2-Methoxyethyl)-2-(1-methyl-1H-imidazol-2-yl)-5,6-diphenylthieno[2,3-d]pyrimidin-4-amine;
N-((1s,3s)-3-methoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine;
rac-N-((1R,3S)-3-Methoxycyclopentyl)-6-(2-methoxypyridin-4-yl)-2-(1-methyl-1H-imidazol-2-yl)-5-phenylthieno[2,3-d]pyrimidin-4-amine;
6-(3-Fluoro-2-methoxypyridin-4-yl)-N-((1r,3r)-3-methoxycyclobutyl)-2-(1-methyl-1H-imidazol-2-yl)-5-phenylthieno[2,3-d]pyrimidin-4-amine;
N-((1r,3r)-3-Methoxycyclobutyl)-6-(2-methoxypyridin-4-yl)-2-(1-methyl-1H-imidazol-2-yl)-5-phenylthieno[2,3-d]pyrimidin-4-amine; and
N-((1R,3R)-3-Methoxycyclopentyl)-6-(2-methoxypyridin-4-yl)-2-(1-methyl-1H-imidazol-2-yl)-5-phenylthieno[2,3-d]pyrimidin-4-amine.

8. The method of any one of claims 1-7, wherein the compound of Formula I or pharmaceutically acceptable salt thereof inhibits phosphorylation of Smad2/3 by 20% or more at 10 μM, according to Phospho-Smad2/3 Inhibition Assay.

9. The method of claim 8, wherein the compound of Formula I or pharmaceutically acceptable salt thereof is selected from the group consisting of:
N-(2-Methoxyethyl)-2-(1-methyl-1H-imidazol-2-yl)-5,6-diphenylthieno[2,3-d]pyrimidin-4-amine;
N-((1s,3s)-3-methoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine;
rac-N-((1R,3S)-3-Methoxycyclopentyl)-6-(2-methoxypyridin-4-yl)-2-(1-methyl-1H-imidazol-2-yl)-5-phenylthieno[2,3-d]pyrimidin-4-amine;
6-(3-Fluoro-2-methoxypyridin-4-yl)-N-((1r,3r)-3-methoxycyclobutyl)-2-(1-methyl-1H-imidazol-2-yl)-5-phenylthieno[2,3-d]pyrimidin-4-amine;
N-((1r,3r)-3-Methoxycyclobutyl)-6-(2-methoxypyridin-4-yl)-2-(1-methyl-1H-imidazol-2-yl)-5-phenylthieno[2,3-d]pyrimidin-4-amine; and
N-((1R,3R)-3-Methoxycyclopentyl)-6-(2-methoxypyridin-4-yl)-2-(1-methyl-1H-imidazol-2-yl)-5-phenylthieno[2,3-d]pyrimidin-4-amine.

10. The method of any one of claims 1-7, wherein the compound of Formula I or pharmaceutically acceptable salt thereof inhibits phosphorylation of Smad2/3 by 30% or more at 10 μM, according to Phospho-Smad2/3 Inhibition Assay.

11. The method of claim 10, wherein the compound of Formula I or pharmaceutically acceptable salt thereof is selected from the group consisting of:
N-(2-Methoxyethyl)-2-(1-methyl-1H-imidazol-2-yl)-5,6-diphenylthieno[2,3-d]pyrimidin-4-amine;
N-((1s,3s)-3-methoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine;
rac-N-((1R,3S)-3-Methoxycyclopentyl)-6-(2-methoxypyridin-4-yl)-2-(1-methyl-1H-imidazol-2-yl)-5-phenylthieno[2,3-d]pyrimidin-4-amine;
6-(3-Fluoro-2-methoxypyridin-4-yl)-N-((1r,3r)-3-methoxycyclobutyl)-2-(1-methyl-1H-imidazol-2-yl)-5-phenylthieno[2,3-d]pyrimidin-4-amine;
N-((1r,3r)-3-Methoxycyclobutyl)-6-(2-methoxypyridin-4-yl)-2-(1-methyl-1H-imidazol-2-yl)-5-phenylthieno[2,3-d]pyrimidin-4-amine; and
N-((1R,3R)-3-Methoxycyclopentyl)-6-(2-methoxypyridin-4-yl)-2-(1-methyl-1H-imidazol-2-yl)-5-phenylthieno[2,3-d]pyrimidin-4-amine.

12. The method of any one of claims 1-7, wherein the compound of Formula I or pharmaceutically acceptable salt thereof inhibits phosphorylation of Smad2/3 by 50% or more at 10 μM, according to Phospho-Smad2/3 Inhibition Assay.

13. The method of claim 12, wherein the compound of Formula I or pharmaceutically acceptable salt thereof is selected from the group consisting of:
N-(2-Methoxyethyl)-2-(1-methyl-1H-imidazol-2-yl)-5,6-diphenylthieno[2,3-d]pyrimidin-4-amine;
N-((1s,3s)-3-methoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine;
rac-N-((1R,3S)-3-Methoxycyclopentyl)-6-(2-methoxypyridin-4-yl)-2-(1-methyl-1H-imidazol-2-yl)-5-phenylthieno[2,3-d]pyrimidin-4-amine;
6-(3-Fluoro-2-methoxypyridin-4-yl)-N-((1r,3r)-3-methoxycyclobutyl)-2-(1-methyl-1H-imidazol-2-yl)-5-phenylthieno[2,3-d]pyrimidin-4-amine;
N-((1r,3r)-3-Methoxycyclobutyl)-6-(2-methoxypyridin-4-yl)-2-(1-methyl-1H-imidazol-2-yl)-5-phenylthieno[2,3-d]pyrimidin-4-amine; and
N-((1R,3R)-3-Methoxycyclopentyl)-6-(2-methoxypyridin-4-yl)-2-(1-methyl-1H-imidazol-2-yl)-5-phenylthieno[2,3-d]pyrimidin-4-amine.

14. The method of any one of claims 1-7, wherein the compound of Formula I or pharmaceutically acceptable salt thereof inhibits phosphorylation of Smad2/3 by 70% or more at 10 μM, according to Phospho-Smad2/3 Inhibition Assay.

15. The method of claim 14, wherein the compound of Formula I or pharmaceutically acceptable salt thereof is selected from the group consisting of:
N-((1s,3s)-3-methoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine;
rac-N-((1R,3S)-3-Methoxycyclopentyl)-6-(2-methoxypyridin-4-yl)-2-(1-methyl-1H-imidazol-2-yl)-5-phenylthieno[2,3-d]pyrimidin-4-amine;
6-(3-Fluoro-2-methoxypyridin-4-yl)-N-((1r,3r)-3-methoxycyclobutyl)-2-(1-methyl-1H-imidazol-2-yl)-5-phenylthieno[2,3-d]pyrimidin-4-amine;
N-((1r,3r)-3-Methoxycyclobutyl)-6-(2-methoxypyridin-4-yl)-2-(1-methyl-1H-imidazol-2-yl)-5-phenylthieno[2,3-d]pyrimidin-4-amine; and
N-((1R,3R)-3-Methoxycyclopentyl)-6-(2-methoxypyridin-4-yl)-2-(1-methyl-1H-imidazol-2-yl)-5-phenylthieno[2,3-d]pyrimidin-4-amine.

16. The method of any one of claims 1-15, wherein the compound of Formula I or pharmaceutically acceptable salt thereof is exclusive of compounds selected from the group consisting of:
N-(2-Methoxyethyl)-N-methyl-2-(1-methyl-1H-imidazol-2-yl)-5,6-diphenylthieno[2,3-d]pyrimidin-4-amine;
N-(3-Methoxypropyl)-2-(1-methyl-1H-imidazol-2-yl)-5,6-diphenylthieno[2,3-d]pyrimidin-4-amine;
5-(2-Chlorophenyl)-N-(2-methoxyethyl)-2-(1-methyl-1H-imidazol-2-yl)-6-phenylthieno[2,3-d]pyrimidin-4-amine;
rac-N-((1R,3S)-3-Methoxycyclopentyl)-2-(1-methyl-1H-imidazol-2-yl)-6-(1-methyl-1H-pyrazol-3-yl)-5-phenylthieno[2,3-d]pyrimidin-4-amine;
4-(4-Methoxypiperidin-1-yl)-2-(1-methyl-1H-imidazol-2-yl)-6-(1-methyl-1H-pyrazol-3-yl)-5-phenylthieno[2,3-d]pyrimidine;
6-(2-Chloro-3-methoxyphenyl)-N-(2-methoxyethyl)-5-(4-methoxyphenyl)-2-(1-methyl-1H-imidazol-2-yl)thieno[2,3-d]pyrimidin-4-amine;
6-(2-Chloro-3-methoxyphenyl)-N-(2-methoxyethyl)-5-(4-methoxyphenyl)-2-(1-methyl-1H-imidazol-4-yl)thieno[2,3-d]pyrimidin-4-amine;
6-(2-Chloro-3-methoxyphenyl)-N-(2-methoxyethyl)-2-(1-methyl-1H-imidazol-2-yl)-5-phenylthieno[2,3-d]pyrimidin-4-amine;
rac-6-(2-Fluoro-3-methoxyphenyl)-N-((1R,3S)-3-methoxycyclopentyl)-5-methyl-2-(1-methyl-1H-imidazol-2-yl)thieno[2,3-d]pyrimidin-4-amine and rac-6-(2-Fluoro-3-methoxyphenyl)-N-((1R,3R)-3-methoxycyclopentyl)-5-methyl-2-(1-methyl-1H-imidazol-2-yl)thieno[2,3-d]pyrimidin-4-amine;
6-(1-Isopropyl-1H-pyrazol-3-yl)-N-((1S,3R)-3-methoxycyclopentyl)-2-(1-methyl-1H-imidazol-2-yl)-5-phenylthieno[2,3-d]pyrimidin-4-amine;
rac-6-(1-Isopropyl-1H-pyrazol-3-yl)-N-((1R,3S)-3-methoxycyclopentyl)-5-methyl-2-(1-methyl-1H-imidazol-2-yl)thieno[2,3-d]pyrimidin-4-amine;
rac-6-(1-Isopropyl-1H-pyrazol-3-yl)-4-(3-methoxypiperidin-1-yl)-5-methyl-2-(1-methyl-1H-imidazol-2-yl)thieno[2,3-d]pyrimidine;
4-(6-(1-Isopropyl-1H-pyrazol-3-yl)-5-methyl-2-(1-methyl-1H-imidazol-2-yl)thieno[2,3-d]pyrimidin-4-yl)-2-(methoxymethyl)morpholine;
6-(1-Isopropyl-1H-pyrazol-3-yl)-N-(3-methoxyphenyl)-5-methyl-2-(1-methyl-1H-imidazol-2-yl)thieno[2,3-d]pyrimidin-4-amine;
6-(1-Isopropyl-1H-pyrazol-3-yl)-N-(6-methoxypyridin-2-yl)-5-methyl-2-(1-methyl-1H-imidazol-2-yl)thieno[2,3-d]pyrimidin-4-amine;
6-(1-Isopropyl-1H-pyrazol-3-yl)-N-(6-methoxypyrimidin-4-yl)-5-methyl-2-(1-methyl-1H-imidazol-2-yl)thieno[2,3-d]pyrimidin-4-amine;
rac-6-(1-Isopropyl-1H-pyrazol-3-yl)-N-((1R,3S)-3-(2-methoxyethoxy)cyclopentyl)-5-methyl-2-(1-methyl-1H-imidazol-2-yl)thieno[2,3-d]pyrimidin-4-amine;
rac-6-(1-(2-Methoxyethyl)-1H-pyrazol-3-yl)-N-(((1S,2R)-2-(methoxymethyl)cyclobutyl)methyl)-5-methyl-2-(1-methyl-1H-imidazol-2-yl)thieno[2,3-d]pyrimidin-4-amine;
6-(2-Fluoro-3-methoxyphenyl)-N-((1r,3r)-3-methoxycyclobutyl)-2-(1-methyl-1H-imidazol-2-yl)-5-phenylthieno[2,3-d]pyrimidin-4-amine;
6-(2-Fluoro-3-methoxyphenyl)-N-((1R,3R)-3-methoxycyclopentyl)-2-(1-methyl-1H-imidazol-2-yl)-5-phenylthieno[2,3-d]pyrimidin-4-amine;
6-(2-Fluoro-3-methoxyphenyl)-N-((1r,3r)-3-methoxycyclobutyl)-5-methyl-2-(1-methyl-1H-imidazol-2-yl)thieno[2,3-d]pyrimidin-4-amine; and
rac-6-(2-Fluoro-3-methoxyphenyl)-N-((1R,3R)-3-methoxycyclopentyl)-5-methyl-2-(1-methyl-1H-imidazol-2-yl)thieno[2,3-d]pyrimidin-4-amine.

17. The method of any one of claims 1-16, wherein the compound of Formula I or pharmaceutically acceptable salt thereof inhibits MAPK p38, according to MAPK p38 Activation Assay.

18. The method of any one of claims 1-17, wherein the compound of Formula I or pharmaceutically acceptable salt thereof activates JNK, according to JNK Activation Assay.

19. The method of any one of claims 1-18, wherein the compound of Formula I or pharmaceutically acceptable salt thereof is a modulator of Ras superfamily activity according to a Ras Superfamily Activity Assay.

20. A method of treating fibrosis in a subject having a fibrotic disease, comprising administering to the subject a compound of Formula II: 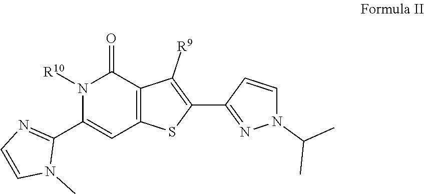
or a pharmaceutically acceptable salt thereof,
wherein:
R⁹ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
R¹⁰ is H, alkyl, alkenyl, alkynyl, aryl, cycloalkyl, heterocyclyl, heteroaryl, C(O)R⁴, or S(O)ₚR⁴;
R³ is hydrogen, alkyl, alkenyl, alkynyl, aryl, heteroaryl, heterocyclyl, cycloalkyl, alkylcarbonyl, cycloalkylcarbonyl or arylcarbonyl;
R⁴ is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy, aralkoxy, or NR⁶R⁷;
each R⁵ is selected from the group consisting of hydrogen, alkyl, alkenyl, alkynyl, aryl, heteroaryl, heterocyclyl, cycloalkyl, alkylcarbonyl, cycloalkylcarbonyl, and arylcarbonyl; and
R⁶ and R⁷ are independently selected from the group consisting of hydrogen, alkyl, alkenyl, alkynyl, aryl, heteroaryl, cycloalkyl, heterocyclyl, alkylcarbonyl, cycloalkylcarbonyl, arylcarbonyl, heteroarylcarbonyl, arylsulfonyl, heteroarylsulfonyl, cycloalkylsulfonyl and alkylsulfonyl, or R⁶ and R⁷ are combined to form a cyclic structure including the nitrogen atom to which they are both attached;
wherein the compound of Formula II or pharmaceutically acceptable salt thereof is inactive according to MAPK p38 Activation Assay.

21. The method of claim 20, wherein:
R⁹ is alkyl; and
R¹⁰ is alkyl.

22. The method of claim 20 or claim 21, wherein R⁹ is methyl.

23. The method of any one of claims 20-22, wherein the compound of Formula II or pharmaceutically acceptable salt thereof is selected from the group consisting of:
6-(1-Isopropyl-1H-pyrazol-3-yl)-3-(3-methoxypropyl)-5-methyl-2-(1-methyl-1H-imidazol-2-yl)thieno[2,3-d]pyrimidin-4(3H)-one;
6-(1-Isopropyl-1H-pyrazol-3-yl)-3-(4-methoxybutyl)-5-methyl-2-(1-methyl-1H-imidazol-2-yl)thieno[2,3-d]pyrimidin-4(3H)-one; and
6-(1-Isopropyl-1H-pyrazol-3-yl)-3-((6-methoxypyrimidin-4-yl)methyl)-5-methyl-2-(1-methyl-1H-imidazol-2-yl)thieno[2,3-d]pyrimidin-4(3H)-one.

24. The method of any one of claims 20-23, wherein the compound of Formula II or pharmaceutically acceptable salt thereof is exclusive of compounds selected from the group consisting of:
6-(1-Isopropyl-1H-pyrazol-3-yl)-3-(2-methoxyethyl)-5-methyl-2-(1-methyl-1H-imidazol-2-yl)thieno[2,3-d]pyrimidin-4(3H)-one; and
6-(1-Isopropyl-1H-pyrazol-3-yl)-3-(3-methoxypropyl)-2-(1-methyl-1H-imidazol-2-yl)-5-phenylthieno[2,3-d]pyrimidin-4(3H)-one.

25. The method of any one of claims 20-24, wherein the compound of Formula II or pharmaceutically acceptable salt thereof inhibits MAPK p38, according to MAPK p38 Activation Assay.

26. The method of any one of claims 20-25, wherein the compound of Formula II or pharmaceutically acceptable salt thereof inhibits JNK, according to JNK Activation Assay.

27. The method of any one of claims 20-26, wherein the compound of Formula II or pharmaceutically acceptable salt thereof is a modulator of Ras superfamily activity according to a Ras Superfamily Activity Assay.

28. A method of treating fibrosis in a subject having a fibrotic disease, comprising administering to the subject a compound of Formula III: 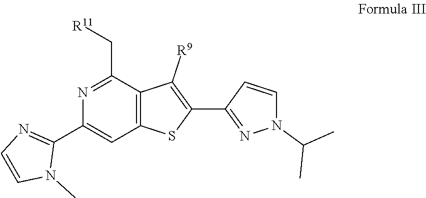
or a pharmaceutically acceptable salt thereof,
wherein:
R⁹ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
R¹¹ is H, alkyl, alkenyl, alkynyl, aryl, cycloalkyl, heterocyclyl, heteroaryl, C(O)R⁴, or S(O)ₚR⁴;
R³ is hydrogen, alkyl, alkenyl, alkynyl, aryl, heteroaryl, heterocyclyl, cycloalkyl, alkylcarbonyl, cycloalkylcarbonyl or arylcarbonyl;
R⁴ is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy, aralkoxy, or NR⁶R⁷;
each R⁵ is selected from the group consisting of hydrogen, alkyl, alkenyl, alkynyl, aryl, heteroaryl, heterocyclyl, cycloalkyl, alkylcarbonyl, cycloalkylcarbonyl, and arylcarbonyl; and
R⁶ and R⁷ are independently selected from the group consisting of hydrogen, alkyl, alkenyl, alkynyl, aryl, heteroaryl, cycloalkyl, heterocyclyl, alkylcarbonyl, cycloalkylcarbonyl, arylcarbonyl, heteroarylcarbonyl, arylsulfonyl, heteroarylsulfonyl, cycloalkylsulfonyl and alkylsulfonyl, or R⁶ and R⁷ are combined to form a cyclic structure including the nitrogen atom to which they are both attached;
wherein the compound of Formula III or pharmaceutically acceptable salt thereof is inactive according to MAPK p38 Activation Assay.

29. The method of claim 28, wherein:
R⁹ is aryl; and
R¹¹ is alkyl.

30. The method of claim 28 or claim 29, wherein R⁹ is methyl.

31. The method of any one of claims 28-30, wherein the compound of Formula III or pharmaceutically acceptable salt thereof is selected from the group consisting of:
6-(1-Isopropyl-1H-pyrazol-3-yl)-4-(2-methoxyethoxy)-5-methyl-2-(1-methyl-1H-imidazol-2-yl)thieno[2,3-d]pyrimidine;
6-(1-Isopropyl-1H-pyrazol-3-yl)-4-(3-methoxypropoxy)-5-methyl-2-(1-methyl-1H-imidazol-2-yl)thieno[2,3-d]pyrimidine; and
6-(1-Isopropyl-1H-pyrazol-3-yl)-4-(4-methoxybutoxy)-5-methyl-2-(1-methyl-1H-imidazol-2-yl)thieno[2,3-d]pyrimidine.

32. The method of any one of claims 28-31, wherein the compound of Formula III or pharmaceutically acceptable salt thereof is exclusive of 6-(1-Isopropyl-1H-pyrazol-3-yl)-4-(3-methoxypropoxy)-2-(1-methyl-1H-imidazol-2-yl)-5-phenylthieno[2,3-d]pyrimidine.

33. The method of any one of claims 28-32, wherein the compound of Formula III or pharmaceutically acceptable salt thereof inhibits INK, according to INK Activation Assay.

34. The method of any one of claims 28-32, wherein the compound of Formula III or pharmaceutically acceptable salt thereof activates INK, according to INK Activation Assay.

35. The method of any one of claims 28-34, wherein the compound of Formula III or pharmaceutically acceptable salt thereof is a modulator of Ras superfamily activity according to a Ras Superfamily Activity Assay.

36. A method of treating fibrosis in a subject having a fibrotic disease, comprising administering to the subject a compound or a pharmaceutically acceptable salt thereof identified as a modulator of Ras superfamily activity according to a Ras Superfamily Activity Assay; wherein the identified Ras superfamily modulating compound or pharmaceutically acceptable salt thereof inhibits phosphorylation of Smad2/3, according to Phospho-Smad2/3 Inhibition Assay, and is inactive according to MAPK p38 Activation Assay.

37. The method of claim 36, wherein the identified Ras superfamily modulating compound or pharmaceutically acceptable salt thereof inhibits phosphorylation of Smad2/3 by 20%, 30%, 50%, 70%, or more at 10 μM, according to Phospho-Smad2/3 Inhibition Assay.

38. The method of claim 36 or claim 37, wherein the identified Ras superfamily modulating compound or pharmaceutically acceptable salt thereof activates INK, according to INK Activation Assay.

39. The method of any one of claims 36-38, wherein the Ras Superfamily Activity Assay comprises:
a) contacting the compound or pharmaceutically acceptable salt thereof with a Ras superfamily protein;
b) incubating a cyanine-labeled GTP with said compound or pharmaceutically acceptable salt thereof and the Ras superfamily protein; and
c) measuring the amount of the cyanine-labeled GTP bound to the Ras superfamily protein.

40. The method of claim 39, wherein the Ras Superfamily Activity Assay is a cell-free assay.

41. The method of claim 39 or claim 40, wherein the Ras Superfamily Activity Assay is a GTP-binding competition assay.

42. The method of any one of claims 36-41, wherein the Ras superfamily protein is a Ras protein, a Rac protein, or a Rho protein.

43. The method of claim 42, wherein the Ras superfamily protein is a Ras protein.

44. The method of claim 43, wherein the Ras Superfamily Activity Assay is a Ras Activity Assay.

45. The method of claim 43 or claim 44, wherein the identified Ras superfamily modulating compound or pharmaceutically acceptable salt thereof is identified as a Ras modulating compound or a pharmaceutically acceptable salt thereof.

46. The method of claim 45, wherein the identified Ras modulating compound or pharmaceutically acceptable salt thereof competitively inhibits GTP binding to the Ras GTP binding domain.

47. The method of claim 45 or claim 46, wherein the identified Ras modulating compound or pharmaceutically acceptable salt thereof binds to the Ras protein GTP binding domain with greater than 25% inhibition at 20 uM.

48. The method of any one of claims 45-47, wherein the identified Ras modulating compound or pharmaceutically acceptable salt thereof has a binding affinity (Kd) to the Ras protein GTP binding domain of less than 10 uM.

49. The method of any one of claims 45-48, wherein the identified Ras modulating compound or pharmaceutically acceptable salt thereof inhibits the Ras activity and has an IC50 value of less than 10 uM.

50. The method of any one of claims 45-49, wherein the identified Ras modulating compound or pharmaceutically acceptable salt thereof inhibits binding of the cyanine labeled GTP with an IC50 value of less than 10 uM.

51. The method of any one of claims 45-50, wherein the cyanine-labeled GTP is a Cy3- or a Cy5-labeled GTP.

52. The method of any one of claims 45-51, wherein the Ras Activity Assay is a GTP-binding competition assay.

53. The method of any one of claims 44-52, wherein the Ras protein is immobilized.

54. The method of any one of claims 44-53, wherein the Ras protein is DIRAS I; DIRAS2; DIRAS3; ERAS; GEM; HRAS; KRAS; MRAS; NKIRASI; NKIRAS2; NRAS; RALA; RALB; RAPIA; RAPIB; RAP2A; RAP2B; RAP2C; RASDI; RASD2; RASLIOA; RASLIOB; RASLI IA; RASLIIB; RASL12; REMI; REM2; RERG; RERGL; RRAD; RRAS; or RRAS2.

55. The method of any one of claims 44-54, wherein the Ras protein is HRAS; KRAS; or NRAS, or a mutant thereof.

56. The method of claim 55, wherein the Ras protein is a KRAS mutant.

57. The method of claim 56, wherein the KRAS mutant is a KRas G12D mutant, KRas G12C mutant, or KRas Q61H mutant.

58. The method of claim 55, wherein the Ras protein is wild-type KRas.

59. The method of claim 55, wherein the Ras protein is HRAS or mutant thereof.

60. The method of claim 55, wherein the Ras protein is NRAS or mutant thereof.

61. The method of claim 42, wherein the Ras superfamily protein is a Rac protein.

62. The method of claim 61, wherein the Ras Superfamily Activity Assay is a Rac Activity Assay.

63. The method of claim 61 or claim 62, wherein the identified Ras superfamily modulating compound or pharmaceutically acceptable salt thereof is identified as a Rac modulating compound or a pharmaceutically acceptable salt thereof.

64. The method of claim 63, wherein the identified Rac modulating compound or pharmaceutically acceptable salt thereof competitively inhibits GTP binding to the Rac GTP binding domain.

65. The method of claim 63 or claim 64, wherein the identified Rac modulating compound or pharmaceutically acceptable salt thereof binds to the Rac protein GTP binding domain with greater than 25% inhibition at 20 uM.

66. The method of any one of claims 63-65, wherein the identified Rac modulating compound or pharmaceutically acceptable salt thereof has a binding affinity (Kd) to the Rac protein GTP binding domain of less than 10 uM.

67. The method of any one of claims 63-66, wherein the identified Rac modulating compound or pharmaceutically acceptable salt thereof inhibits the Rac activity and has an IC50 value of less than 10 uM.

68. The method of any one of claims 63-67, wherein the identified Rac modulating compound or pharmaceutically acceptable salt thereof inhibits binding of the cyanine labeled GTP with an IC50 value of less than 10 uM.

69. The method of any one of claims 63-68, wherein the cyanine-labeled GTP is a Cy3- or a Cy5-labeled GTP.

70. The method of any one of claims 62-69, wherein the Rac Activity Assay is a GTP-binding competition assay.

71. The method of any one of claims 62-70, wherein the Rac protein is immobilized.

72. The method of any one of claims 62-71, wherein the Rac protein is RAC1; RAC2; RAC3; RHOG, or a mutant thereof.

73. The method of claim 72, wherein the Rac protein is wild-type RAC1.

74. The method of claim 63, wherein the Ras superfamily protein is a Rho protein.

75. The method of claim 74, wherein the Ras Superfamily Activity Assay is a Rho Activity Assay.

76. The method of claim 74 or claim 75, wherein the identified Ras superfamily modulating compound or pharmaceutically acceptable salt thereof is identified as a Rho modulating compound or a pharmaceutically acceptable salt thereof.

77. The method of claim 76, wherein the identified Rho modulating compound or pharmaceutically acceptable salt thereof competitively inhibits GTP binding to the Rho GTP binding domain.

78. The method of claim 76 or claim 77, wherein the identified Rho modulating compound or pharmaceutically acceptable salt thereof binds to the Rho protein GTP binding domain with greater than 25% inhibition at 20 uM.

79. The method of any one of claims 76-78, wherein the identified Rho modulating compound or pharmaceutically acceptable salt thereof has a binding affinity (Kd) to the Rho protein GTP binding domain of less than 10 uM.

80. The method of any one of claims 76-79, wherein the identified Rho modulating compound or pharmaceutically acceptable salt thereof inhibits the Rho activity and has an IC50 value of less than 10 uM.

81. The method of any one of claims 76-80, wherein the identified Rho modulating compound or pharmaceutically acceptable salt thereof inhibits binding of the cyanine labeled GTP with an IC50 value of less than 10 uM.

82. The method of any one of claims 76-81, wherein the cyanine-labeled GTP is a Cy3- or a Cy5-labeled GTP.

83. The method of any one of claims 75-82, wherein the Rho Activity Assay is a GTP-binding competition assay.

84. The method of any one of claims 75-83, wherein the Rho protein is immobilized.

85. The method of any one of claims 75-84, wherein the Rho protein is RHOA; RHOB; RHOBTB1; RHOBTB2; RHOBTB3; RHOC; RHOD; RHOF; RHOH; RHOJ; RHOQ; RHOU; RHOV; RND1; RND2; RND3; CDC42, or a mutant thereof.

86. The method of any one of claims 75-85, wherein the Rho protein is wild-type RHOA.

87. The method of any one of claims 1-86, wherein the method treats, prevents, or ameliorates the fibrotic disease in the subject or treats, prevents, or ameliorates one or more symptoms of said fibrotic disease in the subject.

88. The method of any one of claims 1-87, wherein the fibrotic disease is selected from the group consisting of fibrosis of kidney, fibrosis of cardiovascular system, pulmonary fibrosis, cystic fibrosis, idiopathic fibrosis, fibrosis of the lung, bridging fibrosis, fibrosis of the liver, fibrosis of the intestine, fibrosis of the muscular system, fibrosis of the brain, fibrosis of the joints, fibrosis of the skin, fibrosis of the bone marrow, fibrosis of the heart, fibrosis of the soft tissue, fibrosis of the tendons, fibrosis of the lymph nodes, fibrosis of the eyes, retroperitoneum, scleroderma and surgical scarring.

89. The method of claim 88, wherein the fibrotic disease is fibrosis of the kidney.

90. The method of claim 88, wherein the fibrotic disease is fibrosis of the cardiovascular system.

91. The method of claim 88, wherein the fibrotic disease is pulmonary fibrosis.

92. The method of claim 88, wherein the fibrotic disease is cystic fibrosis.

93. The method of claim 88, wherein the fibrotic disease is idiopathic fibrosis.

94. The method of claim 88, wherein the fibrotic disease is fibrosis of the lung.

95. The method of claim 94, wherein the fibrosis of the lung is progressive massive fibrosis and radiation-induced lung injury.

96. The method of claim 88, wherein the fibrotic disease is bridging fibrosis.

97. The method of claim 88, wherein the fibrotic disease is fibrosis of the liver.

98. The method of claim 97, wherein the fibrosis of the liver is cirrhosis.

99. The method of claim 88, wherein the fibrotic disease is fibrosis of the intestine.

100. The method of claim 99, wherein the fibrosis of the intestine is Crohn's disease.

101. The method of claim 88, wherein the fibrotic disease is fibrosis of the muscular system.

102. The method of claim 101, wherein the fibrosis of the muscular system is Duchenne muscular dystrophy (DMD).

103. The method of claim 88, wherein the fibrotic disease is fibrosis of the brain.

104. The method of claim 103, wherein the fibrosis of the brain is glial scar.

105. The method of claim 88, wherein the fibrotic disease is fibrosis of the joints.

106. The method of claim 105, wherein the fibrosis of the joints is arterial stiffness.

107. The method of claim 105 or claim 106, wherein the fibrosis of the joints is fibrosis of the knee.

108. The method of claim 105 or claim 106, wherein the fibrosis of the joints is fibrosis of the shoulder.

109. The method of claim 88, wherein the fibrotic disease is fibrosis of the skin.

110. The method of claim 109, wherein the fibrosis of the skin is Keloid.

111. The method of claim 88, wherein the fibrotic disease is fibrosis of the bone marrow.

112. The method of claim 111, wherein the fibrosis of the bone marrow is Myelofibrosis.

113. The method of claim 88, wherein the fibrotic disease is fibrosis of the heart.

114. The method of claim 113, wherein the fibrosis of the heart is Myocardial fibrosis.

115. The method of claim 88, wherein the fibrotic disease is fibrosis of the soft tissue.

116. The method of claim 88, wherein the fibrotic disease is fibrosis of the tendons.

117. The method of claim 88, wherein the fibrotic disease is fibrosis of the lymph nodes.

118. The method of claim 88, wherein the fibrotic disease is fibrosis of the eyes.

119. The method of claim 88, wherein the fibrotic disease is retroperitoneum.

120. The method of claim 88, wherein the fibrotic disease is scleroderma.

121. The method of claim 88, wherein the fibrotic disease is surgical scarring.

122. The method of claim 102, wherein the Duchenne muscular dystrophy (DMD) is Becker Muscular Dystrophy (BMD), an intermediate clinical presentation between DMD and BMD, or DMD-associated dilated cardiomyopathy.

123. The method of any one of claims 87-122, wherein the method treats, prevents, or ameliorates the fibrotic disease in the subject.

124. The method of any one of claims 87-123, wherein the method treats, prevents, or ameliorates one or more symptoms of said fibrotic disease in the subject.

125. The method of any one of claims 1-124, wherein the method treats, prevents, or inhibits fibrosis in the subject.

126. The method of claim 125, wherein the method inhibits fibrosis in the liver, lung, skin, soft tissue, tendons, lymph nodes, lung, kidney, heart, eye, or retroperitoneum of said subject.

127. A compound of Formula IA: 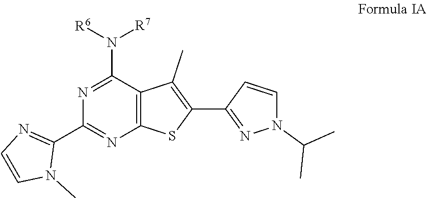
or a pharmaceutically acceptable salt thereof,
wherein:
R⁶ and R⁷ are independently selected from the group consisting of hydrogen, alkyl, alkenyl, alkynyl, aryl, heteroaryl, cycloalkyl, heterocyclyl, alkylcarbonyl, cycloalkylcarbonyl, arylcarbonyl, heteroarylcarbonyl, arylsulfonyl, heteroarylsulfonyl, cycloalkylsulfonyl and alkylsulfonyl, or R⁶ and R⁷ are combined to form a cyclic structure including the nitrogen atom to which they are both attached.

128. The compound of claim 127, wherein:
R⁶ and R⁷ are independently selected from the group consisting of hydrogen, alkyl, aryl, heteroaryl, cycloalkyl, and heterocyclyl, or R⁶ and R⁷ are combined to form a cyclic structure including the nitrogen atom to which they are both attached.

129. The compound of claim 127 or claim 128, wherein the NR⁶R⁷ group is:

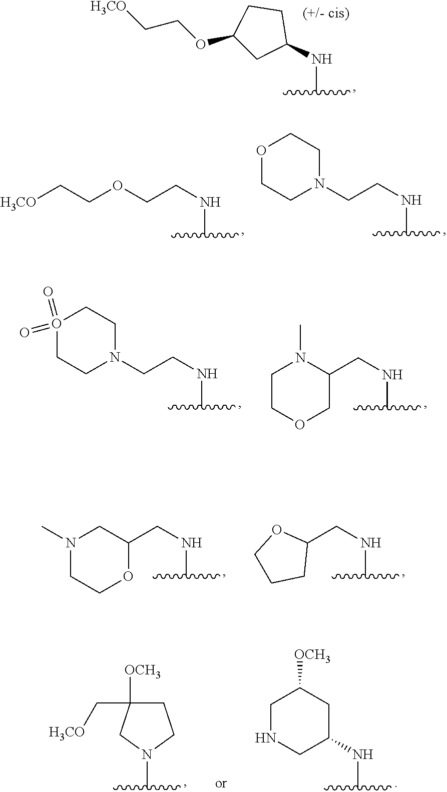

130. A compound selected from the group consisting of: 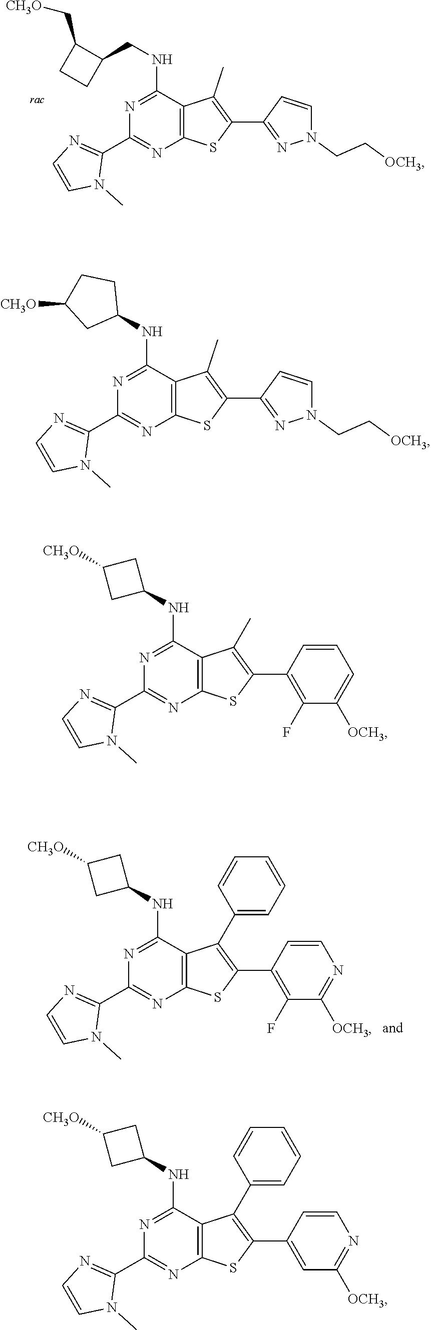
or an enantiomer or a pharmaceutically acceptable salt thereof.

131. A compound of Formula II: 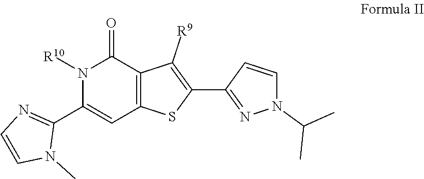
or a pharmaceutically acceptable salt thereof,
wherein:
R⁹ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
R¹⁰ is H, alkyl, alkenyl, alkynyl, aryl, cycloalkyl, heterocyclyl, heteroaryl, C(O)R⁴, or S(O)ₚR⁴;
R³ is hydrogen, alkyl, alkenyl, alkynyl, aryl, heteroaryl, heterocyclyl, cycloalkyl, alkylcarbonyl, cycloalkylcarbonyl or arylcarbonyl;
R⁴ is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy, aralkoxy, or NR⁶R⁷;
each R⁵ is selected from the group consisting of hydrogen, alkyl, alkenyl, alkynyl, aryl, heteroaryl, heterocyclyl, cycloalkyl, alkylcarbonyl, cycloalkylcarbonyl, and arylcarbonyl; and
R⁶ and R⁷ are independently selected from the group consisting of hydrogen, alkyl, alkenyl, alkynyl, aryl, heteroaryl, cycloalkyl, heterocyclyl, alkylcarbonyl, cycloalkylcarbonyl, arylcarbonyl, heteroarylcarbonyl, arylsulfonyl, heteroarylsulfonyl, cycloalkylsulfonyl and alkylsulfonyl, or R⁶ and R⁷ are combined to form a cyclic structure including the nitrogen atom to which they are both attached.

132. The compound of claim 131, wherein:
R⁹ is aryl or alkyl; and
R¹⁰ is alkyl.

133. The compound of claim 131 or claim 132, wherein R⁹ is methyl or phenyl.

134. A compound of Formula III: 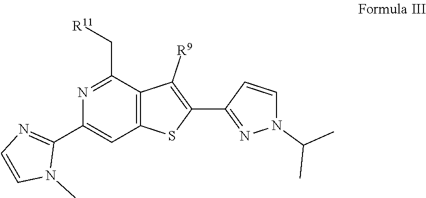
or a pharmaceutically acceptable salt thereof,
wherein:
R⁹ is H, alkyl, alkenyl, alkynyl, aryl, cycloalkyl, heterocyclyl, heteroaryl, halo, pseudohalo, OR³, C(O)R⁴, S(O)ₚR⁴, NR⁵C(O)R⁴, or NR⁶R⁷;
R¹⁰ is H, alkyl, alkenyl, alkynyl, aryl, cycloalkyl, heterocyclyl, heteroaryl, C(O)R⁴, or S(O)ₚR⁴;
R³ is hydrogen, alkyl, alkenyl, alkynyl, aryl, heteroaryl, heterocyclyl, cycloalkyl, alkylcarbonyl, cycloalkylcarbonyl or arylcarbonyl;
R⁴ is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy, aralkoxy, or NR⁶R⁷;
each R⁵ is selected from the group consisting of hydrogen, alkyl, alkenyl, alkynyl, aryl, heteroaryl, heterocyclyl, cycloalkyl, alkylcarbonyl, cycloalkylcarbonyl, and arylcarbonyl; and
R⁶ and R⁷ are independently selected from the group consisting of hydrogen, alkyl, alkenyl, alkynyl, aryl, heteroaryl, cycloalkyl, heterocyclyl, alkylcarbonyl, cycloalkylcarbonyl, arylcarbonyl, heteroarylcarbonyl, arylsulfonyl, heteroarylsulfonyl, cycloalkylsulfonyl and alkylsulfonyl, or R⁶ and R⁷ are combined to form a cyclic structure including the nitrogen atom to which they are both attached.

135. The compound of claim 134, wherein:
R⁹ is aryl or alkyl; and
R¹¹ is alkyl.

136. The compound of claim 134 or claim 135, wherein R⁹ is methyl or phenyl.

137. A compound which binds to the GTP binding domain of one or more members of the Ras superfamily and inhibits the one or more members of the Ras superfamily with an IC50 value of less than 10 micromolar, wherein the compound is the compound or pharmaceutically acceptable derivative of any one of claims 127-136.

138. The compound of claim 137, wherein one or more members of the Ras superfamily is Ras.

139. The compound of claim 137, wherein one or more members of the Ras superfamily is Rho.

140. The compound of claim 137, wherein one or more members of the Ras superfamily is Rac.

141. The compound of claim 138, wherein the Ras is DIRAS1; DIRAS2; DIRAS3; ERAS; GEM; HRAS; KRAS; MRAS; NKIRASI; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; or RRAS2.

142. The compound of claim 141, wherein the Ras is HRAS, KRAS, NRAS, or a mutant thereof.

143. The compound of claim 142, wherein the Ras is HRAS or a mutant thereof.

144. The compound of claim 142, wherein the Ras is KRAS or a mutant thereof.

145. The compound of claim 142, wherein the Ras is NRAS or a mutant thereof.

146. The compound of claim 139, wherein the Rho is RHOA; RHOB; RHOBTB1; RHOBTB2; RHOBTB3; RHOC; RHOD; RHOF; RHOG; RHOH; RHOJ; RHOQ; RHOU; RHOV; RND1; RND2; RND3; RAC1; RAC2; RAC3; CDC42, or a mutant thereof.

147. The compound of claim 139, wherein the Rho is Rac.

148. The compound of claim 140 or 147, wherein the Rae is RAC1; RAC2; RAC3; RHOG, or a mutant thereof.

149. The compound or pharmaceutically acceptable derivative of any one of claims 127-136, wherein the pharmaceutically acceptable derivative of the compound is a pharmaceutically acceptable salt of said compound.

150. A method of inhibiting the function of one or more members of the Ras superfamily, comprising administering to a subject a compound which inhibits the one or more members of the Ras superfamily with an IC₅₀ value of less than 10 μM, wherein the compound is the compound or pharmaceutically acceptable derivative of any one of claims 127-136 or the compound is the compound or pharmaceutically acceptable salt of claim 149.

151. The method of claim 150, wherein one or more members of the Ras superfamily is Ras.

152. The method of claim 150, wherein one or more members of the Ras superfamily is Rho.

153. The method of claim 150, wherein one or more members of the Ras superfamily is Rac.

154. The method of claim 150, wherein the Ras is DIRAS1; DIRAS2; DIRAS3; ERAS; GEM; HRAS; KRAS; MRAS; NKIRASI; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; or RRAS2.

155. The method of claim 154, wherein the Ras is HRAS, KRAS, NRAS or a mutant thereof.

156. The method of claim 154, wherein the Ras is HRAS or a mutant thereof.

157. The method of claim 154, wherein the Ras is KRAS or a mutant thereof.

158. The method of claim 154, wherein the Ras is NRAS or a mutant thereof.

159. The method of claim 152, wherein the Rho is RHOA; RHOB; RHOBTB1; RHOBTB2; RHOBTB3; RHOC; RHOD; RHOF; RHOG; RHOH; RHOJ; RHOQ; RHOU; RHOV; RND1; RND2; RND3; RAC1; RAC2; RAC3; CDC42, or a mutant thereof.

160. The method of claim 169, wherein the Rho is Rac.

161. The method of claim 153 or 160, wherein the Rac is RAC1; RAC2; RAC3; RHOG, or a mutant thereof.

162. The method of claim 150, wherein the inhibiting the function of one or more members of the Ras superfamily is a treatment, prevention or amelioration of one or more symptoms of cancer.

163. The method of any of claims 151 or 154-158, wherein the inhibiting the function of Ras is a treatment, prevention or amelioration of one or more symptoms of cancer.

164. The method of any of claims 152 or 159-160, wherein the inhibiting the function of Rho is a treatment, prevention or amelioration of one or more symptoms of cancer.

165. The method of any of claims 153 or 160-161, wherein the inhibiting the function of Rac is a treatment, prevention or amelioration of one or more symptoms of cancer.

166. The method of any of claims 162-165, wherein the cancer is a solid tumor.

167. The method of claim 166, wherein the solid tumor is hepatocellular carcinoma, prostate cancer, pancreatic cancer, lung cancer, breast cancer, ovarian cancer, colon cancer, small intestine cancer, biliary tract cancer, endometrium cancer, skin cancer (melanoma), cervix cancer, urinary tract cancer, or glioblastoma.

168. The method of claim 167, wherein the solid tumor is pancreatic cancer.

169. The method of claim 167, wherein the solid tumor is colon cancer.

170. The method of claim 167, wherein the solid tumor is small intestine cancer.

171. The method of claim 167, wherein the solid tumor is biliary tract cancer.

172. The method of claim 167, wherein the solid tumor is endometrium cancer.

173. The method of claim 167, wherein the solid tumor is lung cancer.

174. The method of claim 167, wherein the solid tumor is breast cancer.

175. The method of claim 167, wherein the solid tumor is skin cancer.

176. The method of claim 167, wherein the solid tumor is cervix cancer.

177. The method of claim 167, wherein the solid tumor is urinary tract cancer.

178. The method of any of claims 162-165, wherein the cancer is a blood borne tumor.

179. The method of claim 178, wherein the blood borne tumor is a leukemia.

180. The method of claim 178, wherein the blood borne tumor is chronic lymphocytic leukemia (CLL), chronic myelocytic leukemia (CML), acute lymphoblastic leukemia (ALL), acute myeloid leukemia (AML), or acute myeloblastic leukemia (AML).

181. The method of any one of claims 178-180, wherein the blood borne tumor is metastatic.

182. The method of claim 150, wherein the inhibiting the function of one or more members of the Ras superfamily is a treatment, prevention or amelioration of one or more symptoms of an inflammatory disease.

183. The method of any of claims 151 or 154-158, wherein inhibiting the function of Ras is a treatment, prevention or amelioration of one or more symptoms of an inflammatory disease.

184. The method of any of claims 152 or 159-160, wherein the inhibiting the function of Rho is a treatment, prevention or amelioration of one or more symptoms of inflammatory disease.

185. The method of any of claims 153 or 160-161, wherein the inhibiting the function of Rac is a treatment, prevention or amelioration of one or more symptoms of inflammatory disease.

186. The method of any of claims 182-185, wherein the inflammatory disease is gastritis, schistosomiasis, cholangitis, chronic cholecystitis, pelvic inflammatory disease, chronic cervicitis, osteomyelitis, inflammatory bowel disease, reflux esophagitis, Barrett's esophagus, bladder inflammation (cystitis), asbestosis, silicosis, gingivitis, lichen planus, pancreatitis, protease mutation, lichen sclerosis, slaladenitis, bronchitis, Sjogren syndrome or Hashimoto's thyroiditis.

187. The method of any of claims 182-185, wherein the inflammatory disease is Alzheimer's disease (AD), ankylosing spondylitis, arthritis (osteoarthritis, rheumatoid arthritis (RA), psoriatic arthritis), asthma, atherosclerosis, Crohn's disease, colitis, dermatitis, diverticulitis, fibromyalgia, hepatitis, irritable bowel syndrome (IBS), systemic lupus, erythematous (SLE), nephritis, Parkinson's disease, ulcerative colitis.

188. The method of claim 187, wherein the inflammatory disease is Alzheimer's disease (AD).

189. The method of claim 187, wherein the inflammatory disease is ankylosing spondylitis.

190. The method of claim 187, wherein the inflammatory disease is arthritis (osteoarthritis, rheumatoid arthritis (RA), psoriatic arthritis).

191. The method of claim 187, wherein the inflammatory disease is asthma.

192. The method of claim 187, wherein the inflammatory disease is atherosclerosis.

193. The method of claim 187, wherein the inflammatory disease is Crohn's disease.

194. The method of claim 187, wherein the inflammatory disease is colitis.

195. The method of claim 187, wherein the inflammatory disease is dermatitis.

196. The method of claim 187, wherein the inflammatory disease is diverticulitis.

197. The method of claim 187, wherein the inflammatory disease is fibromyalgia.

198. The method of claim 187, wherein the inflammatory disease is hepatitis.

199. The method of claim 187, wherein the inflammatory disease is irritable bowel syndrome (IBS).

200. The method of claim 187, wherein the inflammatory disease is systemic lupus.

201. The method of claim 187, wherein the inflammatory disease is erythematous (SLE).

202. The method of claim 187, wherein the inflammatory disease is nephritis.

203. The method of claim 187, wherein the inflammatory disease is Parkinson's disease.

204. The method of claim 187, wherein the inflammatory disease is ulcerative colitis.

205. The method of claim 150, wherein the inhibiting the function of one or more members of the Ras superfamily is a treatment, prevention or amelioration of one or more symptoms of a rasopathy.

206. The method of any of claims 151 or 154-158, wherein the inhibiting the function of Ras is a treatment for a rasopathy.

207. The method of any of claims 152 or 159-160, wherein the inhibiting the function of Rho is a treatment for a rasopathy.

208. The method of any of claims 153 or 160-161, wherein the inhibiting the function of Rac is a treatment for a rasopathy.

209. The method of any of claims 205-208, wherein the rasopathy is neurofibromatosis type 1, Noonan's syndrome or Costello syndrome.

210. The method of any of claims 151 or 154-158, wherein the inhibiting the function of Ras is a treatment for Ras-associated autoimmune leukoproliferative disorder.

211. The method of claim 150, wherein the inhibiting the function of one or more members of the Ras superfamily is a treatment, prevention or amelioration of one or more symptoms of a fibrotic disease.

212. The method of any of claims 151 or 154-158, wherein the inhibiting the function of Ras is a treatment, prevention or amelioration of one or more symptoms of a fibrotic disease.

213. The method of any of claims 152 or 159-160, wherein the inhibiting the function of Rho is a treatment, prevention or amelioration of one or more symptoms of a fibrotic disease.

214. The method of any of claims 152 or 159-160, wherein the inhibiting the function of Rac is a treatment, prevention or amelioration of one or more symptoms of a fibrotic disease.

215. The method of any one of claims 162, 182, 205, or 211, wherein one or more members of the Ras superfamily is Ras.

216. The method of any one of claims 162, 182, 205, or 211, wherein one or more members of the Ras superfamily is Rho.

217. The method of any one of claims 162, 182, 205, or 211, wherein one or more members of the Ras superfamily is Rac.

218. A pharmaceutical composition, comprising the compound or pharmaceutically acceptable derivative of any one of claims 127-136, and a pharmaceutically acceptable carrier.

219. The pharmaceutical composition of claim 218, wherein the pharmaceutical composition comprises a therapeutic amount of said compound or pharmaceutically acceptable derivative thereof.

220. A pharmaceutical composition, comprising the compound or pharmaceutically acceptable salt of claim 149, and a pharmaceutically acceptable carrier.

221. The pharmaceutical composition of claim 220, wherein the pharmaceutical composition comprises a therapeutic amount of said compound or pharmaceutically acceptable salt thereof.

222. A method of inhibiting the function of one or more members of the Ras superfamily, comprising administering to a subject the pharmaceutical composition of any one of claims 218-221.

223. A method of inhibiting the function of one or more members of the Ras superfamily, comprising administering to a subject the compound or pharmaceutically acceptable derivative of any one of claims 127-136.
